(19) United States
(12) Patent Application Publication
(10) Pub. No.: US 2022/0347306 A1
(43) Pub. Date: Nov. 3, 2022

(54) SELECTIVE DELIVERY OF THERAPEUTIC AND IMAGING AGENTS

(71) Applicant: Avelas Biosciences, Inc., La Jolla, CA (US)

(72) Inventors: Jesus E. GONZALEZ, Carlsbad, CA (US); Junjie LIU, San Diego, CA (US); Marcel MIAMPAMBA, San Diego, CA (US); Ning ZOU, La Jolla, CA (US); Andrew GALE, San Diego, CA (US)

(21) Appl. No.: 17/048,080

(22) PCT Filed: Apr. 16, 2019
(86) PCT No.: PCT/US2019/027764

§ 371 Date: Oct. 15, 2020

Related U.S. Application Data
(60) Provisional application No. 62/658,433, filed on Apr. 16, 2018.

(51) Int. Cl. A61K 47/64; A61K 45/06; A61K 47/65; C07K 7/06; C07K 7/08

(52) U.S. Cl. CPC A61K 47/645; A61K 45/06; C07K 7/08; C07K 7/06; A61K 47/65

(57) ABSTRACT

Described herein are methods and compositions for the targeted delivery of selective delivery molecule therapeutic agents and imaging agents.

### CROSS-REFERENCE

This application claims the benefit of U.S. Provisional Application No. 62/658,433, filed on Apr. 16, 2018, which is incorporated herein by reference in its entirety.

### BACKGROUND

Described herein are methods and compositions for the targeted delivery of therapeutic agents and imaging agents.

### SUMMARY

Described herein are compositions and methods for the selective delivery of imaging agents and therapeutic agents.

Disclosed herein, in certain embodiments, is a selective delivery molecule of Formula I, having the structure:

[(S)_{w}-A-c_{A}-(cₚ-M)ᵤ-X—B-c_{B}-D_{B}]-(N—Z)ᵥ Formula I

wherein,
S is an electrophilic substituent bound to the amino terminus of A or c_{A};
A is a peptide with a sequence comprising 5 to 9 acidic amino acids;
c_{A} is a bond or a single amino acid linker;
cₚ is a linker;
M is a macromolecule;
X is a cleavable linker;
B is a peptide with a sequence comprising 5 to 20 basic amino acids;
c_{B} is a single amino acid linker;
D_{B} is a therapeutic agent or an imaging agent;
each N is independently selected from a bond and a linker;
each Z is independently a peptide with a sequence comprising 3 to 10 amino acids;
v is 1 or 2; and
w and u are independently 0 or 1;
   wherein each N is independently bound to c_{A}, c_{B}, or the amino terminus of A, S is bound to the amino terminus of A, and cₚ is bound to c_{A}; and
   wherein if S is present, S and N are not bound at the same position; and if cₚ is present, cₚ and N are not bound at the same position.

In some embodiments, X is cleavable by an extracellular protease. In some embodiments, Z comprises a receptor binding peptide. In some embodiments, Z comprises a urokinase type plasminogen activator receptor (uPAR) peptide. In some embodiments, Z comprises SRSRY. SRNRY, SRGRY, SQSRY, SQNRY, SQGRY, PRSRY, PRNRY, PRGRY, PQSRY, PQNRY, or PQGRY In some embodiments, Z comprises a series of 4 Phe residues. In some embodiments, Z is a peptide with a sequence selected from the group consisting of:

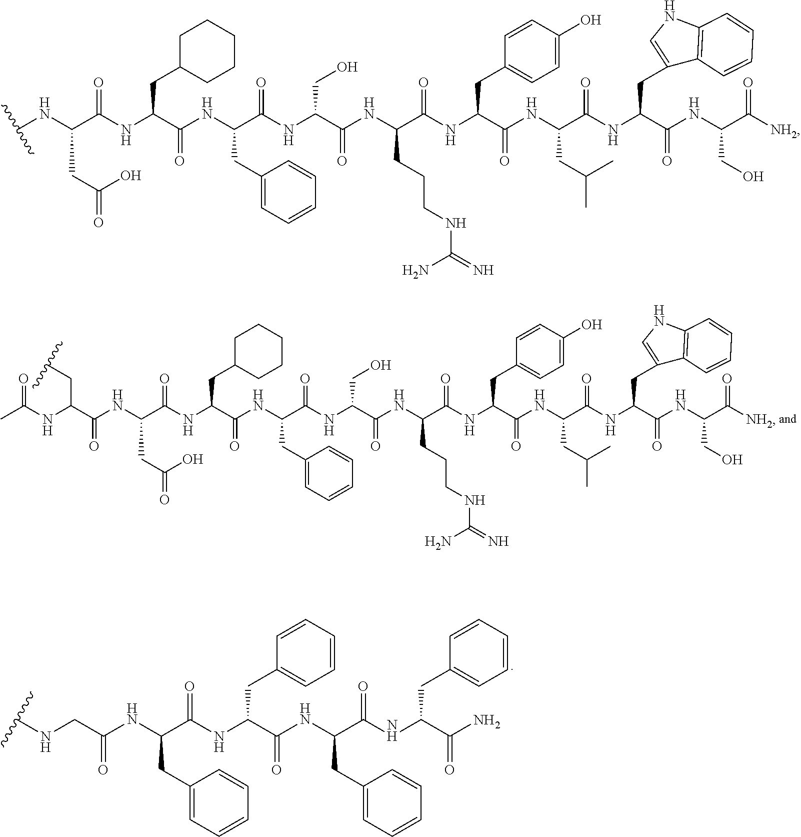

In some embodiments, if v is 2, the two N are not identical. In some embodiments, N is a bond or a linker selected from the group consisting of:

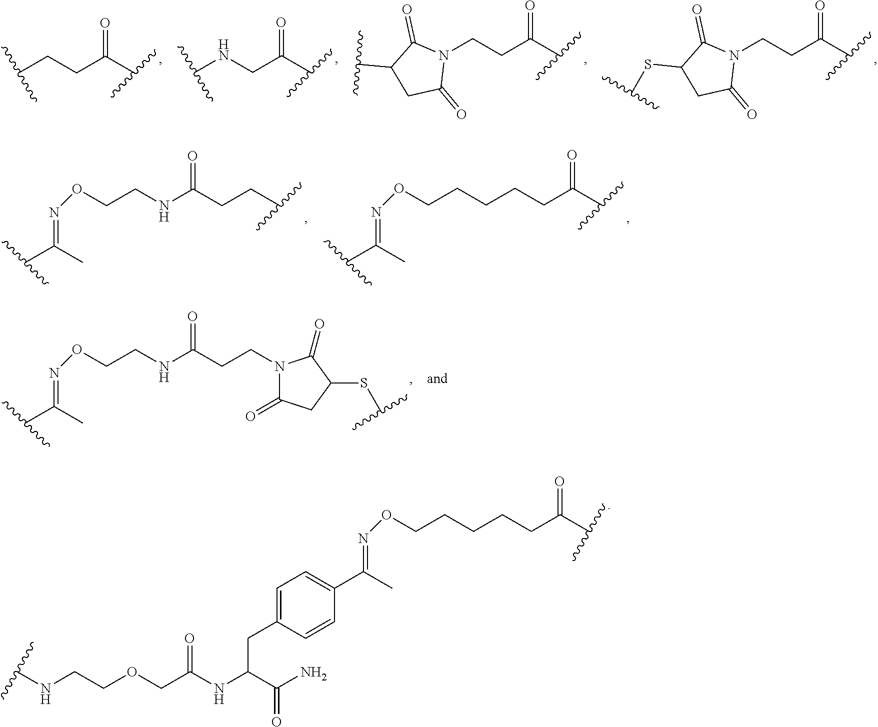

In some embodiments, c_{A} is a bond,

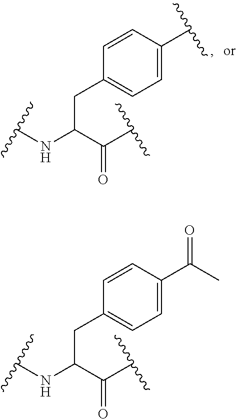 [SMILES: *NC(Cc1ccc(*)cc1)C(*)=O ; *NC(Cc1ccc(C(C)=O)cc1)C(*)=O]

In some embodiments, cₚ is selected from:

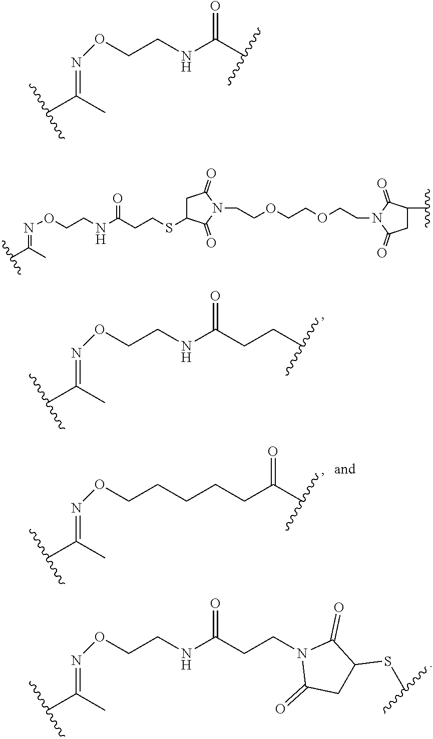 [SMILES: *C(=O)NCCO/N=C(/*)C ; */C(C)=N/OCCNC(=O)CCSC1CC(=O)N(CCOCCOCCN2C(=O)CC(*)C2=O)C1=O ; *CCC(=O)NCCO/N=C(/*)C ; *C(=O)CCCCCO/N=C(/*)C ; *SC1CC(=O)N(CCC(=O)NCCO/N=C(/*)C)C1=O]

In some embodiments, M is a polyethylene glycol substituent. In some embodiments, X comprises PLGLAG, PLG-C(me)-AG, RPLALWRS, ESPAYYTA, DPRSFL, PPRSFL, RLQLKL, or RLQLK(Ac)L. In some embodiments, X comprises —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, or —NHCH₂CH₂OCH₂CH₂OCH₂C(O)NHCH₂CH₂OCH₂CH₂OCH₂C(O)—, In some embodiments, B is a peptide with a sequence comprising 7 to 9 basic amino acids. In some embodiments, S is an N-maleimide or N-succinamide substituent. In some embodiments, S is

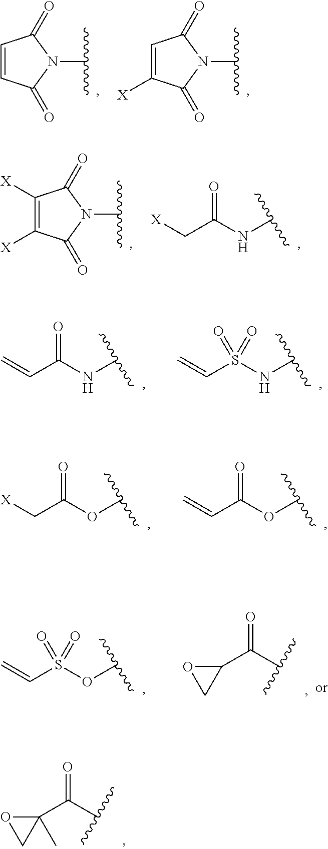

wherein each X is independently —Cl, —Br, —I, or —S-phenyl. In some embodiments, S is

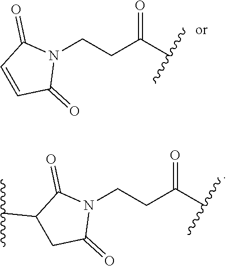 [SMILES: *C(=O)CCN1C(=O)C=CC1=O ; *C(=O)CCN1C(=O)CC(*)C1=O]

In some embodiments, S is bound to the amino terminus of A. In some embodiments, S is further conjugated to an albumin. In some embodiments, c_{B} is

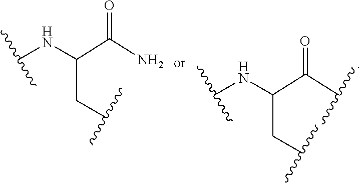 [SMILES: *CC(N*)C(N)=O ; *CC(N*)C(*)=O]

In some embodiments, D_{B} is an auristatin-related therapeutic agent or a cyanine-related imaging agent. In some embodiments, c_{A} is a bond,

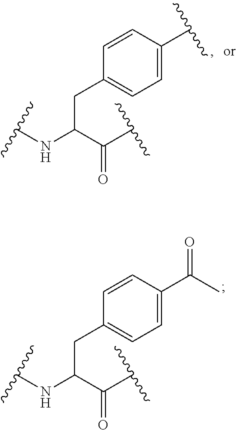 [SMILES: *NC(Cc1ccc(*)cc1)C(*)=O ; *NC(Cc1ccc(C(C)=O)cc1)C(*)=O]

cₚ is selected from:

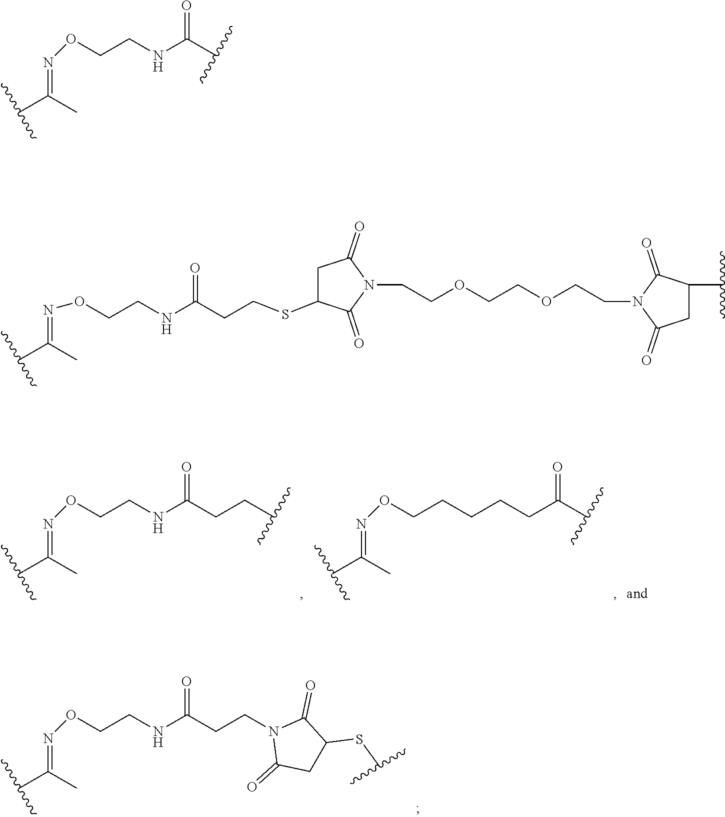 [SMILES: *C(=O)NCCO/N=C(/*)C ; */C(C)=N/OCCNC(=O)CCSC1CC(=O)N(CCOCCOCCN2C(=O)CC(*)C2=O)C1=O ; *CCC(=O)NCCO/N=C(/*)C ; *C(=O)CCCCCO/N=C(/*)C ; *SC1CC(=O)N(CCC(=O)NCCO/N=C(/*)C)C1=O]

M is a polyethylene glycol substituent; X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—; B is a peptide with a sequence comprising 7 to 9 basic amino acids; c_{B} is

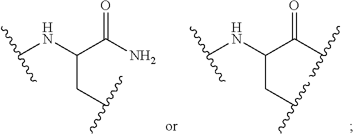 [SMILES: *CC(N*)C(N)=O ; *CC(N*)C(*)=O]

D_{B} is an auristatin-related therapeutic agent or a cyanine-related imaging agent; N is a bond or a linker selected from the group consisting of:

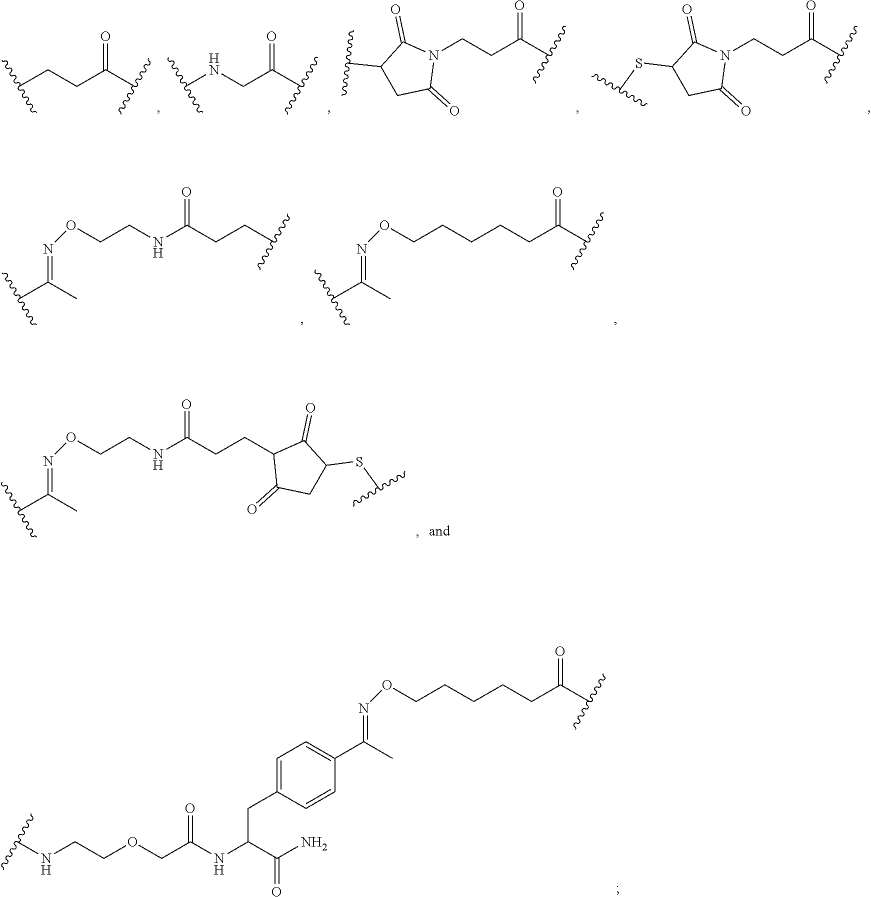

Z is a peptide with a sequence selected from the group consisting of:

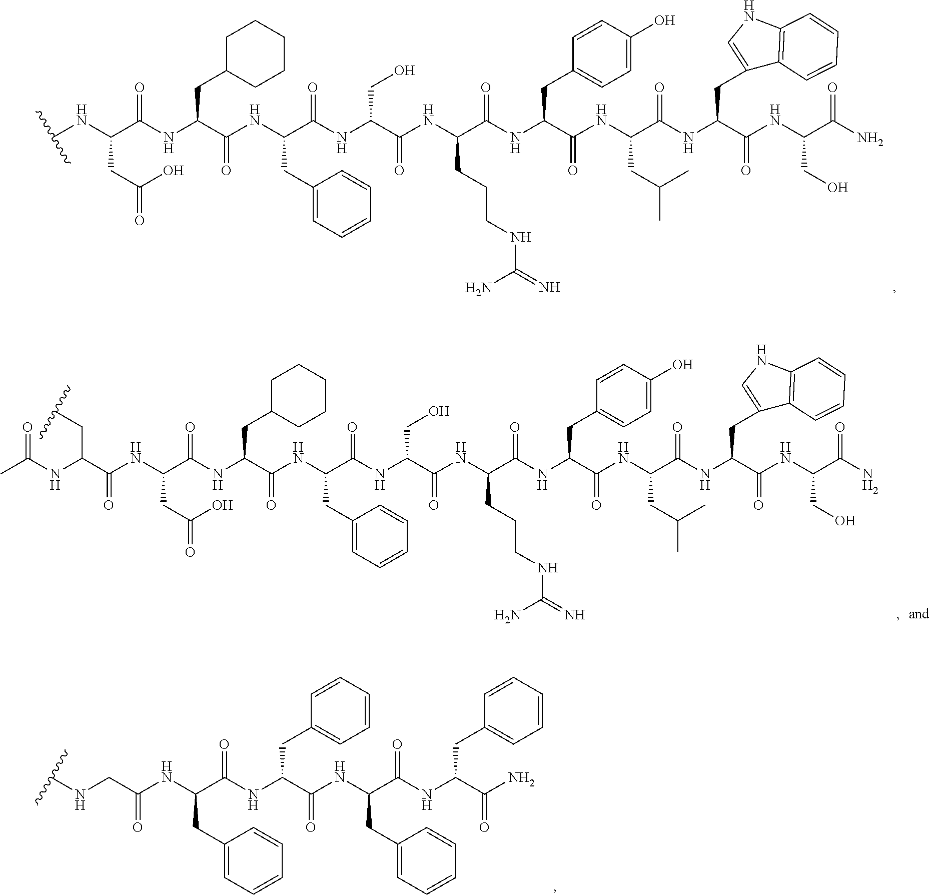

In some embodiments, D_{B} is G-T-Q-Y-D; G is selected from the following substituents:

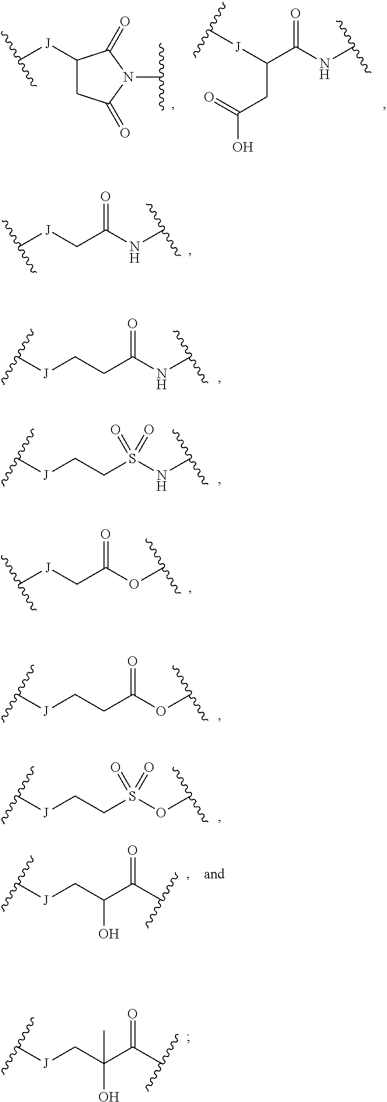

J is —O—, —NH—, or —S—; T is an optionally substituted C₁-C₈ alkylene, optionally substituted C₁-C₈ alkylene-C(O)—, optionally substituted C₃-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—(CH₂)ₘC(O)—; R^{1B} is —H, —CH₃, —CH₂CH₃, or —CH₂CH₂NH₂; each n is independently an integer ranging from 1 to 25; each m is independently an integer ranging from 1 to 10; Q is a bond or 1-3 amino acids selected from the group consisting of:

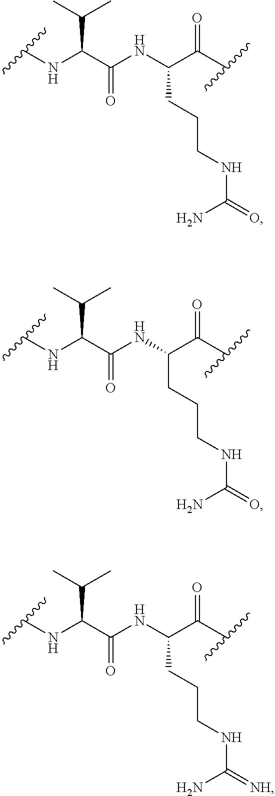 [SMILES: *N[C@H](C(=O)N[C@@H](CCCNC(N)=O)C(*)=O)C(C)C ; *N[C@H](C(=O)N[C@@H](CCCNC(N)=O)C(*)=O)C(C)C ; *N[C@H](C(=O)N[C@@H](CCCNC(=N)N)C(*)=O)C(C)C]

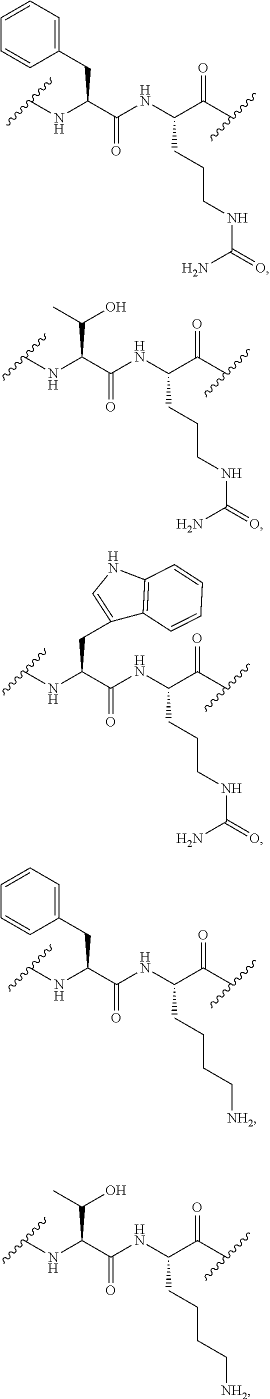 [SMILES: *N[C@@H](Cc1ccccc1)C(=O)N[C@@H](CCCNC(N)=O)C(*)=O ; *N[C@H](C(=O)N[C@@H](CCCNC(N)=O)C(*)=O)C(C)O ; *N[C@@H](Cc1c[nH]c2ccccc12)C(=O)N[C@@H](CCCNC(N)=O)C(*)=O ; *N[C@@H](Cc1ccccc1)C(=O)N[C@@H](CCCCN)C(*)=O ; *N[C@H](C(=O)N[C@@H](CCCCN)C(*)=O)C(C)O]

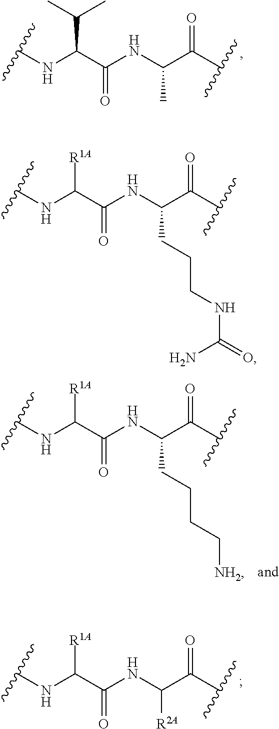

R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl; R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂. —CH₂CH₂CH₂NHC(═NH)NH₂, or —CH₂CH₂CH₂NHC(O)NH₂; Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—; R²⁸ is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂; R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂; D is U or Z²;
U is a fragment having the structure of Formula (IA) or Formula (IB):

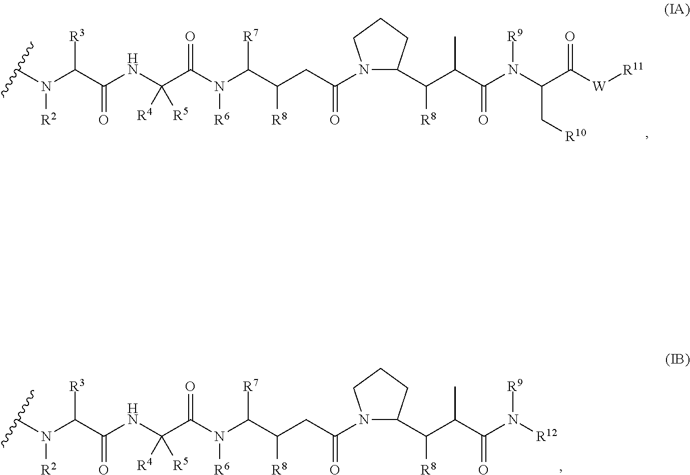

wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is an integer ranging between 0 to 6;
t is an integer ranging between 0 to 6;
Z² is a fragment having the structure of Formula (IC):

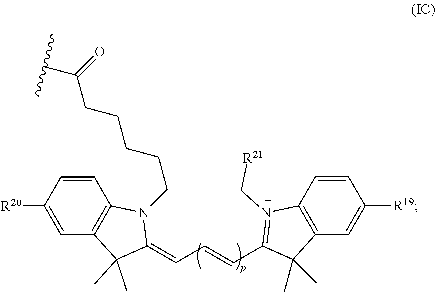

wherein,
each R¹⁹ and R²⁰ are independently —H, —SO₃⁻, —SO₃H or C₁-C₈ alkyl, wherein at least one of R¹⁹ and R²⁰ is —SO₃⁻;
R²¹ is —H or C₁-C₈ alkyl; and
p is an integer ranging from 0 to 3.

In some embodiments, G is selected from the following substituents:

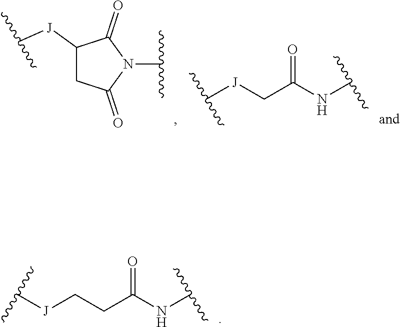

In some embodiments, J is —O— or —S—. In some embodiments, T is an optionally substituted C₁-C₈ alkylene-C(O)—. In some embodiments, Q is selected from the following substituents:

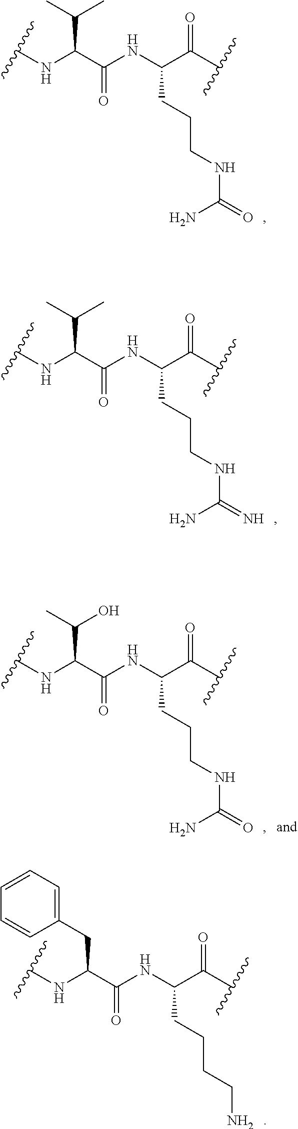 [SMILES: *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@H](C(=O)N[C@@H](CCCNC(N)=O)C(*)=O)C(C)O ; *N[C@@H](Cc1ccccc1)C(=O)N[C@@H](CCCCN)C(*)=O]

In some embodiments, Y is a bond, —NHCH₂C(O)—, —NHCH(CH₃)C(O)—, or —NHCH₂CH₂—. In some embodiments, R² is —H or optionally substituted C₁-C₈ alkyl; R³ is —H, or optionally substituted C₁-C₈ alkyl; R⁴ is —H, or optionally substituted C₁-C₈ alkyl; R⁵ is —H or —CH₃; or R⁴ and R⁵ jointly form an optionally substituted C₃-C₈ carbocyclyl; R⁶ is —H or optionally substituted C₁-C₈ alkyl; R⁷ is —H, optionally substituted C₁-C₈ alkyl, or optionally substituted C₃-C₈ carbocyclyl; each R⁸ is independently selected from —H, —OH, optionally substituted C₁-C₈ alkyl, optionally substituted C₃-C₈ carbocyclyl, and —O-(optionally substituted C₁-C₈ alkyl)-; R⁹ is —H; R¹⁰ is optionally substituted C₆-C₁₀ aryl; W is —O—; R¹¹ is —H; and R¹² is —C(R⁸)₂—C(R⁸)₂—(C₆-C₁₀ aryl). In some embodiments, G is selected from the following substituents:

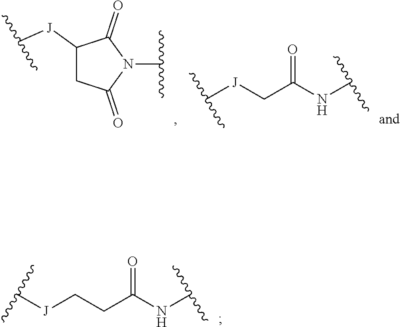

J is —O— or —S—; T is an optionally substituted C₁-C₈ alkylene-C(O)—; Q is selected from the following substituents:

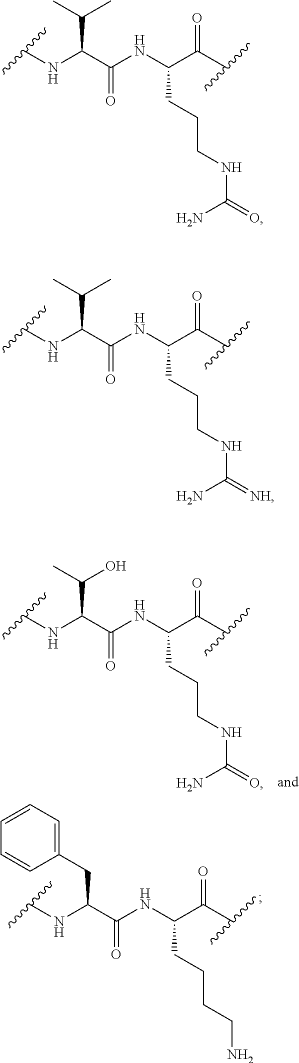 [SMILES: *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@H](C(=O)N[C@@H](CCCNC(N)=O)C(*)=O)C(C)O ; *N[C@@H](Cc1ccccc1)C(=O)N[C@@H](CCCCN)C(*)=O]

Y is a bond, —NHCH₂C(O)—, —NHCH(CH₃)C(O)—, or —NHCH₂CH₂—; R² is —H or optionally substituted C₁-C₈ alkyl; R³ is —H, or optionally substituted C₁-C₈ alkyl; R⁴ is —H, or optionally substituted C₁-C₈ alkyl; R⁵ is —H or —CH₃; or R⁴ and R⁵ jointly form an optionally substituted C₃-C₈ carbocyclyl; R⁶ is —H or optionally substituted C₁-C₈ alkyl; R⁷ is —H, optionally substituted C₁-C₈ alkyl, or optionally substituted C₃-C₈ carbocyclyl; each R⁸ is independently selected from —H, —OH, optionally substituted C₁-C₈ alkyl, optionally substituted C₃-C₈ carbocyclyl, and —O-(optionally substituted C₁-C₈ alkyl)-; R⁹ is —H; R¹⁰ is optionally substituted C₆-C₁₀ aryl; W is —O—; R¹¹ is —H; and R¹² is —C(R⁸)₂—C(R⁸)₂—(C₆-C₁₀ aryl). In some embodiments, U is monomethyl auristatin F (MMAF). In some embodiments, U is monomethyl auristatin E (MMAE). In some embodiments, Z² is cyanine-5, or a derivative thereof. In some embodiments, the selective delivery molecule is: SDM-177, SDM-179, SDM-180, SDM-183, SDM-184, SDM-186, SDM-209, SDM-258, SDM-259, or SDM-260. In some embodiments, A and B do not have an equal number of acidic and basic amino acids. In some embodiments, the number of basic amino acids in B is greater than the number of acidic amino acids in A. In some embodiments, A and B are independently selected from natural amino acids, unnatural amino acids, or a combination thereof. In some embodiments, A is a peptide comprising 5 or 9 consecutive glutamates. In some embodiments, A is a peptide comprising 5 consecutive glutamates. In some embodiments, A is a peptide comprising 9 consecutive glutamates. In some embodiments, B is a peptide comprising 8 or 9 consecutive arginines. In some embodiments, B is a peptide comprising 8 consecutive arginines. In some embodiments, B is a peptide comprising 9 consecutive arginines. In some embodiments, X is cleavable by a matrix metalloproteinase. In some embodiments, X comprises an amino acid sequence that is cleavable by MMP2, MMP7, MMP9, or MMP14.

Disclosed herein, in certain embodiments, is a selective delivery molecule of Formula II, having the structure:

M²ₒ-S_{w}-A-c_{A}-(c_{P}-M¹)ᵤ-X—B-c_{B}-G-T-Q-Y-D Formula II

wherein,
M¹ and M² are each independently a macromolecule;
S is an N-maleimide substituent or N-succinamide linker bound to the amino terminus of A;
A is a peptide with a sequence comprising 5 to 9 acidic amino acids;
c_{A} is a bond or a single amino acid;
cₚ is a linker bound to c_{A};
X is a cleavable linker;
B is a peptide with a sequence comprising 5 to 20 basic amino acids;
c_{B} is a single amino acid;
G is a linker;
T is a spacer;
Q is a bond or 1-3 amino acids;
Y is 1-3 amino acids or an amino alkylene;
D is an auristatin related therapeutic agent; and
w, u, and o are independently 0 or 1.

In some embodiments, M² is a polyethylene glycol substituent or an albumin substituent. In some embodiments, if M² is absent, S comprises

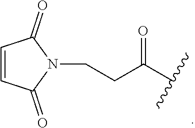 [SMILES: *C(=O)CCN1C(=O)C=CC1=O]

In some embodiments, if M² is present, M²-S is selected from:

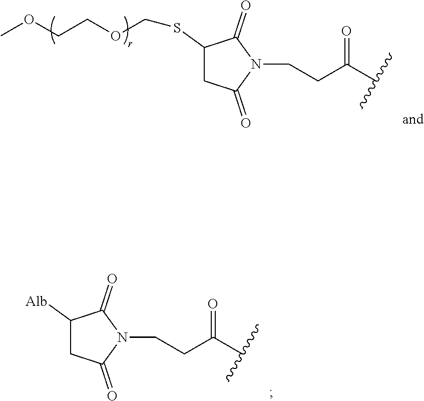

wherein Alb is an albumin protein and r is independently an integer ranging from 40-1,100. In some embodiments, cₚ is selected from:

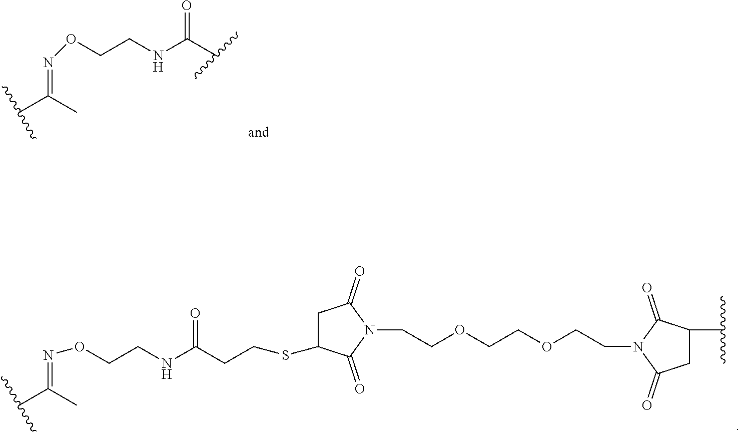 [SMILES: *C(=O)NCCO/N=C(/*)C ; */C(C)=N/OCCNC(=O)CCSC1CC(=O)N(CCOCCOCCN2C(=O)CC(*)C2=O)C1=O]

In some embodiments, cₚ-M¹ is selected from:

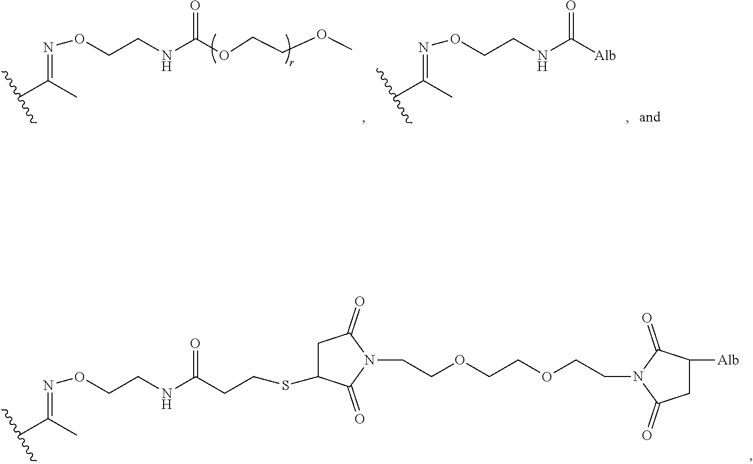

wherein Alb is an albumin protein and each r is independently an integer ranging from 40-1,100. In some embodiments, X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—. In some embodiments, c_{B} is

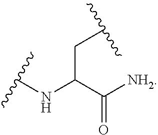 [SMILES: *CC(N*)C(N)=O]

In some embodiments, B is a peptide with a sequence comprising 7 to 9 basic amino acids. In some embodiments, G is selected from the following substituents:

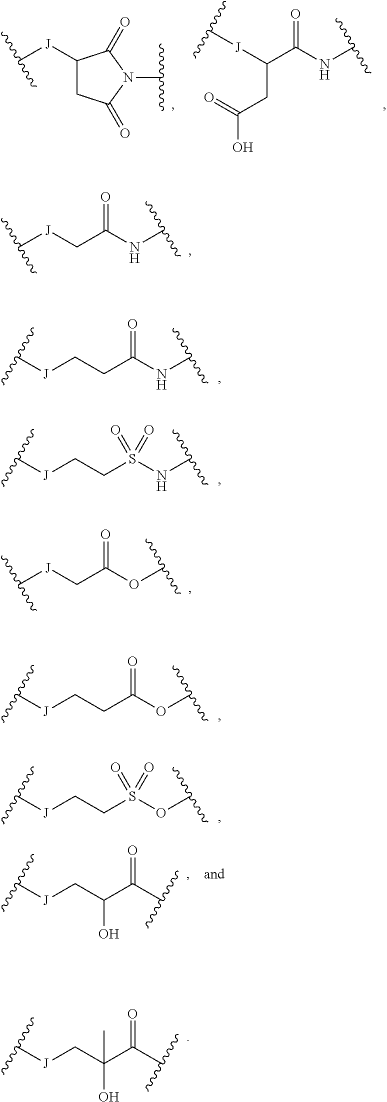

In some embodiments, J is —O—, —NH—, or —S—. In some embodiments, T is an optionally substituted C₁-C₈ alkylene, optionally substituted C₁-C₈ alkylene-C(O)—, optionally substituted C₅-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)₁—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—(CH₂)ₘC(O)— wherein R^{1B} is —H, —CH₃, —CH₂CH₃, or —CH₂CH₂NH₂. In some embodiments, Q is a bond or selected from the group consisting of:

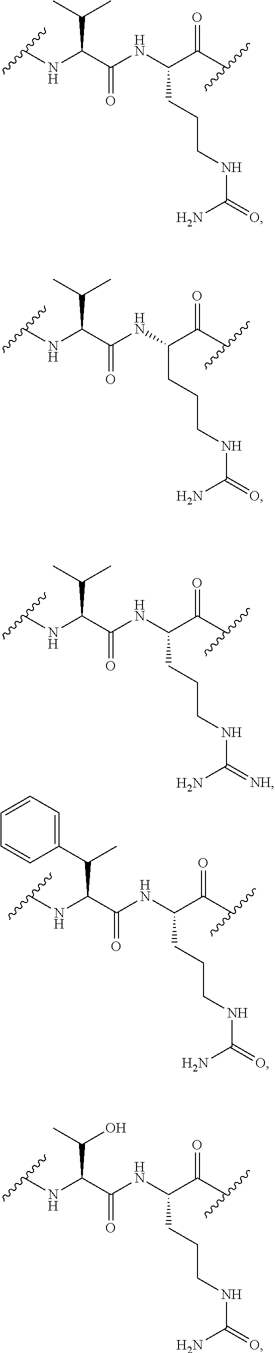

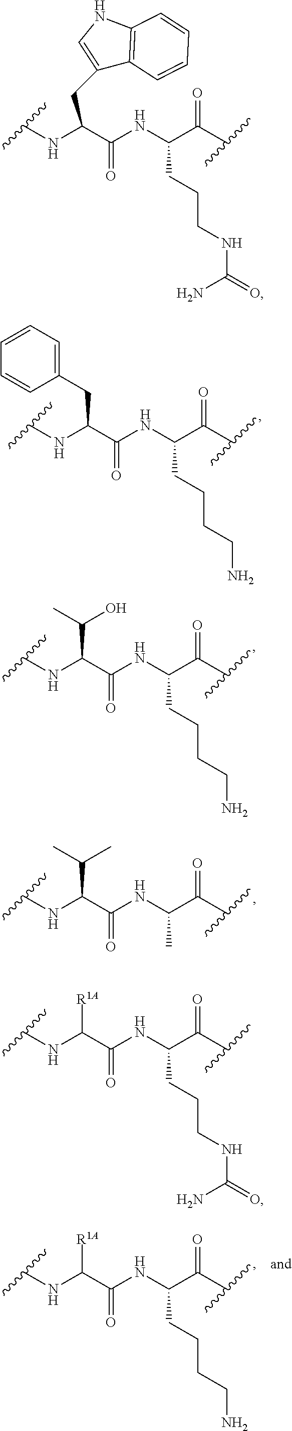

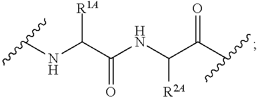

wherein, R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl; and R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂, —CH₂CH₂CH₂NHC(═NH)NH₂, or —CH₂CH₂CH₂NHC(O)NH₂. In some embodiments, Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—; wherein, R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂; R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH. In some embodiments, D is U. In some embodiments, U is a fragment having the structure of Formula (IIA) or Formula (IIB):

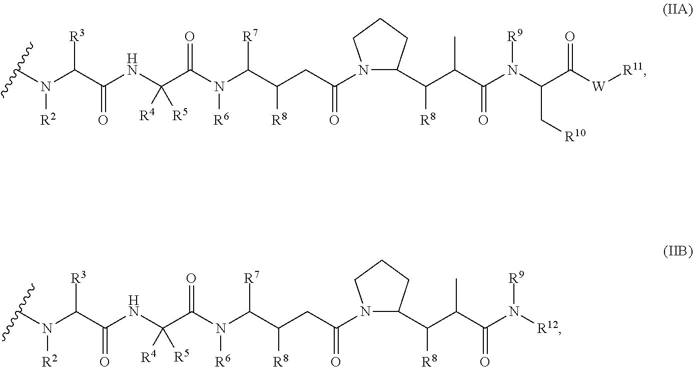

wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₁-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is and integer ranging between 0 to 6; and
t is and integer ranging between 0 to 6.

In some embodiments, M²-S is selected from the group consisting of:

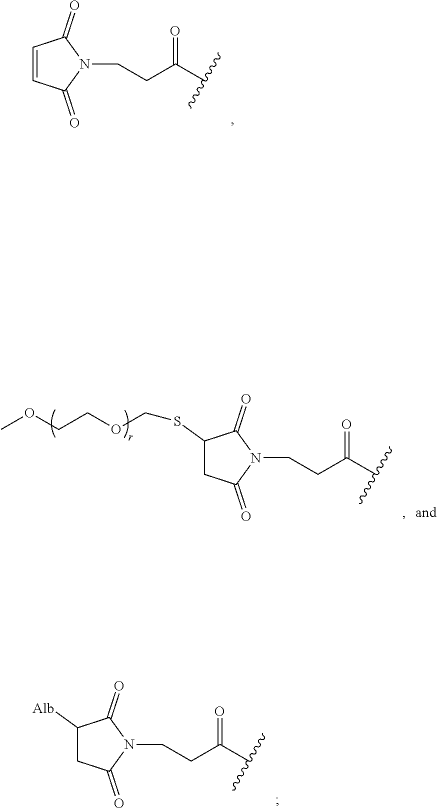

c_{A} is a bond,

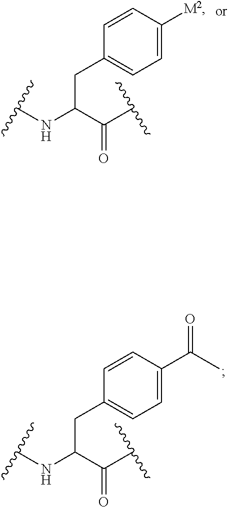

wherein,
cₚ-M¹ is selected from the group consisting of:

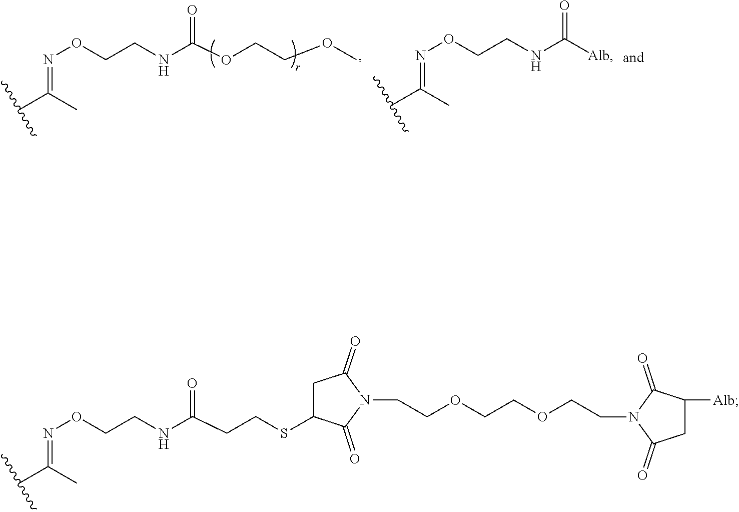

Alb is an albumin protein;
each r is independently an integer ranging from 40-1,100;
X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—;
c_{B} is

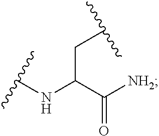 [SMILES: *CC(N*)C(N)=O]

B is a peptide with a sequence comprising 7 to 9 basic amino acids;
G is selected from the following substituents:

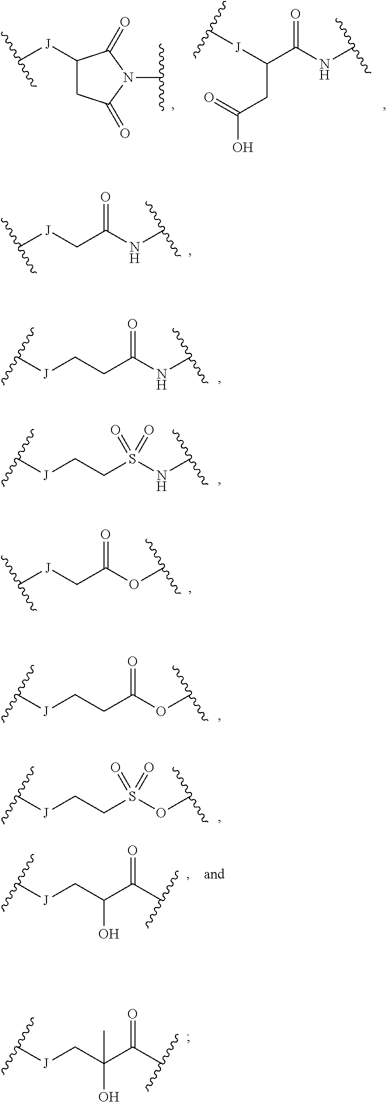

J is —O—, —NH—, or —S—;
T is an optionally substituted C₁-C₈ alkylene, optionally substituted C₁-C₈ alkylene-C(O)—, optionally substituted C₃-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—(CH₂)ₘC(O)—;
R^{1B} is —H, —CH₃, —CH₂CH₃, or —CH₂CH₂NH₂;
each n is independently an integer ranging from 1 to 25;
each m is independently an integer ranging from 1 to 10;
Q is a bond or selected from the group consisting of:

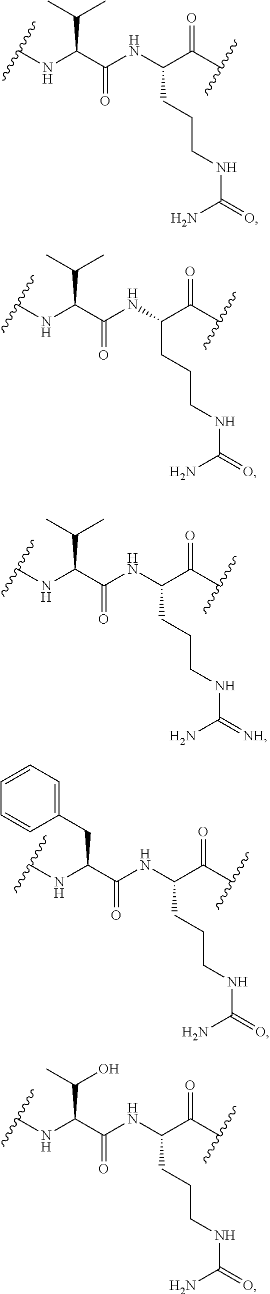 [SMILES: *N[C@H](C(=O)N[C@@H](CCCNC(N)=O)C(*)=O)C(C)C ; *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@@H](Cc1ccccc1)C(=O)N[C@@H](CCCNC(N)=O)C(*)=O ; *N[C@H](C(=O)N[C@@H](CCCNC(N)=O)C(*)=O)C(C)O]

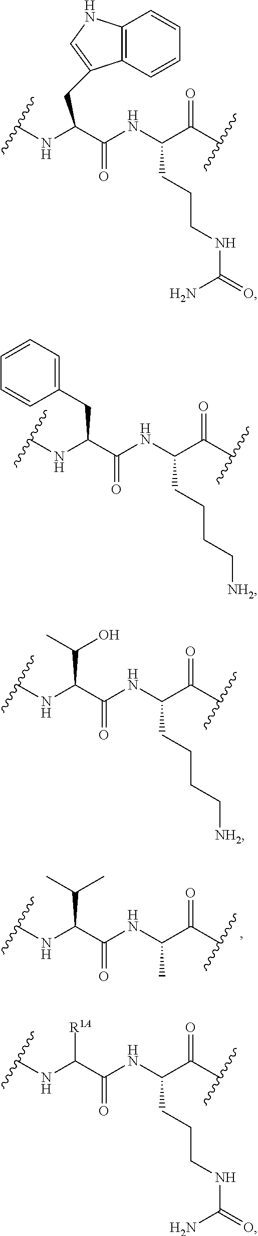

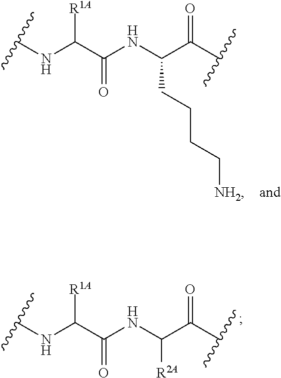

R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂—CH₂CH₂CH₂NHC(═NH)NH₂, or —CH₂CH₂CH₂NHC(O)NH₂;
Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—;
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
D is U;
U is a fragment having the structure of Formula (IIA) or Formula (IIB):

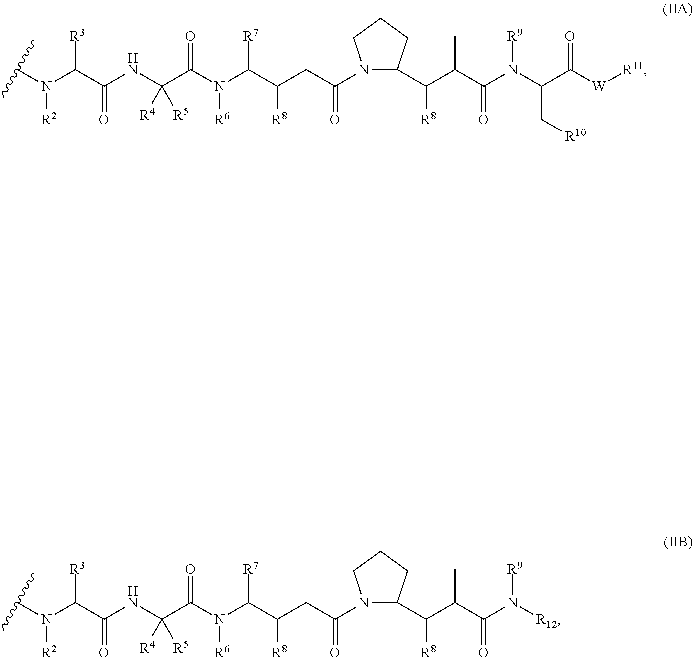

wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is and integer ranging between 0 to 6; and
t is and integer ranging between 0 to 6.

In some embodiments, G is selected from the following substituents:

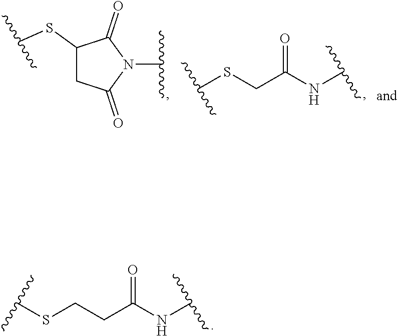

In some embodiments, T is an optionally substituted C₁-C₈ alkylene-C(O)—, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene —C(O)—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, or —(CH₂—CH₂—NR^{1B})ₙ—(CH₂)ₘC(O)—. In some embodiments, T is an optionally substituted C₁-C₈ alkylene-C(O)—, optionally substituted C₃-C₈ carbocyclylene-C(O, optionally substituted C₆-C₁₀ arylene —C(O)—, —(CH₂—CH₂—O)ₙ—(CH₂)ₘC(O)—, or —(CH₂—CH₂—NR^{1B})ₙ—(CH₂)ₘC(O)—. In some embodiments, T is an optionally substituted C₁-C₈ alkylene-C(O)—. In some embodiments, Q is a bond or

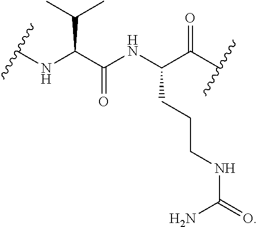 [SMILES: *N[C@H](C(=O)N[C@@H](CCCNC(N)=O)C(*)=O)C(C)C]

In some embodiments, Y is —NHCH₂C(O)— or —NHCH(CH₃)C(O)—. In some embodiments, U is monomethyl auristatin F (MMAF). In some embodiments, U is monomethyl auristatin E (MMAE). In some embodiments, M²-S is selected from the group consisting of:

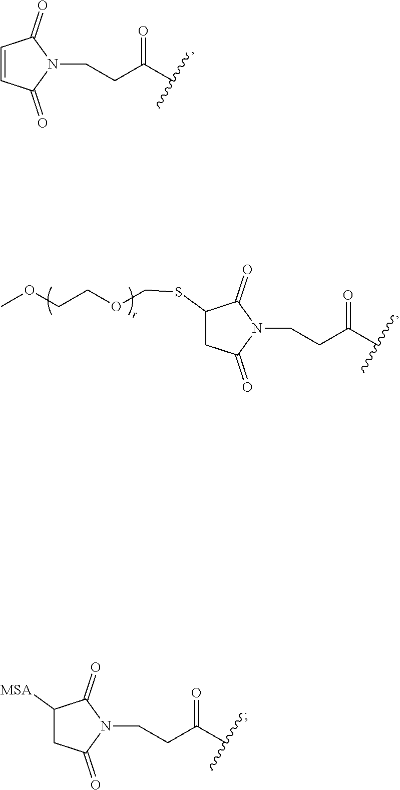

c_{A} is a bond,

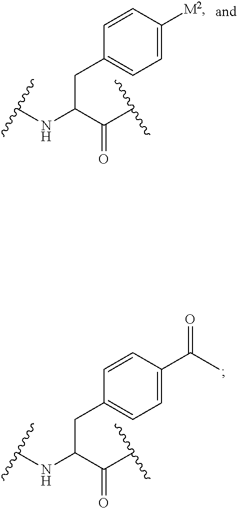

wherein,
cₚ-M¹ is selected from the group consisting of:

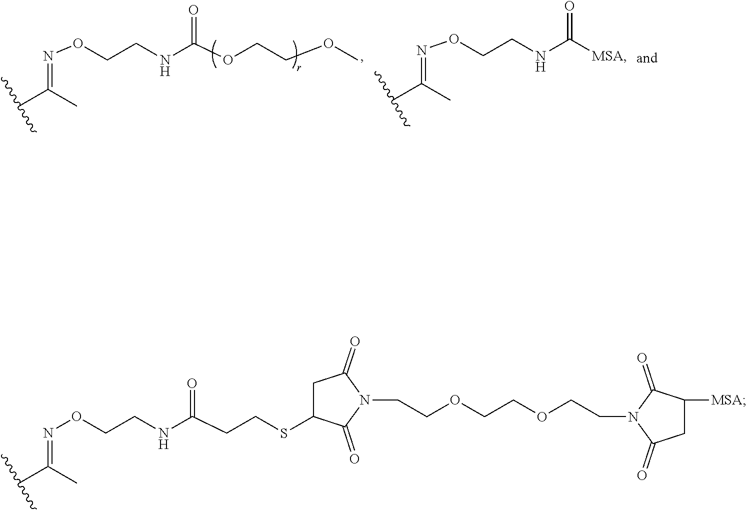

each r is independently an integer ranging from 40-1,100;
MSA is mouse serum albumin;
G is selected from the following substituents:

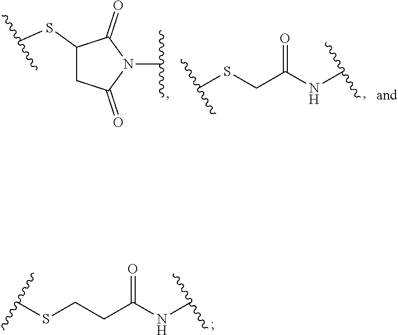

T is an optionally substituted C₁-C₈ alkylene-C(O)—;
Q is a bond or

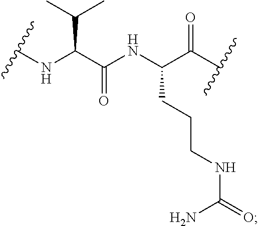 [SMILES: *N[C@H](C(=O)N[C@@H](CCCNC(N)=O)C(*)=O)C(C)C]

Y is —NHCH₂C(O)— or —NHCH(CH₃)C(O)—;
D is U; and
U is MMAE or MMAF.

In some embodiments, the selective delivery molecule is: SDM-173, SDM-176, SDM-178, SDM-181, SDM-182, SDM-189, SDM-190, SDM-191, SDM-195, SDM-199, SDM-200, SDM-210, SDM-211, SDM-212, SDM-218, SDM-219, SDM-220, SDM-221, SDM-222, SDM-225, SDM-227, SDM-228, SDM-229, SDM-230, SDM-231, SDM-257, or SDM-266. In some embodiments, A and B do not have an equal number of acidic and basic amino acids. In some embodiments, the number of basic amino acids in B is greater than the number of acidic amino acids in A. In some embodiments, A and B are independently selected from natural amino acids, unnatural amino acids, or a combination thereof. In some embodiments, A is a peptide comprising 5 or 9 consecutive glutamates. In some embodiments, A is a peptide comprising 5 consecutive glutamates. In some embodiments, A is a peptide comprising 9 consecutive glutamates. In some embodiments, B is a peptide comprising 8 or 9 consecutive arginines. In some embodiments, B is a peptide comprising 8 consecutive arginines. In some embodiments, B is a peptide comprising 9 consecutive arginines. In some embodiments, X is cleavable by a matrix metalloproteinase. In some embodiments, X comprises an amino acid sequence that is cleavable by MMP2, MMP7, MMP9, or MMP14.

Disclosed herein, in certain embodiments, is a selective delivery molecule of Formula III, having the structure:

[G-T-c_{A}-Q-Y-D]-(—N—Z)ᵥ Formula III

wherein
G is a linker or a reactive group;
T is a spacer;
c_{A} is a bond or a single amino acid;
Q is a bond or 1-3 amino acids;
Y is 1-3 amino acids or an amino alkylene;
D is an auristatin related therapeutic agent or an imaging agent;
each N is independently a bond or a linker;
Z is a peptide with a sequence comprising 3 to 10 amino acids or polyethylene glycol substituent;
wherein each N is independently bound to G or c_{A}; and
v is 1 or 2.

In some embodiments, G is a reactive group. In some embodiments, the reactive group is an N-maleimide. In some embodiments, G is

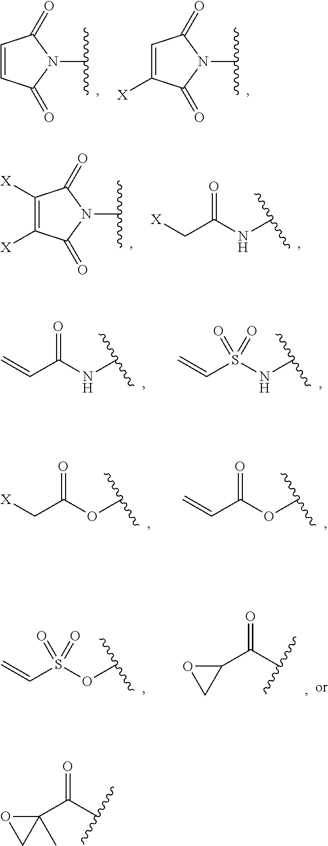

wherein each X is independently —Cl, —Br, —I, or —S-phenyl. In some embodiments, G is

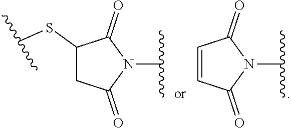

In some embodiments, T is an optionally substituted C₁-C₈ alkylene-C(O)—. In some embodiments, c_{A} is

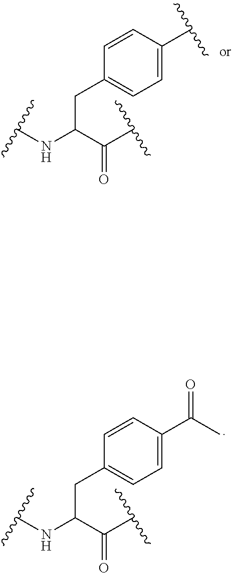 [SMILES: *NC(Cc1ccc(*)cc1)C(*)=O ; *NC(Cc1ccc(C(C)=O)cc1)C(*)=O]

In some embodiments, Q is

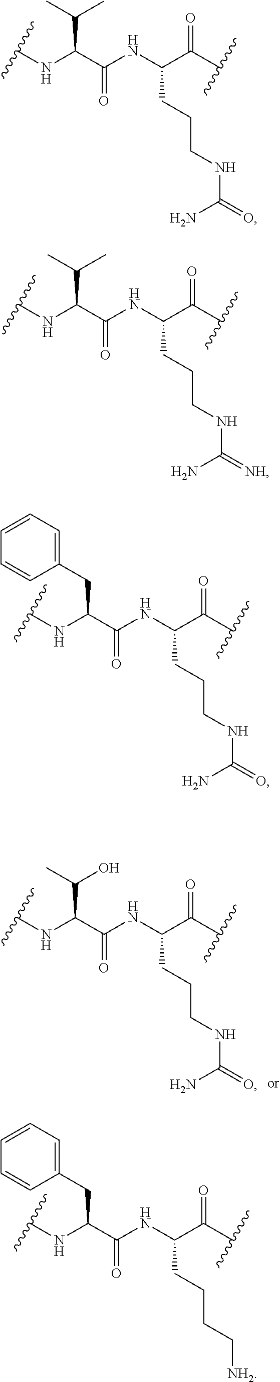 [SMILES: *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@@H](Cc1ccccc1)C(=O)N[C@@H](CCCNC(N)=O)C(*)=O ; *N[C@H](C(=O)N[C@@H](CCCNC(N)=O)C(*)=O)C(C)O ; "or" ; *N[C@@H](Cc1ccccc1)C(=O)N[C@@H](CCCCN)C(*)=O]

In some embodiments, Y is —NHCH₂C(O)— or —NHCH(CH₃)C(O)—. In some embodiments, D is MMAE or MMAF. In some embodiments, N is a bond,

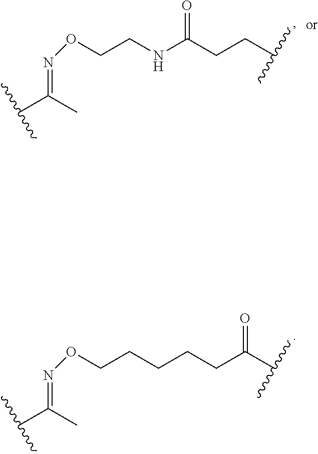 [SMILES: *CCC(=O)NCCO/N=C(/*)C ; *C(=O)CCCCCO/N=C(/*)C]

In some embodiments, Z is

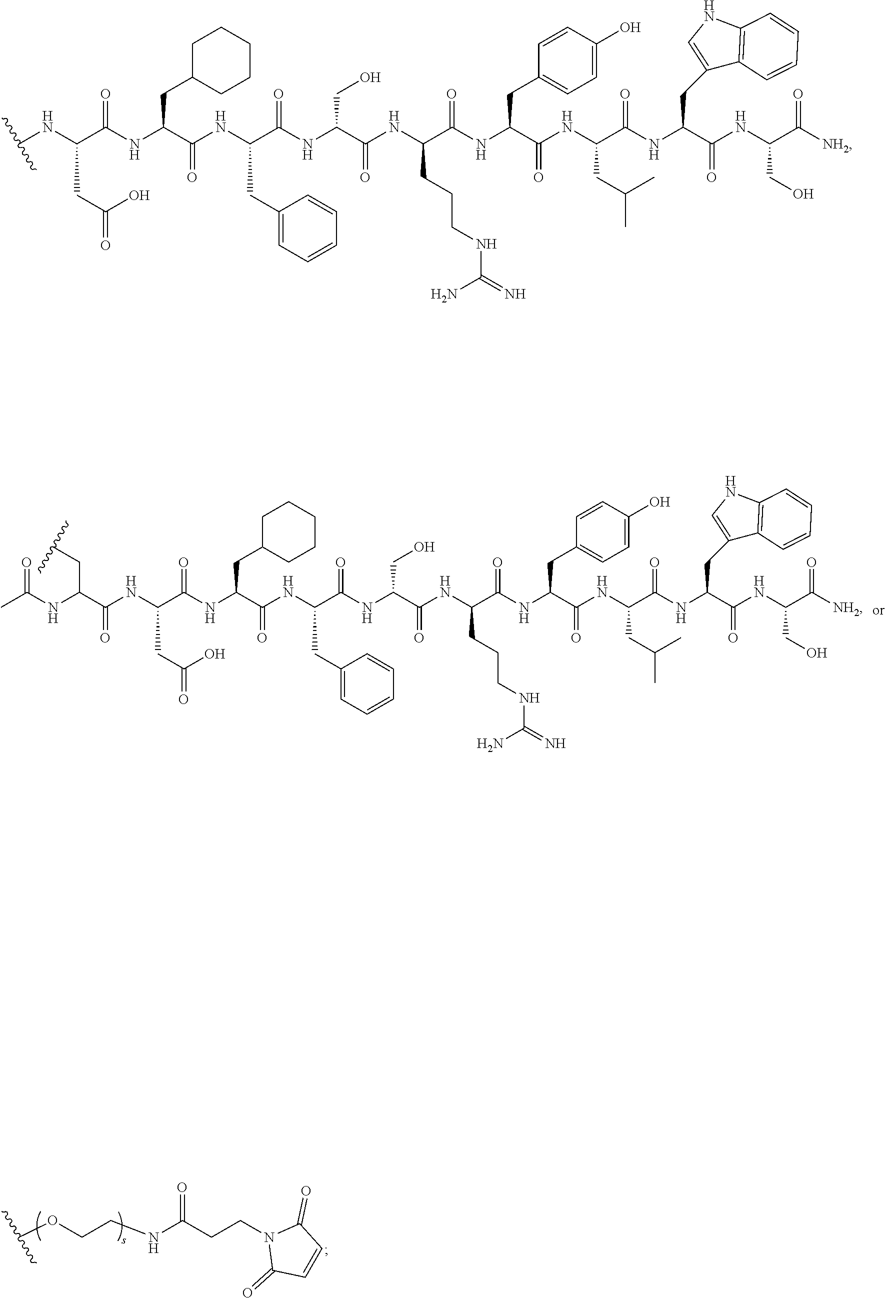

and wherein s is 1 to 20. In some embodiments, G is

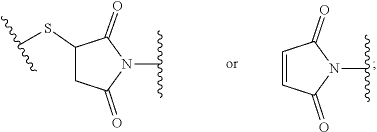 [SMILES: *SC1CC(=O)N(*)C1=O ; *N1C(=O)C=CC1=O]

T is an optionally substituted C₁-C₈ alkylene-C(O)—; c_{A} is

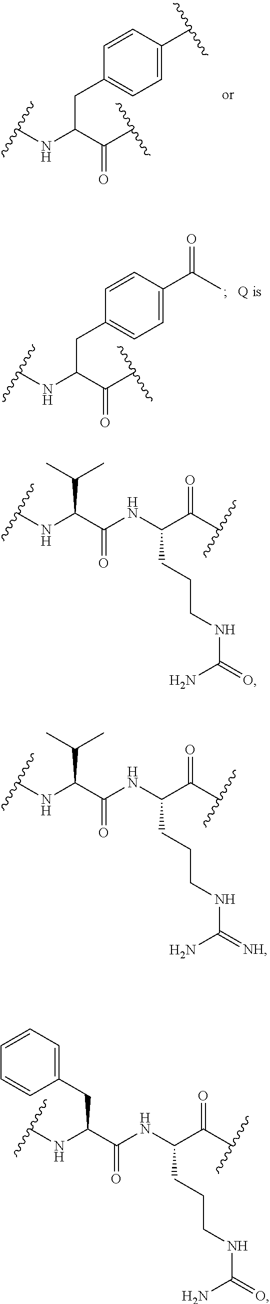

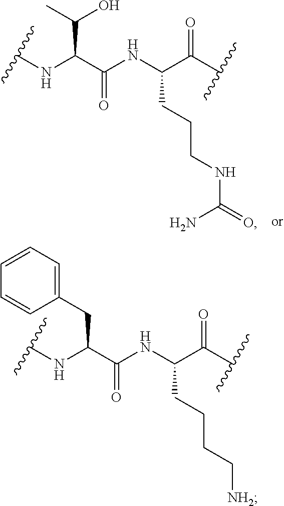 [SMILES: *N[C@H](C(=O)N[C@@H](CCCNC(N)=O)C(*)=O)C(C)O ; "or" ; *N[C@@H](Cc1ccccc1)C(=O)N[C@@H](CCCCN)C(*)=O]

Y is —NHCH₂C(O)— or —NHCH(CH₃)C(O)—; D is MMAE or MMAF; N is a bond,

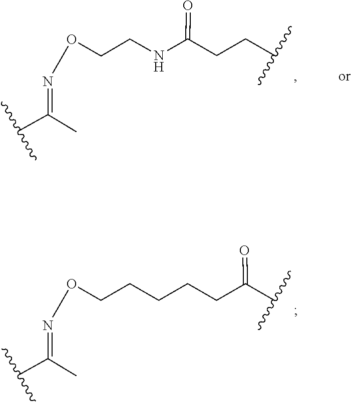 [SMILES: *CCC(=O)NCCO/N=C(/*)C ; *C(=O)CCCCCO/N=C(/*)C]

### Z is

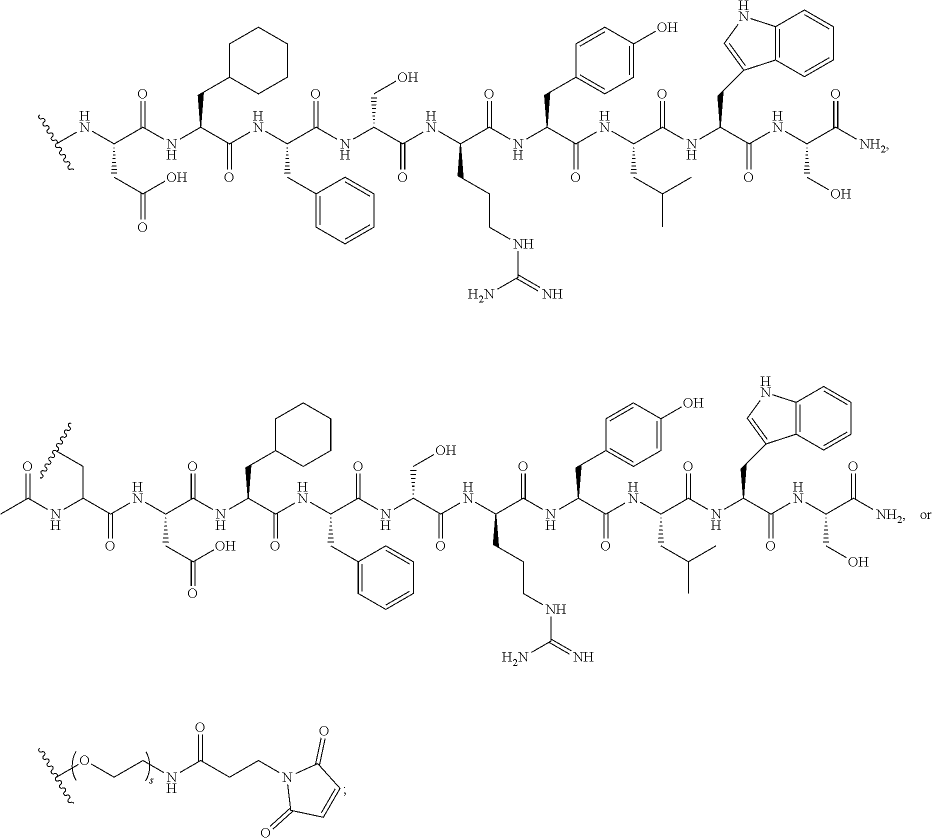

and wherein s is 1 to 20. In some embodiments, the selective delivery molecule is: SDM-185 or SDM-193.

Disclosed herein, in certain embodiments, is a selective delivery molecule, wherein the selective delivery molecule is SDM-201, SDM-202, SDM-203, SDM-204, SDM-208, SDM-213, SDM-214, SDM-215, SDM-216, SDM-223, SDM-224, SDM-226, SDM-232, SDM-234, SDM-235, SDM-261, SDM-262, SDM-263, SDM-264, or SDM-265.

Disclosed herein, in certain embodiments, is a selective delivery molecule, wherein the selective delivery molecule is SDM-187 or SDM-188.

Disclosed herein, in certain embodiments, is a selective delivery molecule, wherein die selective delivery molecule is SDM-192 or SDM-236.

Disclosed herein, in certain embodiments, is a pharmaceutical composition comprising of a compound of Formula (I), or a pharmaceutically acceptable salt thereof, and a pharmaceutically acceptable excipient. In some embodiments, the pharmaceutical composition is administered intravenously.

Disclosed herein, in certain embodiments, is a pharmaceutical composition comprising of a compound of Formula (II), or a pharmaceutically acceptable salt thereof, and a pharmaceutically acceptable excipient. In some embodiments, the pharmaceutical composition is administered intravenously.

Disclosed herein, in certain embodiments, is a pharmaceutical composition comprising of a compound of Formula (III), or a pharmaceutically acceptable salt thereof, and a pharmaceutically acceptable excipient. In some embodiments, the pharmaceutical composition is administered intravenously.

Disclosed herein, in certain embodiments, is a method of treating cancer in an individual in need thereof comprising administering to the individual a pharmaceutical composition comprising a compound of Formula (I), Formula (II), or Formula (III), or a pharmaceutically acceptable salt thereof, and a pharmaceutically acceptable excipient. In some embodiments, the cancer is breast cancer, colorectal cancer, squamous cell carcinoma, skin cancer, prostate cancer, melanoma, thyroid cancer, ovarian cancer, cervical cancer, lung cancer, pancreatic cancer, head and neck cancer, esophageal cancer, or sarcoma. In some embodiments, the cancer is breast cancer. In some embodiments, the cancer is colorectal cancer. In some embodiments, the cancer is prostate cancer. In some embodiments, the cancer is lung cancer. In some embodiments, the cancer is squamous cell carcinoma. In some embodiments, the cancer is sarcoma. In some embodiments, the pharmaceutical composition is administered intravenously. In some embodiments, the individual is a human.

Disclosed herein, in certain embodiments, is a method of treating cancer in an individual in need thereof comprising administering to the individual a pharmaceutical composition comprising SDM-201, SDM-202, SDM-203, SDM-204, SDM-208, SDM-213, SDM-214, SDM-215, SDM-216, SDM-223, SDM-224, SDM-226, SDM-232, SDM-234, SDM-235, SDM-261, SDM-262, SDM-263, SDM-264, or SDM-265, or a pharmaceutically acceptable salt thereof, and a pharmaceutically acceptable excipient. In some embodiments, the cancer is breast cancer, colorectal cancer, squamous cell carcinoma, skin cancer, prostate cancer, melanoma, thyroid cancer, ovarian cancer, cervical cancer, lung cancer, pancreatic cancer, head and neck cancer, esophageal cancer, or sarcoma. In some embodiments, the cancer is breast cancer. In some embodiments, the cancer is colorectal cancer. In some embodiments, the cancer is prostate cancer. In some embodiments, the cancer is lung cancer. In some embodiments, the cancer is squamous cell carcinoma. In some embodiments, the cancer is sarcoma. In some embodiments, die pharmaceutical composition is administered intravenously. In some embodiments, the individual is a human.

Disclosed herein, in certain embodiments, is a method of treating cancer in an individual in need thereof comprising administering to the individual a pharmaceutical composition comprising SDM-187 or SDM-188, or a pharmaceutically acceptable salt thereof, and a pharmaceutically acceptable excipient. In some embodiments, the cancer is breast cancer, colorectal cancer, squamous cell carcinoma, skin cancer, prostate cancer, melanoma, thyroid cancer, ovarian cancer, cervical cancer, lung cancer, pancreatic cancer, head and neck cancer, esophageal cancer, or sarcoma. In some embodiments, the cancer is breast cancer. In some embodiments, the cancer is colorectal cancer. In some embodiments, the cancer is prostate cancer. In some embodiments, the cancer is lung cancer. In some embodiments, the cancer is squamous cell carcinoma. In some embodiments, the cancer is sarcoma. In some embodiments, the pharmaceutical composition is administered intravenously. In some embodiments, the individual is a human.

Disclosed herein, in certain embodiments, is a method of visualizing a tissue of interest in an individual in need thereof, comprising administering to the individual a pharmaceutical composition comprising SDM-192 or SDM-236, or a pharmaceutically acceptable salt thereof, and a pharmaceutically acceptable excipient. In some embodiments, the individual has a cancer. In some embodiments, the method further comprises surgically removing the tissue of interest from the individual. In some embodiments, the visualizing is used to guide surgery, reduce positive margins, to stage cancer tissue, to stage lymph nodes, to reduce reoperations or allows a surgeon to minimize the removal of healthy tissue.

In some embodiments, the cancer is breast cancer, colorectal cancer, squamous cell carcinoma, skin cancer, prostate cancer, melanoma, thyroid cancer, ovarian cancer, cervical cancer, lung cancer, pancreatic cancer, head and neck cancer, esophageal cancer, or sarcoma. In some embodiments, the cancer is breast cancer. In some embodiments, the cancer is colorectal cancer. In some embodiments, the cancer is prostate cancer. In some embodiments, the cancer is lung cancer. In some embodiments, the cancer is squamous cell carcinoma. In some embodiments, the cancer is sarcoma. In some embodiments, the pharmaceutical composition is administered intravenously. In some embodiments, the individual is a human.

### BRIEF DESCRIPTION OF THE DRAWINGS

Various aspects of the disclosure are set forth with particularity in the appended claims. The patent application file contains at least one drawing executed in color. Copies of this patent application with color drawing(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIG. 1 provides an example of SDM compound dose responses and EC₅₀ determination of therapeutic compounds in cell viability assay with HT1080 fibrosarcoma cells.

FIG. 2 provides an example of active SDMs in human fibrosarcoma xenograft model demonstrating complete block of tumor growth compared to vehicle control.

FIG. 3A and FIG. 3B provide the experimental results from dosing of SDM compounds in HT-1080 human fibrosarcoma xenograft models. FIG. 3A illustrates the percentage change in tumor volume. FIG. 3B illustrates the percentage change in body weight.

FIG. 4 provides the experimental results from dosing compound SDM-154 in a HT-1080 human fibrosarcoma xenograft model and monitoring the concentration of both glycine-MMAF and MMAF.

### DETAILED DESCRIPTION OF THE INVENTION

Improving the delivery of drugs and other agents to the target cells, tissues and tumors to achieve maximal efficacy and minimal toxicity has been the focus of considerable research for many years. Though many attempts have been made to develop effective methods for importing biologically active molecules into cells, both in vivo and in vitro, none has proved to be entirely satisfactory. Optimizing the association of the drug with its intracellular target, while minimizing intercellular redistribution of the drug, e.g., to neighboring cells, is often difficult or inefficient. Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., chemotherapeutic (anti-cancer), cytotoxic, enzyme inhibitor agents and antiviral or antimicrobial drugs) that can be administered. By comparison, although oral administration of drugs is considered to be a convenient and economical mode of administration, it shares the same concerns of non-specific toxicity to unaffected cells once the drug has been absorbed into the systemic circulation. Further complications involve problems with oral bioavailability and residence of drug in the gut leading to additional exposure of gut to the drug and hence risk of gut toxicities. Accordingly, a major goal has been to develop methods for specifically targeting therapeutic and imaging agents to cells and tissues. The benefits of such treatment include avoiding the general physiological effects of inappropriate delivery of such agents to other cells and tissues, such as uninfected cells. Intracellular targeting may be achieved by methods, compounds and formulations which allow accumulation or retention of biologically active agents, i.e. active metabolites, inside cells. There is a clear need in the art for therapeutic auristatin derivatives and cyanine based imaging agents having significantly lower toxicity, yet useful therapeutic efficiency. These and other limitations and problems of the past are addressed by the present invention.

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information. Generally, the procedures for cell culture, cell infection, antibody production and molecular biology methods are methods commonly used in the art. Such standard techniques can be found, for example, in reference manual, such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristics) of the claimed invention.

As used herein, ranges and amounts can be expressed as “about” a particular value or range. About also includes the exact amount. Hence “about 40 mg” means “about 40 mg” and also “40 mg.” Generally, the terms “about” and “approximately” includes an amount that would be expected to be within experimental error.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

As used herein, the term “delivery molecule” refers to any agent (e.g., peptide, protein, nucleic acid polymer, aptamer, or small molecule) that associates with (e.g., binds to) a target of interest. The target of interest may be a tissue, a cell, a cellular structure (e.g., an organelle), a protein, a peptide, a polysaccharide, or a nucleic acid polymer.

The terms “polypeptide,” “peptide” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds.

Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see. e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acids are grouped as hydrophobic amino acids, polar amino acids, non-polar amino acids, and charged amino acids. Hydrophobic amino acids include small hydrophobic amino acids and large hydrophobic amino acids. Small hydrophobic amino acid can be glycine, alanine, proline, and analogs thereof. Large hydrophobic amino acids can be valine, leucine, isoleucine, phenylalanine, methionine, tryptophan, and analogs thereof. Polar amino acids can be serine, threonine, asparagine, glutamine, cysteine, tyrosine, and analogs thereof. Non-polar amino acids can be glycine, alanine, valine, leucine, isoleucine, methionine, phenylalanine, tryptophan, proline, and analogs thereof. Charged amino acids can be lysine, arginine, histidine, aspartate, glutamate, and analogs thereof. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids are either D amino acids or L amino acids.

In some instances, one or more of the amino acid residues in the Formulas (I), (II), (III), (IV), or (V) described herein is modified to a polar amino acid. As discussed above, exemplary polar amino acids include serine, threonine, asparagine, glutamine, cysteine, tyrosine, and analogs thereof.

In other instances, one or more of the amino acid residues in the Formulas (I), (II), (III), (IV), or (V) described herein is modified to a non-polar amino acid. Exemplary non-polar amino acids include glycine, alanine, valine, leucine, isoleucine, methionine, phenylalanine, tryptophan, proline, and analogs thereof.

In some cases, one or more of the amino acid residues in the Formulas (I), (II), (III), (IV), or (V) described herein is modified a hydrophobic amino acids. Exemplary hydrophobic amino acids include small hydrophobic amino acid such as glycine, alanine, proline, and analogs thereof; and large hydrophobic amino acids such as valine, leucine, isoleucine, phenylalanine, methionine, tryptophan, and analogs thereof.

In other cases, one or more of the amino acid residues in the Formulas (I), (II), (III), (IV), or (V) described herein is modified to a charged amino acid. Exemplary charged amino acids include lysine, arginine, histidine, aspartate, glutamate, and analogs thereof.

In some embodiments, one of skill will recognize that one or more of the amino acid residues described herein may be conservatively modified. Conservative substitution tables providing functionally similar amino acids are well known in the art. For examples, the following table illustrates exemplary conservative substitutions.

| Original Residue | Conserved Substitutions |
|---|---|
| Ala | Ser, Gly, Thr, Cys, Val |
| Arg | Lys, Gln, His, Asn, Glu |
| Asn | Gln, His, Asp, Lys, Ser, Thr, Arg, Glu |
| Asp | Glu, Asn, Gln, Ser |
| Cys | Ser, Ala |
| Gln | Asn, Arg, Glu, His, Lys Met, Asp, Ser |
| Glu | Asp, Gln, Lys, Arg, Asn, His, Ser |
| Gly | Pro, Ala, Ser |
| His | Asn, Gln, Arg, Tyr, Glu |
| Ile | Leu, Val, Met, Phe |
| Leu | Ile, Val, Met, Phe |

In some cases, such conservatively modified variants are in addition to and do not exclude polymorphic variants, interspecies homologs, and alleles of the invention.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₃ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Carbocyclylalkynyl” refers to a radical of the formula —R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

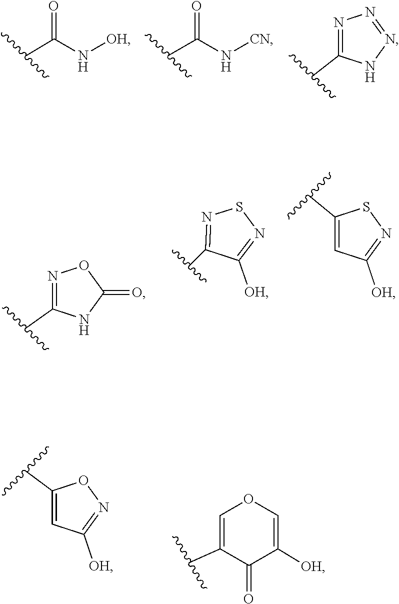

and the like.

“Halo” or “halogen” refers to bromo, chloro, fluoro or iodo substituents. In some embodiments the halogen is chloro. In some embodiments the halogen is fluoro. In some embodiments the halogen is iodo. In some embodiments the halogen is bromo.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An A-heterocyclyl radical is optionally substituted as described above for heterocyclyl radicals. Examples of such W-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) w-electron system in accordance with the Hückel theory.

Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]diieno[2,3-d]pyrimidinyl, 5,6,7,8-tetrahydropyrido[4,5-c]pyridazinyl, thiazolyl, diiadiazolyl, triazolyl, tetrazolyl, triazinyl, thieno[2,3-d]pyrimidinyl, diieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An A-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain.

The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or tram) Likewise, all possible isomers, as well as their racemic and optically pure forms, and all tautomeric forms are also intended to be included. The term “geometric isomer” refers to E or Z geometric isomers (e.g., cis or tram) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

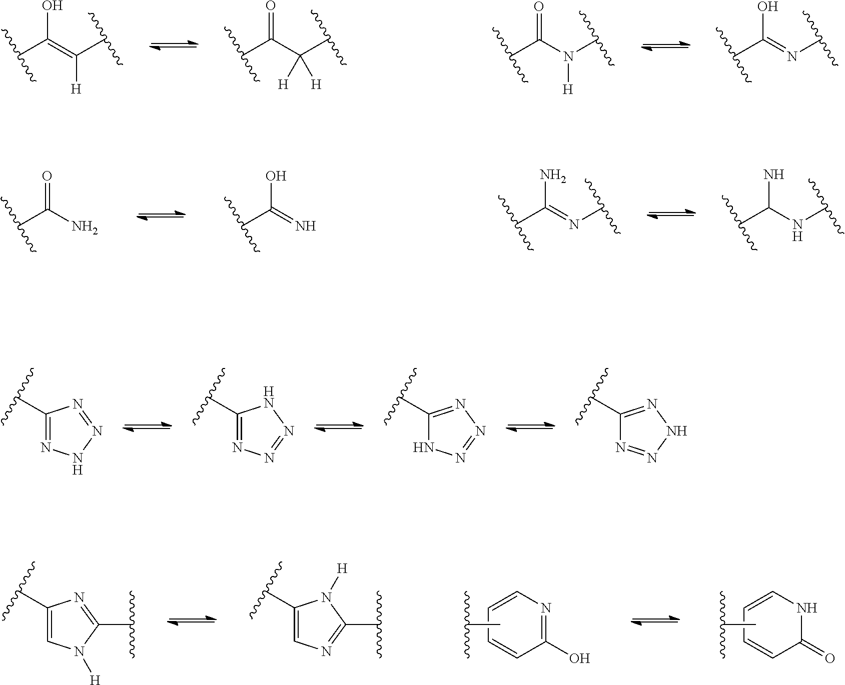

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²³I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989,45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

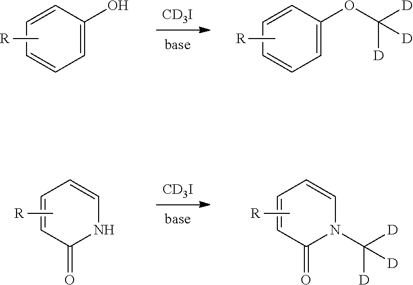

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

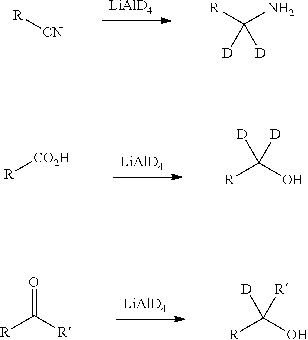

Deuterium gas and palladium catalyst are employed to reduce unsaturated carbon-carbon linkages and to perform a reductive substitution of aryl carbon-halogen bonds as illustrated, by way of example only, in the reaction schemes below.

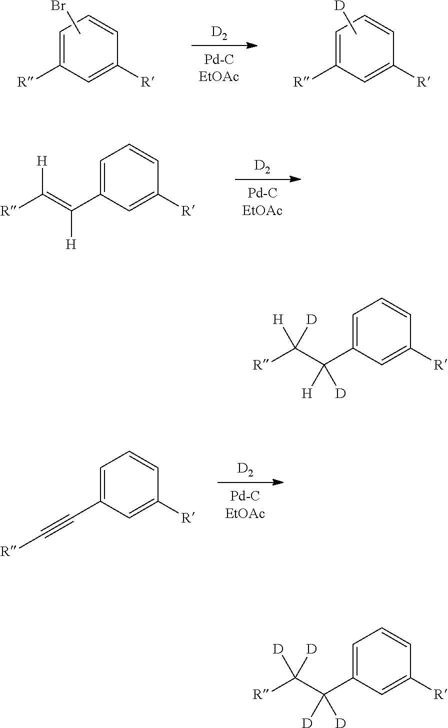

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the selective delivery molecules described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicaiboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, p-toluene sulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fiimarates, maleates, mandelates, benzoates, chlorobenzoates, methyibenzoates, dinitrobenzoates, phthalates, benzene sulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science. 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, AZN-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Selective Delivery Molecules

Disclosed herein are selective delivery molecules (SDMs) that allow the targeted delivery of therapeutic agents and/or imaging agents to specific cells and/or tissues. In some embodiments, selective delivery molecules comprise (a) an acidic sequence (portion of A), (b) a cleavable linker X located between portion of A and portion of B, (c) a basic sequence (portion B), (d) at least one therapeutic agent or imaging agent (e.g., portion D_{B} or D) bound to portion of B, and optionally (e) a peptide bound to portion of A or B directly or through a linker. In some embodiments, cleavage of the X linker allows the separation of portion of A from portion of B, thereby promoting the uptake or retention of portion of B and the attached drug agent or imaging agent into cells or tissues. In some embodiments, the therapeutic agent is a chemotherapeutic agent. In some embodiments, the therapeutic agent is a cytotoxin.

In some embodiments, selective delivery molecules comprise (a) a N-maleimide substituent or N-succinamide linker (portion S), (b) an acidic sequence (portion of A), (c) an optional amino acid linker (portion c_{A}), (d) an optional linker (portion cₚ) bound to a macromolecule (portion M), a cleavable linker X located between A and B or between c_{A} and B, (e) a basic sequence (portion B), (f) a single amino acid linker (portion c_{B}) boud to B and D_{B}, (g) at least one therapeutic agent or imaging agent (e.g., portion D_{B}) bound to c_{B}, and (h) at least one linker (portion N) bound to a peptide (portion Z), wherein N also bound to one or more of S, c_{A}, and c_{B}.

In some embodiments, selective delivery molecules comprise (a) an optional N-maleimide substituent or optional N-succinamide linker (portion S), (b) an acidic sequence (portion of A) of 5 to 9 acidic amino acids, (c) an optional single amino acid linker (portion c_{A}), (d) an optional linker (portion cₚ) bound to a macromolecule (portion M), a cleavable linker X located between A and B or between c_{A} and B, (e) a basic sequence (portion B) of 5 to 20 basic amino acids, (f) a single amino acid linker (portion c_{B}) boud to B and D_{B}, (g) a therapeutic agent or imaging agent (portion D_{B}) bound to c_{B}, and (h) at least one linker (portion N) bound to a peptide (portion Z), wherein N also bound to one or more of S, c_{A}, and c_{B}.

In some embodiments, selective delivery molecules comprise (a) an optional N-maleimide substituent or optional N-succinamide linker (portion S), wherein the succinimide is bound to a macromolecule (portion M²), (b) an acidic sequence (portion of A) of 5 to 9 acidic amino acids, (c) an optional single amino acid linker (portion c_{A}), (d) an optional linker (portion cₚ) bound to a macromolecule (portion M¹), a cleavable linker X located between A and B or between c_{A} and B, (e) a basic sequence (portion B) of 5 to 20 basic amino acids, (f) a single amino acid linker (portion c_{B}) boud to B and G, (g) a linker (portion G), (h) a spacer (portion T), (i) a sequence between 1 and 3 amino acids (portion Q) which is cleavable by an enzyme, (j) a modifier (portion Y) of the therapeutic agent or imaging agent, and (k) a therapeutic agent or imaging agent (portion D). In some embodiments, D is a cytotoxin. In some embodiments, Y-D is an auristatin derivative. In some embodiments, Y-D is fragment K.

In some embodiments, disclosed here are selective delivery molecules of Formula I, having the structure:

[(S)_{w}-A-c_{A}-(cₚ-M)ᵤ-X—B-c_{B}-D_{B}]-(N—Z)ᵥ Formula I

in which S is an N-maleimide or N-succinamide substituent bound to the amino terminus of A; A is a peptide with a sequence comprising 5 to 9 acidic amino acids; c_{A} is a bond or a single amino acid linker; cₚ is a linker; M is a macromolecule; X is a cleavable linker; B is a peptide with a sequence comprising 5 to 20 basic amino acids; c_{B} is a single amino acid linker; D_{B} is a therapeutic agent or an imaging agent; each N is independently selected from a bond and a linker; each Z is independently a peptide with a sequence comprising 3 to 10 amino acids; v is 1 or 2; and w and u are independently 0 or 1; wherein each N is independently bound to c_{A}, c_{B}, or the amino terminus of A, S is bound to the amino terminus of A, and cₚ is bound to c_{A}; and wherein if S is present, S and N are not bound at the same position; and if cₚ is present, cₚ and N are not bound at the same position.

In some embodiments, disclosed here are selective delivery molecules of Formula II, having the structure:

M²ₒ-S_{w}-A-c_{A}-(c_{P}-M¹)ᵤ-X—B-c_{B}-G-T-Q-Y-D Formula II

in which M¹ and M² are each independently a macromolecule; S is an N-maleimide substituent or N-succinamide linker bound to the amino terminus of A; A is a peptide with a sequence comprising 5 to 9 acidic amino acids; c_{A} is a bond or a single amino acid; cₚ is a linker bound to c_{A}; X is a cleavable linker; B is a peptide with a sequence comprising 5 to 20 basic amino acids; c_{B} is a single amino acid; G is a linker; T is a spacer; Q is a bond or 1-3 amino acids; Y is 1-3 amino acids or an amino alkylene; D is an auristatin related therapeutic agent; and w, u, and o are independently 0 or 1. In some embodiments, Y-D is fragment K.

In some embodiments, cleavage of the Q linker allows the separation of portion of Y-D from portion of G-T or M-G-T, thereby promoting the uptake or retention of portion of the therapeutic agent Y-D or imaging agent Y-D into cells or tissue retention. In some embodiments, the therapeutic agent is a chemotherapeutic agent. In some embodiments, the therapeutic agent is a cytotoxin. In some embodiments, the therapeutic agent is a modified auristatin.

In some embodiments, therapeutic agent Y-D or imaging agent Y-D has superior therapeutic or imaging properties to the free therapeutic agent D or imaging agent D. In some embodiments, Y-D is non-hydrolyzable under physiological conditions. In some embodiments, Y is a single amino acid. In some embodiments, Y is a dipeptide (two amino acids). In some embodiments, Y is not an amino acid. In some embodiments, Y is an amino acid such as alanine or glycine. In some embodiments, Y is a non-amino acid modifier that consists of 15 atoms or less. In some embodiments, Y is a non-amino acid modifier that consists of 10 atoms or less.

### Portion A

In some embodiments, A is a peptide with a sequence comprising 2 to 20 acidic amino acids. In some embodiments, peptide portion of A comprises between about 2 to about 20 acidic amino acids. In some embodiments, peptide portion of A comprises between about 5 to about 20 acidic amino acids. In some embodiments, A has a sequence comprising 5 to 9 acidic amino acids. In some embodiments, A has a sequence comprising 5 to 8 acidic amino acids. In some embodiments, A has a sequence comprising 5 to 7 acidic amino acids. In some embodiments, A has a sequence comprising 5 acidic amino acids. In some embodiments, A has a sequence comprising 6 acidic amino acids. In some embodiments, A has a sequence comprising 7 acidic amino acids. In some embodiments, A has a sequence comprising 8 acidic amino acids. In some embodiments, A has a sequence comprising 9 acidic amino acids. In some embodiments, A is a peptide with a sequence comprising 5 to 9 acidic amino acids.

In some embodiments, peptide portion of A comprises between about 2 to about 20 consecutive acidic amino acids. In some embodiments, peptide portion of A comprises between about 5 to about 20 consecutive acidic amino acids. In some embodiments, A has a sequence comprising 5 to 9 consecutive acidic amino acids. In some embodiments, A has a sequence comprising 5 to 8 consecutive acidic amino acids. In some embodiments, A has a sequence comprising 5 to 7 consecutive acidic amino acids. In some embodiments, A has a sequence comprising 5 consecutive acidic amino acids. In some embodiments, A has a sequence comprising 6 consecutive acidic amino acids. In some embodiments, A has a sequence comprising 7 consecutive acidic amino acids. In some embodiments, A has a sequence comprising 8 consecutive acidic amino acids. In some embodiments, A has a sequence comprising 9 consecutive acidic amino acids.

In some embodiments, peptide portion of A comprises between about 2 to about 20 acidic amino acids selected from, aspartates and glutamates. In some embodiments, peptide portion of A comprises between about 5 to about 20 acidic amino acids selected from, aspartates and glutamates. In some embodiments, A has a sequence comprising 5 to 9 acidic amino acids selected from, aspartates and glutamates. In some embodiments, A has a sequence comprising 5 to 8 acidic amino acids selected from, aspartates and glutamates. In some embodiments, A has a sequence comprising 5 to 7 acidic amino acids selected from, aspartates and glutamates. In some embodiments, A has a sequence comprising 5 acidic amino acids selected from, aspartates and glutamates. In some embodiments, A has a sequence comprising 6 acidic amino acids selected from, aspartates and glutamates. In some embodiments, A has a sequence comprising 7 acidic amino acids selected from, aspartates and glutamates. In some embodiments, A has a sequence comprising 8 acidic amino acids selected from, aspartates and glutamates. In some embodiments, A has a sequence comprising 9 acidic amino acids selected from, aspartates and glutamates.

In some embodiments, peptide portion of A comprises between about 2 to about 20 consecutive acidic amino acids selected from, aspartates and glutamates. In some embodiments, peptide portion of A comprises between about 5 to about 20 consecutive acidic amino acids selected from, aspartates and glutamates. In some embodiments, A has a sequence comprising 5 to 9 consecutive acidic amino acids selected from, aspartates and glutamates. In some embodiments, A has a sequence comprising 5 to 8 consecutive acidic amino acids selected from, aspartates and glutamates. In some embodiments, A has a sequence comprising 5 to 7 consecutive acidic amino acids selected from, aspartates and glutamates. In some embodiments, A has a sequence comprising 5 consecutive acidic amino acids selected from, aspartates and glutamates. In some embodiments, A has a sequence comprising 6 consecutive acidic amino acids selected from, aspartates and glutamates. In some embodiments, A has a sequence comprising 7 consecutive acidic amino acids selected from, aspartates and glutamates. In some embodiments, A has a sequence comprising 8 consecutive acidic amino acids selected from, aspartates and glutamates. In some embodiments, A has a sequence comprising 9 consecutive acidic amino acids selected from, aspartates and glutamates.

In some embodiments, peptide portion of A comprises between about 2 to about 20 glutamates. In some embodiments, peptide portion of A comprises between about 5 to about 20 glutamates. In some embodiments, A has a sequence comprising 5 to 9 glutamates. In some embodiments, A has a sequence comprising 5 to 8 glutamates. In some embodiments, A has a sequence comprising 5 to 7 glutamates. In some embodiments, A has a sequence comprising 5 glutamates. In some embodiments, A has a sequence comprising 6 glutamates. In some embodiments, A has a sequence comprising 7 glutamates. In some embodiments, A has a sequence comprising 8 glutamates. In some embodiments, A has a sequence comprising 9 glutamates.

In some embodiments, peptide portion of A comprises between about 2 to about 20 consecutive glutamates. In some embodiments, peptide portion of A comprises between about 5 to about 20 consecutive glutamates. In some embodiments, A has a sequence comprising 5 to 9 consecutive glutamates. In some embodiments, A has a sequence comprising 5 to 8 consecutive glutamates. In some embodiments, A has a sequence comprising 5 to 7 consecutive glutamates. In some embodiments, A has a sequence comprising 5 consecutive glutamates. In some embodiments, A has a sequence comprising 6 consecutive glutamates. In some embodiments, A has a sequence comprising 7 consecutive glutamates. In some embodiments, A has a sequence comprising 8 consecutive glutamates. In some embodiments, A has a sequence comprising 9 consecutive glutamates.

In some embodiments, portion of A comprises 5 consecutive glutamates (i.e., EEEEE or eeeee). In some embodiments, portion of A comprises 9 consecutive glutamates (i.e., EEEEEEEEE or eeeeeeeee).

In some embodiments, A is a peptide comprising a series of 5 to 20 glutamates. In some embodiments, A is a peptide comprising a series of 5 or 9 glutamates. In some embodiments, A is a peptide comprising a series of 5 glutamates. In some embodiments, A is a peptide comprising a series of 9 glutamates.

An acidic portion of A may include amino acids that are not acidic. Acidic portion of A may comprise other moieties, such as negatively charged moieties. In embodiments of a selective delivery molecule disclosed herein, an acidic portion of A may be a negatively charged portion, preferably having about 2 to about 20 negative charges at physiological pH that does not include an amino acid.

In some embodiments, the amount of negative charge in portion of A is approximately the same as the amount of positive charge in portion of B. In some embodiments, the amount of negative charge in portion of A is not the same as the amount of positive charge in portion of B. In some embodiments, improved tissue uptake is seen in a selective delivery molecule wherein the amount of negative charge in portion of A is not the same as the amount of positive charge in portion of B. In some embodiments, improved solubility is observed in a selective delivery molecule wherein the amount of negative charge in portion of A is not the same as the amount of positive charge in portion of B. In some embodiments, faster tissue uptake is seen in a selective delivery molecule wherein the amount of negative charge in portion of A is not the same as the amount of positive charge in portion of B. In some embodiments, greater tissue uptake is seen in a selective delivery molecule wherein the amount of negative charge in portion of A is not the same as the amount of positive charge in portion of B.

Portion of A is either L-amino acids or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines. In some instances, the amino acid is L-amino acid (e.g., 2 to 20 acidic L-amino acids). In other instances, the amino acid is D-amino acid (e.g., 2 to 20 D-amino acids). In additional instances, the 2 to 20 amino acids comprise a mixture of L- and D-amino acids. In some instances, A comprises a series of L-aspartates, D-aspartates, or a mixture of L- and D-aspartates. In some instances, A comprises a series of L-glutamates, D-glutamates, or a mixture of L- and D-glutamates. In some cases, portion of A comprises 5 consecutive L-glutamates. In some cases, portion of A comprises 5 consecutive D-glutamates. In some cases, portion of A comprises 5 consecutive glutamates that are a mixture of L- and D-glutamates. In some cases, portion of A comprises 9 consecutive L-glutamates. In some cases, portion of A comprises 9 consecutive D-glutamates. In some cases, portion of A comprises 9 consecutive glutamates that are a mixture of L- and D-glutamates.

It will be understood that portion of A may include non-standard amino acids, such as, for example, hydroxylysine, desmosine, isodesmosine, or other non-standard amino acids. Portion of A may include modified amino acids, including post-translationally modified amino acids such as, for example, methylated amino acids (e.g., methyl histidine, methylated forms of lysine, etc.), acetylated Amino acids, amidated amino acids, formylated amino acids, hydroxylated amino acids, phosphorylated amino acids, or other modified amino acids. Portion of A may also include peptide mimetic moieties, including portions linked by non-peptide bonds and amino acids linked by or to non-amino acid portions.

The selective delivery molecules disclosed herein are effective where A is at the amino terminus or where A is at the carboxy terminus, i.e., either orientation of the peptide bonds is permissible.

### Portion B

In some embodiments, B is a peptide with a sequence comprising 5 to 20 basic amino acids. In some embodiments, peptide portion of B comprises between about 5 to about 15 basic amino acids. In some embodiments, peptide portion of B comprises between about 5 to about 12 basic amino acids. In some embodiments, peptide portion of B comprises between about 7 to about 9 basic amino acids. In some embodiments, peptide portion of B comprises between about 7 to about 8 basic amino acids. In some embodiments, peptide portion of B comprises 9 basic amino acids. In some embodiments, peptide portion of B comprises 8 basic amino acids. In some embodiments, peptide portion of B comprises 7 basic amino acids.

In some embodiments, peptide portion of B comprises between about 5 to about 20 consecutive basic amino acids. In some embodiments, peptide portion of B comprises between about 5 to about 12 consecutive basic amino acids. In some embodiments, peptide portion of B comprises between about 7 to about 9 consecutive basic amino acids. In some embodiments, peptide portion of B comprises between about 7 to about 8 consecutive basic amino acids. In some embodiments, peptide portion of B comprises 9 consecutive basic amino acids. In some embodiments, peptide portion of B comprises 8 consecutive basic amino acids. In some embodiments, peptide portion of B comprises 7 consecutive basic amino acids.

In some embodiments, peptide portion of B comprises between about 5 to about 20 basic amino acids selected from arginines, histidines, and lysines. In some embodiments, peptide portion of B comprises between about 5 to about 12 basic amino acids selected from arginines, histidines, and lysines. In some embodiments, peptide portion of B comprises between about 7 to about 9 basic amino acids selected from arginines, histidines, and lysines. In some embodiments, peptide portion of B comprises between about 7 to about 8 basic amino acids selected from arginines, histidines, and lysines. In some embodiments, peptide portion of B comprises 9 basic amino acids selected from arginines, histidines, and lysines. In some embodiments, peptide portion of B comprises 8 basic amino acids selected from arginines, histidines, and lysines. In some embodiments, peptide portion of B comprises 7 basic amino acids selected from arginines, histidines, and lysines.

In some embodiments, peptide portion of B comprises between about 5 to about 20 consecutive basic amino acids selected from arginines, histidines, and lysines. In some embodiments, peptide portion of B comprises between about 5 to about 12 consecutive basic amino acids selected from arginines, histidines, and lysines. In some embodiments, peptide portion of B comprises between about 7 to about 9 consecutive basic amino acids selected from arginines, histidines, and lysines. In some embodiments, peptide portion of B comprises between about 7 to about 8 consecutive basic amino acids selected from arginines, histidines, and lysines. In some embodiments, peptide portion of B comprises 9 consecutive basic amino acids selected from arginines, histidines, and lysines. In some embodiments, peptide portion of B comprises 8 consecutive basic amino acids selected from arginines, histidines, and lysines.

In some embodiments, peptide portion of B comprises between about 5 to about 20 arginines. In some embodiments, peptide portion of B comprises between about 5 to about 12 arginines. In some embodiments, peptide portion of B comprises between about 7 to about 9 arginines. In some embodiments, peptide portion of B comprises between about 7 to about 8 arginines. In some embodiments, peptide portion of B comprises 9 arginines. In some embodiments, peptide portion of B comprises 8 arginines. In some embodiments, peptide portion of B comprises 7 arginines.

In some embodiments, peptide portion of B comprises between about 5 to about 20 consecutive arginines. In some embodiments, peptide portion of B comprises between about 5 to about 12 consecutive arginines. In some embodiments, peptide portion of B comprises between about 7 to about 9 consecutive arginines. In some embodiments, peptide portion of B comprises between about 7 to about 8 consecutive arginines. In some embodiments, peptide portion of B comprises 9 consecutive arginines. In some embodiments, peptide portion of B comprises 8 consecutive arginines. In some embodiments, peptide portion of B comprises 7 consecutive arginines.

In some embodiments, peptide portion of B comprises a series of between about 5 to about 20 arginines. In some embodiments, peptide portion of B comprises a series of between about 5 to about 12 arginines. In some embodiments, peptide portion of B comprises a series of between about 7 to about 9 arginines. In some embodiments, peptide portion of B comprises a series of between about 7 to about 8 arginines. In some embodiments, peptide portion of B comprises a series of 9 arginines. In some embodiments, peptide portion of B comprises a series of 8 arginines. In some embodiments, peptide portion of B comprises a series of 7 arginines.

A basic portion of B may include amino acids that are not basic. Basic portion of B may comprise other moieties, such as positively charged moieties. In embodiments, a basic portion of B may be a positively charged portion, preferably having between about 5 and about 20 positive charges at physiological pH that does not include an amino acid. In some embodiments, the amount of negative charge in portion of A is approximately the same as the amount of positive charge in portion of B. In some embodiments, the amount of negative charge in portion of A is not the same as the amount of positive charge in portion of B.

Portion of B is either L-amino acids or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines. In some instances, the amino acid is L-amino acid (e.g., 5 to 20 basic L-amino acids). In other instances, the amino acid is D-amino acid (e.g., 5 to 20 basic D-amino acids). In additional instances, the 5 to 20 amino acids comprise a mixture of L- and D-amino acids. In some instances, B comprises a series of L-arginines, D-arginines, or a mixture of L- and D-arginines. In some instances, B comprises a series of L-histidines, D-histidines, or a mixture of L- and D-histidines. In some instances, B comprises a series of L-lysines, D-lysines, or a mixture of L- and D-lysines. In some cases, peptide portion of B comprises a series of 8 L-arginines or 9 L-arginines. In some cases, peptide portion of B comprises a series of 8 D-arginines or 9 D-arginines. In some cases, peptide portion of B comprises a series of 8 arginines or 9 arginines that is a mixture of L- and D-arginines.

It will be understood that portion of B may include non-standard amino acids, such as, for example, hydroxylysine, desmosine, isodesmosine, or other non-standard amino acids. Portion of B may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion of B may also include peptide mimetic moieties, including portions linked by non-peptide bonds and amino acids linked by or to non-amino acid portions.

In embodiments where X is a peptide cleavable by a protease, it may be preferable to join the C-terminus of X to the N-terminus of B, so that the new amino terminus created by cleavage of X contributes an additional positive charge that adds to the positive charges already present in B.

In some embodiments, B is a peptide with a sequence comprising 5 to 20 basic amino acids. In some embodiments, B is a peptide with a sequence comprising 7 to 9 basic amino acids.

In some embodiments, B is a peptide comprising 8 or 9 consecutive arginines. In some embodiments, B is a peptide comprising 8 consecutive arginines. In some embodiments, B is a peptide comprising 9 consecutive arginines

Conjugation Group (c_{A}, c_{P}, and c_{B})

### Portion c_{A}

In some embodiments, c_{A} is a bond or a single amino acid linker. In some embodiments, c_{A} is a bond. In some embodiments, c_{A} is a single amino acid linker.

In some embodiments, N is bound to c_{A}. In some embodiments c_{A} is bound to cₚ. In some embodiments c_{A} is bound to X. In some embodiments c_{A} is bound to Q. In some embodiments c_{A} is bound to T.

In some embodiments, c_{A} is a bond,

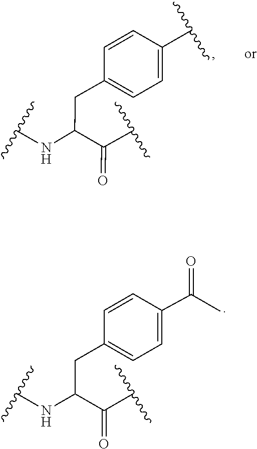 [SMILES: *NC(Cc1ccc(*)cc1)C(*)=O ; *NC(Cc1ccc(C(C)=O)cc1)C(*)=O]

In some embodiments, c_{A} is a bond. In some embodiments, c_{A} is

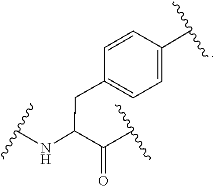 [SMILES: *NC(Cc1ccc(*)cc1)C(*)=O]

In some embodiments, c_{A} is

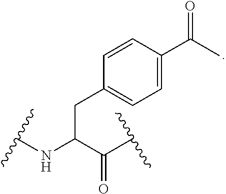 [SMILES: *NC(Cc1ccc(C(C)=O)cc1)C(*)=O]

In some embodiments, c_{A} comprises 0-10 amino acids. In some embodiments, c_{A} comprises 0-1 amino acids. In some embodiments, c_{A} comprises 0-3 amino acids. In some embodiments, c_{A} is a bond. In some embodiments, c_{A} comprises 1 amino acid. In some embodiments, c_{A} comprises 2 amino acids. In some embodiments, c_{A} comprises 3 amino acids. In some embodiments, c_{A} comprises 4 amino acid. In some embodiments, c_{A} comprises 5 amino acids. In some embodiments, c_{A} comprises 6 amino acids. In some embodiments, c_{A} comprises 7 amino acids. In some embodiments, c_{A} comprises 8 amino acids. In some embodiments, c_{A} comprises 9 amino acids. In some embodiments, c_{A} comprises 10 amino acids.

In some embodiments, c_{A} comprises a derivatized amino acid.

In some embodiments, c_{A} comprises a naturally-occurring amino acid or a non-naturally-occurring amino acid. In some embodiments, c_{A} is selected from a D amino acid, a L amino acid, an ex-amine) acid, a β-amino acid, or a r-amino acid. In some embodiments, c_{A} comprises any amino acid having a free thiol group, any amino acid containing a free amine group, any amino acid having a N-terminal amine group, or any amino acid with a side chain capable of forming an oxime or hydrazone bond upon reaction with a hydroxylamine or hydrazine group. c_{A} comprises D-cysteine, D-glutamate, lysine, or para-4-acetyl L-phenylalanine.

### Portion c_{P}

In some embodiments, cₚ is a linker. In some embodiments, cₚ is bound to c_{A}. In some embodiments, cₚ and N are not bound at the same positions.

In some embodiments, cₚ is selected from:

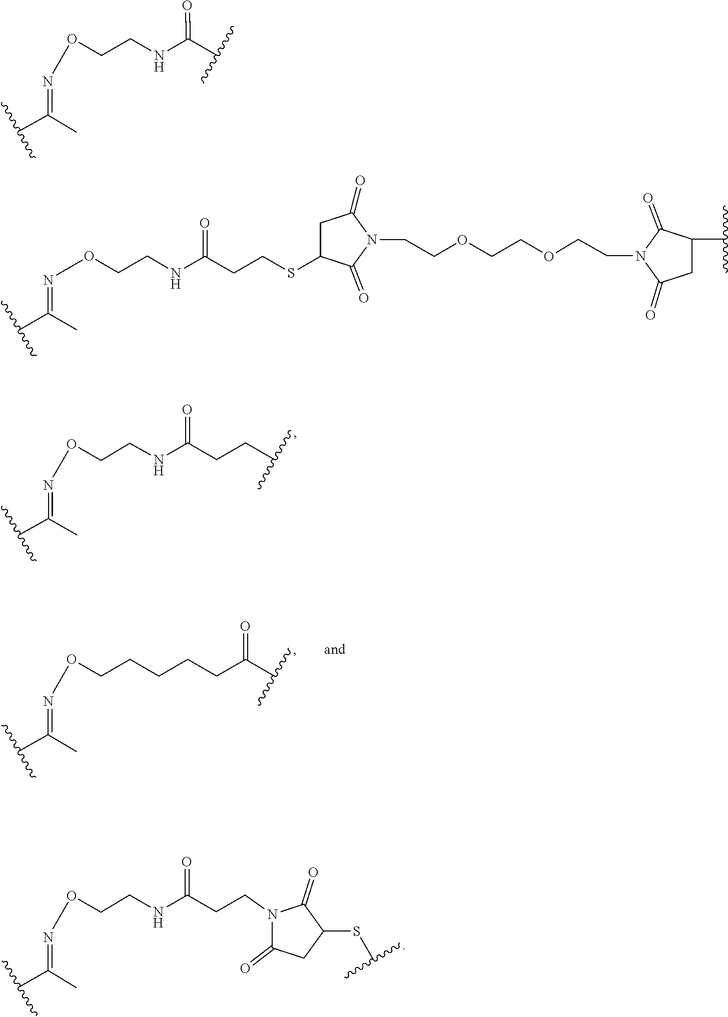 [SMILES: *C(=O)NCCO/N=C(/*)C ; */C(C)=N/OCCNC(=O)CCSC1CC(=O)N(CCOCCOCCN2C(=O)CC(*)C2=O)C1=O ; *CCC(=O)NCCO/N=C(/*)C ; *C(=O)CCCCCO/N=C(/*)C ; *SC1CC(=O)N(CCC(=O)NCCO/N=C(/*)C)C1=O]

In some embodiments, cₚ is selected from:

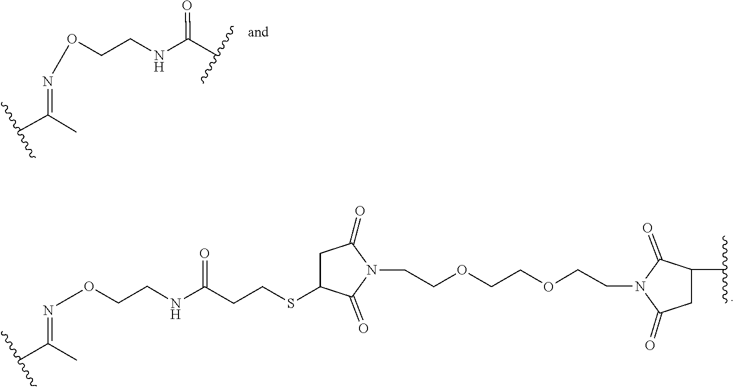 [SMILES: *C(=O)NCCO/N=C(/*)C ; */C(C)=N/OCCNC(=O)CCSC1CC(=O)N(CCOCCOCCN2C(=O)CC(*)C2=O)C1=O]

In some embodiments, cₚ is

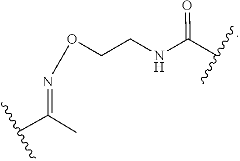 [SMILES: *C(=O)NCCO/N=C(/*)C]

In some embodiments, cₚ is

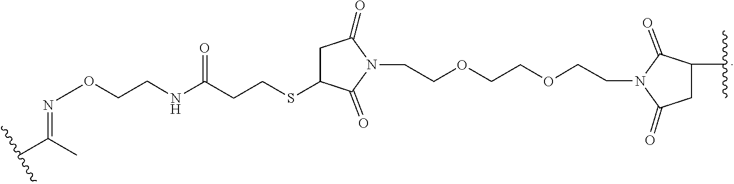 [SMILES: */C(C)=N/OCCNC(=O)CCSC1CC(=O)N(CCOCCOCCN2C(=O)CC(*)C2=O)C1=O]

In some embodiments, cₚ is

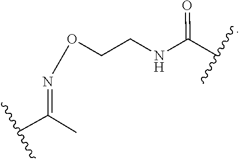 [SMILES: *C(=O)NCCO/N=C(/*)C]

In some embodiments, cₚ is

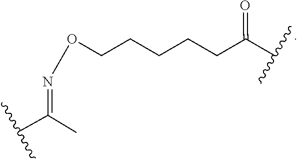 [SMILES: *C(=O)CCCCCO/N=C(/*)C]

In some embodiments, cₚ is

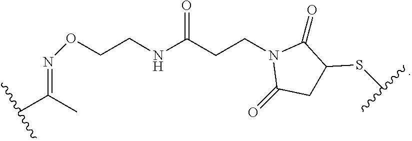 [SMILES: *SC1CC(=O)N(CCC(=O)NCCO/N=C(/*)C)C1=O]

In some embodiments, cₚ comprises 0-10 amino acids. In some embodiments, cₚ comprises 0-1 amino acids. In some embodiments, cₚ comprises 0-3 amino acids. In some embodiments, cₚ is a bond. In some embodiments, cₚ comprises 1 amino acid. In some embodiments, cₚ comprises 2 amino acids. In some embodiments, cₚ comprises 3 amino acids. In some embodiments, cₚ comprises 4 amino acid. In some embodiments, cₚ comprises 5 amino acids. In some embodiments, cₚ comprises 6 amino acids. In some embodiments, cₚ comprises 7 amino acids. In some embodiments, cₚ comprises 8 amino acids. In some embodiments, cₚ comprises 9 amino acids. In some embodiments, cₚ comprises 10 amino acids.

In some embodiments, cₚ comprises a derivatized amino acid.

In some embodiments, cₚ comprises a naturally-occurring amino acid or a non-naturally-occurring amino acid. In some embodiments, cₚ is selected from a D amino acid, a L amino acid, an ex-amine) acid, a β-amino acid, or a γ-amino acid. In some embodiments, cₚ comprises any amino acid having a free thiol group, any amino acid containing a free amine group, any amino acid having a N-terminal amine group, or any amino acid with a side chain capable of forming an oxime or hydrazone bond upon reaction with a hydroxylamine or hydrazine group. cₚ comprises D-cysteine, D-glutamate, lysine, or para-4-acetyl L-phenylalanine.

### Portion c_{B}

In some embodiments, c_{B} comprises 0-10 amino acids. In some embodiments, c_{B} comprises 0-1 amino acids. In some embodiments, c_{B} comprises 0-3 amino acids. In some embodiments, c_{B} is a bond. In some embodiments, c_{B} comprises 1 amino acid. In some embodiments, c_{B} comprises 2 amino acids. In some embodiments, c_{B} comprises 3 amino acids. In some embodiments, c_{B} comprises 4 amino acid. In some embodiments, c_{B} comprises 5 amino acids. In some embodiments, c_{B} comprises 6 amino acids. In some embodiments, c_{B} comprises 7 amino acids. In some embodiments, c_{B} comprises 8 amino acids. In some embodiments, c_{B} comprises 9 amino acids. In some embodiments, c_{B} comprises 10 amino acids.

In some embodiments, c_{B} comprises a derivatized amino acid.

In some embodiments, c_{B} comprises a naturally-occurring amino acid or a non-naturally-occurring amino acid. In some embodiments, c_{B} is selected from a D amino acid, a L amino acid, an α-amino acid, a β-amino acid, or a γ-amino acid. In some embodiments, c_{B} comprises any amino acid having a free thiol group, any amino acid containing a free amine group, any amino acid having a N-terminal amine group, or any amino acid with a side chain capable of forming an oxime or hydrazone bond upon reaction with a hydroxylamine or hydrazine group. c_{B} comprises D-cysteine, D-glutamate, lysine, or para-4-acetyl L-phenylalanine.

### Portion S

In some embodiments, S is an electrophilic substituted. In some embodiments, S is bound to the amino terminus of A or c_{A}. In some embodiments, S is an N-maleimide or N-succinamide substituent. In some embodiments, S is bound to the amino terminus of A. In other embodiments, S is bound to c_{A}. In some embodiments, S is bound to the amino terminus of A and S is bound to N. In some embodiments wherein S is present, S and N are not bound at the same position. In some embodiments, S is optionally present. In some embodiments, w is 0. In some embodiments, w is 1.

In some embodiments, S is

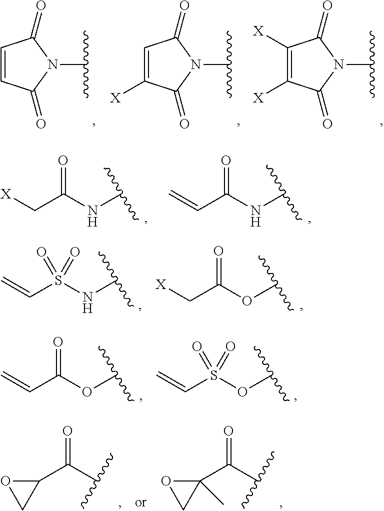

wherein each X is independently —Cl, —Br, —I, or —S-phenyl.

In some embodiments, S is

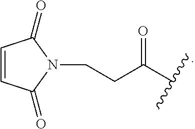 [SMILES: *C(=O)CCN1C(=O)C=CC1=O]

In some embodiments, S is

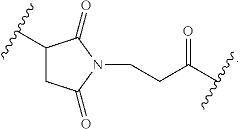 [SMILES: *C(=O)CCN1C(=O)CC(*)C1=O]

In some embodiments, S is further conjugated to an albumin.

### Linker X

In some embodiments, X is a linker consisting of one or more amino acids is used to join peptide sequence A and peptide sequence B. In some instances, the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In live cells, an intact selective delivery molecule disclosed herein may not be able to enter the cell because of the presence of portion of A. Thus, a strictly intracellular process for cleaving X would be ineffective to cleave X in healthy cells since portion of A, preventing uptake into cells, would not be effectively cleaved by intracellular enzymes in healthy cells since it would not be taken up and would not gain access to such intracellular enzymes. However, where a cell is injured or diseased (e.g., cancerous cells, hypoxic cells, ischemic cells, apoptotic cells, necrotic cells) such intracellular enzymes leak out of the cell and cleavage of A would occur, allowing entry of portion of B and/or D_{B} into the cell, effecting targeted delivery of portion of B and/or D_{B} to neighboring cells. In some embodiments, X is cleaved in the extracellular space.

In some embodiments, the fact that capillaries are often leaky around tumors and other trauma sites enhances the ability of high molecular weight molecules (e.g., molecular weight of about 30 kDa or more) to reach the interstitial compartment. In some embodiments, cells that do not express the relevant protease but that are immediately adjacent to expressing cells pick up D_{B} from a selective delivery molecule because linkage of a X linker is typically extracellular. In some embodiments, such bystander targeting is beneficial in the treatment of tumors because of the heterogeneity of cell phenotypes and the wish to eliminate as high a percentage of suspicious cells as possible.

In some embodiments, X is a cleavable linker.

In some embodiments, the X linker is flexible. In some embodiments, the linker is rigid.

In some embodiments, the X linker comprises a linear structure. In some embodiments, the X linker comprises a non-linear structure. In some embodiments, the X linker comprises a branched structure. In some embodiments, the X linker comprises a cyclic structure.

In some embodiments, X is about 5 to about 30 atoms in length. In some embodiments, X is about 6 atoms in length. In some embodiments, X is about 8 atoms in length. In some embodiments, X is about 10 atoms in length. In some embodiments, X is about 12 atoms in length. In some embodiments, X is about 14 atoms in length. In some embodiments, X is about 16 atoms in length. In some embodiments, X is about 18 atoms in length. In some embodiments, X is about 20 atoms in length. In some embodiments, X is about 25 atoms in length. In some embodiments, X is about 30 atoms in length.

In some embodiments, the linker binds peptide portion of A (i.e., the peptide sequence which prevents cellular uptake) to peptide portion of B (i.e., the delivery sequence) by a covalent linkage. In some embodiments, the covalent linkage comprises an ether bond, thioether bond, amine bond, amide bond, oxime bond, hydrazone bond, carbon-carbon bond, carbon-nitrogen bond, carbon-oxygen bond, or carbon-sulfur bond.

In some embodiments, X comprises a peptide linkage. The peptide linkage comprises L-amino acids and/or D-amino acids. In embodiments of the invention, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a X linker is designed for cleavage in the presence of particular conditions or in a particular environment. In preferred embodiments, a X linker is cleavable under physiological conditions. Cleavage of such a X linker may, for example, be enhanced or may be affected by particular pathological signals or a particular environment related to cells in which D_{B} delivery is desired. The design of a X linker for cleavage by specific conditions, such as by a specific enzyme, allows the targeting of cellular uptake to a specific location where such conditions obtain. Thus, one important way that selective delivery molecules provide specific targeting of cellular uptake to desired cells, tissues, or regions is by the design of the linker portion X to be cleaved by conditions near such targeted cells, tissues, or regions.

In some embodiments, X is a pH-sensitive linker. In some embodiments, X is cleaved under basic pH conditions. In some embodiments, X is cleaved under acidic pH conditions. In some embodiments, X is cleaved by a protease, a matrix metalloproteinase, a serine protease, or a combination thereof. In some embodiments, X is cleaved by a reducing agent. In some embodiments, X is cleaved by an oxidizing agent or oxidative stress.

In some embodiments, X is cleaved by an MMP. The hydrolytic activity of matrix metalloproteinases (MMPs) has been implicated in the invasive migration of metastatic tumor cells. In certain instances, MMPs are found near sites of inflammation. In certain instances, MMPs are found near sites of stroke (i.e., a disorder characterized by brain damage following a decrease in blood flow). Thus, uptake of molecules having features of the invention are able to direct cellular uptake of D_{B} (at least one D moiety) to specific cells, tissues, or regions having active MMPs in the extracellular environment. In some embodiments, a X linker that includes the amino-acid sequences PLG-C(Me)-AG, PLGLAG which are cleaved by the metalloproteinase enzymes MMP-2, MMP-9, or MMP-7 (MMPs involved in cancer and inflammation).

In some embodiments, X is cleaved by proteolytic enzymes or reducing environment, as may be found near cancerous cells. Such an environment, or such enzymes, are typically not found near normal cells.

In some embodiments, X is cleaved by serine proteases including but not limited to thrombin and cathepsins. In some embodiments, X is cleaved by cathepsin K, cathepsin S, cathepsin D, cathepsin E, cathepsin W, cathepsin F, cathepsin A, cathepsin C, cathepsin H, cathepsin Z, or any combinations thereof. In some embodiments, X is cleaved by cathepsin K and/or cathepsin S.

In some embodiments, X is cleaved in or near tissues suffering from hypoxia. In some embodiments, cleavage in or near hypoxic tissues enables targeting of cancer cells and cancerous tissues, infarct regions, and other hypoxic regions. In some embodiments, X comprises a disulfide bond. In some embodiments, a linker comprising a disulfide bond is preferentially cleaved in hypoxic regions and so targets D_{B} delivery to cells in such a region. Hypoxia is thought to cause cancer cells to become more resistant to radiation and chemotherapy, and also to initiate angiogenesis. In a hypoxic environment in the presence of, for example, leaky or necrotic cells, free thiols and other reducing agents become available extracellularly, while the O₂ that normally keeps the extracellular environment oxidizing is by definition depleted. In some embodiments, this shift in the redox balance promotes reduction and cleavage of a disulfide bond within a X linker. In addition to disulfide linkages which take advantage of thiol-disulfide equilibria, linkages including quinones that fall apart when reduced to hydroquinones are used in a X linker designed to be cleaved in a hypoxic environment.

In some embodiments, X is cleaved in a necrotic environment. Necrosis often leads to the release of enzymes or other cell contents that may be used to trigger cleavage of a X linker. In some embodiments, cleavage of X by necrotic enzymes (e.g., by calpains) allows D_{B} to be taken up by diseased cells and by neighboring cells that had not yet become fully leaky.

In some embodiments, X is an acid-labile linker. In some embodiments, X comprises an acetal or vinyl ether linkage. Acidosis is observed in sites of damaged or hypoxic tissue, due to the Warburg shift from oxidative phosphorylation to anaerobic glycolysis and lactic acid production. In some embodiments, acidosis is used as a trigger of D_{B} uptake by replacing some of the arginines within B by histidines, which only become cationic below pH 7.

It will be understood that a linker X disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

In some embodiments, the linker X comprises an amino acid sequence selected from: PLGLAG, PLG-C(me)-AG, RPLALWRS, ESPAYYTA, DPRSFL, PPRSFL, RLQLKL, and RLQLK(Ac)L. In some embodiments, the linker X comprises the amino acid sequence PLGLAG. In some embodiments, the linker X comprises the amino acid sequence PLG-C(me)-AG. In some embodiments, the linker X comprises the amino acid sequence PLGxAG, wherein x is any amino acid (naturally-occuring or non-naturally occurring). In some embodiments, the linker X comprises the amino acid sequence RPLALWRS. In some embodiments, the linker X comprises the amino acid sequence ESPAYYTA. In some embodiments, the linker X comprises the amino acid sequence DPRSFL. In some embodiments, the linker X comprises the amino acid sequence PPRSFL. In some embodiments, the linker X comprises the amino acid sequence RLQLKL. In some embodiments, the linker X comprises the amino acid sequence RLQLK(Ac)L.

In some embodiments, X comprises —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—,

In some embodiments, the linker X comprises a peptide selected from: PR(S/T)(L/I)(S/T), where the letters in parentheses indicate that either one of the indicated amino acids may be at that position in the sequence); GGAANLVRGG; SGRIGFLRTA; SGRSA; GFLG; ALAL; FK; PIC(Et)F—F, where C(Et) indicates S-ethylcysteine (a cysteine with an ethyl group attached to the thiol) and the indicates the typical cleavage site in this and subsequent sequences); GGPRGLPG; HSSKLQ; LVLA-SSSFGY; GVSQNY-PIVG; GWQA-SCRLA; f(Pip)R—S, where “f” indicates D-phenylalanine and “Pip” indicates piperidine-2-carboxylic acid (pipecolinic acid, a proline analog having a six-membered ring); DEVD; GWEHDG; RPLALWRS, or a combination thereof.

In some embodiments, X is cleaved under hypoxic conditions. In some embodiments, X comprises a disulfide linkage. In some embodiments, X comprises a quinine.

In some embodiments, X is cleaved under necrotic conditions. In some embodiments, X comprises a molecule cleavable by a cal pain.

In some embodiments, X comprises 6-aminohexanoyl, 5-(amino)-3-oxapentanoyl, or a combination thereof. In some embodiments, X comprises a disulfide linkage.

In some embodiments, the linker is an alkyl. In some embodiments, the linker is heteroalkyl.

In some embodiments, the linker is an alkylene. In some embodiments, the linker is an alkenylene. In some embodiments, the linker is an alkynylene. In some embodiments, the linker is a heteroalkylene.

In some embodiments, a selective delivery molecules disclosed herein comprises a single of linker. Use of a single mechanism to mediate uptake of both imaging and therapeutic cargoes is particularly valuable, because imaging with noninjurious tracer quantities can be used to test whether a subsequent therapeutic dose is likely to concentrate correctly in the target tissue.

In some embodiments, a selective delivery molecules disclosed herein comprises a plurality of linkers. Where a selective delivery molecule disclosed herein includes multiple X linkages, separation of portion of A from the other portions of the molecule requires cleavage of all X linkages. Cleavage of multiple X linkers may be simultaneous or sequential. Multiple X linkages may include X linkages having different specificities, so that separation of portion of A from the other portions of the molecule requires that more than one condition or environment (“extracellular signals”) be encountered by the molecule. Cleavage of multiple X linkers thus serves as a detector of combinations of such extracellular signals. For example, a selective delivery molecule may include two linker portions Xa and Xb connecting basic portion of B with acidic portion of A. Both linkers Xa and Xb must be cleaved before acidic portion of A is separated from basic portion of B allowing entry of portion of B and cargo moiety D_{B} to enter a cell. It will be understood that a linker region may link to either a basic portion of B or a cargo moiety D_{B} independently of another linker that may be present, and that, where desired, more than two linker regions X may be included.

Combinations of two or more X linkers may be used to further modulate the targeting and delivery of molecules to desired cells, tissue or regions. Combinations of extracellular signals are used to widen or narrow the specificity of the cleavage of X linkers if desired. Where multiple X linkers are linked in parallel, the specificity of cleavage is narrowed, since each X linker must be cleaved before portion of A may separate from the remainder of the molecule. Where multiple X linkers are linked in series, the specificity of cleavage is broadened, since cleavage of any one X linker allows separation of portion of A from the remainder of the molecule. For example, in order to detect either a protease OR hypoxia (i.e., to cleave X in the presence of either protease or hypoxia), a X linker is designed to place the protease-sensitive and reduction-sensitive sites in tandem, so that cleavage of either would suffice to allow separation of the acidic portion of A. Alternatively, in order to detect the presence of both a protease AND hypoxia (i.e., to cleave X in the presence of both protease and hypoxia but not in the presence of only one alone), a X linker is designed to place the protease sensitive site between at least one pair of cysteines that are disulfide-bonded to each other. In that case, both protease cleavage and disulfide reduction are required in order to allow separation of portion of A.

### Portion N

In some embodiments, each N is independently selected from a bond and a linker. In some embodiments, each N is independently bound to c_{A}, c_{B}, or the amino terminus of A. In some embodiments, S and N are not bound at the same position. In some embodiments, cₚ and N are not bound at the same position. In some embodiments, N is independently bound to G or c_{A}. In some embodiments, wherein if v is 2, the two N are not identical.

In some embodiments, N is a bond or a linker selected from the group consisting of:

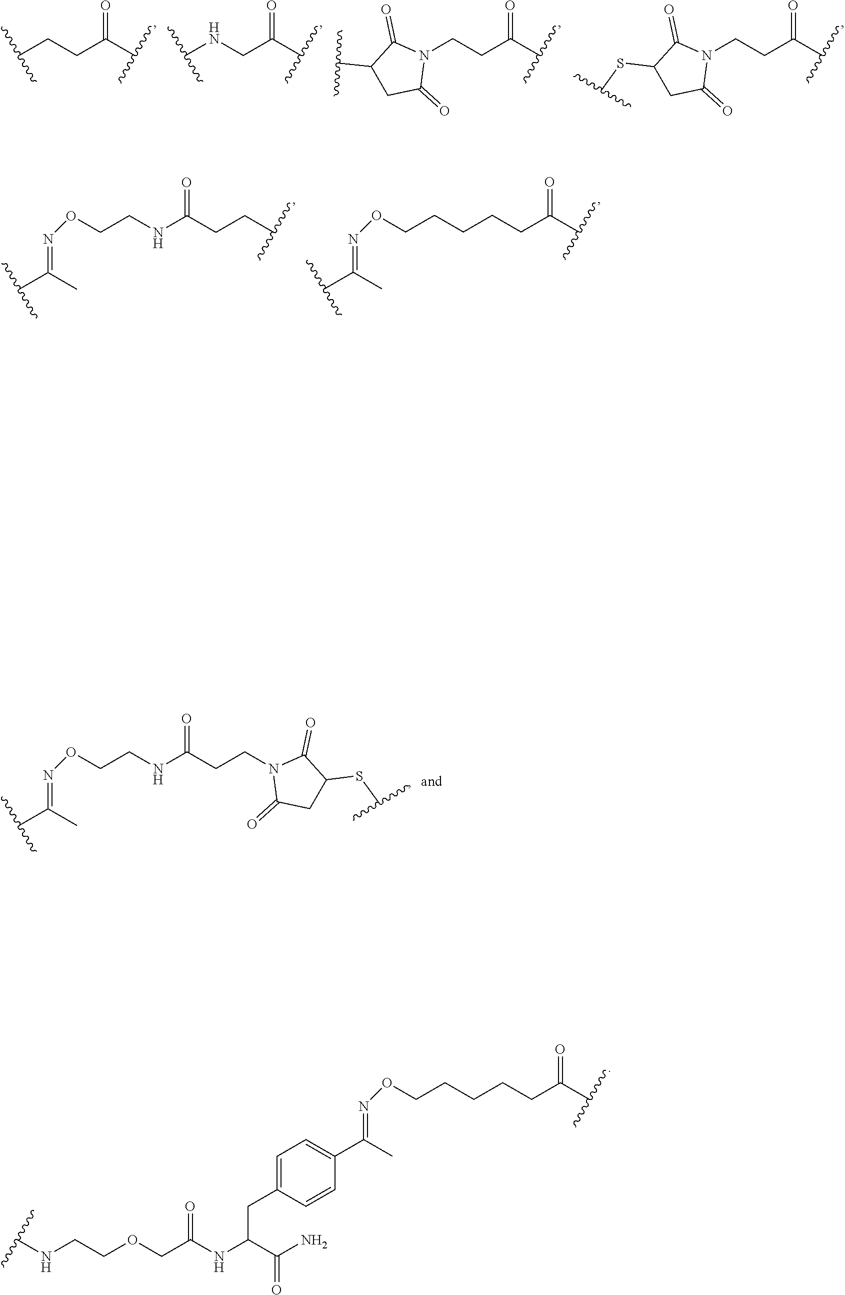

In some embodiments, wherein N is a bond,

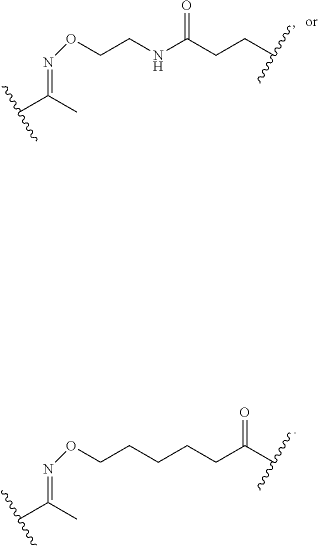 [SMILES: *CCC(=O)NCCO/N=C(/*)C ; *C(=O)CCCCCO/N=C(/*)C]

In some embodiments, N is

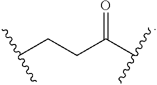 [SMILES: *CCC(*)=O]

In some embodiments, N is

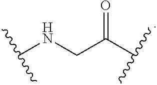 [SMILES: *NCC(*)=O]

In some embodiments, N is

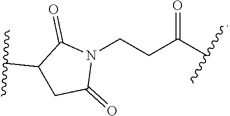 [SMILES: *C(=O)CCN1C(=O)CC(*)C1=O]

In some embodiments, N is

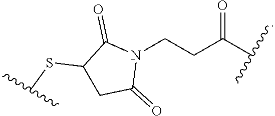 [SMILES: *SC1CC(=O)N(CCC(*)=O)C1=O]

In some embodiments, N is

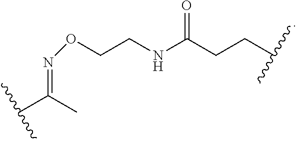 [SMILES: *CCC(=O)NCCO/N=C(/*)C]

In some embodiments, N is

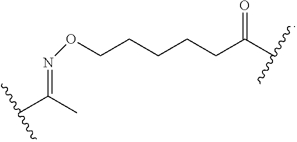 [SMILES: *C(=O)CCCCCO/N=C(/*)C]

In some embodiments, N is

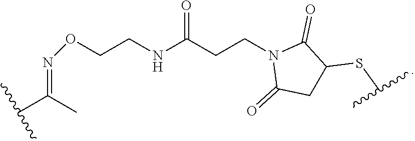 [SMILES: *SC1CC(=O)N(CCC(=O)NCCO/N=C(/*)C)C1=O]

In some embodiments, N is

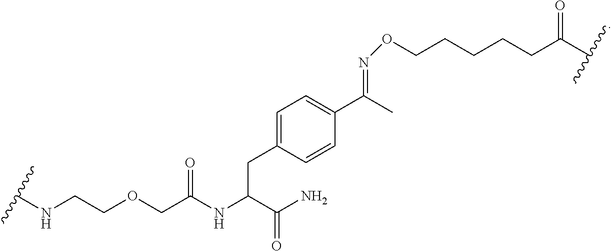 [SMILES: *NCCOCC(=O)NC(Cc1ccc(/C(C)=N/OCCCCCC(*)=O)cc1)C(N)=O]

### Portion Z

In some embodiments, each Z is independently a peptide with a sequence comprising 1 to 10 amino acids. In some embodiments, each Z is independently a peptide with a sequence comprising 2 to 10 amino acids. In some embodiments, each Z is independently a peptide with a sequence comprising 3 to 10 amino acids. In some embodiments, each Z is independently a peptide with a sequence comprising 4 to 10 amino acids. In some embodiments, each Z is independently a peptide with a sequence comprising 5 to 10 amino acids. In some embodiments, each Z is independently a peptide with a sequence comprising 1 to 20 amino acids.

In some embodiments, Z comprises a receptor binding peptide. Exemplary receptor binding peptide comprises an urokinase type plasminogen activator receptor (uPAR) peptide.

In some embodiments, Z comprises a urokinase type plasminogen activator receptor (uPAR) peptide. Exemplary uPAR peptides include, but are not limited to, SRSRY. SRNRY. SRGRY, SQSRY, SQNRY, SQGRY, PRSRY, PRNRY, PRGRY, PQSRY. PQNRY, or PQGRY. In some embodiments, Z comprises SRSRY, SRNRY, SRGRY, SQSRY, SQNRY, SQGRY, PRSRY, PRNRY, PRGRY, PQSRY, PQNRY, or PQGRY.

In some embodiments, Z comprises a series of 4 Phe residues.

In some embodiments, Z is a peptide with a sequence selected from the group consisting of:

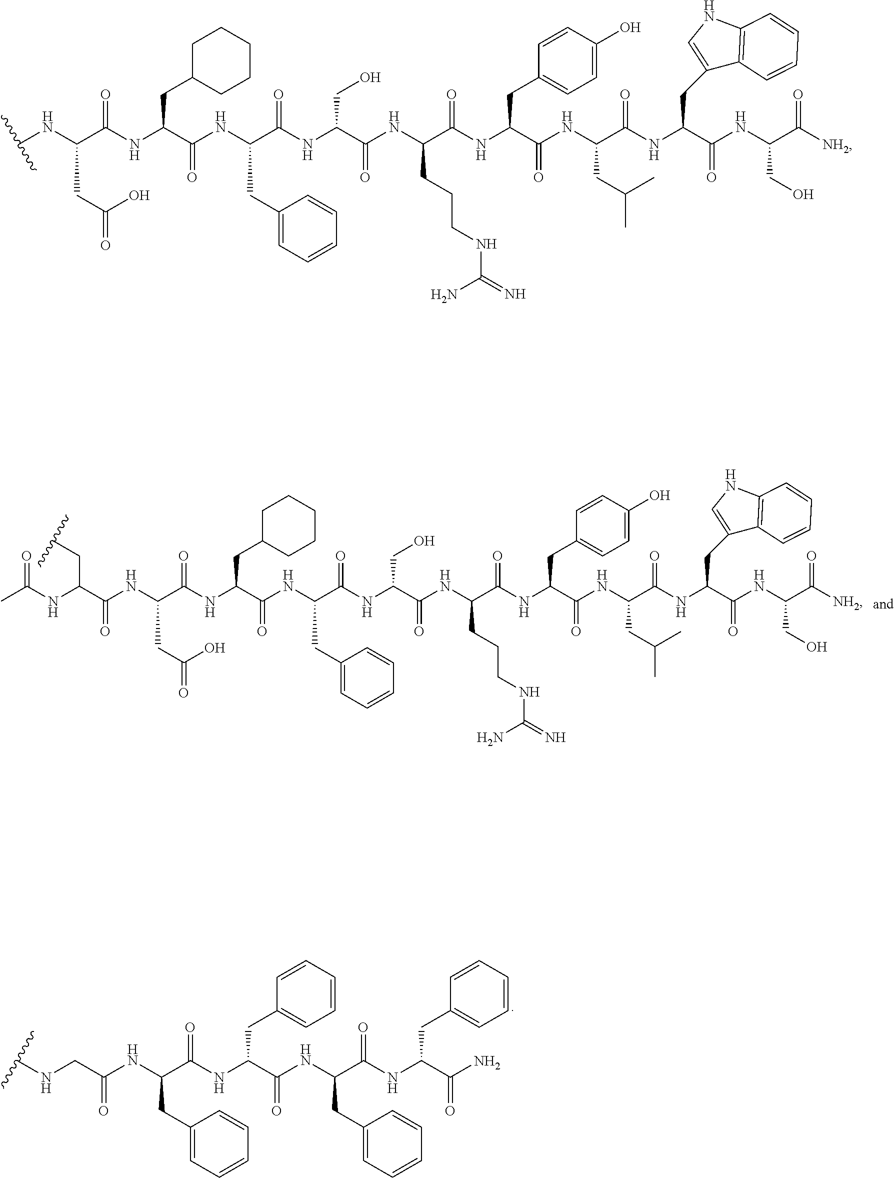

In some embodiments, Z is

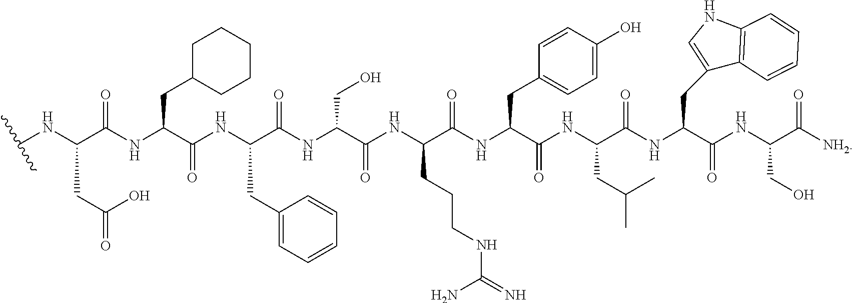 [SMILES: *N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(O)cc1)C(=O)N[C@@H](CC(C)C)C(=O)N[C@@H](Cc1c[nH]c2ccccc12)C(=O)N[C@@H](CO)C(N)=O]

In some embodiments, Z is

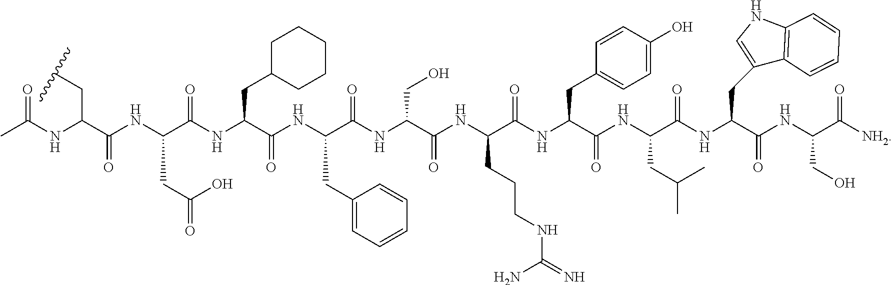 [SMILES: *CC(NC(C)=O)C(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(O)cc1)C(=O)N[C@@H](CC(C)C)C(=O)N[C@@H](Cc1c[nH]c2ccccc12)C(=O)N[C@@H](CO)C(N)=O]

In some embodiments, Z is

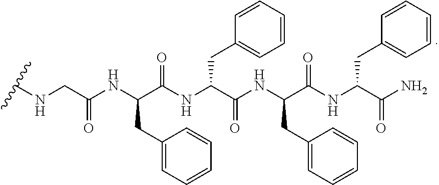 [SMILES: *NCC(=O)N[C@H](Cc1ccccc1)C(=O)N[C@H](Cc1ccccc1)C(=O)N[C@H](Cc1ccccc1)C(=O)N[C@H](Cc1ccccc1)C(N)=O]

In some embodiments, Z is

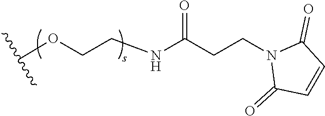

wherein s is 1 to 20.

In some embodiments, Z is a polyethylene glycol substituent.

Carriers (M, M¹, or M²)

In some embodiments, M, M¹, and M² are each independently a carrier. In some embodiments, a carrier is a macromolecule such as a protein, a synthetic or natural polymer, or a dendrimer. In some embodiments, M, M¹, and M² are each independently selected from dextran, a PEG polymer (e.g., a PEG polymer having an average molecular weight of approximately 0.5 kDa (PEG 0.5 kDa), approximately 1 kDa (PEG 1 kDa), approximately 2 kDa (PEG 2 kDa), approximately (PEG 3 kDa), approximately 4 kDa (PEG 4 kDa), approximately 5 kDa (PEG 5 kDa), approximately 10 kDa (PEG 10 kDa), approximately 12 kDa (PEG 12 kDa), approximately 15 kDa (PEG 15 kDa), approximately 20 kDa (PEG 20 kDa), approximately 30 kDa (PEG 30 kDa), or approximately 40 kDa (PEG 40 kDa)), or albumin. In some embodiments, M, M¹, and M² are each independently a PEG polymer.

Polymers are characterized by a distribution of molecular weights, and, as such, the molecular weight, presented herein for polymers, is only an approximate average molecular weight of a distribution of molecular weights of individual polymers. Unless stated otherwise, the molecular weight of a polymeric component will have a typical (i.e., as known in the art) error and standard deviation.

In some embodiments, the molecular weight of a polyethylene glycol substituted (PEG) is about 200; 300; 400; 500; 600; 700; 800; 900; 1000; 1100; 1200; 1300; 1400; 1450; 1500; 1600; 1700; 1800; 1900; 2000; 2100; 2200; 2300; 2400; 2500; 2600; 2700; 2800; 2900; 3000; 3250; 3350; 3500; 3750; 4000; 4250; 4500; 4600; 4750; 5000; 5500; 6000; 6500; 7000; 7500, 8000; 10,000; 12,000, 20,000; 35,000; 40,000; 50,000; 60,000; or 100,000 Da.

In some embodiments, M is a polyethylene glycol substituent with a substituent mass of at least 500 Daltons. In some embodiments, M is a polyethylene glycol substituent with a substituent mass of at least 1,000 Daltons. In some embodiments, M is a polyethylene glycol substituent with a substituent mass of at least 2,000 Daltons. In some embodiments, M is a polyethylene glycol substituent with a substituent mass of at least 3,000 Daltons. In some embodiments, M is a polyethylene glycol substituent with a substituent mass of at least 4,000 Daltons. In some embodiments, M is a polyethylene glycol substituent with a substituent mass of at least 5,000 Daltons. In some embodiments, M is a polyethylene glycol substituent with a substituent mass of at least 10,000 Daltons. In some embodiments, M is a polyethylene glycol substituent with a substituent mass of at least 15,000 Daltons. In some embodiments, M is a polyethylene glycol substituent with a substituent mass of at least 20,000 Daltons. In some embodiments, M is a polyethylene glycol substituent with a substituent mass of at least 25,000 Daltons. In some embodiments, M is a polyethylene glycol substituent with a substituent mass of at least 30,000 Daltons. In some embodiments, M is a polyethylene glycol substituent with a substituent mass of at least 35,000 Daltons. In some embodiments, M is a polyethylene glycol substituent with a substituent mass of at least 40,000 Daltons. In some embodiments, M is a polyethylene glycol substituent with a substituent mass of at least 45,000 Daltons. In some embodiments, M is a polyethylene glycol substituent with a substituent mass of at least 50,000 Daltons. In some embodiments, M is a polyethylene glycol substituent with a substituent mass of at least 100,000 Daltons.

In some embodiments, M is a polyethylene glycol substituent with a substituent mass of approximately 500 to approximately 100,000 Daltons. In some embodiments, M is a polyethylene glycol substituent with a substituent mass of approximately 1,000 to approximately 50,000 Daltons. In some embodiments, M is a polyethylene glycol substituent with a substituent mass of approximately 2,000 to approximately 40,000 Daltons.

In some embodiments, M is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3,4, 5, 6, 7, 8, 9, 10, 11, 12,13, 14, 15, 16, 17,18, 19, 20, 22, 24, 26, 28, 30, 35,40,42,48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, M¹ is a polyethylene glycol substituent with a substituent mass of at least 500 Daltons. In some embodiments, M¹ is a polyethylene glycol substituent with a substituent mass of at least 1,000 Daltons. In some embodiments, M¹ is a polyethylene glycol substituent with a substituent mass of at least 2,000 Daltons. In some embodiments, M¹ is a polyethylene glycol substituent with a substituent mass of at least 3,000 Daltons. In some embodiments, M¹ is a polyethylene glycol substituent with a substituent mass of at least 4,000 Daltons. In some embodiments, M¹ is a polyethylene glycol substituent with a substituent mass of at least 5,000 Daltons. In some embodiments, M¹ is a polyethylene glycol substituent with a substituent mass of at least 10,000 Daltons. In some embodiments, M¹ is a polyethylene glycol substituent with a substituent mass of at least 15,000 Daltons. In some embodiments, M¹ is a polyethylene glycol substituent with a substituent mass of at least 20,000 Daltons. In some embodiments, M¹ is a polyethylene glycol substituent with a substituent mass of at least 25,000 Daltons. In some embodiments, M¹ is a polyethylene glycol substituent with a substituent mass of at least 30,000 Daltons. In some embodiments, M¹ is a polyethylene glycol substituent with a substituent mass of at least 35,000 Daltons. In some embodiments, M¹ is a polyethylene glycol substituent with a substituent mass of at least 40,000 Daltons. In some embodiments, M¹ is a polyethylene glycol substituent with a substituent mass of at least 45,000 Daltons. In some embodiments, M¹ is a polyethylene glycol substituent with a substituent mass of at least 50,000 Daltons. In some embodiments, M¹ is a polyethylene glycol substituent with a substituent mass of at least 100,000 Daltons.

In some embodiments, M¹ is a polyethylene glycol substituent with a substituent mass of approximately 500 to approximately 100,000 Daltons. In some embodiments, M¹ is a polyethylene glycol substituent with a substituent mass of approximately 1,000 to approximately 50,000 Daltons. In some embodiments, M¹ is a polyethylene glycol substituent with a substituent mass of approximately 2,000 to approximately 40,000 Daltons.

In some embodiments, M¹ is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3,4, 5, 6, 7, 8, 9, 10, 11, 12,13, 14, 15,16, 17, 18, 19,20, 22, 24, 26, 28, 30, 35,40,42,48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, M² is a polyethylene glycol substituent with a substituent mass of at least 500 Daltons. In some embodiments, M² is a polyethylene glycol substituent with a substituent mass of at least 1,000 Daltons. In some embodiments, M² is a polyethylene glycol substituent with a substituent mass of at least 2,000 Daltons. In some embodiments, M² is a polyethylene glycol substituent with a substituent mass of at least 3,000 Daltons. In some embodiments, M² is a polyethylene glycol substituent with a substituent mass of at least 4,000 Daltons. In some embodiments, M² is a polyethylene glycol substituent with a substituent mass of at least 5,000 Daltons. In some embodiments, M² is a polyethylene glycol substituent with a substituent mass of at least 10,000 Daltons. In some embodiments, M² is a polyethylene glycol substituent with a substituent mass of at least 15,000 Daltons. In some embodiments, M² is a polyethylene glycol substituent with a substituent mass of at least 20,000 Daltons. In some embodiments, M² is a polyethylene glycol substituent with a substituent mass of at least 25,000 Daltons. In some embodiments, M² is a polyethylene glycol substituent with a substituent mass of at least 30,000 Daltons. In some embodiments, M² is a polyethylene glycol substituent with a substituent mass of at least 35,000 Daltons. In some embodiments, M² is a polyethylene glycol substituent with a substituent mass of at least 40,000 Daltons. In some embodiments, M² is a polyethylene glycol substituent with a substituent mass of at least 45,000 Daltons. In some embodiments, M² is a polyethylene glycol substituent with a substituent mass of at least 50,000 Daltons. In some embodiments, M² is a polyethylene glycol substituent with a substituent mass of at least 100,000 Daltons.

In some embodiments, M² is a polyethylene glycol substituent with a substituent mass of approximately 500 to approximately 100,000 Daltons. In some embodiments, M² is a polyethylene glycol substituent with a substituent mass of approximately 1,000 to approximately 50,000 Daltons. In some embodiments, M² is a polyethylene glycol substituent with a substituent mass of approximately 2,000 to approximately 40,000 Daltons.

In some embodiments, M² is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3,4, 5, 6, 7, 8, 9, 10, 11, 12,13, 14, 15, 16, 17,18, 19, 20, 22, 24, 26, 28, 30, 35,40,42,48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, M is selected from a protein, a synthetic or natural polymer, or a dendrimer. In some embodiments, M is an albumin protein. In some embodiments, M is mouse serum albumin. In other embodiments, M is human serum albumin. In certain instances, albumin is excluded from the glomerular filtrate under normal physiological conditions. In some embodiments, the G comprises a reactive group such as maleimide that forms a covalent conjugate with an albumin. A selective delivery molecule comprising albumin results in enhanced accumulation of cleaved selective delivery molecules in tumors in a cleavage dependent manner. In some embodiments, albumin conjugates have good pharmacokinetic properties. Albumin is a carrier for tumor targeting because it accumulates in solid tumors due to the pathophysiology of tumor tissue, characterized by a high metabolic turnover, angiogenesis, hypervasculature, a defective vascular architecture and an impaired lymphatic drainage. The unique free sulfhydryl group (Cys-34) of albumin, which is not present in the majority of circulating serum proteins, is accessible for selective modifications. Albumin-drug conjugates show improved the pharmacokinetic profiles. However, albumin conjugates have limited tumor penetration and distribution due to their big molecular size and the tumor tissue's microenvironment, such as increased interstitial fluid pressure and dense extracellular matrix. In some embodiments, thiol-reactive SDMs provided herein form albumin conjugates in vivo. In some embodiments, the albumin carrier increases the drug's tumor penetration. In some embodiments, the albumin carrier improves the drug's distribution and activity. In some embodiments, after injected into blood stream, thiol-reactive SDMs react with the free Cys34 thiol of the circulating albumin. The albumin-SDM conjugate is then transported and accumulated in the tumor tissues.

In some embodiments, M¹ is selected from a protein, a synthetic or natural polymer, or a dendrimer. In some embodiments, M¹ is an albumin protein. In some embodiments, M¹ is mouse serum albumin. In other embodiments, M¹ is human serum albumin. In certain instances, albumin is excluded from the glomerular filtrate under normal physiological conditions. In some embodiments, the G comprises a reactive group such as maleimide that forms a covalent conjugate with an albumin. A selective delivery molecule comprising albumin results in enhanced accumulation of cleaved selective delivery molecules in tumors in a cleavage dependent manner. In some embodiments, albumin conjugates have good pharmacokinetic properties. Albumin is a carrier for tumor targeting because it accumulates in solid tumors due to the pathophysiology of tumor tissue, characterized by a high metabolic turnover, angiogenesis, hypervasculature, a defective vascular architecture and an impaired lymphatic drainage. The unique free sulfhydryl group (Cys-34) of albumin, which is not present in the majority of circulating serum proteins, is accessible for selective modifications. Albumin-drug conjugates show improved the pharmacokinetic profiles. However, albumin conjugates have limited tumor penetration and distribution due to their big molecular size and the tumor tissue's microenvironment, such as increased interstitial fluid pressure and dense extracellular matrix. In some embodiments, thiol-reactive SDMs provided herein form albumin conjugates in vivo. In some embodiments, the albumin carrier increases the drug's tumor penetration. In some embodiments, the albumin carrier improves die drug's distribution and activity. In some embodiments, after injected into blood stream, thiol-reactive SDMs react with the free Cys34 thiol of the circulating albumin. The albumin-SDM conjugate is then transported and accumulated in the tumor tissues.

In some embodiments, cₚ-M¹ is selected from the group consisting of:

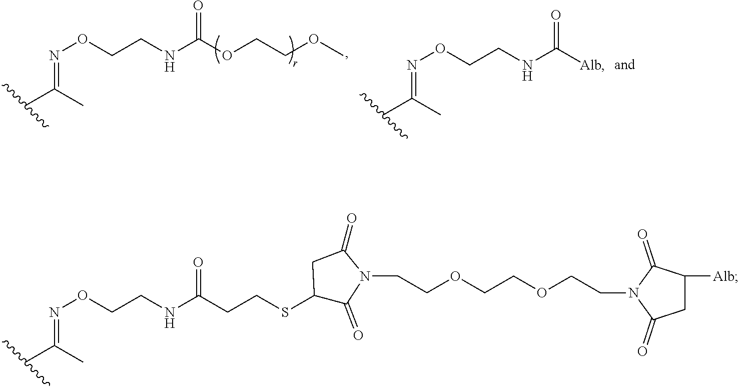

Wherein Alb is an albumin protein and each r is independently an integer ranging from 40-1,100. In some embodiments, cₚ-M¹ is selected from the group consisting of:

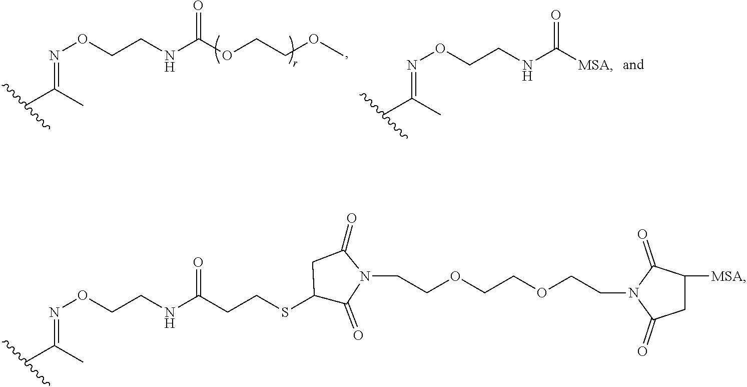

In some embodiments, M² is selected from a protein, a synthetic or natural polymer, or a dendrimer. In some embodiments, M² is an albumin protein. In some embodiments, M² is mouse serum albumin. In other embodiments, M² is human serum albumin. In certain instances, albumin is excluded from the glomerular filtrate under normal physiological conditions. In some embodiments, the G comprises a reactive group such as maleimide that forms a covalent conjugate with an albumin. A selective delivery molecule comprising albumin results in enhanced accumulation of cleaved selective delivery molecules in tumors in a cleavage dependent manner. In some embodiments, albumin conjugates have good pharmacokinetic properties. Albumin is a carrier for tumor targeting because it accumulates in solid tumors due to the pathophysiology of tumor tissue, characterized by a high metabolic turnover, angiogenesis, hypervasculature, a defective vascular architecture and an impaired lymphatic drainage. The unique free sulfhydryl group (Cys-34) of albumin, which is not present in the majority of circulating serum proteins, is accessible for selective modifications. Albumin-drug conjugates show improved the pharmacokinetic profiles. However, albumin conjugates have limited tumor penetration and distribution due to their big molecular size and the tumor tissue's microenvironment, such as increased interstitial fluid pressure and dense extracellular matrix. In some embodiments, thiol-reactive SDMs provided herein form albumin conjugates in vivo. In some embodiments, the albumin carrier increases the drug's tumor penetration. In some embodiments, the albumin carrier improves the drug's distribution and activity. In some embodiments, after injected into blood stream, thiol-reactive SDMs react with the free Cys34 thiol of the circulating albumin. The albumin-SDM conjugate is then transported and accumulated in the tumor tissues.

In some embodiments, M²-S is selected from:

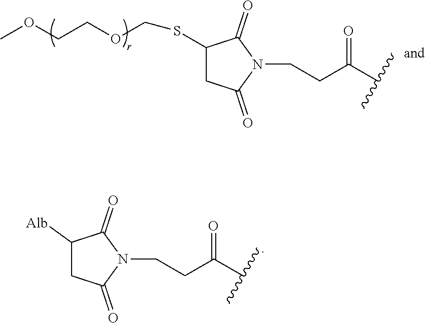

In some embodiments, M²-S is selected from the group consisting of:

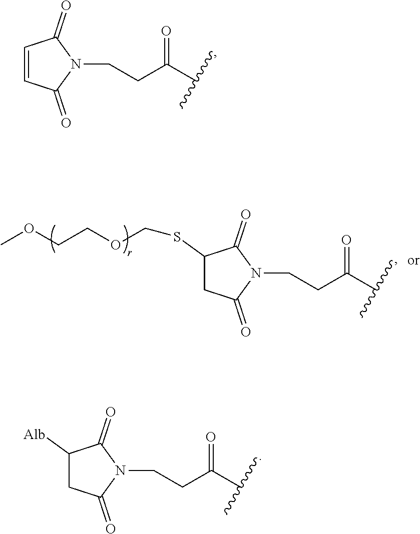

In some embodiments, M²-S is selected from the group consisting of:

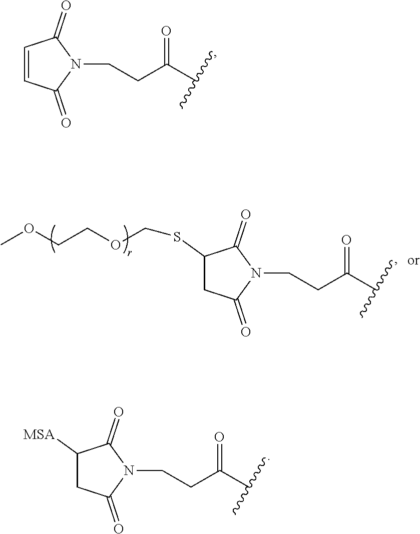

In some embodiments, r is independently an integer ranging from 40-1,100.

In some embodiments, a carrier (e.g., M, M¹, or M²) modulates plasma half-life of a selective delivery molecule disclosed herein. In some embodiments, a carrier (e.g., M, M¹, or M²) modulates solubility of a selective delivery molecule disclosed herein. In some embodiments, a carrier (e.g., M, M¹, or M²) modulates bio-distribution of a selective delivery molecule disclosed herein.

In some embodiments, a carrier (e.g., M, M¹, or M²) decreases uptake of a selective delivery molecule by non-target cells or tissues. In some embodiments, a carrier (e.g., M, M¹, or M²) decreases uptake of a selective delivery molecule into cartilage. In some embodiments, a carrier (e.g., M, M¹, or M²) decreases uptake of a selective delivery molecule into joints relative to target tissue.

In some embodiments, a carrier (e.g., M, M¹, or M²) increases uptake of a selective delivery molecule by target cells or tissues. In some embodiments, a carrier (e.g., M, M¹, or M²) decreases uptake of a selective delivery molecule into the liver relative to target tissue. In some embodiments, a carrier (e.g., M, M¹, or M²) decreases uptake of a selective delivery molecule into kidneys. In some embodiments, a carrier (e.g., M, M¹, or M²) enhances uptake into cancer tissue. In some embodiments, a carrier (e.g., M, M¹, or M²) enhances uptake into lymphatic channels and/or lymph nodes.

In some embodiments, a carrier (e.g., M, M¹, or M²) increases plasma half-life by reducing glomerular filtration. In some embodiments, a carrier (e.g., M, M¹, or M²) modulates plasma half-life by increasing or decreases metabolism or protease degradation. In some embodiments, a carrier (e.g., M, M¹, or M²) increases tumor uptake due to enhanced permeability and retention (EPR) of tumor vasculature. In some embodiments, a carrier (e.g., M, M¹, or M²) increases the aqueous solubility of a selective delivery molecule.

### Cargo

Disclosed herein, in certain embodiments, is the use of a selective delivery molecule disclosed herein for delivering a therapeutic agent or an imaging agent to a tissue or a plurality of cells. In some instances, the cargo is a therapeutic agent. In other instances, the cargo is an imaging agent. In some cases, the cargo comprises D_{B}. In additional cases, D_{B} is G-T-Q-Y-D, in which D is a therapeutic agent or an imaging agent. In additional cases, D_{B} is G-T-Q-K.

### Therapeutic Agents

Disclosed herein, in certain embodiments, is the use of a selective delivery molecule disclosed herein for delivering a therapeutic agent to a tissue or a plurality of cells. In some embodiments, the therapeutic agent is an anti-inflammatory agent. In some embodiments, the therapeutic agent is an anti-cancer agent. In some embodiments, the selective delivery molecule is used to treat colorectal cancer.

In some embodiments, a D_{B} moiety is independently a therapeutic agent. In some embodiments, a D_{B} moiety comprises two or more therapeutic agents. In some embodiments, the two or more therapeutic agents are the same therapeutic agent. In some embodiments, the two or more therapeutic agents are different therapeutic agents. In some embodiments, a D_{B} moiety comprises 2,3,4, 5, 6, 7, 8, 9, 10, 11, 12 or more therapeutic agents. In some embodiments, the therapeutic agent is selected from: a chemotherapeutic agent, a steroid, an immunotherapeutic agent, a targeted therapy, an anti-inflammatory agent, or a combination thereof. In some embodiments, the therapeutic agent is a radiotherapeutic agent. In some embodiments, the therapeutic agent is a cytotoxin.

In some embodiments, D_{B} is G-T-Q-Y-D. In such cases, D is a therapeutic agent.

In some embodiments, the therapeutic agent is a B cell receptor pathway inhibitor. In some embodiments, the therapeutic agent is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCP inhibitor, or a combination thereof. In some embodiments, the therapeutic agent is an antibody, B cell receptor signaling inhibitor, a PI3K inhibitor, an LAP inhibitor, an mTOR inhibitor, a radioimmunotherapeutic, a DNA damaging agent, a proteosome inhibitor, a histone deacytlase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof. In some embodiments, the therapeutic agent is selected from: chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, bendamustine, chlorambucil, chlormethine, cyclophosphamide, ifosfamide, melphalan, prednimustine, trofosfamide, busulfan, mannosulfan, treosulfan, carboquone, thiotepa, triaziquone, carmustine, fotemustine, lomustine, nimustine, ranimustine, semustine, streptozocin, etoglucid, dacarbazine, mitobronitol, pipobroman, temozolomide, methotrexate, permetrexed, pralatrexate, raltitrexed, cladribine, clofarabine, fludarabine, mercaptopurine, nelarabine, tioguanine, azacitidine, capecitabine, carmofur, cytarabine, decitabine, fluorouracil, gemcitabine, tegafur, vinblastine, vincristine, vindesine, vinflunine, vinorelbine, etoposide, teniposide, demecolcine, docetaxel, paclitaxel, paclitaxel poliglumex, trabectedin, dactinomycin, aclarubicin, daunorubicin, doxorubicin, epirubicin, idarubicin, mitoxantrone, pirarubicin, valrubicin, zorubincin, bleomycin, ixabepilone, mitomycin, plicamycin, carboplatin, cisplatin, oxaliplatin, satraplatin, procarbazine, aminolevulinic acid, efaproxiral, methyl aminolevulinate, porfimer sodium, temoporfin, dasatinib, erlotinib, everolimus, gefitinib, imatinib, lapatinib, nilotinib, pazonanib, sorafenib, sunitinib, temsirolimus, alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat, diethylstilbenol, ethinylestradiol, fosfestrol, polyestradiol phosphate, gestonorone, medroxyprogesterone, megestrol, buserelin, goserelin, leuprorelin, triptorelin, fulvestrant, tamoxifen, toremifene, bicalutamide, flutamide, nilutamide, aminoglutethimide, anastrozole, exemestane, formestane, letrozole, vorozole, abarelix, degarelix, histamine dihydrochloride, mifamurtide, pidotimod, plerixafor, roquinimex, thymopentin, everolimus, gusperimus, leflunomide, mycophenolic acid, sirolimus, ciclosporin, tacrolimus, azathioprine, lenalidomide, methotrexate, thalidomide, iobenguane, ancestim, filgrastim, lenograstim, molgramostim, pegfilgrastim, sargramostim, interferon alfa natural, interferon alfa-2a, interferon alfa-2b, interferon alfacon-1, interferon alfa-nl, interferon beta natural, interferon beta-1a, interferon beta-1b, interferon gamma, peginterferon alfa-2a, peginterferon alfa-2b, aldesleukin, oprelvekin, BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin, abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus, adalimumab, afelimomab, certolizumab pegol, etanercept, golimumab, infliximab, anakinra, basiliximab, canakinumab, daclizumab, mepolizumab, rilonacept, tocilizumab, ustekinumab, ciclosporin, tacrolimus, azathioprine, lenalidomide, methotrexate, thalidomide, adalimumab, alemtuzumab, bevacizumab, cetuximab, certolizumab pegol, eculizumab, efalizumab, gemtuzumab, ibritumomab tiuxetan, muromonab-CD3, natalizumab, panitumumab, ranibizumab, rituximab, tositumomab, trastuzumab, catumaxomab, edrecolomab, ofatumumab, muromab-CD3, afelimomab, golimumab, ibritumomab tiuxetan, abagovomab, adecatumumab, alemtuzumab, anti-CD30 monoclonal antibody Xmab2513, anti-MET monoclonal antibody MetMab, apolizumab, apomab, arcitumomab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab, a syk inhibitor (e.g., R788), enzastaurin, dasatinib, erlotinib, everolimus, gefitinib, imatinib, lapatinib, nilotinib, pazonanib, sorafenib, sunitinib, temsirolimus, an angiogenesis inhibitor (e.g., GT-111, JI-101, R1530), a kinase inhibitors (e.g., AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, S3333333CH 727965, SGI-1176, SGX523, SNS-314, TAK-593, TAK-901, TKI258, TLN-232, TTP607, XL147, XL228, XL281RO5126766, XL418, XL765), an inhibitor of mitogen-activated protein kinase signaling (e.g., U0126, PD98059, PD184352, PD0325901, ARRY-142886, SB239063, SP600125, BAY 43-9006, wortmannin, or LY294002), adriamycin, dactinomycin, bleomycin, vinblastine, cisplatin, acivicin, aclarubicin, acodazole hydrochloride, acronine, adozelesin, aldesleukin, altretamine, ambomycin, ametantrone acetate, aminoglutethimide, amsacrine, anastrozole, andiramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, iimofosine, interleukin II (including recombinant interleukin II, or rlL2), interferon alfa-2a, interferon alfa-2b, interferon alfa-n1, interferon alfa-n3, interferon beta-1 a, interferon gamma-1 b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazoie, nogalamycin, ormaplatin, oxisuran, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinoielbine tartrate, vinrosidine sulfate, vinzolidine sulfate, vorozole, zeniplatin, zinostatin, zorubicin hydrochloride. In some embodiments, the therapeutic agent is selected from: 20-epi-1, 25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorlns, chloroquinoxaline sulfonamide, defrost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, 9-dioxamycin, diphenyl spiromustine, docosanol, dolasetron, doxifluridine, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-such as for example growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, 06-benzylguanine, octreotide, okicenone, oligonucleotides, onapristone, ondansetron, ondansetron, oracin, oral cytokine inducer, ormaplatin, osaterone, oxaliplatin, oxaunomycin, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras famesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen-binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer, mechloroethamine, cyclophosphamide, chlorambucil, busulfan, carmustine, lomusitne, decarbazine, methotrexate, cytarabine, mercaptopurine, thioguanine, pentostatin, mechloroethamine, cyclophosphamide, chlorambucil, meiphalan, ethylenimine, methylmelamine, hexamethlymelamine, thiotepa, busulfan, carmustine, lomusitne, semustine, streptozocin, decarbazine, fluorouracil, floxouridine, cytarabine, mercaptopurine, thioguanine, pentostatin, erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-LA ABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

In some embodiments, the therapeutic agent is an anti-inflammatory agent. In some embodiments, the therapeutic agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent, an alkylating agent, an anti-metabolite, a plant alkaloid, a terpenoids, a topoisomerase inhibitor, an antitumour antibiotic, an antibody, a hormonal therapy, an anti-diabetes agent, a leukotriene inhibitor, or combinations thereof. In some embodiments, the therapeutic agent is selected from: alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, UP 394 (abetimus sodium), UP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Argam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, bal sal azide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1 beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimethyl-3-thiomorpholine carboxamide (apratastat), golimumab (CNTO 148), Onercept, BG9924 (Biogen Idee), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca),), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, Medlmmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furcate, paramethasone, prednicarbate, prednisone, rimexolone, tixocortol, triamcinolone, ulobetasol, Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (^{M}6-OXO^{M}; 1,4,6-androstatrien-3,17-dione (ATD); formestane; testolactone; fadrozole; A-81834 (3-(3-(l,l-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy] acetic acid); SA6541 ((R)—S-[[4-(dimethylamino)phenyl]methyl]-N-(3-mercapto-2methyl-1-oxopropyl-L-cycteine); SC-56938 (ethyl-1-[2-[4-(phenylmethyl)phenoxy] ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinolinone); doxycycline; or combinations thereof.

In some embodiments, the therapeutic agent contains a radioactive moiety, for example a radioactive isotope such as ²¹¹At, ¹³¹I, ¹²³I, ⁹⁰Y, ¹⁸⁶Re, ¹⁸⁸Re, ¹⁵³Sm, ²¹²Bi, ³²P, ⁶⁴Cu radioactive isotopes of Lu, and others.

### Portion U

In some embodiments, D_{B} is G-T-Q-Y-D, and D is a therapeutic agent. In some embodiments, D is further defined as U, wherein U is a therapeutic agent. In some embodiments, U is a therapeutic agent is selected from: a chemotherapeutic agent, a steroid, an immunotherapeutic agent, a targeted therapy, an anti-inflammatory agent, or a combination thereof.

In some embodiments, U is a fragment having the structure of Formula (IA) or Formula (IB):

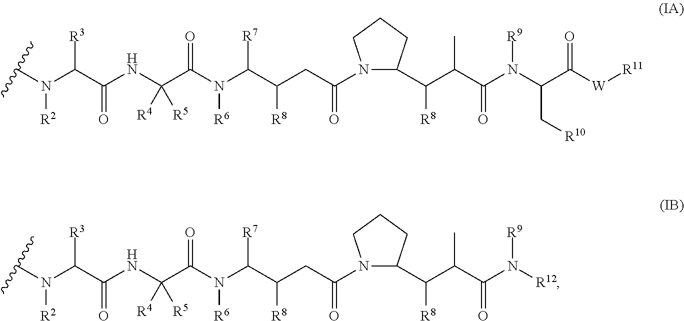

wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, or —O-optionally substituted C₁-C₈ alkylene;
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkylene;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is an integer ranging between 0 to 6; and
t is an integer ranging between 0 to 6.

In some embodiments, U is a fragment having the structure of Formula (IA) or Formula (IB):

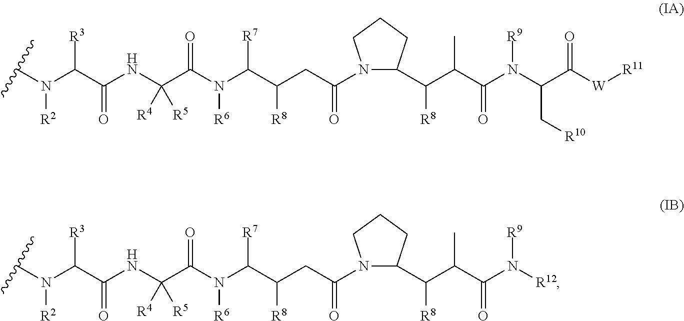

wherein,
R² is —H or optionally substituted C₁-C₈ alkyl;
R³ is —H, or optionally substituted C₁-C₈ alkyl;
R⁴ is —H, or optionally substituted C₁-C₈ alkyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, or optionally substituted C₃-C₈ carbocyclyl; each R⁸ is independently selected from —H, —OH, optionally substituted C₁-C₈ alkyl, optionally substituted C₃-C₈ carbocyclyl, or —O-(optionally substituted C₁-C₈ alkyl);
R⁹ is —H;
R¹⁰ is optionally substituted C₆-C₁₀ aryl;
W is —O—;
R¹¹ is —H; and
R¹² is —C(R⁸)₂—C(R⁸)₂—(C₆-C₁₀ aryl).

In some cases, D is further defined as C. In some embodiments, C is a small molecule cytotoxic agent. In some embodiments, a small molecule cytotoxic agent is a derivative of actinomycin; bleomycin; bortezomib; daunorubicin; docetaxel; doxifluridine; doxorubicin; epirubicin; epothilone; etoposide; irinotecan; paclitaxel; teniposide; topotecan; valrubicin; vinblastine; vincristine; vindesine; vinorelbine, desoxyvincaminol, vincaminol, vincamajine, vineridine, vinbumine, vinpocetine, vincamine, 2-methoxyestradiol, chalcones, colchicine, combretastatin, dictyostatin, discodermolide, eleutherobin, laulimalide, peloruside, podophyllotoxin, taxane, cryptophycin, halichondrin, maytansine, phomopsin, rhizoxin, spongistatin, tubulysin, vinca alkaloid, noscapinoid, auristatin, dolastain, ombrabulin, epothilone B, patupilone, ixabepilone, sagopilone, ansamitocin, auristadn E (AE), auristadn F (AF), auristadn E5-benzoylvaleric acid ester (AEVB), monomethyl auristadn E (MMAE), monomethyl auristadn F (MMAF), monomethyl auristadn D (MMAD), auristadn PE, auristadn PYE, amsacrine, anthracycline, camptothecin, duocarmycin, enediyne, indolinobenzodiazepine, netropsin, idarubicin, mitomycin-C, dactinomycin, mithramycin, nemorubicin, pixantrone, sabarubicin, silatecan, cositecan, exatecan, lurtotecan, gimatecan, belotecan, rubitecan, duocarmycin A, duocarmycin B1, duocarmycin B2, duocarmycin C1, duocarmycin C2, duocarmycin D, duocarmycin SA, calicheamicin, esperamicin, dynemicin A, pyrrolobenzodiazepine, pyrrolobenzodiazepine dimer anthramycin, abbeymycin, chicamycin, mazethramycin, neothramycins A, neothramycin B, porothramycin, prothracarcin, sibanomicin, sibiromycin, or tomaymycin.

In some embodiments, a cytotoxin is a derivative of a microtubule disrupting agent, dolastadn, auristadn, DNA modifying agent, or pyrrolobenzodiazepine.

In some embodiments, the small molecule cytotoxic agent comprises a microtubule disrupting agent. Exemplary microtubule disrupting agents include, but are not limited to, 2-methoxyestradiol, chalcones, colchicine, combretastatin, dictyostatin, discodermolide, eleutherobin, epothilone, laulimalide, peloruside, podophyllotoxin, taxane, cryptophycin, halichondrin, maytansine, phomopsin, rhizoxin, spongistatin, tubulysin, vinca alkaloid, noscapinoid, auristadn, dolastain, or derivatives or analogs thereof. In some embodiments, the small molecule cytotoxic agent is combretastatin or a derivative or analog thereof. In some embodiments, an analog of combretastatin is ombrabulin. In some embodiments, the epothilone is epothilone B, patupilone, ixabepilone, sagopilone, BMS-310705, or BMS-247550. In some embodiments, the tubulysin is a tubulysin analog or derivative such as described in U.S. Pat. Nos. 8,580,820 and 8,980,833 and in U.S. Publication Nos. 20130217638, 20130224228, and 201400363454. In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268. In some embodiments, the taxane is paclitaxel or docetaxel. In some embodiments, the vica alkaloid is vinblastine, vincristine, vindesine, vinorelbine, desoxyvincaminol, vincaminol, vincamajine, vineridine, vinbumine, vinpocetine, or vincamine.

In some embodiments, the small molecule cytotoxic agent is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714 and 8,871,720. In some embodiments, the dolastatin 15 analog is cemadotin or tasidotin.

In some embodiments, the small molecule cytotoxic agent comprises a DNA modifying agent. In some embodiments, the DNA modifying agent comprises amsacrine, anthracycline, camptothecin, doxorubicin, duocarmycin, enediyne, etoposide, indolinobenzodiazepine, netropsin, teniposide, pyrrolobenzodiazepine, or derivatives or analogs thereof. In some embodiments, the anthracycline is doxorubicin, daunorubicin, epirubicin, idarubicin, mitomycin-C, dactinomycin, mithramycin, nemorubicin, pixantrone, sabarubicin, or valrubicin. In some embodiments, the analog of camptothecin is topotecan, irinotecan, silatecan, cositecan, exatecan, lurtotecan, gimatecan, belotecan, rubitecan, or SN-38. In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

Pyrrolobenzodiazepine (PBDs) are a class of sequence-selective DNA minor-groove binding crosslinking agents. PBD dimers are particularly potent because of their cell cycle-independent activity and because their integration minimally distorts DNA, increasing the likelihood of evasion of DNA damage repair responses.

In some embodiments, the small molecule cytotoxic agent is pyrrolobenzodiazepine. In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-123 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, C is not monomethyl auristatin E (MMAE). In some embodiments, C is not monomethyl auristatin F (MMAF).

In some embodiments, C is Y—U. In some embodiments, C is a derivative of MMAE. In some embodiments, C is derivative of MMAF.

### Portion K

In some embodiments, D_{B} is G-T-Q-K,

In some embodiments, K is a fragment having the structure of Formula (VIA) or Formula (VIB):

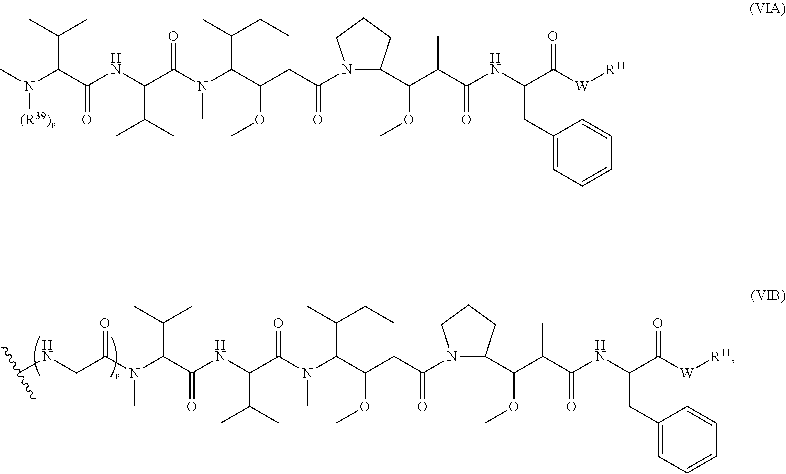

wherein,
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³O)ₜ—CH(R¹⁵)₂;
R¹³ is optionally substituted C₁-C₈ alkylene;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R³⁹ is —NHCH₂CH₂—, —OCH₂CH₂—, —NHCH₂S(O)₂—, —NHCR^{2B}R^{3B}C(O)—, —NHCR^{2B}R^{3B}CH₂—NHCH₂C(O)NHCH₂CH₂—, or —NHCH(CH₂CH₂CH₂NHC(═NH)NH₂)C(O)—;
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂ q is an integer ranging between 0 to 6;
v is an integer ranging between 0 to 3;
t is an integer ranging between 0 to 6; and
provided that R^{2B} and R^{3B} are not both H when W is —O— and R¹¹ is H

In some embodiments, K is a fragment having the structure of Formula (VIA) or Formula (VIB):

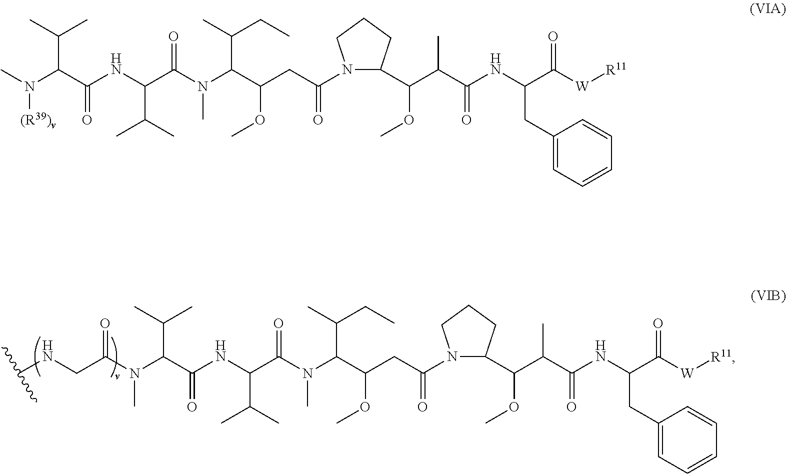

wherein,
W is —O—;
R¹¹ is —H or an optionally substituted C₁-C₂₀ alkyl;
R³⁹ is —NHCH₂CH₂—, —OCH₂CH₂—, —NHCR^{2B}R^{3B}C(O)—, —NHCR^{2B}R^{3B}CH₂—NHCH₂C(O)NHCH₂CH₂—, or —NHCH(CH₂CH₂CH₂NHC(═NH)NH₂)C(O)—;
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
q is an integer ranging between 0 to 6;
v is an integer ranging between 0 and 3;
t is an integer ranging between 0 to 6; and
provided that R^{2B} and R^{3B} are not both H when W is —O— and R¹¹ is H.

In some embodiments, W is —O—. In some embodiments, W is —O— and R¹¹ is —H or an optionally substituted C₁-C₂₀ alkyl. In some embodiments, W is —O— and R¹¹ is —H. In some embodiments, W is —O— and R¹¹ is optionally substituted C₁-C₂₀ alkyl. In some embodiments, W is —O— and R¹¹ is —CH₃. In some embodiments, W is —NR¹²—. In some embodiments, W is —NR¹²—, wherein R¹² is —H, and R¹¹ is —H or an optionally substituted C₁-C₂₀ alkyl. In some embodiments, W is —NR¹²—, R¹² is —H, and R¹¹ is —H. In some embodiments, W is —NR¹²—, R¹² is —H, and R¹¹ is optionally substituted C₁-C₂₀ alkyl. In some embodiments, W is —NR¹²—, R¹² is —H, and R¹¹ is —CH₃. In some embodiments, W is —NR¹²—, R¹² is —CH₃, and R¹¹ is —CH₃. In some embodiments, W is —S—. In some embodiments, W is —S— and R¹¹ is H or an optionally substituted C₁-C₂₀ alkyl. In some embodiments, W is —S— and R¹¹ is —H. In some embodiments, W is —S— and R¹¹ is optionally substituted C₁-C₂₀ alkyl. In some embodiments, W is —S— and R¹¹ is —CH₃.

In some embodiments, R³⁹ is —NHCH₂CH₂—, —OCH₂CH₂—, —NHCR^{2B}R^{3B}C(O)—, —NHCR^{2B}R^{3B}CH₂—, or —NHCH₂C(O)NHCH₂CH₂NH—. In some embodiments, R³⁹ is —NHCH₂CH₂—, —NHCR^{2B}R^{3B}C(O)—, or —NHCR^{2B}R^{3B}CH₂—.

In some embodiments, R³⁹ is —NHCH(CF₃)C(O)—. In some embodiments, R³⁹ is —NHCH₂S(O)₂—. In some embodiments, R³⁹ is —NHCH₂CH₂—. In some embodiments, R³⁹ is —OCH₂CH₂—. In some embodiments, R³⁹ is —NHCH(CH₃)C(O)—. In some embodiments, R³⁹ is is —NHCH(CH₂CH₃)C(O)—. In some embodiments, R³⁹ is —NHCH(CH(CH₃)₂)C(O)—. In some embodiments, R³⁹ is —NHCH(CH₂CH₂C(O)OH)C(O)—. In some embodiments, R³⁹ is —NHCH(CH₂CH₂CH₂NHC(═NH)NH₂)C(O)—.

In some embodiments, R³⁹ is —NHCH₂C(O)—, —NHCHR^{2A}C(O)—, —NHCH₂S(O)₂—, —NHCH₂CH₂—, —NHCHR^{2B}CH₂—, —OCH₂CH₂—, or —NHCH₂C(O)NHCH₂CH₂NH—, wherein R^{2B} is —CH₃, —CH₂CH₃, —CF₃, —CH(CH₃)₂, —CH(OH)CH₃, —CH₂OH, —CH₂CH₂C(O)OH, or —CH₂CH₂CH₂NHC(═NH)NH₂.

In some embodiments, R³⁹ is a bond.

In some embodiments, R³⁹ is —NHCH₂C(O)—, —NHCHR^{2B}C(O)—, —NHCH₂S(O)₂—, —NHCH₂CH₂—, —NHCHR¹CH₂—, —OCH₂CH₂—, or —NHCH₂C(O)NHCH₂CH₂NH—, wherein R^{2B} is —CH₃, —CH₂CH₃, —CF₃, —CH(CH₃)₂, —CH(OH)CH₃, —CH₂OH, —CH₂CH₂C(O)OH, or —CH₂CH₂CH₂NHC(═NH)NH₂.

In some embodiments R³⁹ is —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—, wherein R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂; and R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂.

In some embodiments, R^{3B} is not H.

In some embodiments, each R^{2B} and R^{3B} is independently in either stereochemical configuration D or L.

In some embodiments, R³⁹ is an amino acid, for example, —NHCH(CH₃)C(O)— corresponds to the amino acid Alanine (Ala). In some embodiments, the amino acid is a D-amino acid. In some embodiments, the amino acid is an L-amino acid.

In some embodiments v is 1. In some embodiments v is 2. In some embodiments, v is 3.

Disclosed herein are compounds, or a pharmaceutically acceptable salt thereof, having the structure of Formula (IV):

[(S)_{w}-A-c_{A}-(c_{P}-M)ᵤ-X—B-c_{B}-G-T-Q-C]—(N—Z)ᵥ (IV).

Disclosed herein are compounds, or a pharmaceutically acceptable salt thereof, having the structure of Formula (V):

M²ₒ-S_{w}-A-c_{A}-(c_{P}-M¹)ᵤ-X—B-c_{B}-G-T-Q-C (V).

### Imaging Agents

In some instances, the cargo is an imaging agent. In some cases, the imaging cargo comprises D_{B}. In additional cases, D_{B} is G-T-Q-Y-D, in which D is an imaging agent.

In some embodiments, the imaging agent is conjugated to portion of A, portion of B or both portions A and B. In some embodiments, the imaging agent is conjugated to the target ligand.

In some embodiments, an imaging agent is a dye. In some embodiments, an imaging agent is a fluorescent moiety. In some embodiments, a fluorescent moiety is selected from: a fluorescent protein, a fluorescent peptide, a fluorescent dye, a fluorescent material or a combination thereof.

All fluorescent moieties are encompassed within the term “fluorescent moiety.” Specific examples of fluorescent moieties given herein are illustrative and are not meant to limit the fluorescent moieties for use with the targeting molecules disclosed herein.

Examples of fluorescent dyes include, but are not limited to, xanthenes (e g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenandirecene; pyrene; butadiene; stilbene; porphyrin; pthalocyanine; lanthanide metal chelate complexes; rare-earth metal chelate complexes; and derivatives of such dyes.

Examples of fluorescein dyes include, but are not limited to, 5-carboxyfluorescein, fluorescein-5-isothiocyanate, fluorescein-6-isothiocyanate and 6-carboxyfluorescein.

Examples of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the trade name of TEXAS RED®).

Examples of cyanine dyes include, but are not limited to, Cy3, Cy3B, Cy3.5, Cy5, Cy5.5, Cy7, IRDYE680, Alexa Fluor 750, IRDye800CW, ICG.

Examples of fluorescent peptides include GFP (Green Fluorescent Protein) or derivatives of GFP (e.g., EBFP, EBFP2, Azurite, mKalamal, ECFP, Cerulean, CyPet, YFP, Citrine, Venus, YPet).

Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc.

In some embodiments, the imaging agent is labeled with a positron-emitting isotope (e.g., ¹⁸F) for positron emission tomography (PET), gamma-ray isotope (e.g., ^{99m}Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the imaging agent is labeled with: a gadolinium chelate, an iron oxide particle, a super paramagnetic iron oxide particle, an ultra small paramagnetic particle, a manganese chelate or gallium containing agent.

Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the imaging agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indium chelate.

Examples of Tc chelates include, but are not limited to HYNIC, DTP A, and DOTA.

In some embodiments, the imaging agent contains a radioactive moiety, for example a radioactive isotope such as ²¹¹At, ¹³¹I, ¹²³I, ⁹⁰Y, ¹⁸⁶Re, ¹⁸⁸Re, ¹⁵³Sm, ²¹²Bi, ³²P, ⁶⁴Cu radioactive isotopes of Lu, and others.

In some embodiments, a selective delivery molecule according to Formulas I-V comprising an imaging agent is employed in guided surgery. In some embodiments, the selective delivery molecule preferentially localized to cancerous or other undesirable tissues (i.e. necrotic tissues). In some embodiments, a selective delivery molecule according to Formula I comprising an imaging agent is employed in a guided surgery to remove colorectal cancer. In some embodiments, guided surgery employing the selective delivery molecule allows a surgeon to excise as little healthy (i.e., non-cancerous) tissue as possible. In some embodiments, guided surgery employing the selective delivery molecule allows a surgeon to visualize and excise more cancerous tissue than the surgeon would have been able to excise without the presence of the selective delivery molecule. In some embodiments, the surgery is fluorescence-guided surgery.

### Portion Z²

In some embodiments, D is an imaging agent. In some embodiments, D is further defined as Z², wherein Z² is an imaging agent. In some embodiments, Z² is a dye. In some embodiments, Z² is a fluorescent moiety. In some embodiments, the fluorescent moiety is selected from: a fluorescent protein, a fluorescent peptide, a fluorescent dye, a fluorescent material or a combination thereof.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes.

Examples of fluorescein dyes include, but are not limited to, 5-carboxyfluorescein, fluorescein-5-isothiocyanate, fluorescein-6-isothiocyanate and 6-carboxyfluorescein.

Examples of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the trade name of TEXAS RED®).

Examples of cyanine dyes include, but are not limited to, Cy3, Cy3B, Cy3.5, Cy5, Cy5.5, Cy7, IRDYE680, Alexa Fluor 750, IRDye800CW, and ICG.

Examples of fluorescent peptides include GFP (Green Fluorescent Protein) or derivatives of GFP (e.g., EBFP, EBFP2, Azurite, mKalamal, ECFP, Cerulean, CyPet, YFP, Citrine, Venus, YPet).

Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc.

In some embodiments, the imaging agent is labeled with a positron-emitting isotope (e.g., ¹⁸F) for positron emission tomography (PET), gamma-ray isotope (e.g., ^{99m}Tc) for single photon emission computed tomography (SPECT), or a paramagnetic molecule or nanoparticle (e.g., Gd³⁺ chelate or coated magnetite nanoparticle) for magnetic resonance imaging (MRI).

In some embodiments, the imaging agent is labeled with: a gadolinium chelate, an iron oxide particle, a super paramagnetic iron oxide particle, an ultra small paramagnetic particle, a manganese chelate or gallium containing agent.

Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA).

In some embodiments, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, the imaging agent is a nuclear probe. In some embodiments, the imaging agent is a SPECT or PET radionuclide probe. In some embodiments, the radionuclide probe is selected from: a technetium chelate, a copper chelate, a radioactive fluorine, a radioactive iodine, a indium chelate.

Examples of Tc chelates include, but are not limited to HYNIC, DTPA, and DOTA.

In some embodiments, the imaging agent contains a radioactive moiety, for example a radioactive isotope such as ²¹¹At, ¹³¹I, ¹²³I, ⁹⁰Y, ¹⁸⁶Re, ¹⁸⁸Re, ¹⁵³Sm, ²¹²Bi, ³²P, ⁶⁴Cu radioactive isotopes of Lu, and others.

In some embodiments, Z² is a fragment having the structure of Formula (C):

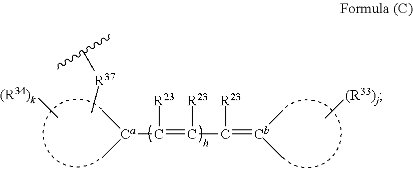

wherein
the dotted lines encircling XX and YY are each independently selected from atoms necessary for the formation of one ring to three fused rings having 4 to 7 atoms in each ring;
at least one atom in the ring comprising C^{a} is an optionally cationic nitrogen;
at least one atom in the ring comprising C^{b} is an optionally cationic nitrogen;
k and j are integers independently selected from 0 to the number of atoms necessary for the formation of XX or YY, with the proviso that k and j cannot both be 0;
each R²³ is independently selected from —H, —OR³⁴, —SR³⁴, —NR³⁴R³⁴, halogen, —CN, optionally substituted C₁-C₈ alkyl, optionally substituted C₃-C₈ carbocyclyl, optionally substituted C₃-C₈ heterocyclyl, optionally substituted C₆-C₁₀ aryl, and optionally substituted C₆-C₁₀ heteroaryl;
two R²³ groups, together with the atoms to which they are attached, can be optionally joined to form a ring;
h is an integer selected from 0, 1, 2, 3 and 4;
each R³⁵ and R³⁶ is independently selected from —H, optionally substituted C₁-C₈ alkyl, optionally substituted C₃-C₈ carbocyclyl, optionally substituted C₃-C₈ heterocyclyl, optionally substituted C₆-C₁₀ aryl, optionally substituted C₆-C₁₀ heteroaryl, halogen, —SO₃⁻, —SO₃H, —NO₂⁻, —CN, —P(O)(OR²⁴)(OR²³), -D¹R²⁶, —NR²⁷R²⁸ and —C(D²)R²⁹;
R³⁷ is selected from optionally substituted C₁-C₈ alkylene, optionally substituted C₁-C₈ alkylene-C(O)—, optionally substituted C₁-C₈ alkylene-C(O)NHCH₂CH₂NH—, optionally substituted C₁-C₈ alkylene-C(O)—(NHCH₂C(O))ₙ—, —(CH₂—CH₂—O)ₙ—, —(CH₂—CH₂—O)ₙ—(CH₂)ₘC(O)—, —(CH₂—CH₂—NH)ₙ—, —(NH—CH₂—CH₂)ₙ—, or —(CH₂—CH₂—NH)ₙ—(CH₂)ₘC(O)—;
each D¹ is independently selected from —O— and —S—;
each D² is independently selected from —O—, —S— and —NH—;
each R²⁴ and R²⁵ is independently selected from H and optionally substituted C₁-C₄ alkyl;
at least one of R²⁴ and R²⁵ is —H;
each R²⁶, R²⁷ and R²⁸ is independently selected from —H and optionally substituted C₁-C₈ alkyl;
each R²⁷ and R²⁸, together with the nitrogen to which they are attached, can be optionally joined to form a member selected from a reactive functional group, —NHNH₂, —N═N═N, —N═C═S and —N═C═O;
each R²⁹ is independently selected from —H, optionally substituted C₁-C₈ alkyl, a reactive functional group, —NR³⁰R³¹ and —OR³²;
each R³⁰ and R³¹ is independently selected from —H and optionally substituted C₁-C₈ alkyl;
each R³² is independently selected from —H, optionally substituted C₁-C₈ alkyl, optionally substituted C₃-C₈ carbocyclyl, optionally substituted C₃-C₈ heterocyclyl, optionally substituted C₆-C₁₀ aryl, optionally substituted C₆-C₁₀ heteroaryl and —C(O)R³³;
each R³³ is independently an optionally substituted C₁-C₈ alkyl; and
each R³⁴ is independently selected from —H and optionally substituted C₁-C₈ alkyl.

In some embodiments, Z² is a cyanine based imaging agent fragment having the structure of Formula (IC):

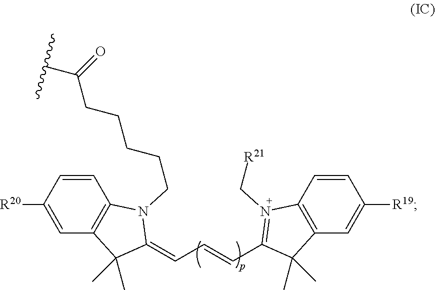

wherein,
each R¹⁹ and R²⁰ are independently —H, —SO₃—SO₃H, or C₁-C₈ alkyl, wherein at least one of R¹⁹ and R²⁰ is SO₃⁻;
R²¹ is —H or C₁-C₈ alkyl; and
p is an integer ranging from 0 to 3.

In some embodiments, p is 0. In some embodiments, p is 1. In some embodiments, p is 2. In some embodiments, p is 3.

In some embodiments, Z² is the fragment

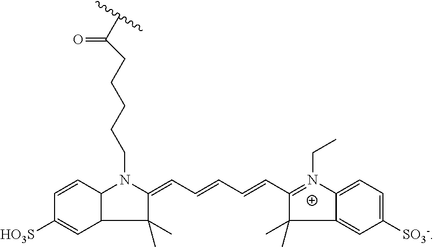

In some embodiments, Z² is the fragment

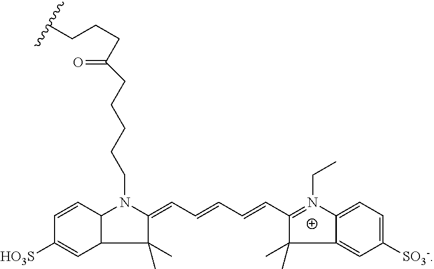

### Portion Y

In some embodiments, Y comprises 1-5 amino acids, e.g., 1, 2, 3,4, or 5 amino acids. In some embodiments Y comprises 1-3 amino acids, e.g., 1, 2, or 3 amino acids. In some embodiments Y comprises 1-3 L-amino acids (e.g., 1, 2, or 3 L-amino acids), 1-3 D-amino acids (e.g., 1, 2, or 3 D-amino acids), or in which the 1-3 amino acids are a mixture of D- and L-amino acids.

In some embodiments, the amino acids are selected from polar residues, nonpolar residues, basic residues, or acidic residues. Exemplary polar residues comprise Tyr, Ser, Thr, Asn, Gin, and Cys. Exemplary nonpolar residues comprise Trp, Phe, Gly, Ala, Val, lie, Leu, Met, and Pro. Exemplary basic residues comprise Lys, Arg, and His. Exemplary acidic residues comprise Asp and Glu. In some instances, Y comprises a polar residue, a nonpolar residue, a basic residue, an acidic residue, or a combination thereof.

In some embodiments, Y are selected from: glycine, alanine, valine, serine, threonine, arginine, lysine, aspartic acid, or glutamic acid. In some cases, Y comprises one or more of glycine, alanine, valine, serine, threonine, arginine, lysine, aspartic acid, or glutamic acid. In some embodiments, Y comprises one or more glycine residues. In some embodiments, Y comprises one or more alanine residues. In some embodiments, Y comprises one or more valine residues. In some embodiments, Y comprises one or more serine residues. In some embodiments, Y comprises one or more threonine residues. In some embodiments, Y comprises one or more aspartic acid residues. In some embodiments, Y comprises one or more glutamic acid residues. In some embodiments, Y comprises one or more lysine residues. In some embodiments, Y comprises one or more arginine residues.

In some embodiments, Y comprises 1-3 glycine residues, e.g., 1, 2, or 3 glycine residues.

In some instances, Y comprises 1-3 alanine residues, e.g., 1,2, or 3 alanine residues. In some instances, Y comprises 1-3 L-alanine residues, e.g., 1,2, or 3 L-alanine residues. In other instances, Y comprises 1-3 D-alanine residues, e.g., 1,2, or 3 D-alanine residues. In additional instances, Y comprises 2 or 3 alanine residues, in which the alanine residues are a mixture of L- and D-alanine residues. In some embodiments, Y is an amide. In some embodiments, Y is an amino substituted C₁-C₈ alkylene. In some embodiments, Y is a C₁-C₈ alkoxylene.

In some embodiments, Y is a bond, —NHCH₂C(O)—, —NHCH(CH₃)C(O)—, —NHCH(CH₂CH₃)C(O)—, —NHCH(CF₃)C(O)—, —NHCH(CH(CH₃)₂)C(O)—, —NHCH(CH(OH)CH₃)C(O)—, —NHCH(CH₂OH)C(O)—, —NHCH₂S(O)₂—, —NHCH₂CH₂—, —NHCH(CH₃)CH₂—, —OCH₂CH₂—, or —NHCH₂C(O)NHCH₂CH₂NH—. In some embodiments, Y is a bond. In some embodiments, Y is —NHCH₂C(O)—. In some embodiments, Y is —NHCH(CH₃)C(O)—. In some embodiments, Y is —NHCH(CH₂CH₃)C(O)—. In some embodiments, Y is —NHCH(CF₃)C(O)—. In some embodiments, Y is —NHCH₂S(O)₂—. In some embodiments, Y is —NHCH₂CH₂—. In some embodiments, Y is —NHCH(CH₃)CH₂—. In some embodiments, Y is —OCH₂CH₂—. In some embodiments, Y is —NHCH₂C(O)NHCH₂CH₂NH—. In some embodiments, Y is —NHCH(CH(CH₃)₂)C(O)—. In some embodiments, Y is —NHCH(CH(OH)CH₃)C(O)—. In some embodiments, Y is —NHCH(CH₂OH)C(O)—.

In some embodiments, Y is a bond, —NHCH₂C(O)—, —NHCHR¹C(O)—, —NHCH₂S(O)₂—, —NHCH₂CH₂—, —NHCHR¹CH₂—, —OCH₂CH₂—, or —NHCH₂C(O)NHCH₂CH₂NH—, wherein R¹ is —CH₃, —CH₂CH₃, —CF₃, —CH(CH₃)₂, —CH(OH)CH₃, —CH₂OH, —CH₂CH₂C(O)OH, or —CH₂CH₂CH₂NHC(═NH)NH₂.

In some embodiments, Y is —NHCH₂C(O)—, —NHCHR¹C(O)—, —NHCH₂S(O)₂—, —NHCH₂CH₂—, —NHCHR¹CH₂—, —OCH₂CH₂—, or —NHCH₂C(O)NHCH₂CH₂NH—, wherein R¹ is —CH₃, —CH₂CH₃, —CF₃, —CH(CH₃)₂, —CH(OH)CH₃, —CH₂OH, —CH₂CH₂C(O)OH, or —CH₂CH₂CH₂NHC(═NH)NH₂.

In some embodiments, Y is a bond.

In some embodiments Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—, wherein R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂; and R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂.

In some embodiments, Y is a modifier In some embodiments, Y modifies a therapeutic agent U or Y modifies an imaging agent Z². In some embodiments, Y modifies a therapeutic agent U. In some embodiments, Y modifies an imaging agent Z². In some embodiments, the modification of U or Z² by Y results in a modulation in the pharmacokinetics of therapeutic agent U or imaging agent Z².

### Portion Q

In some embodiments, Q is a linker. In some embodiments, Q is -valine-citrulline-. In some embodiments, Q is -phenylalanine-citrulline-. In some embodiments, Q is -threonine-citrulline-. In some embodiments, Q is -tryptophan-citrulline-.

In some embodiments, Q is -valine-lysine-. In some embodiments, Q is -phenylalanine-lysine-. In some embodiments, Q is -threonine-lysine-. In some embodiments, Q is -tryptophan-lysine-.

In some embodiments, Q is -valine-alanine-. In some embodiments, Q is -phenylalanine-alanine-. In some embodiments, Q is -threonine-alanine-. In some embodiments, Q is -tryptophan-alanine-.

In some embodiments, Q is a bond.

In some embodiments, Q is a bond or selected from the following group:

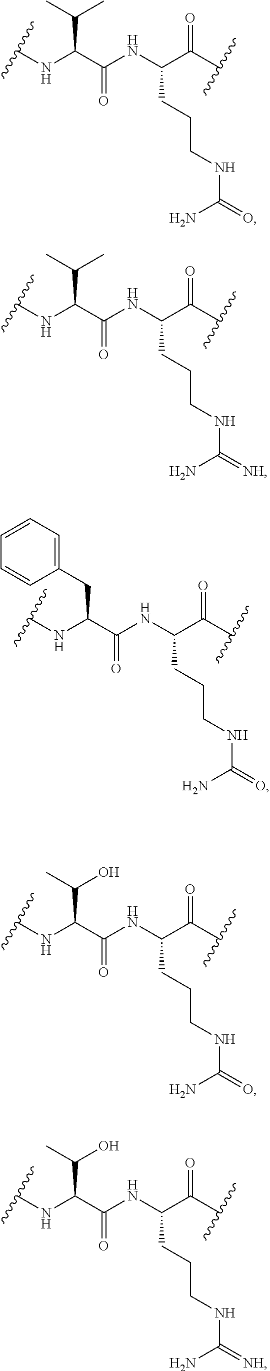

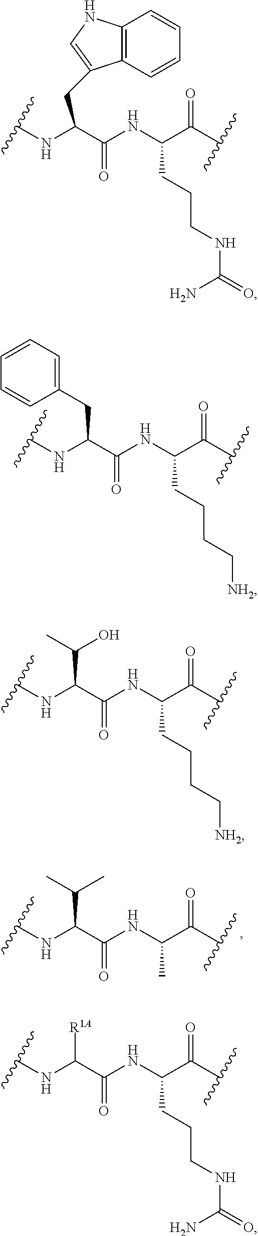

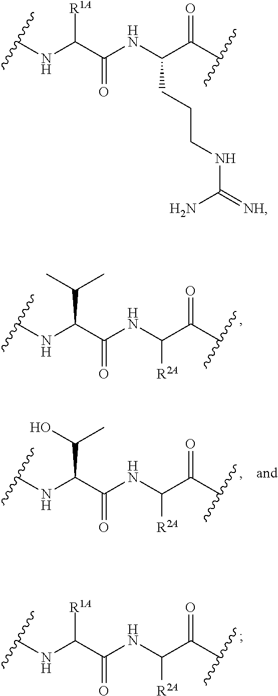

wherein,
R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂, or —CH₂CH₂CH₂NHC(O)NH₂.

In some embodiments, Q is selected from the following group:

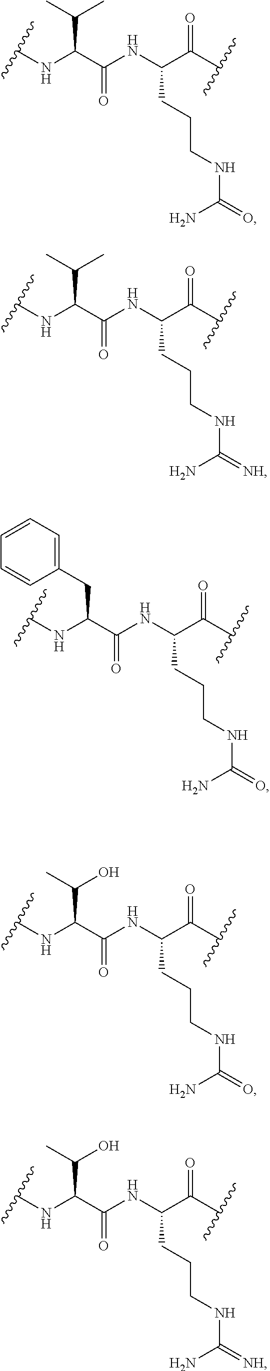 [SMILES: *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@@H](Cc1ccccc1)C(=O)N[C@@H](CCCNC(N)=O)C(*)=O ; *N[C@H](C(=O)N[C@@H](CCCNC(N)=O)C(*)=O)C(C)O ; *N[C@H](C(=O)N[C@@H](CCCNC(=N)N)C(*)=O)C(C)O]

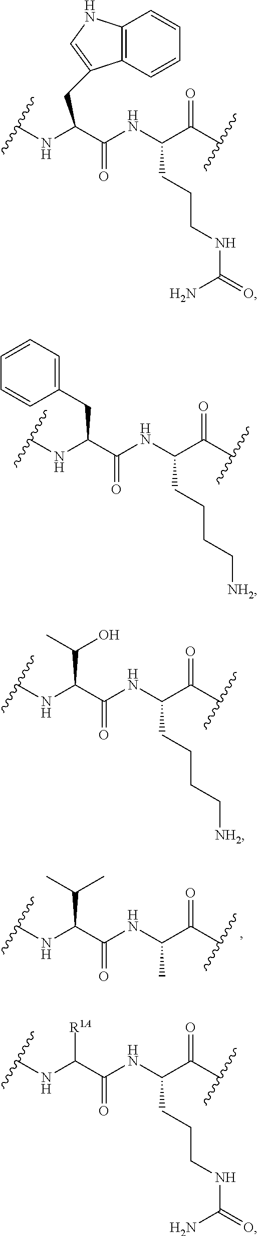

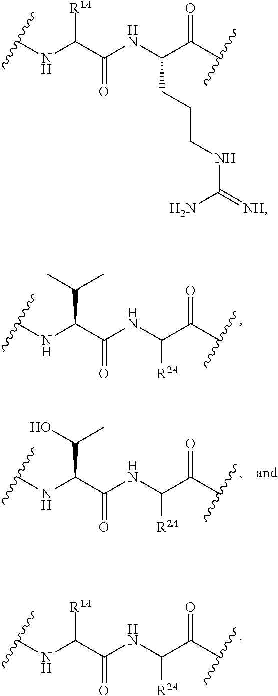

In some embodiments, Q is a bond. In some embodiments, Q is

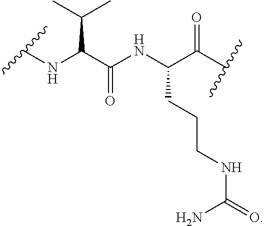

In some embodiments, Q is

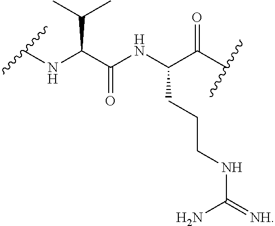 [SMILES: *N[C@H](C(=O)N[C@@H](CCCNC(=N)N)C(*)=O)C(C)C]

In some embodiments, Q is

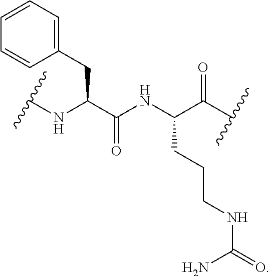 [SMILES: *N[C@@H](Cc1ccccc1)C(=O)N[C@@H](CCCNC(N)=O)C(*)=O]

In some embodiments, Q is

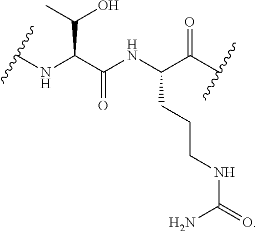 [SMILES: *N[C@H](C(=O)N[C@@H](CCCNC(N)=O)C(*)=O)C(C)O]

In some embodiments, Q is

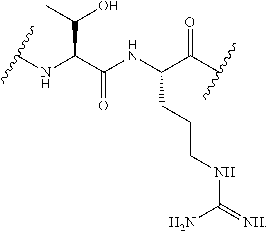 [SMILES: *N[C@H](C(=O)N[C@@H](CCCNC(=N)N)C(*)=O)C(C)O]

In some embodiments, Q is

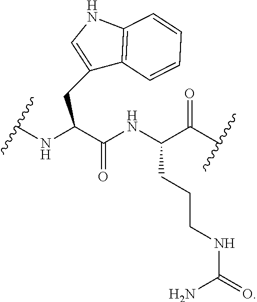 [SMILES: *N[C@@H](Cc1c[nH]c2ccccc12)C(=O)N[C@@H](CCCNC(N)=O)C(*)=O]

In some embodiments, Q is

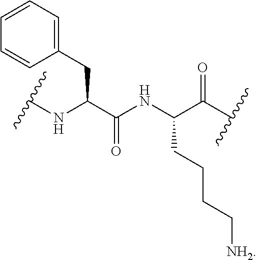 [SMILES: *N[C@@H](Cc1ccccc1)C(=O)N[C@@H](CCCCN)C(*)=O]

In some embodiments, Q is

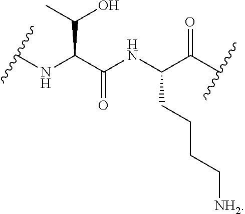 [SMILES: *N[C@H](C(=O)N[C@@H](CCCCN)C(*)=O)C(C)O]

In some embodiments, Q is

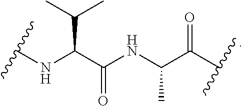 [SMILES: *N[C@H](C(=O)N[C@@H](C)C(*)=O)C(C)C]

In some embodiments, Q is

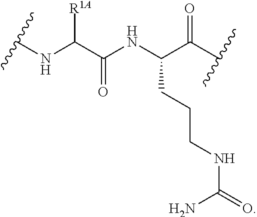

In some embodiments, Q is

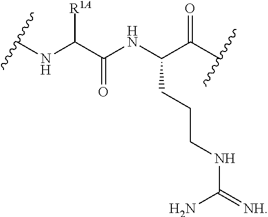

In some embodiments, Q is

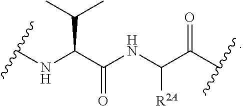

In some embodiments, Q is

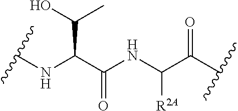

In some embodiments, Q is

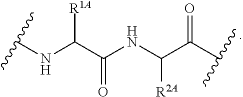

In some embodiments, R^{1A} is —H. In some embodiments, R^{1A} is optionally substituted C₁-C₈ alkyl. In some embodiments, R^{1A} is optionally substituted C₃-Gt carbocyclyl. In some embodiments, R^{1A} is optionally substituted C₆-C₁₀ aryl. In some embodiments, R^{1A} is optionally substituted C₇-C₁₂ aralkyl. In some embodiments, R^{1A} is optionally substituted C₃-C₈ heterocyclyl.

In some embodiments, R^{2A} is —H. In some embodiments, R^{2A} is optionally substituted C₁-C₈ alkyl. In some embodiments, R^{2A} is optionally substituted C₃-C₈ carbocyclyl. In some embodiments, R^{2A} is optionally substituted C₆-C₁₀ aryl. In some embodiments, R^{2A} is optionally substituted C₇-C₁₂ aralkyl. In some embodiments, R^{2A} is optionally substituted C₃-C₈ heterocyclyl. In some embodiments, R^{2A} is amino substituted C₁-C₈ alkyl. In some embodiments, R^{2A} is —CH₂CH₂CH₂CH₂NH₂. In some embodiments, R^{2A} is —CH₂CH₂CH₂NHC(O)NH₂. In some embodiments R^{2A} is —CH₂CH₂CH₂NHC(═NH)NH₂.

In some embodiments, Q is a linker consisting of one or more amino acids is used to join Y-D_{B} to the remainder of the SDM. In some embodiments, Q is a linker consisting of one or more amino acids is used to join Y-D_{B} to portion T. Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In some embodiments, the Q linker is flexible. In some embodiments, the Q linker is rigid. In some embodiments, the Q linker comprises a linear structure. In some embodiments, the Q linker comprises a non-linear structure. In some embodiments, the Q linker comprises a branched structure. In some embodiments, Q linker comprises a cyclic structure.

In some embodiments, Q linker comprises a peptide linkage. The peptide linkage comprises L-amino acids and/or D-amino acids. In embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a Q linker is designed for cleavage in the presence of particular conditions or in a particular environment. In some embodiments, a Q linker is cleavable by an intracellular protease. In some embodiments, Q is cleavable by an intracellular protease. In some embodiments, a Q linker is cleavable by a lysosomal protease. In some embodiments, the intracellular protease is a cysteine protease. In some embodiments, the intracellular protease is an aspartyl protease. In some embodiments, the intracellular protease is a serine protease. In some embodiments, the cysteine protease is a caspase, a cathepsin, calpain, papain or a legumain. In some embodiments, the intracellular protease is an initiator caspase. In some embodiments, the intracellular protease is an effector caspase. In some embodiments, the Q linker is cleavable by a protease selected from among cathepsin B, cathepsin L, cathepsin H, cathepsin K, cathepsin W, cathepsin C, cathepsin F, cathepsin V, cathepsin X, cathepsin S, cathepsin D, cathepsin G, HCP-1, HCP-2, dipeptidyl-peptidase I, MEROPS C13, CED-3 peptidase, caspase 2, caspase 3, caspase 6, caspase 7, caspase 8, caspase 9, caspase 10, caspase 11; caspase 12, caspase 13, and caspase 14. In some embodiments, the Q linker is cleavable by a protease selected from among cathepsin B, cathepsin L, caspase 3, caspase 7, caspase 8, and caspase 9. In some embodiments, a Q linker is cleavable by Cathepsin B a dipeptidyl carboxypeptidase. In some embodiments the linker has a lysine, citrulline, or arginine residue at the P1 position and a large hydrophobic residue at the P1′ position.

In some embodiments, the Q linker comprises an acid sensitive chemical linker. In some embodiments, acid sensitive chemical linker is hydrazone or a derivative thereof. In some embodiments, a Q linker comprises a self-immolative spacer. In some embodiments, the self-immolative spacer is of sufficient length to prevent the occurrence of steric hindrance between the B portion of the SDM and the therapeutic cargo. In some embodiments, Q comprises a p-aminobenzyl alcohol (PABOH) spacer or a derivative thereof. In some embodiments, Q comprises a p-aminobenzyl carbonyl (PABC) spacer or a derivative thereof. In some embodiments, Q comprises a branched bis(hydroxymethyl)styrene (BHMS) spacer or a derivative thereof. In some embodiments, Q comprises a 2-aminoimidazol-5-methanol derivative or an ortho or para-aminobenzylacetal spacer. In some embodiments Q comprises 2,6-bishydroxymethyl-p-cresol or hemithioaminal derivatives.

In some embodiments, the Q linker comprises the lysosomally cleavable peptide. In some embodiments, the Q linker comprises the lysosomally cleavable dipeptide Phe-Arg. In some embodiments, the Q linker comprises the lysosomally cleavable dipeptide Phe-Lys. In some embodiments, the Q linker comprises the lysosomally cleavable dipeptide Val-Cit (1-citrulline). In some embodiments, the Q linker comprises the lysosomally cleavable tetrapeptide Gly-Phe-Leu-Gly. In some embodiments, the Q linker comprises the lysosomally cleavable tetrapeptide Ala-Leu-Ala-Leu.

In some embodiments, the Q linker comprises the lysosomally cleavable peptide and a self-immolative spacer.

In some embodiments, Q is a pH-sensitive linker. In some embodiments, Q is cleaved under acidic pH conditions. In some embodiments, Q is cleaved under acidic pH conditions of the lysosome.

It will be understood that a Q linker disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

### Portion T

In some embodiments, T is a spacer. In some embodiments, T is an optionally substituted Cᵣ C₈ alkylene, optionally substituted C₁-C₈ alkylene-C(O)—, optionally substituted C₃-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O)ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, —(CH₂)ₘ—(NR^{1B}—CH₂—CH₂)ₙ—, or —(CH₂—CH₂—NR^{1B})ₙ—(CH₂)ₘC(O)—, wherein each R^{1B} is independently is —H, —CH₃, —CH₂CH₃, or —CH₂CH₂NH₂. In some embodiments, each n is independently an integer ranging from 1 to 30. In some embodiments, each n is independently an integer ranging from 1 to 25. In some embodiments, each n is independently an integer ranging from 1 to 20. In some embodiments, each m is independently an integer ranging from 1 to 15. In some embodiments, each m is independently an integer ranging from 1 to 10. In some embodiments, each m is independently an integer ranging from 1 to 8. In some embodiments, each m is independently an integer ranging from 1 to 5.

In some embodiments, T is an optionally substituted C₁-C₈ alkylene. In some embodiments, T is an optionally substituted C₁-C₈ alkylene-C(O)—. In some embodiments, T is an optionally substituted C₃-C₈ carbocyclyl. In some embodiments, T is an optionally substituted C₃-C₈ carbocyclyl-C(O)—. In some embodiments, T is an optionally substituted C₁-C₈ alkylene-C(O)NHCH₂C(O)—. In some embodiments, T is an optionally substituted C₁-C₈ alkylene-C(O)—(NHCH₂C(O)ₙ—. In some embodiments, T is an optionally substituted C₆-C₁₀ arylene. In some embodiments, T is an optionally substituted C₆-C₁₀ arylene —C(O)—. In some embodiments, T is —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₙC(O)— or optionally substituted C₁-C₈ alkylene —C(O)NH—(CH₂—CH₂—O)ₙ—(CH₂)ₘC(O)—. In some embodiments, T is —(CH₂—CH₂—NR^{1B})ₙ—. In some embodiments T is —(CH₂)ₘ—(NR^{1B}—CH₂—CH₂)₁—. In some embodiments T is —(CH₂—CH₂—NR^{1B})ₙ—(CH₂)ₘC(O)—. In some embodiments, R^{1B} is —H. In some embodiments, R^{1B} is —CH₃. In some embodiments, R^{1B} is —CH₂CH₃. In some embodiments, R^{1B} is —CH₂CH₂NH₂. In some embodiments, each n is independently an integer ranging from 1 to 30. In some embodiments, each n is independently an integer ranging from 1 to 25. In some embodiments, each n is independently an integer ranging from 1 to 20. In some embodiments, each m is independently an integer ranging from 1 to 15. In some embodiments, each m is independently an integer ranging from 1 to 10. In some embodiments, each m is independently an integer ranging from 1 to 8. In some embodiments, each m is independently an integer ranging from 1 to 5.

### Portion G

In some embodiments, G is bound to c_{B}. In some embodiments, G is selected from the following substituents:

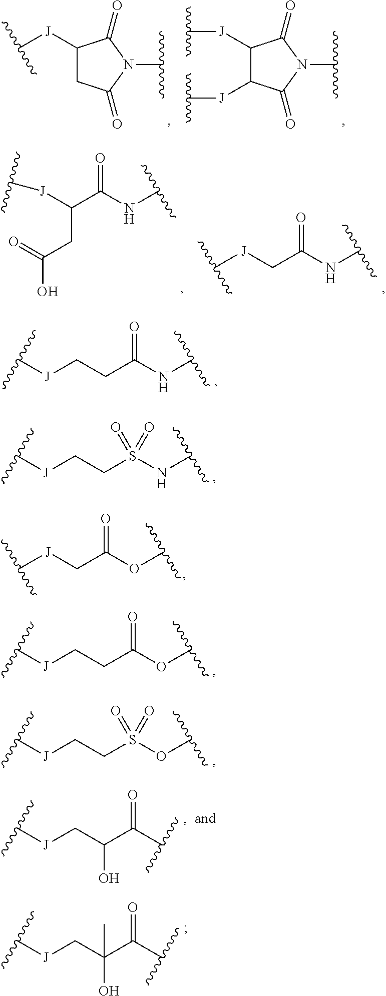

wherein
J is —O—, —S—, —C(R²²)₂— or —NR²²—, wherein each R²² is independently H or optionally substituted C₁-C₈ alkyl. In some embodiments, J is —O—, —NH— and —S—.

In some embodiments, G is bound to M. In some embodiments, G is

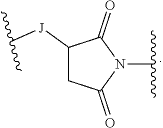

In some embodiments, G is

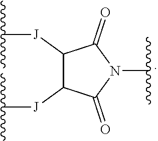

In some embodiments, G is

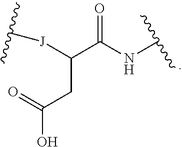

In some embodiments, G is

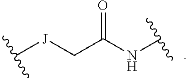

In some embodiments, G is

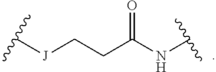

In some embodiments, G is

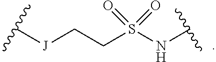

In some embodiments, G is

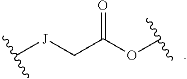

In some embodiments, G is

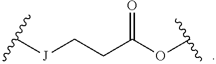

In some embodiments, G is

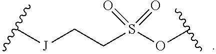

In some embodiments, G is

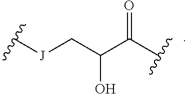

In some embodiments, G is

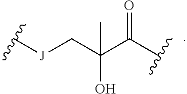

In some embodiments, J is —O—. In some embodiments, J is —S—. In some embodiments, J is —C(R²²)₂—. In some embodiments, J is —NR²²—.

In some embodiments —R²²— is —H. In some embodiments —R²²— is optionally substituted C₁-C₈ alkyl.

In certain embodiments, disclosed herein is a selective delivery molecule of Formula III, having the structure:

[G-T-c_{A}-Q-Y-D]-(—N—Z)ᵥ Formula III

in which G is a linker or a reactive group; T is a spacer; c_{A} is a bond or a single amino acid; Q is a bond or 1-3 amino acids; Y is 1-3 amino acids or an amino alkylene; D is an auristatin related therapeutic agent or an imaging agent; each N is independently a bond or a linker; Z is a peptide with a sequence comprising 3 to 10 amino acids or polyethylene glycol substituent; wherein each N is independently bound to G or c_{A}; and v is 1 or 2.

In some instances, G is a linker. In other instances, G is a reactive group. In some cases, the reactive group is an N-maleimide. In some cases, G is

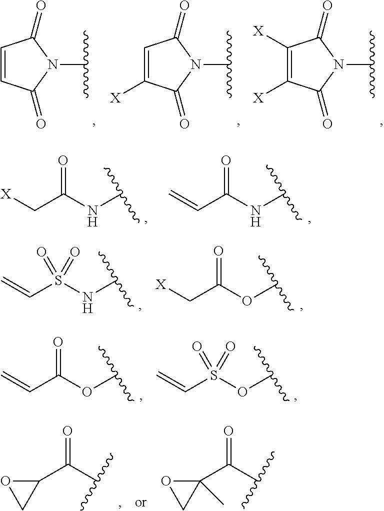

wherein each X is independently —Cl, —Br, —I, or —S-phenyl. In some cases, G is

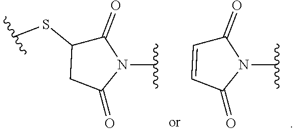

In some instances, T is an optionally substituted C₁-C₈ alkylene-C(O)—.

In some instances, c_{A} is

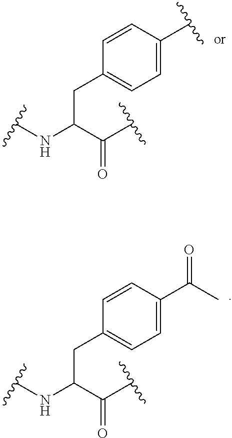

In some cases, Q is

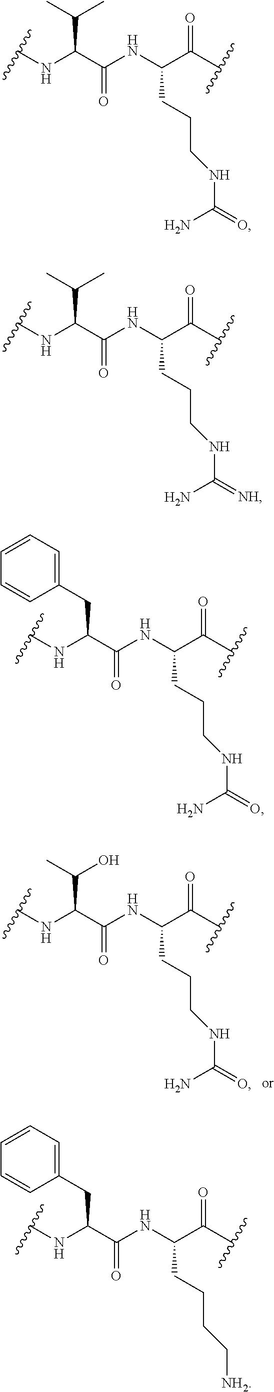 [SMILES: CN[C@H](C(=O)N[C@@H](CCCNC(N)=O)C(C)=O)C(C)C ; CN[C@H](C(=O)N[C@@H](CCCNC(=N)N)C(C)=O)C(C)C ; CN[C@@H](Cc1ccccc1)C(=O)N[C@@H](CCCNC(N)=O)C(C)=O ; CN[C@H](C(=O)N[C@@H](CCCNC(N)=O)C(C)=O)C(C)O ; CN[C@@H](Cc1ccccc1)C(=O)N[C@@H](CCCCN)C(C)=O]

In some embodiments, Y is —NHCH₂C(O)— or —NHCH(CH₃)C(O)—.

In some embodiments, D is MMAE or MMAF.

In some embodiments, N is a bond,

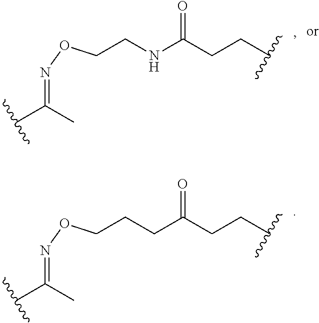

In some embodiments, Z is

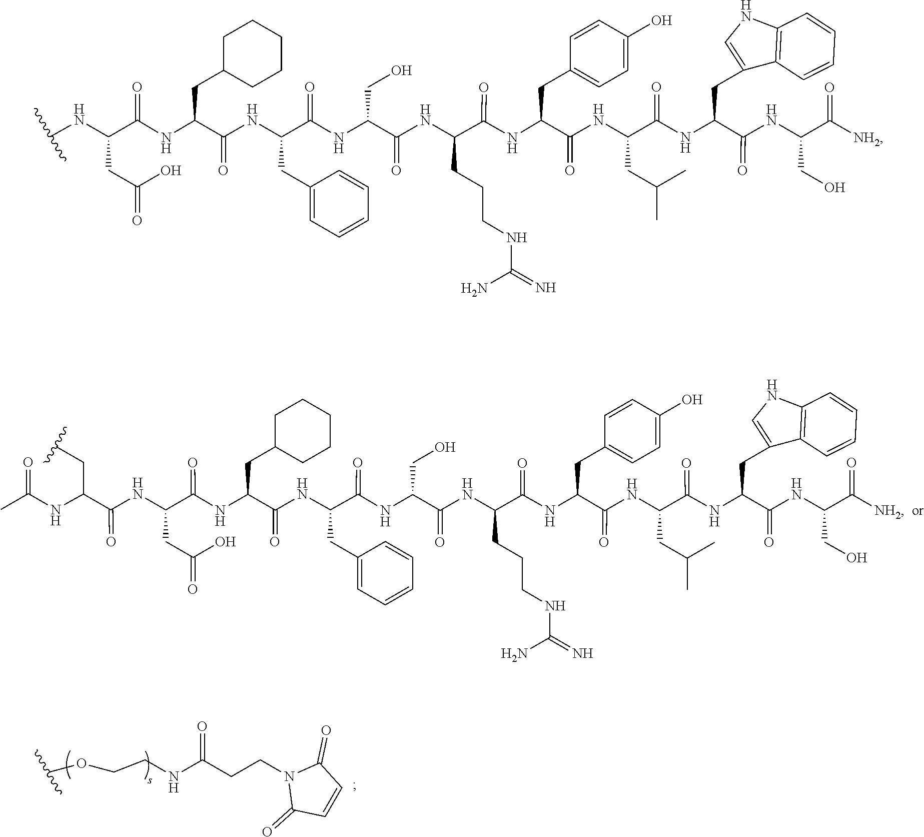

and wherein s is 1 to 20.

In some embodiments, G is

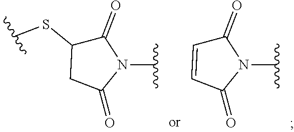

T is an optionally substituted C₁-C₈ alkylene-C(O)—; c_{A} is

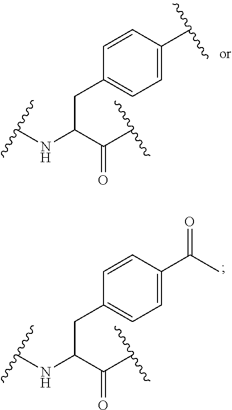 [SMILES: *NC(Cc1ccc(*)cc1)C(*)=O ; *NC(Cc1ccc(C(C)=O)cc1)C(*)=O]

### Q is

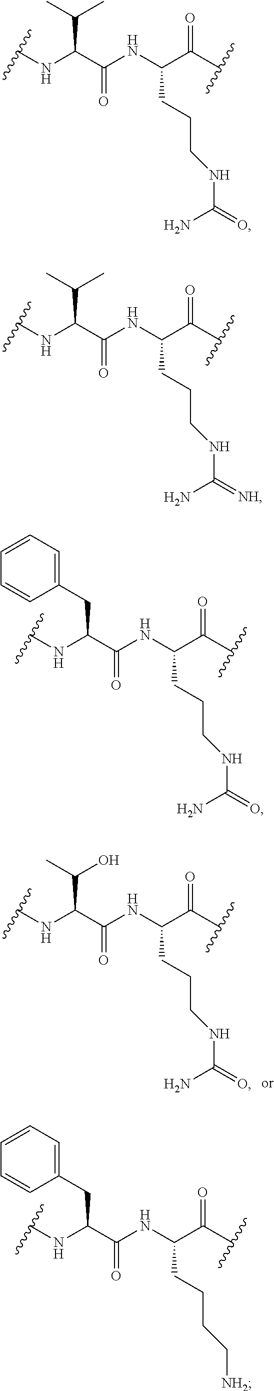 [SMILES: CN[C@H](C(=O)N[C@@H](CCCNC(N)=O)C(C)=O)C(C)C ; CN[C@H](C(=O)N[C@@H](CCCNC(=N)N)C(C)=O)C(C)C ; CN[C@@H](Cc1ccccc1)C(=O)N[C@@H](CCCNC(N)=O)C(C)=O ; CN[C@H](C(=O)N[C@@H](CCCNC(N)=O)C(C)=O)C(C)O ; CN[C@@H](Cc1ccccc1)C(=O)N[C@@H](CCCCN)C(C)=O]

Y is —NHCH₂C(O)— or —NHCH(CH₃)C(O)—; D is MMAE or MMAF; N is a bond,

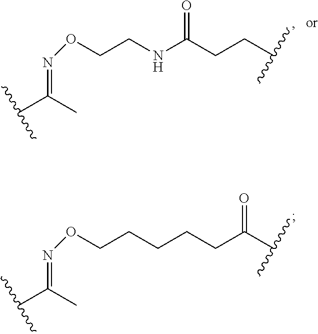 [SMILES: *CCC(=O)NCCO/N=C(/*)C ; *C(=O)CCCCCO/N=C(/*)C]

### Z is

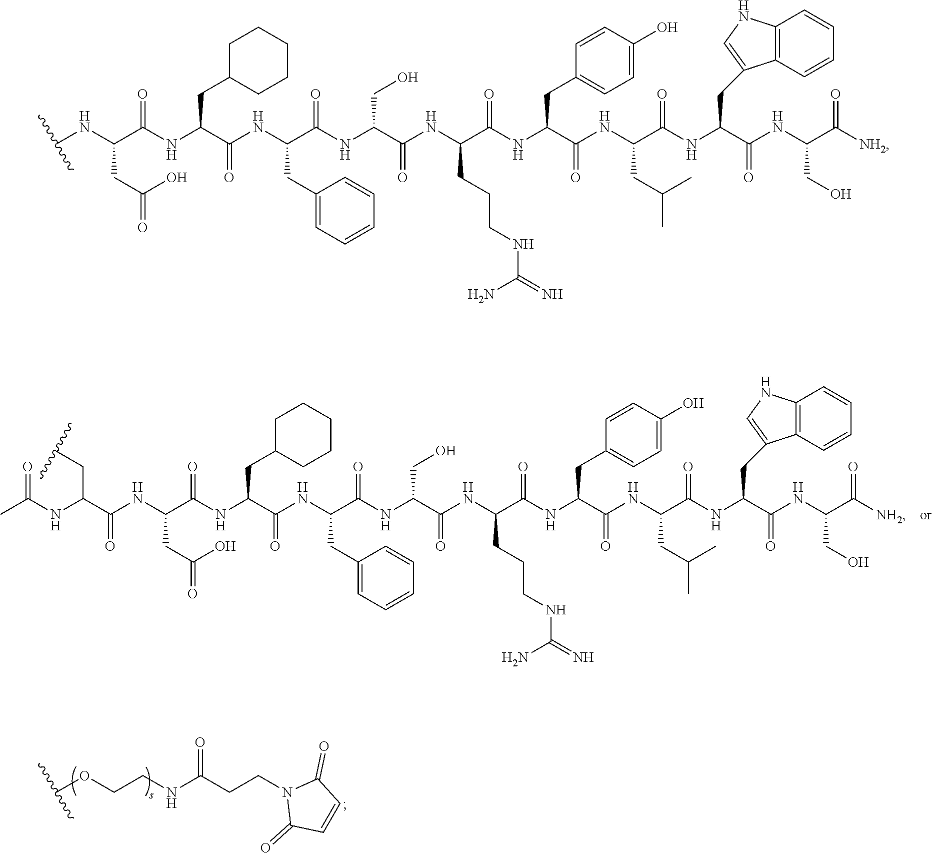

and wherein s is 1 to 20.

In some embodiments, the selective delivery molecule is: SDM-185.

In some embodiments, the selective delivery molecule is SDM-193.

### Exemplary Compounds

In some embodiments, the selective delivery molecule described herein has a structure provided in Tables 1-8.

TABLE 1

| Com- pound Name | Chemical Structure 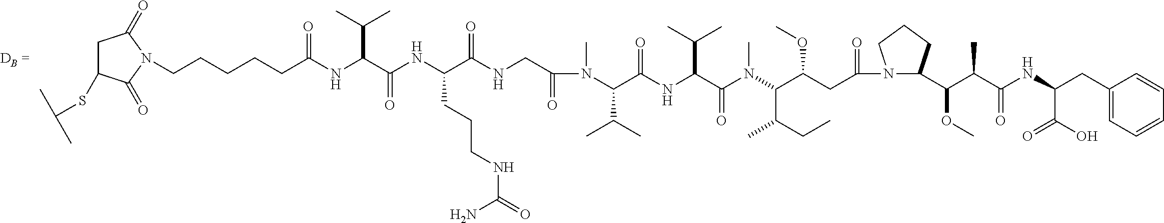 |
|---|---|
| SDM- 173 | 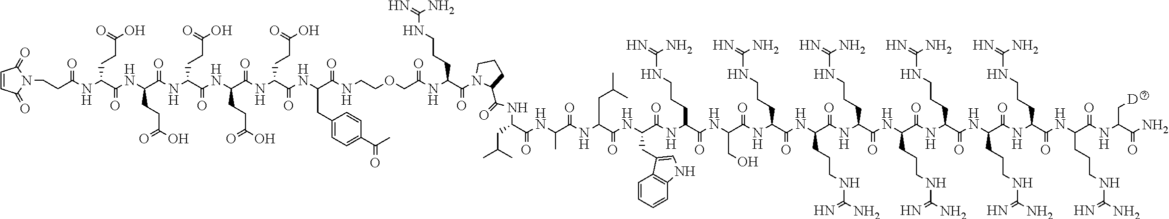 |
| SDM- 178 | 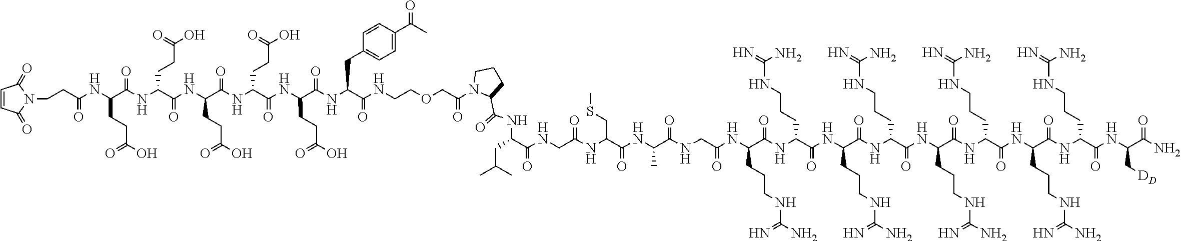 |
| SDM- 181 | 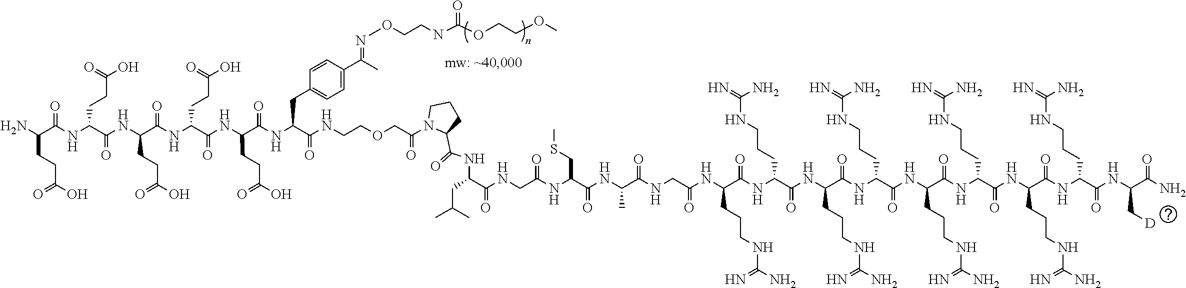 |
| SDM- 182 | 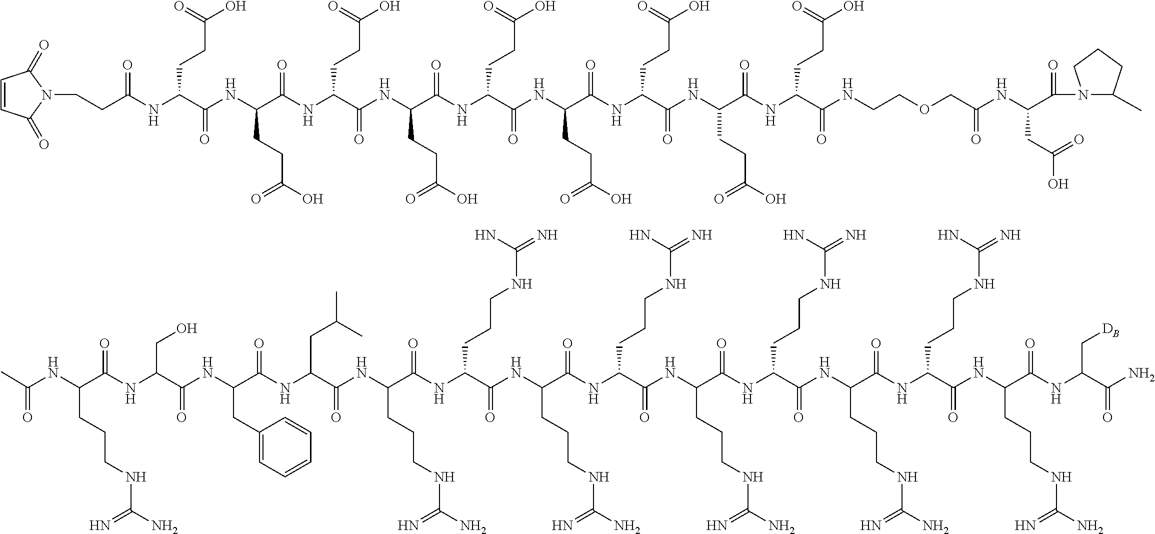 |
| SDM- 189 | 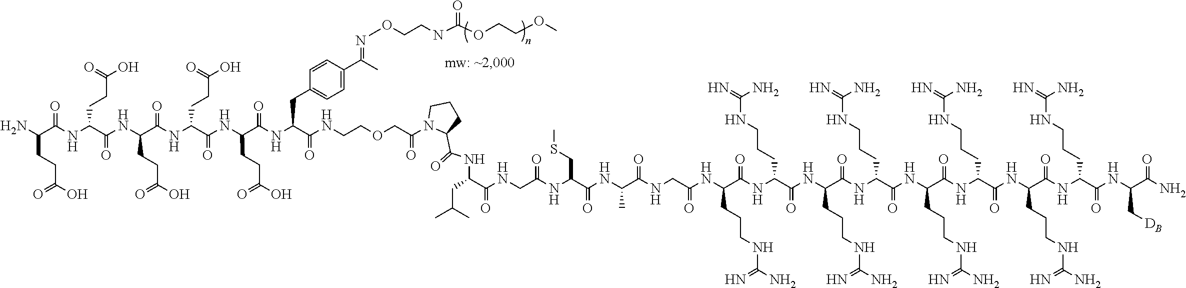 |
| SDM- 190 | 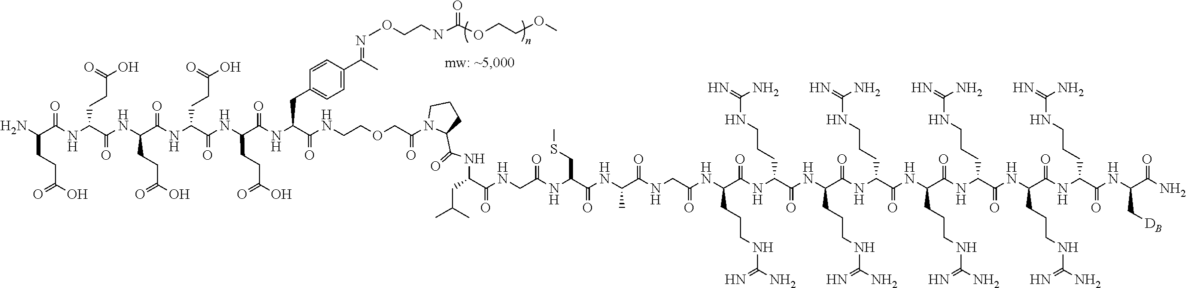 |
| SDM- 191 | 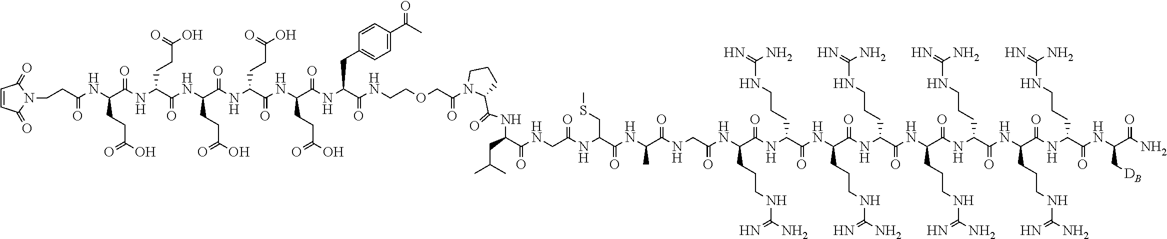 |
| SDM- 195 | 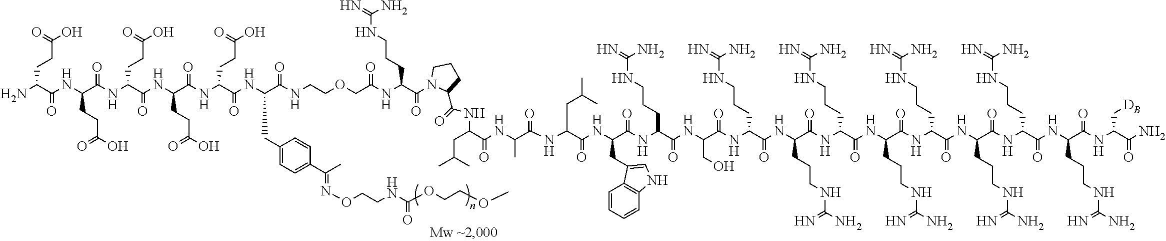 |
| SDM- 199 | 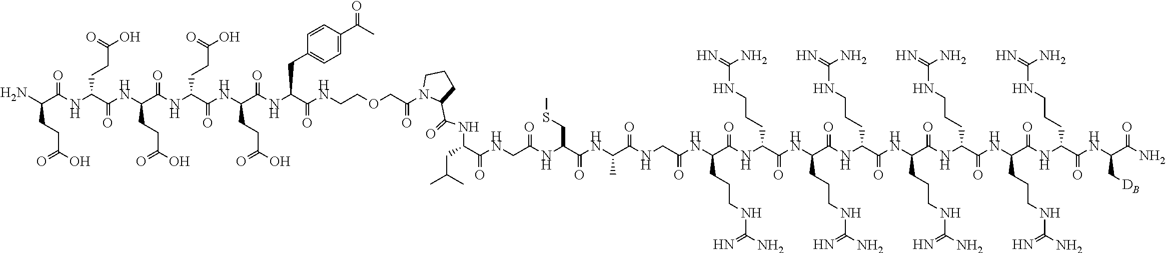 |
| SDM- 200 | 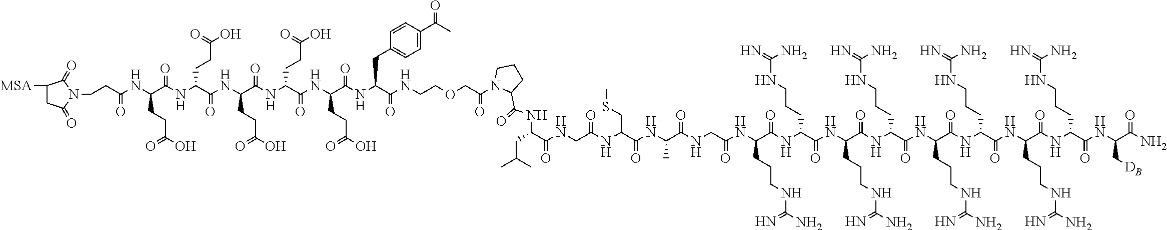 |
| SDM- 210 | 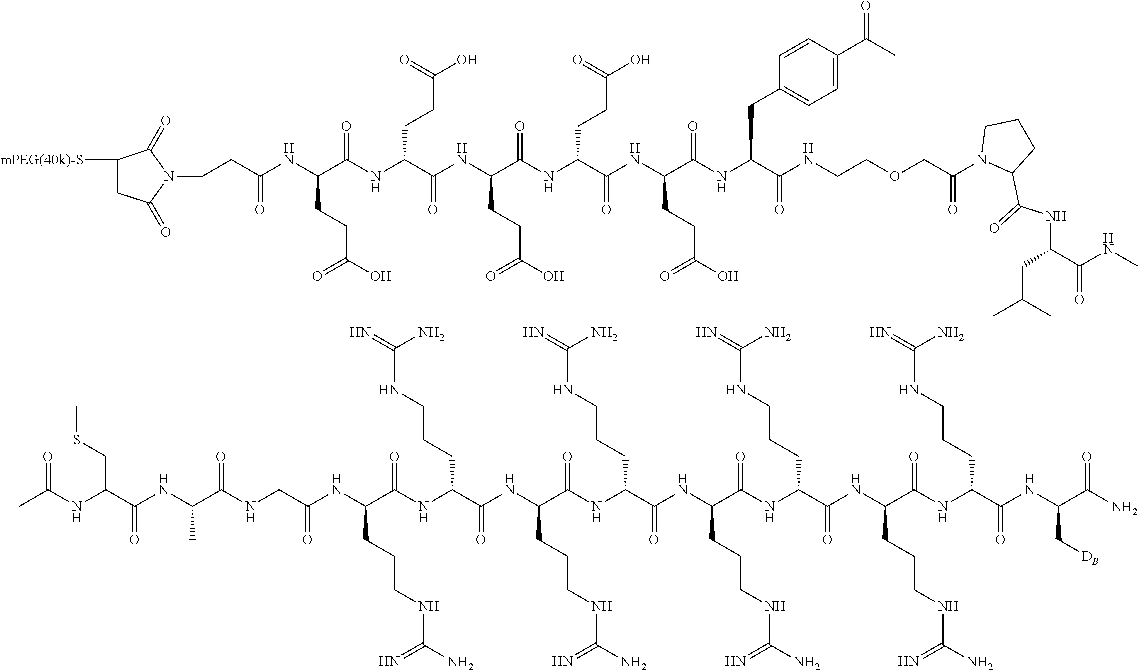 |
| SDM- 211 | 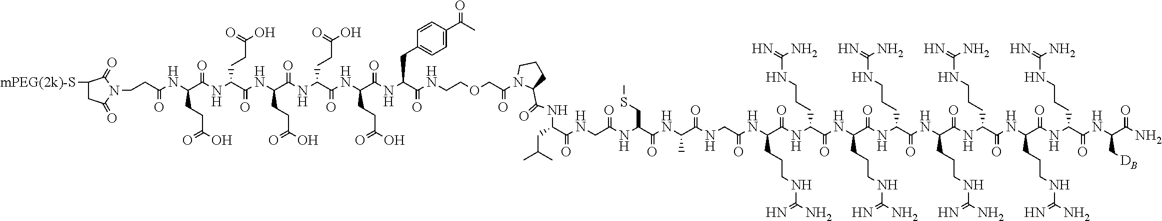 |
| SDM- 212 | 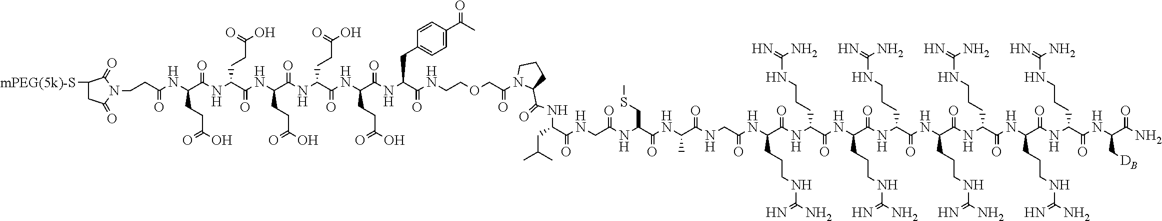 |
| SDM- 218 | 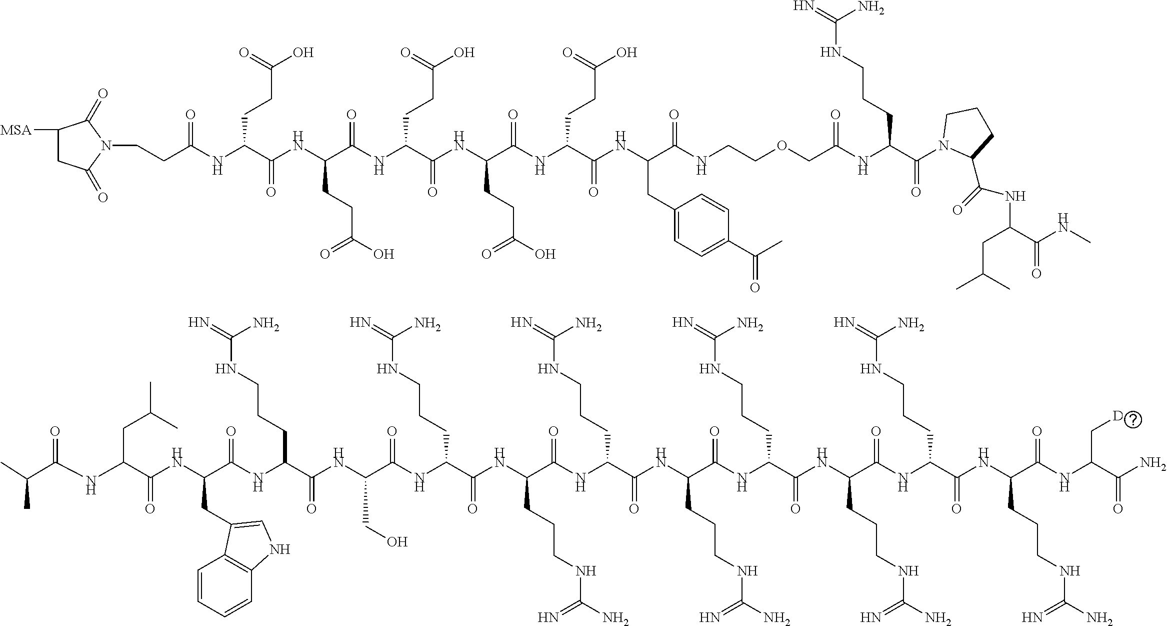 |
| SDM- 219 | 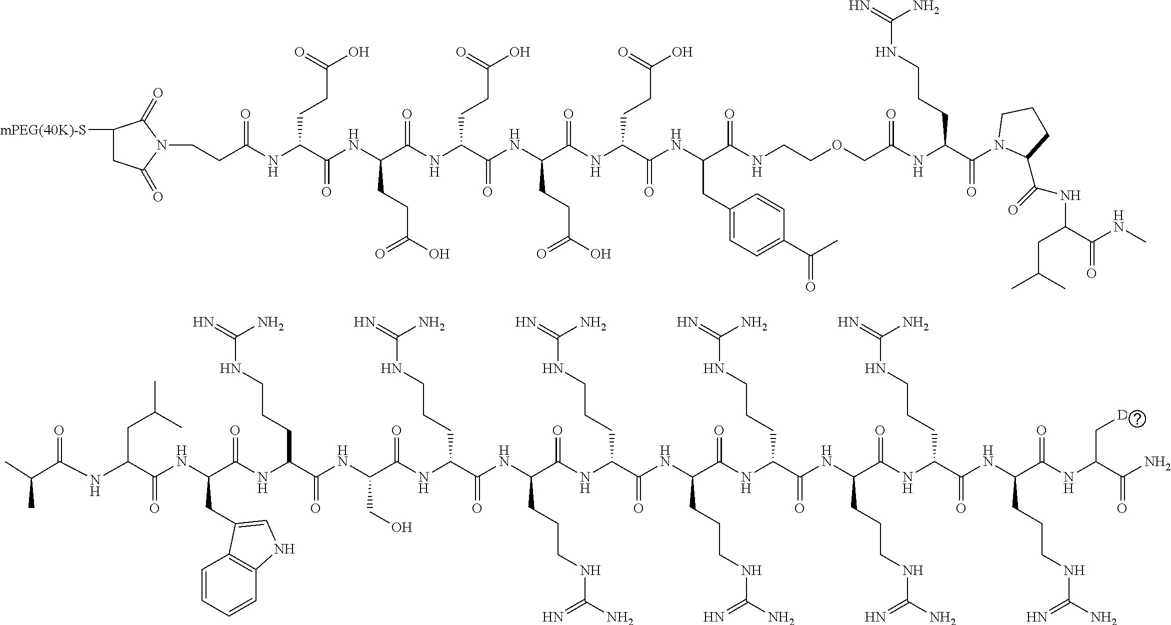 |
| SDM- 220 | 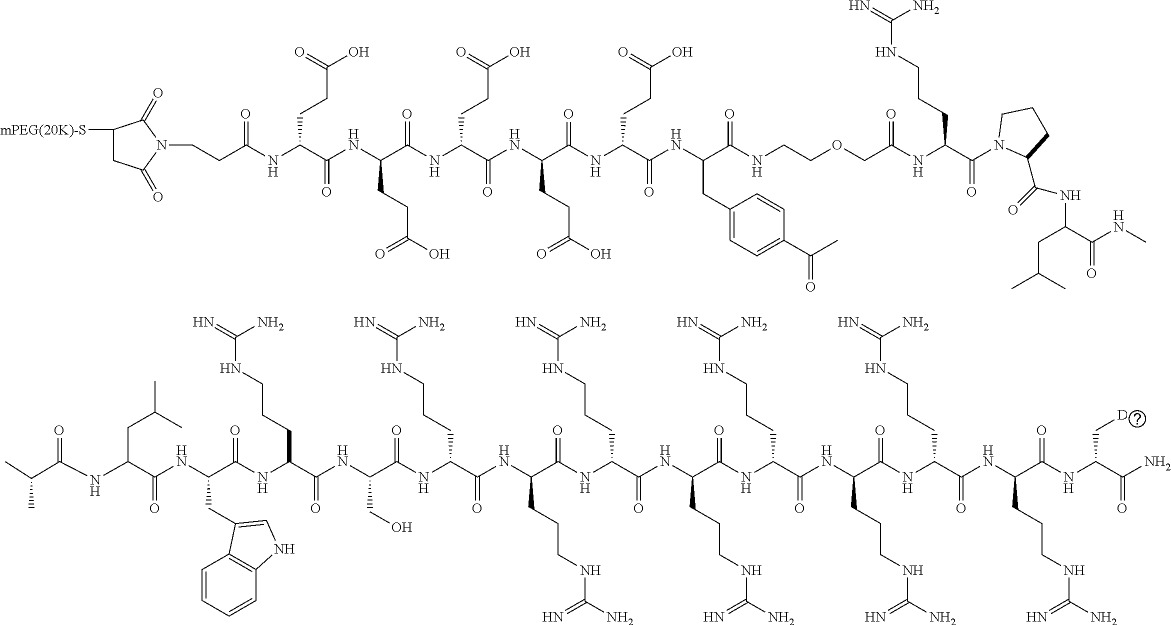 |
| SDM- 221 | 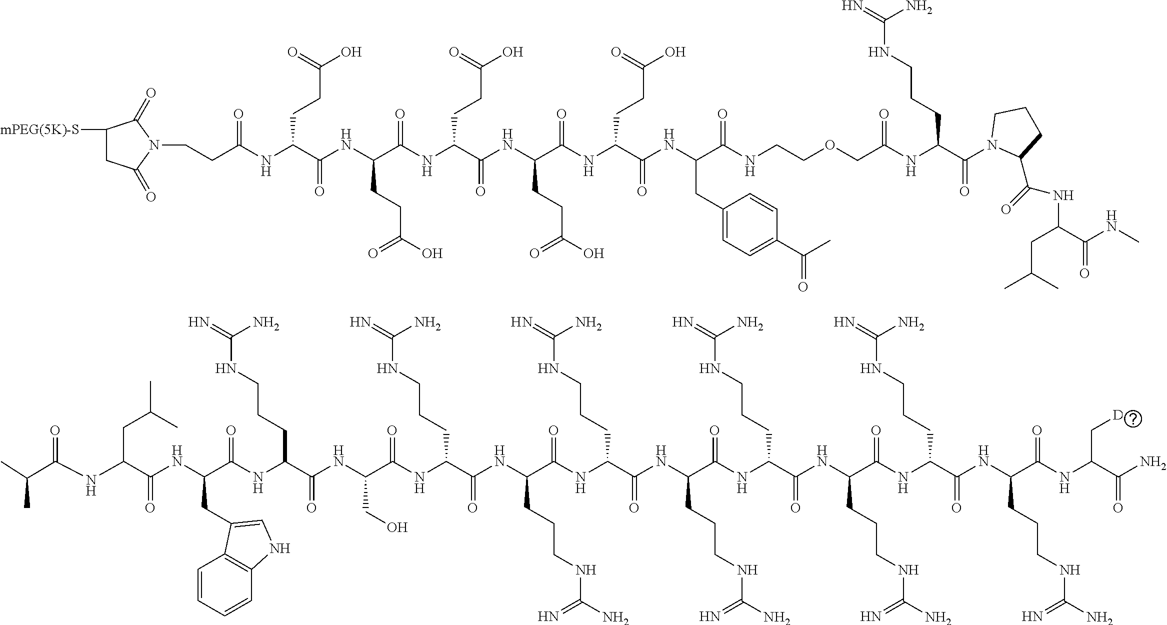 |
| SDM- 222 | 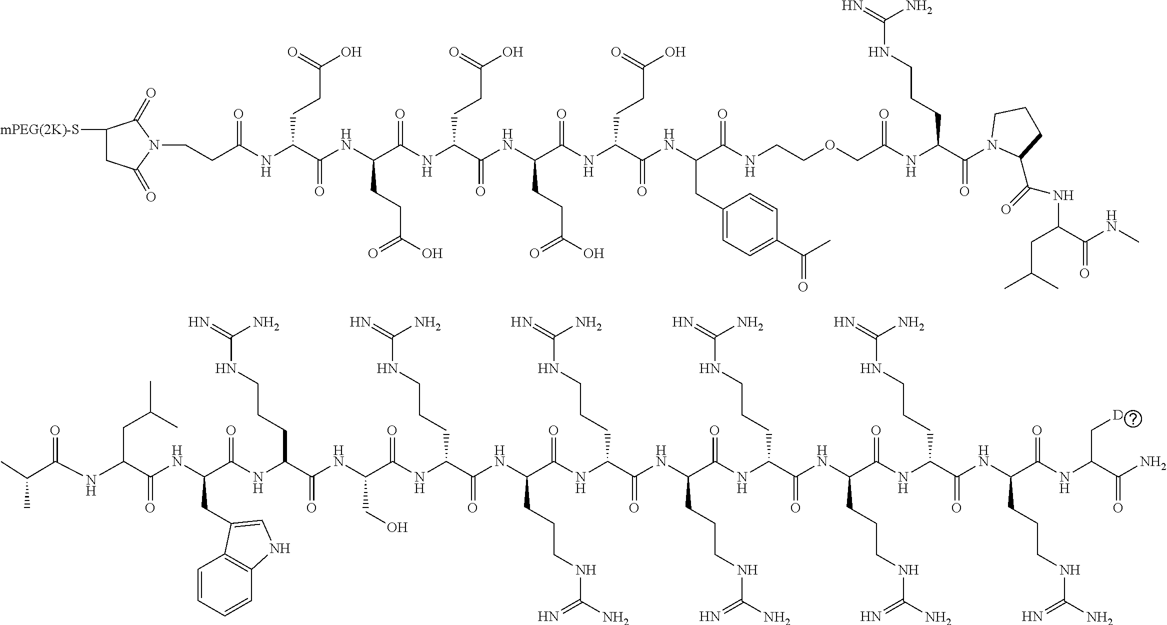 |
| SDM- 225 | 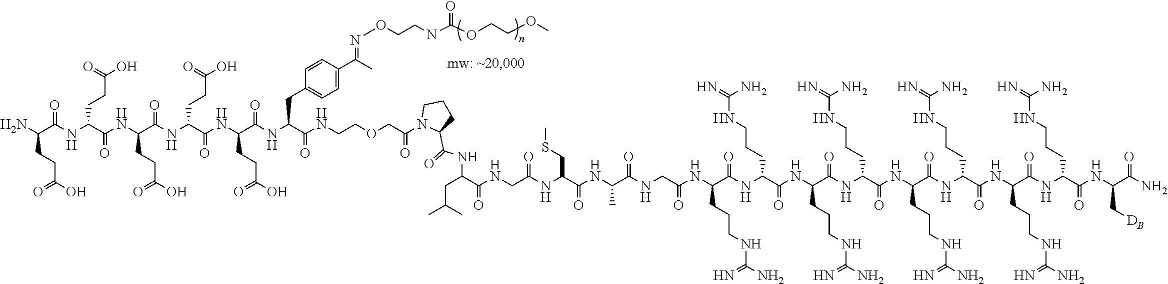 |
| SDM- 227 | 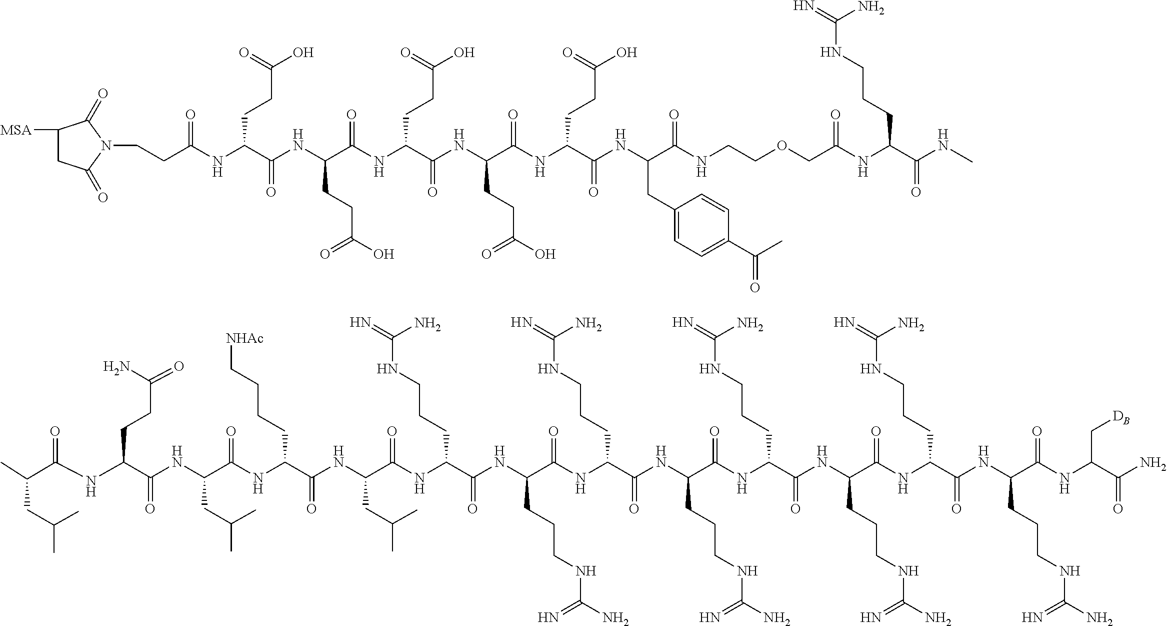 |
| SDM- 228 | 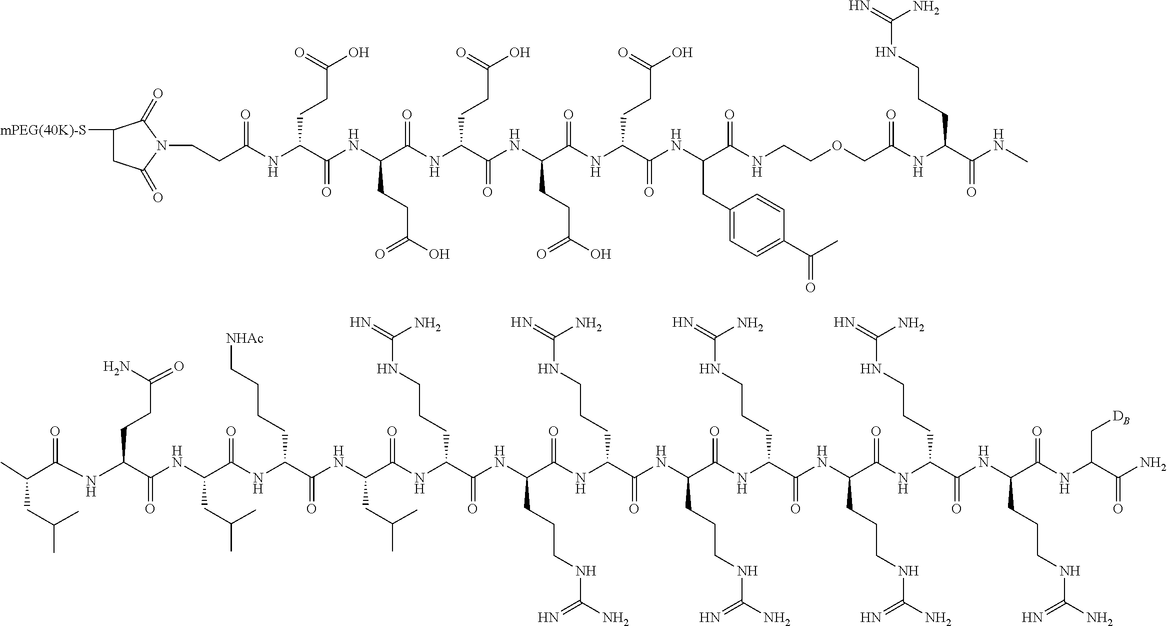 |
| SDM- 229 | 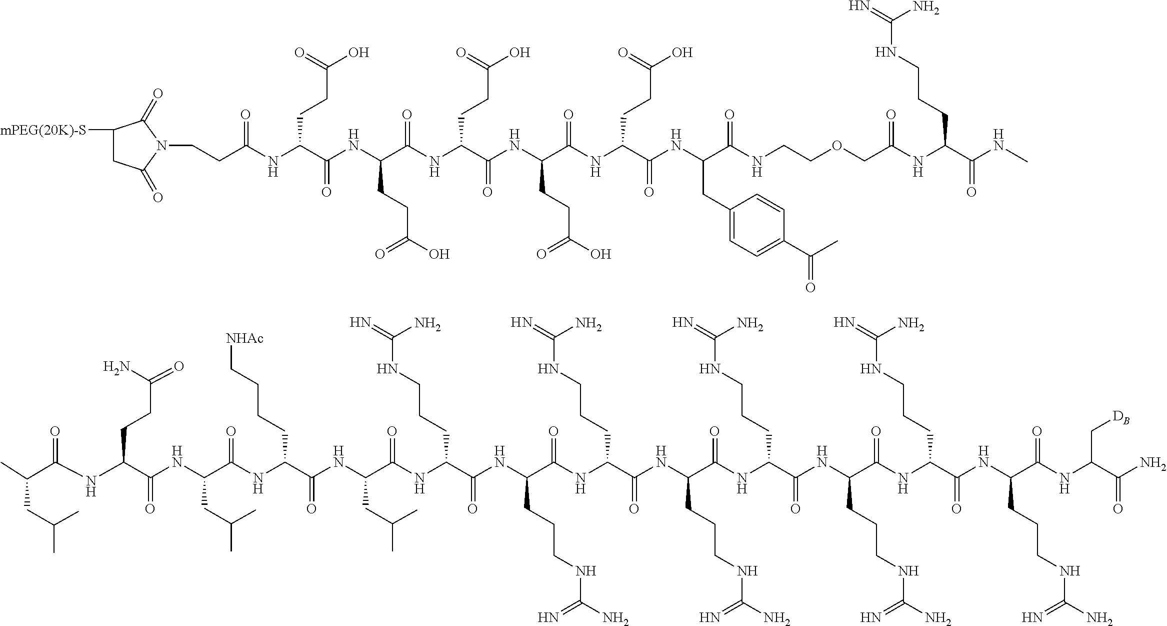 |
| SDM- 230 | 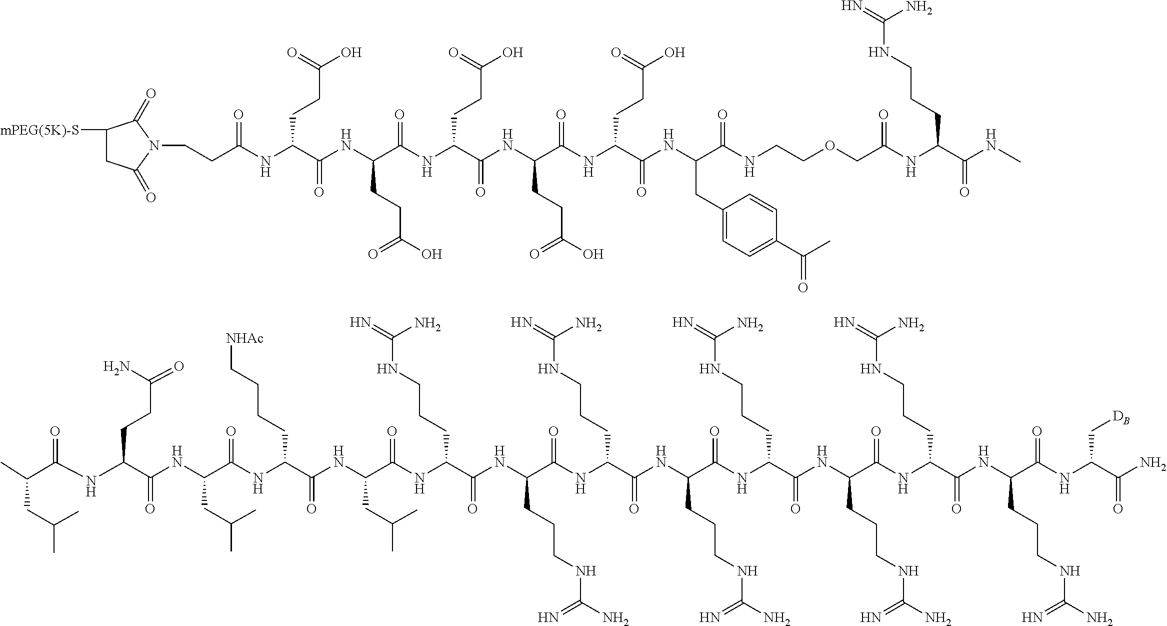 |
| SDM- 231 | 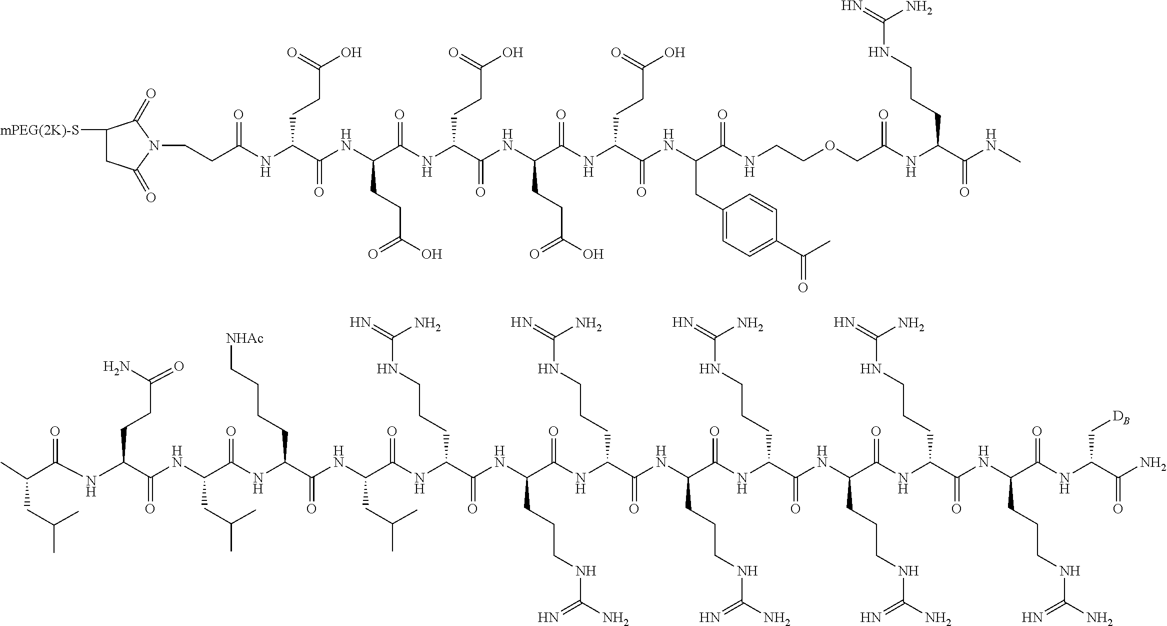 |
| SDM- 257 | 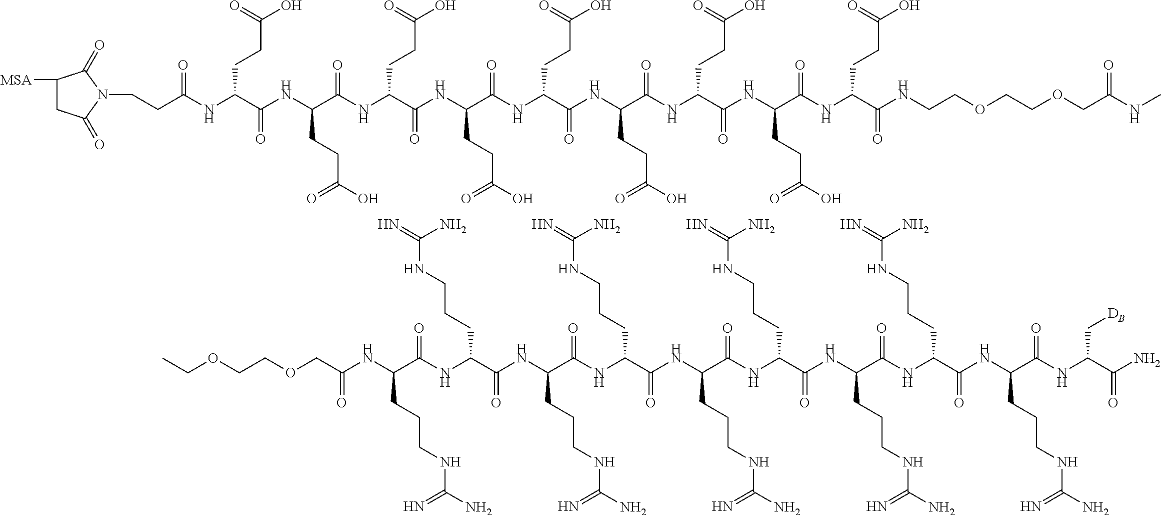 |
| SDM- 266 | 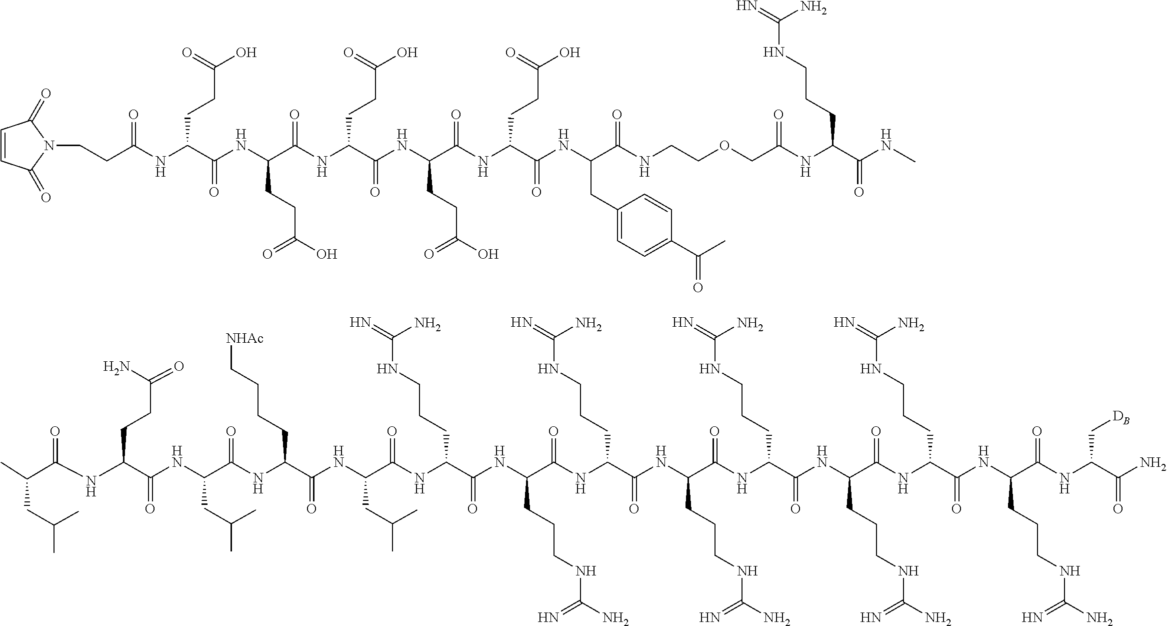 |
| SDM- 176 (nnon- cleav- able con- trol | 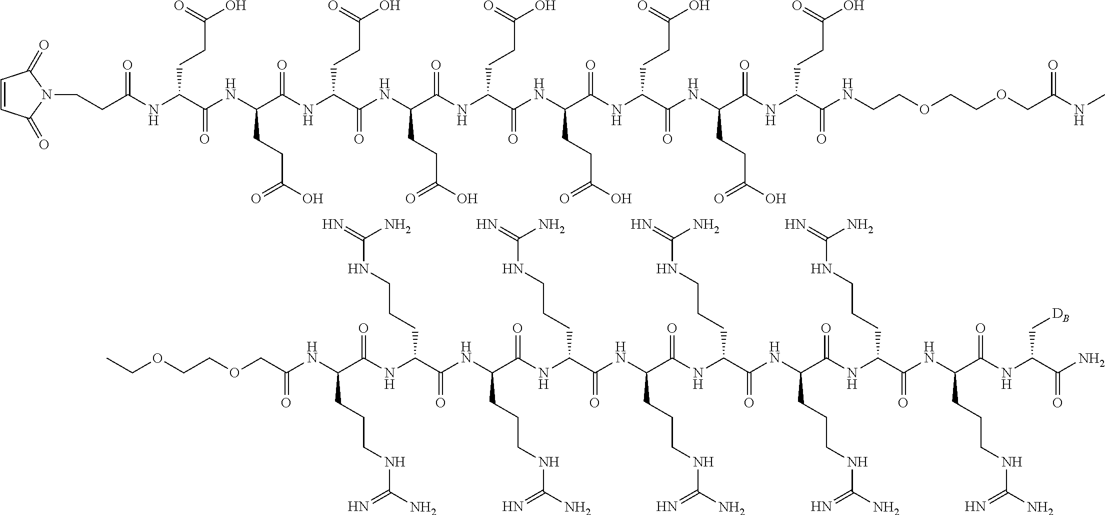 |

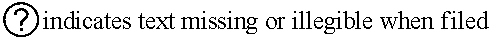

TABLE 2

| Com- pound Name | Chemical Structure 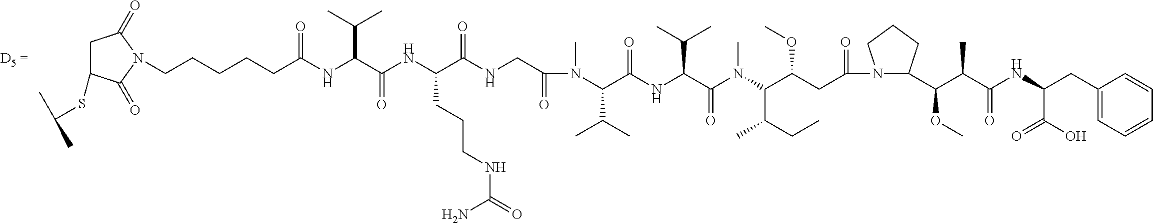 |
|---|---|
| SDM- 177 | 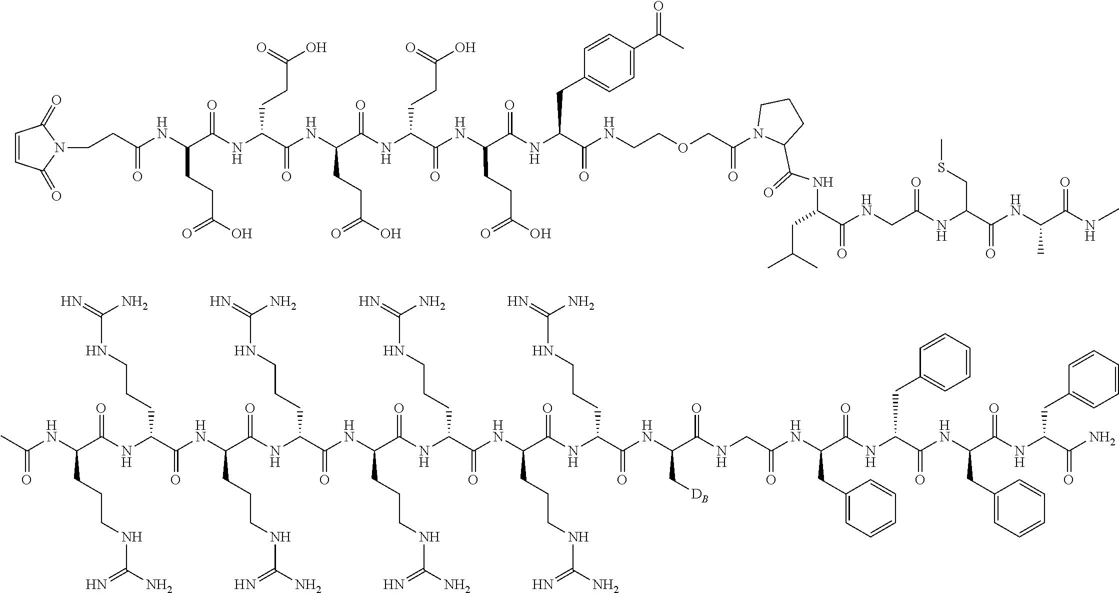 |
| SDM- 179 | 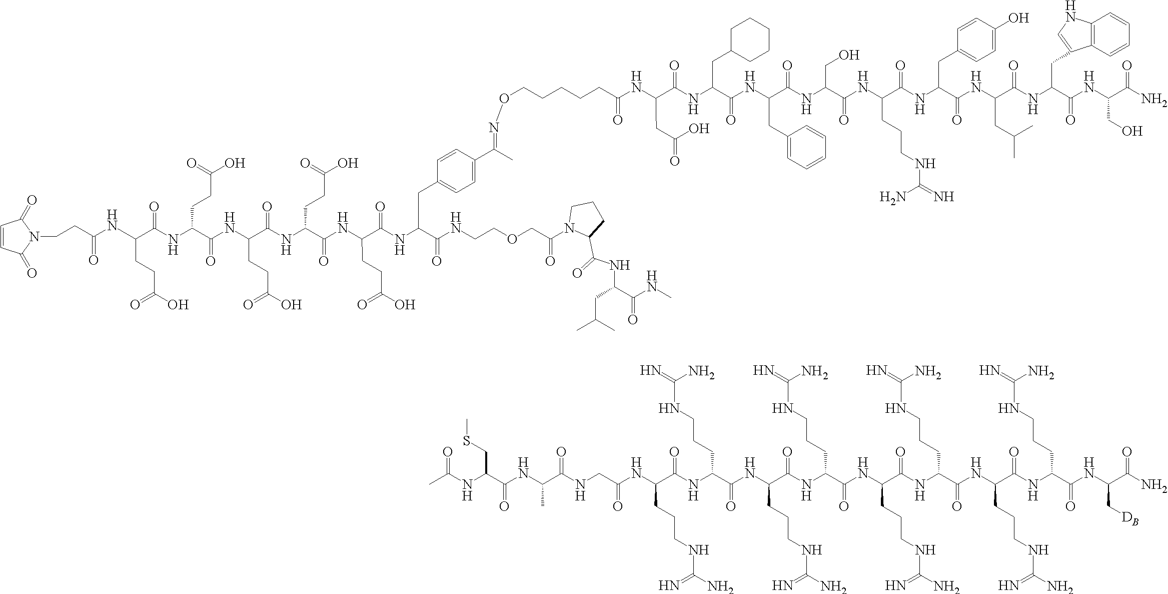 |
| SDM- 180 | 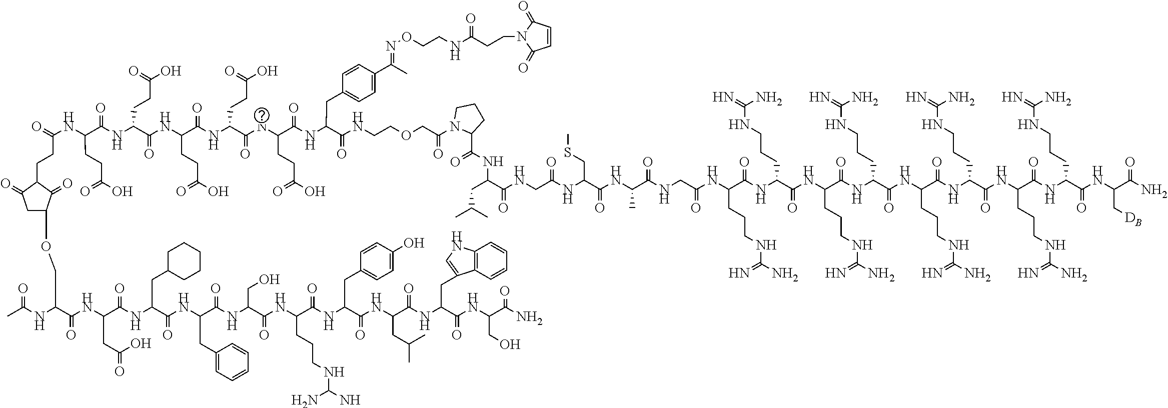 |
| SDM- 183 | 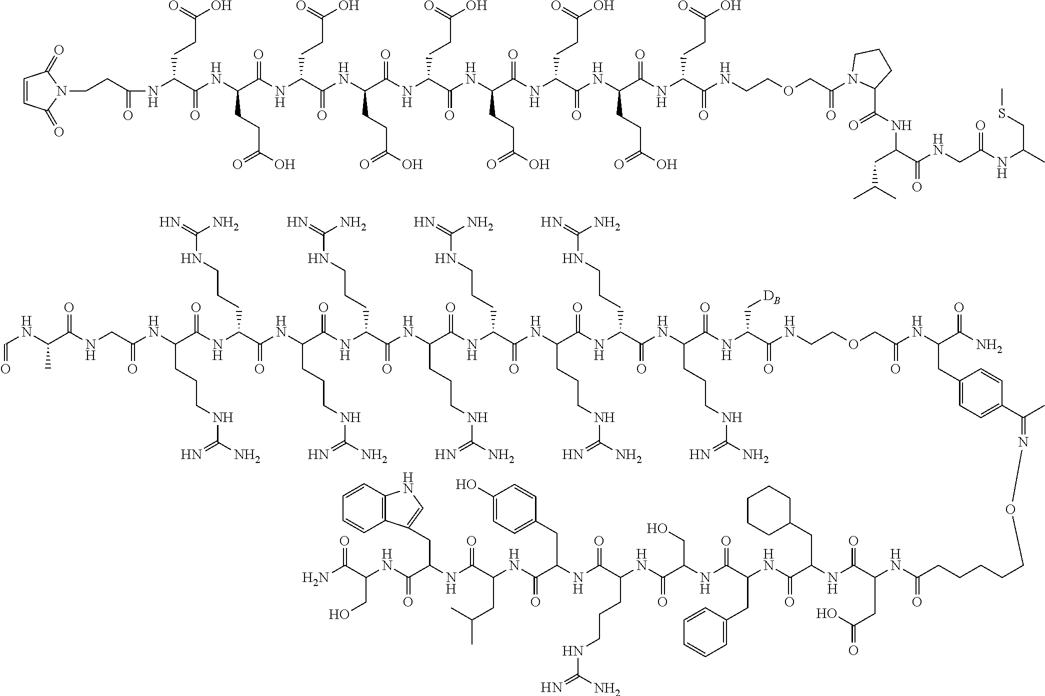 |
| SDM- 184 | 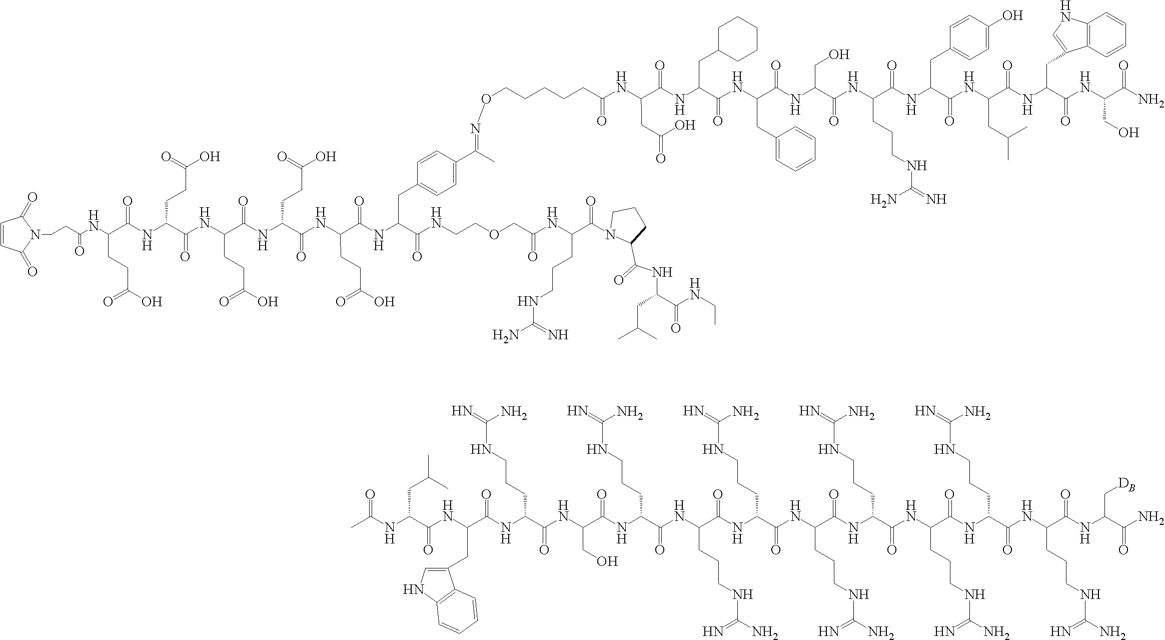 |
| SDM- 186 | 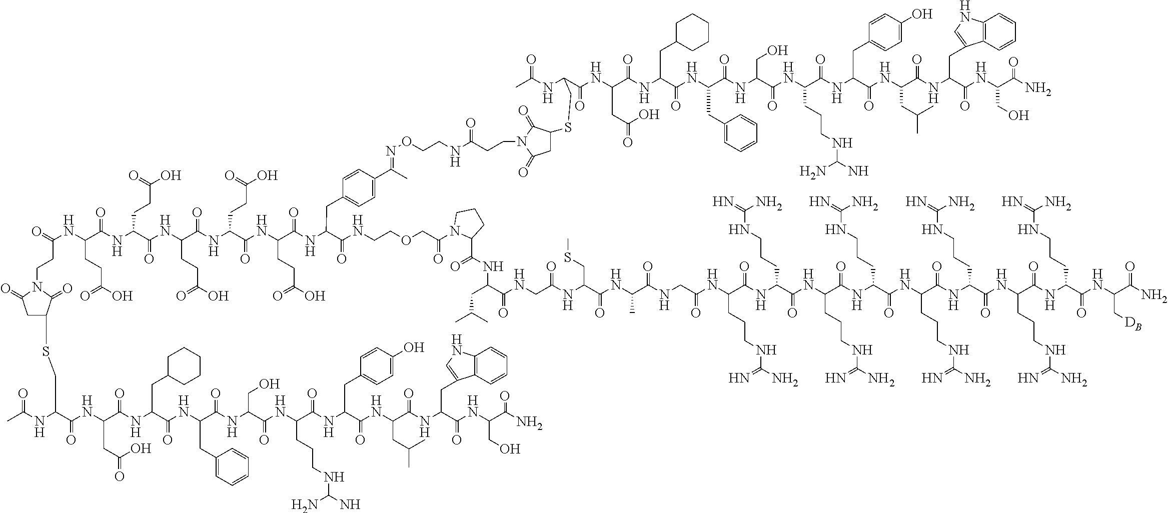 |
| SDM- 209 | 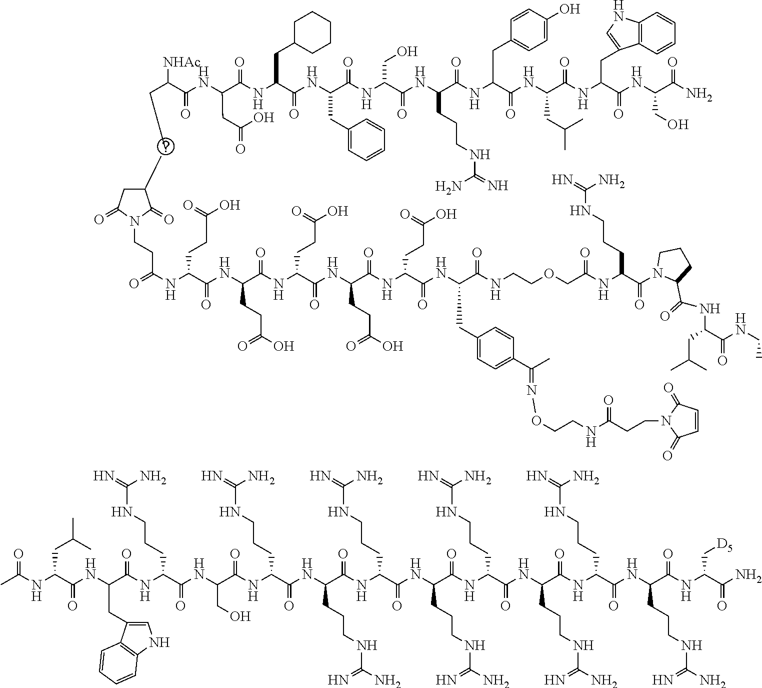 |
| SDM- 258 | 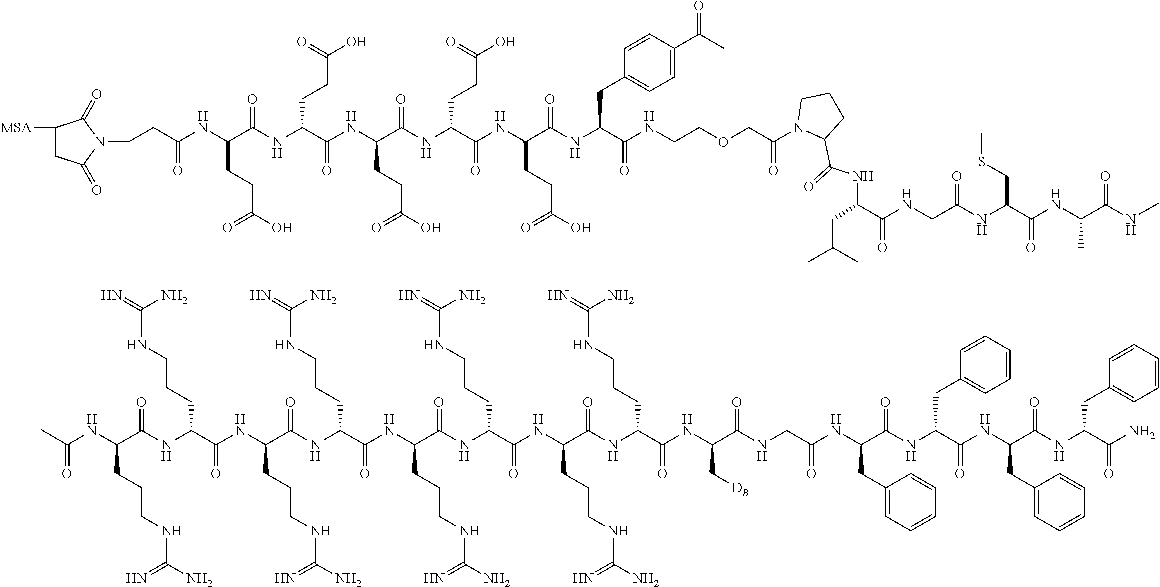 |
| SDM- 259 | 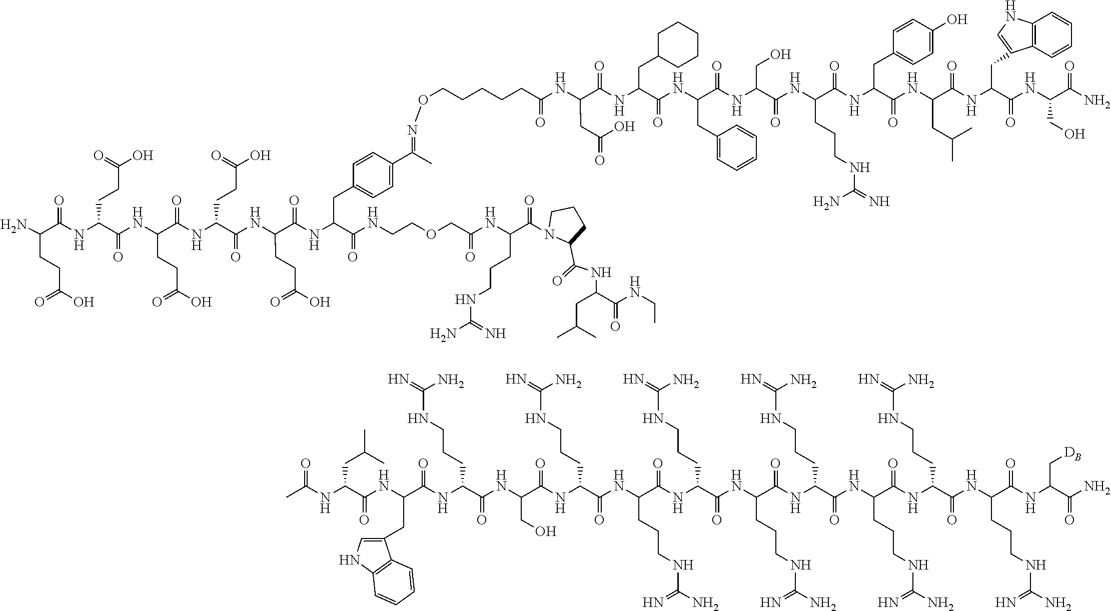 |
| SDM- 260 | 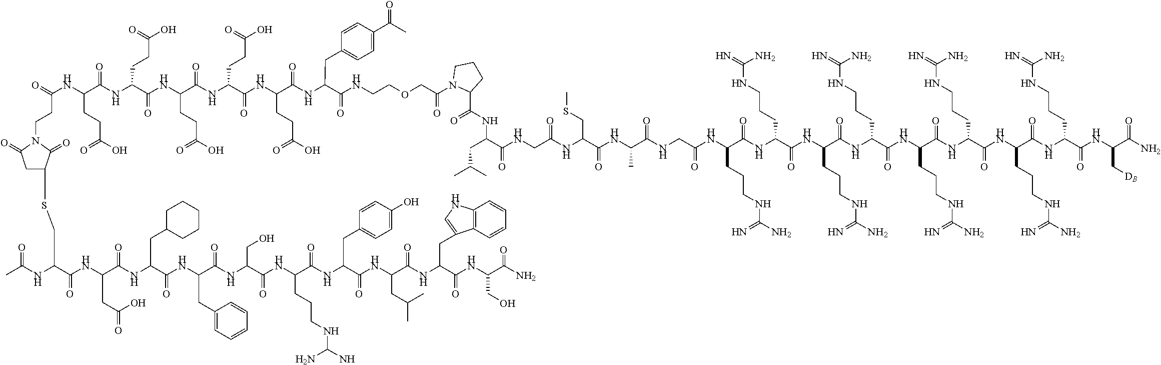 |

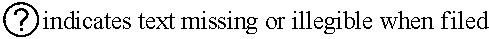

TABLE 3

| Compound | |
|---|---|
| Name | Chemical Structure |
| SDM-185 | 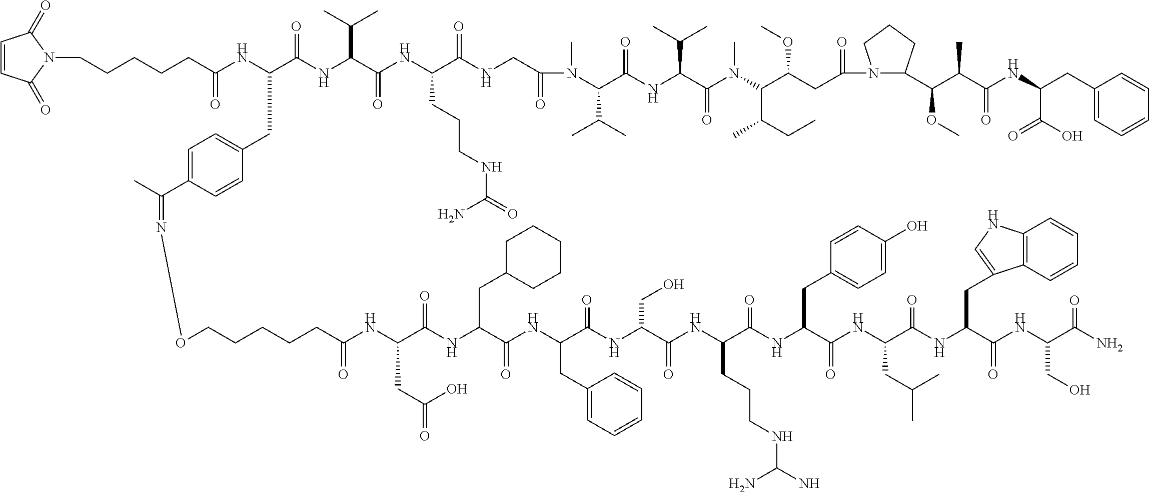 |
| SDM-193 | 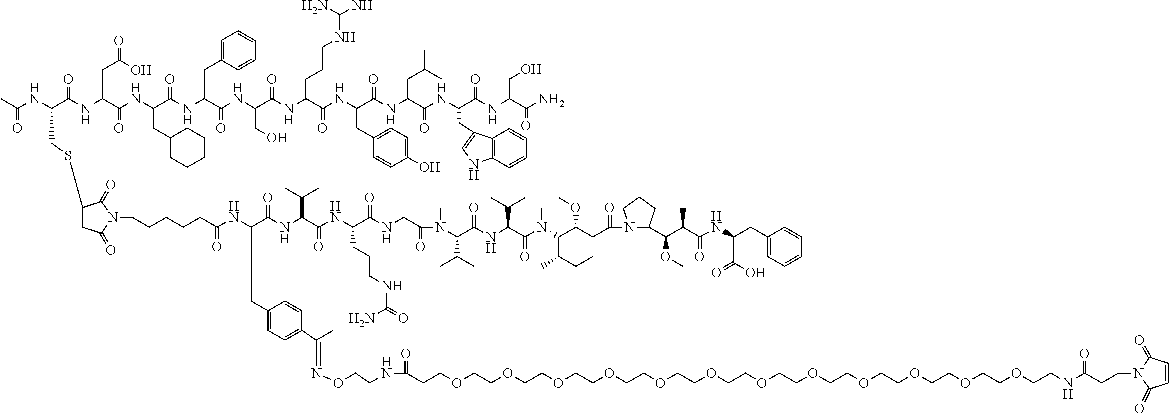 |

TABLE 4

| | |
|---|---|
| Compound Name | Chemical Structure 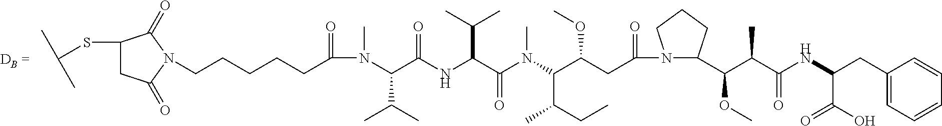 |
| SDM-201 | 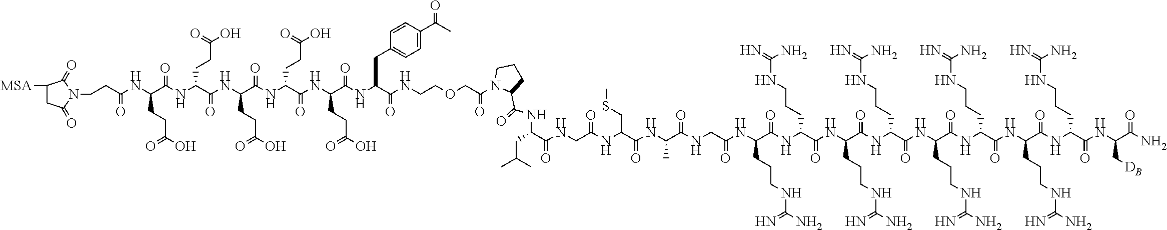 |
| SDM-202 | 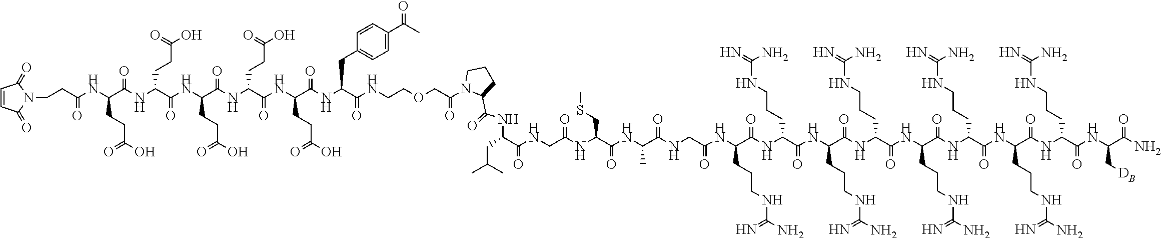 |

TABLE 5

| Compound Name | Chemical Structure 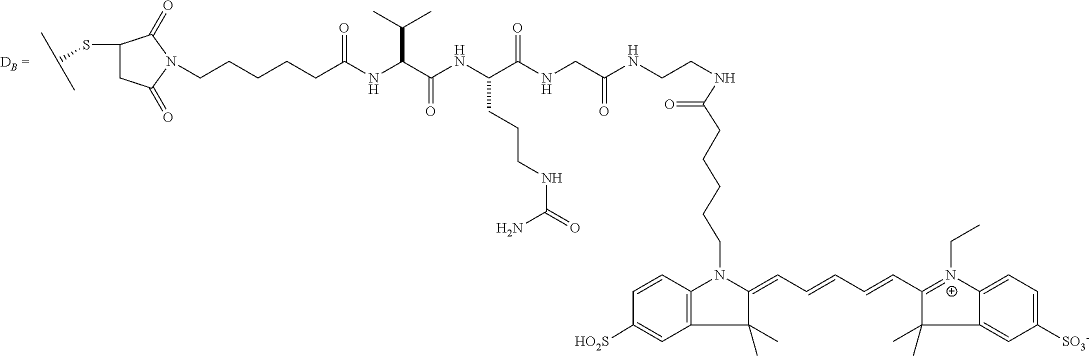 |
|---|---|
| SDM-192 | 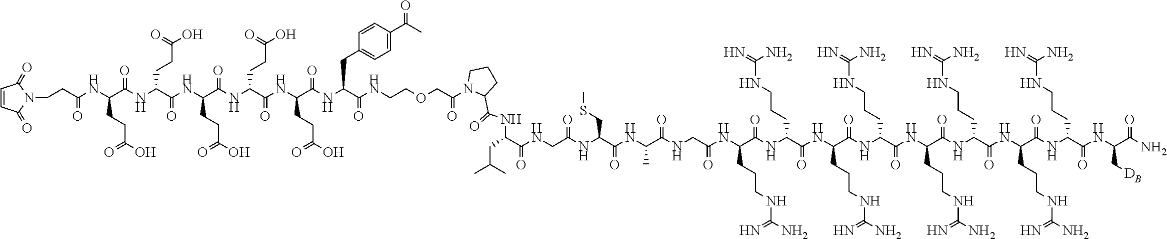 |
| SDM-236 | 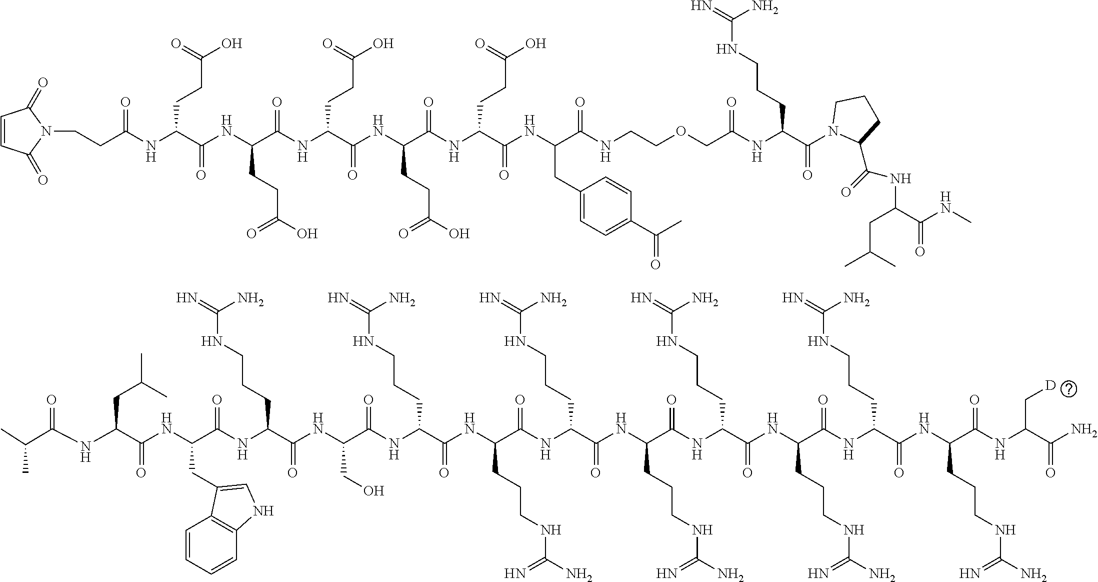 |

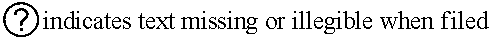

TABLE 6

| | |
|---|---|
| Compound Name | Chemical Structure 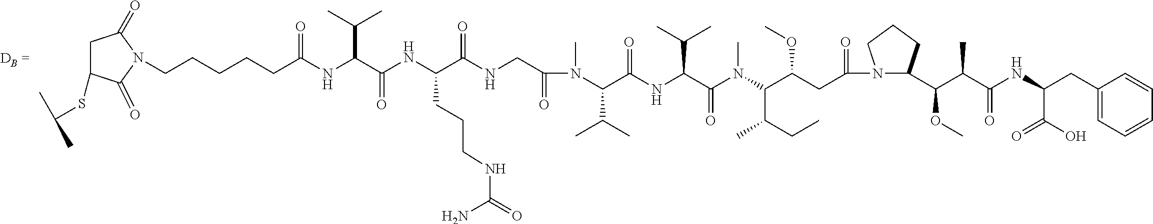 |
| SDM-187 | 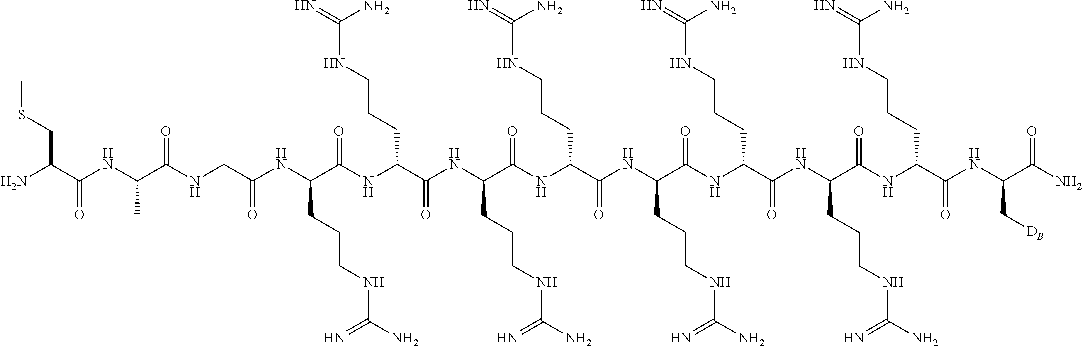 |

TABLE 7

| Compound Name | Chemical Structure 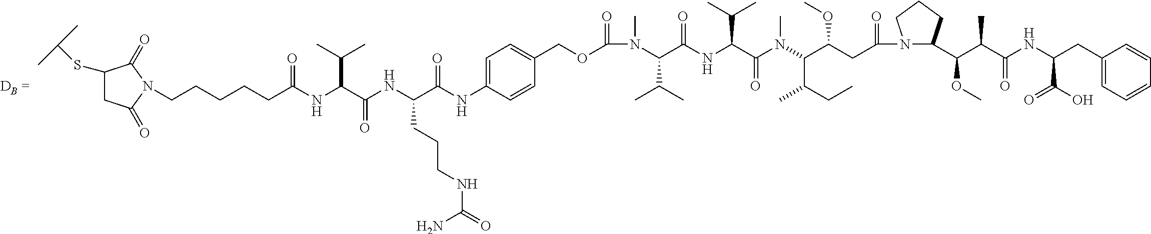 |
|---|---|
| SDM-203 | 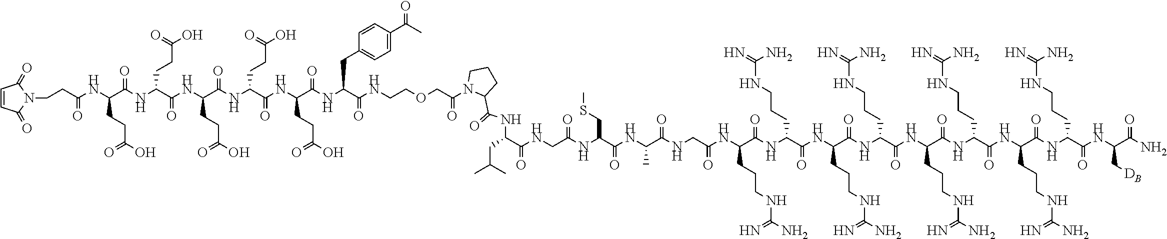 |
| SDM-204 | 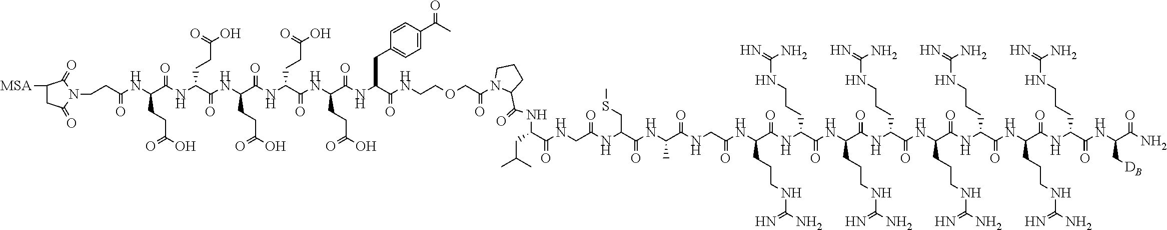 |
| SDM-208 | 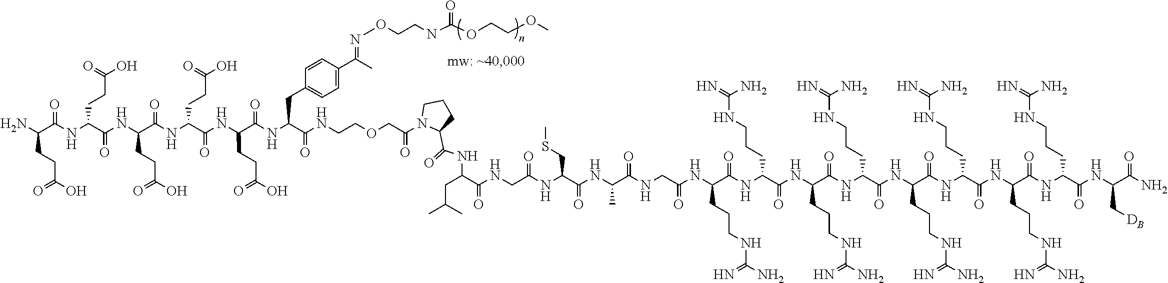 |
| SDM-213 | 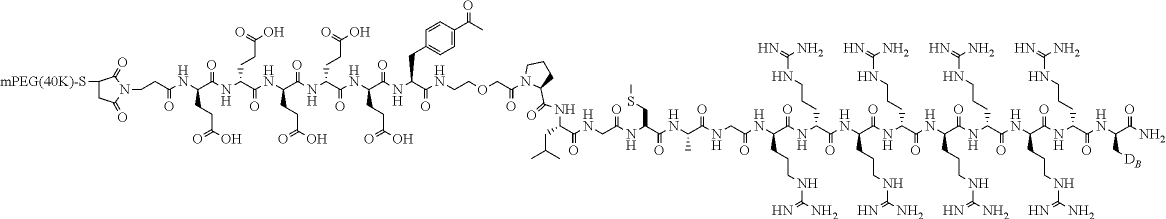 |
| SDM-214 | 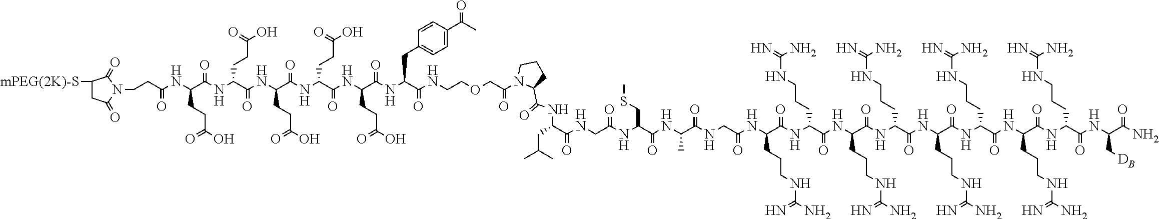 |
| SDM-215 | 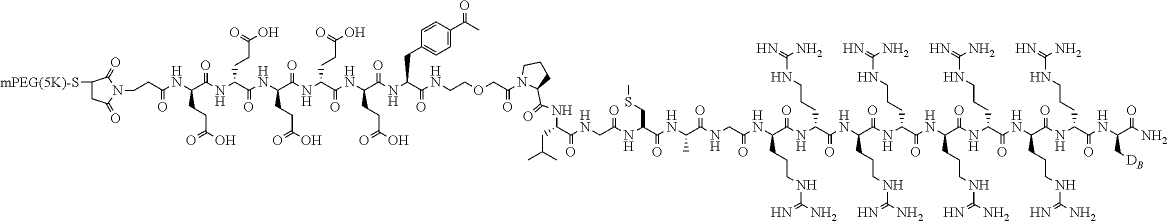 |
| SDM-216 | 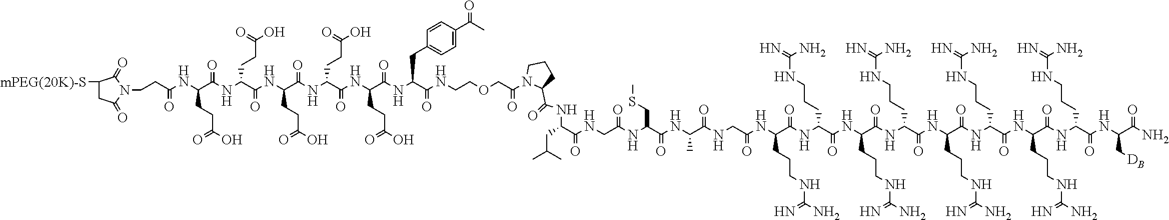 |
| SDM-223 | 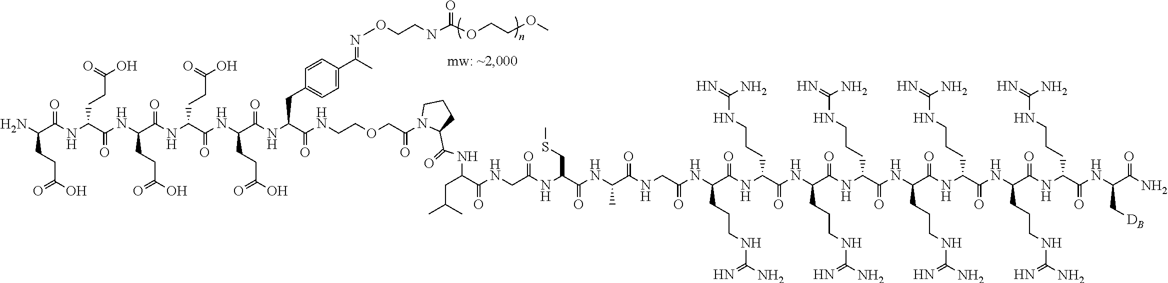 |
| SDM-224 | 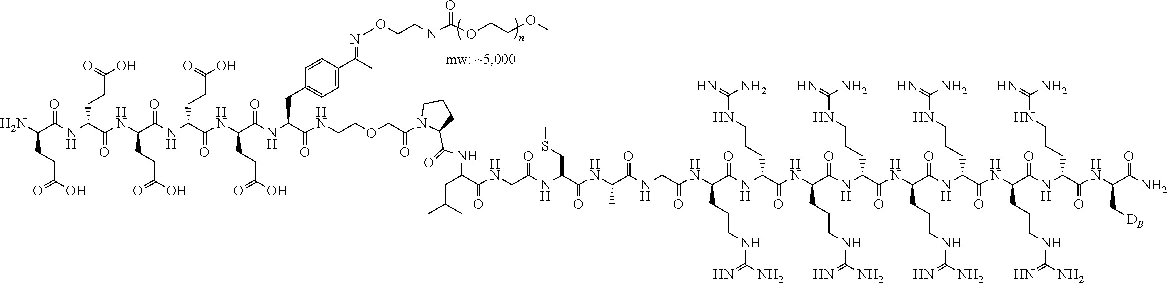 |
| SDM-226 | 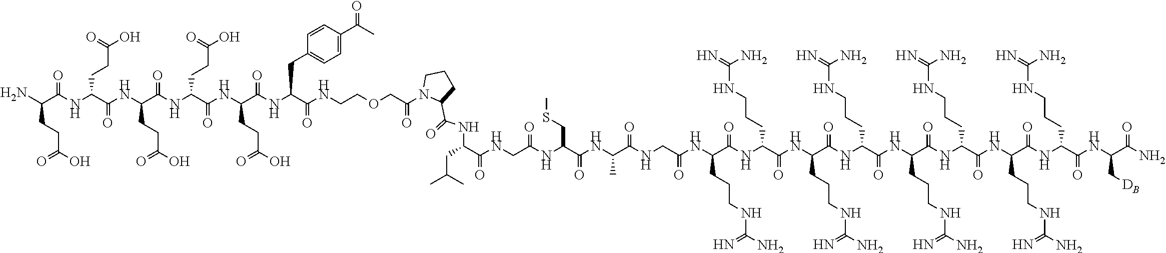 |
| SDM-232 | 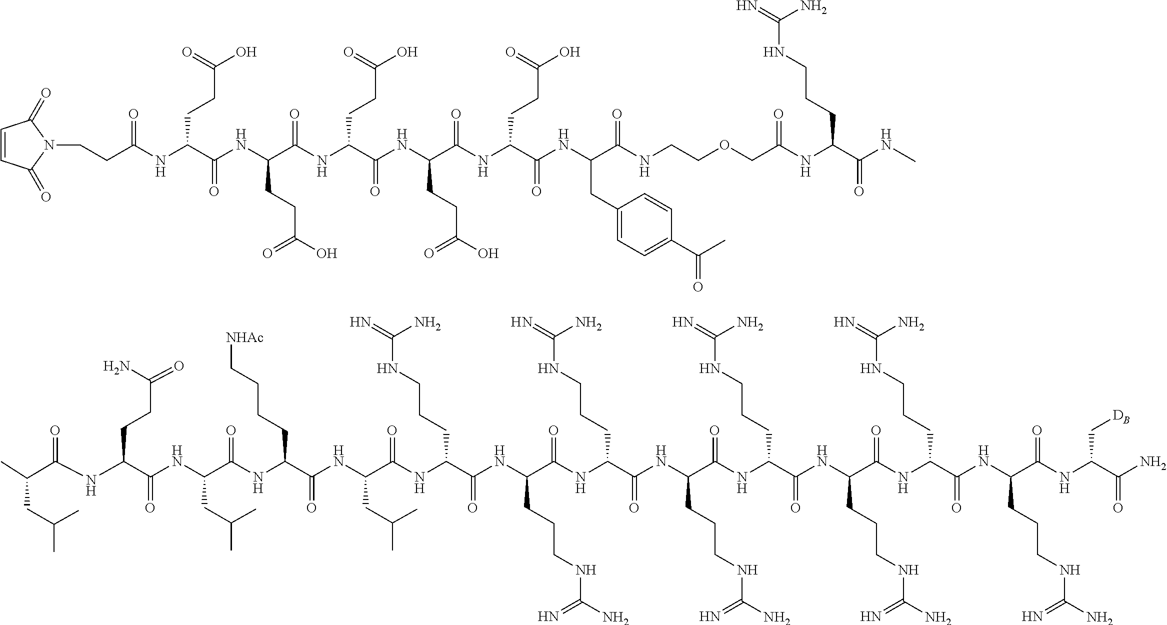 |
| SDM-233 | 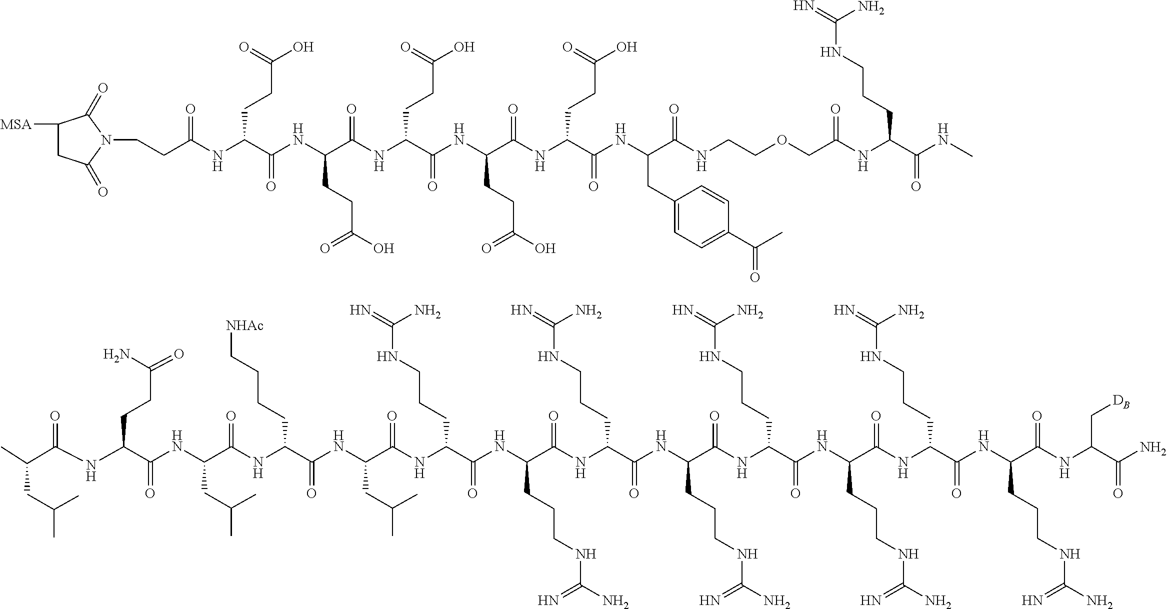 |
| SDM-234 | 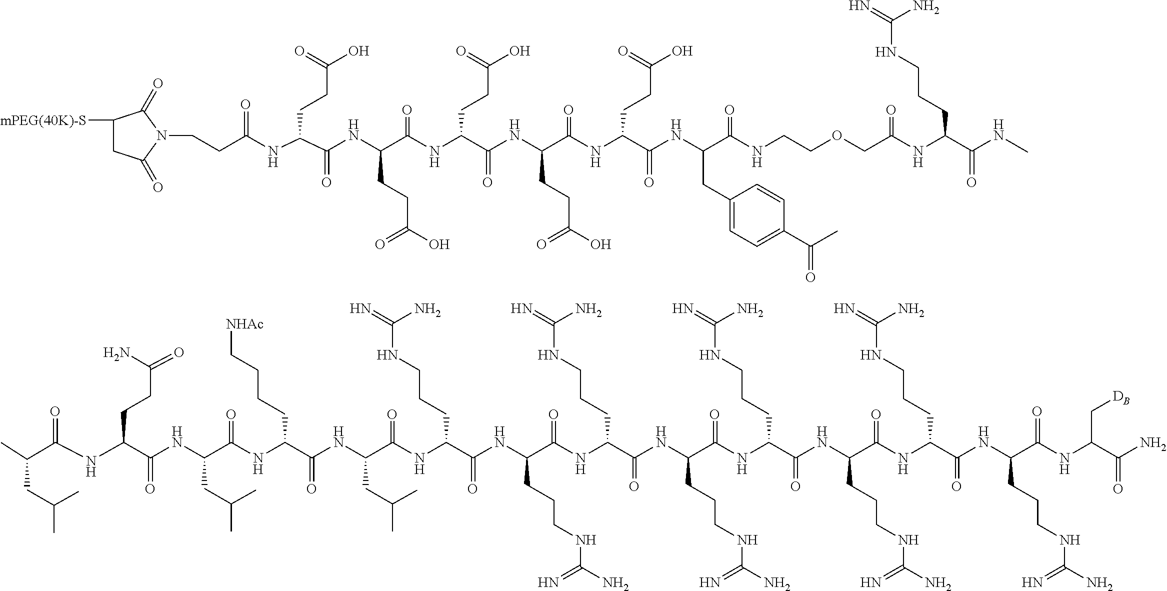 |
| SDM-235 | 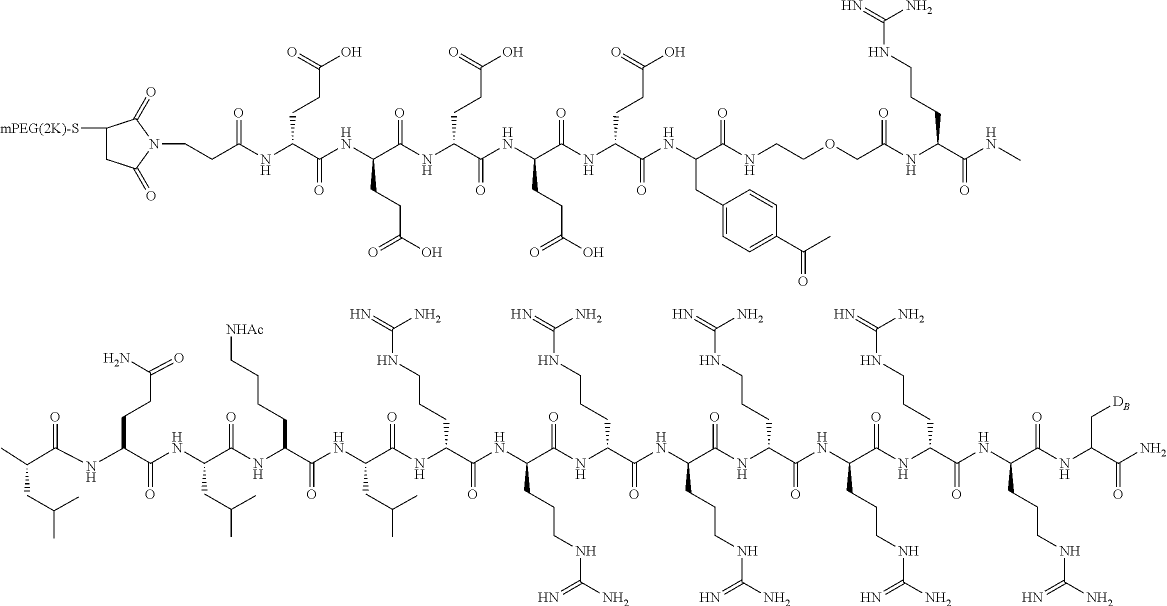 |
| SDM-188 | 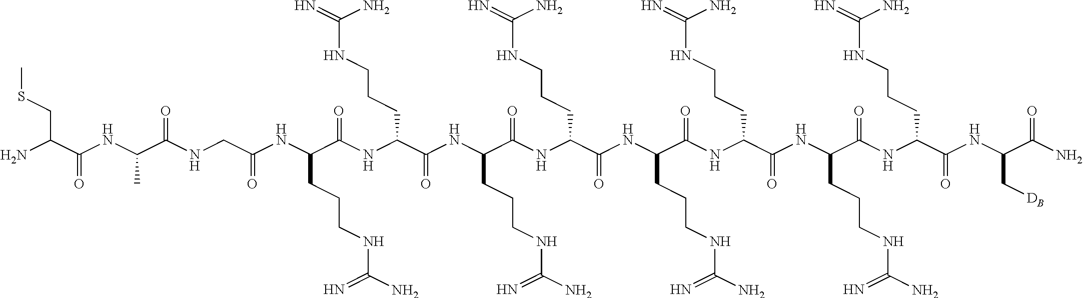 |
| SDM-347 | 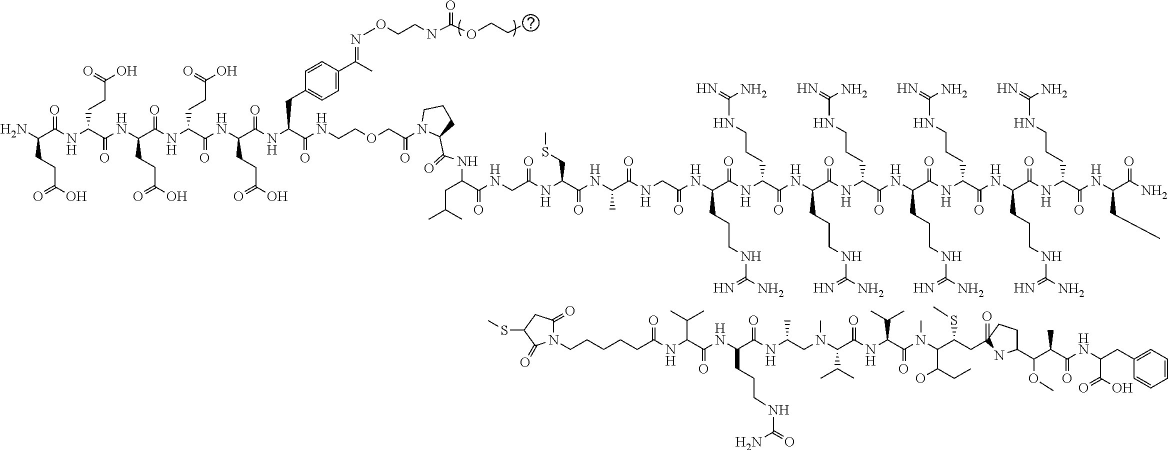 |

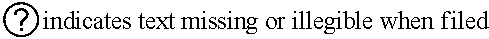

TABLE 8

| Compound Name | Chemical Structure 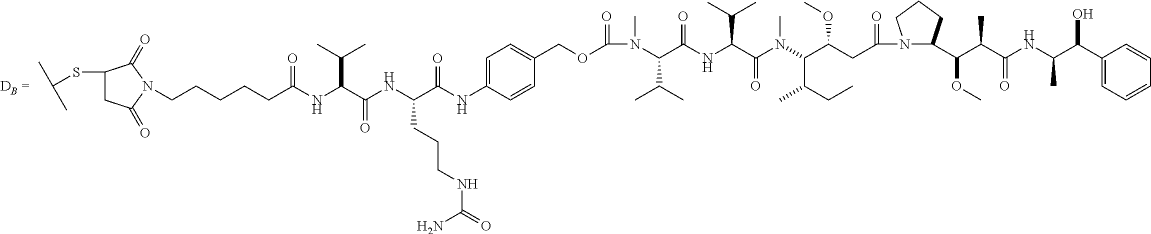 |
|---|---|
| SDM-261 | 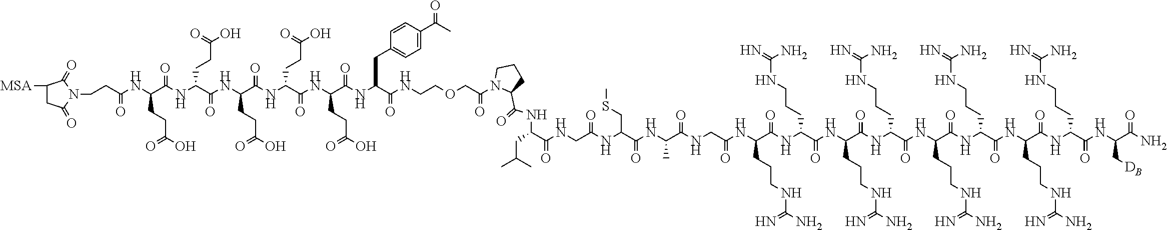 |
| SDM-262 | 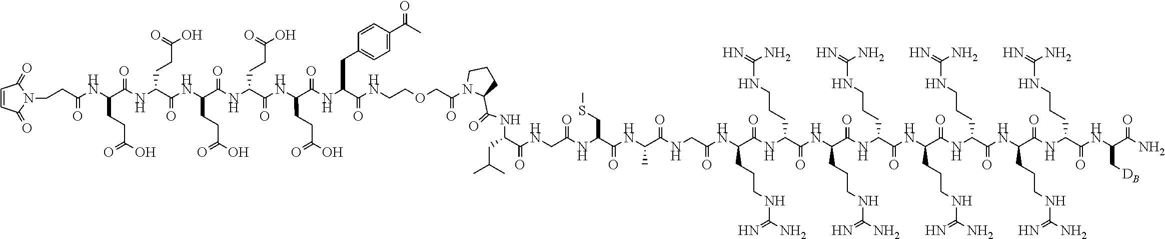 |
| SDM-263 | 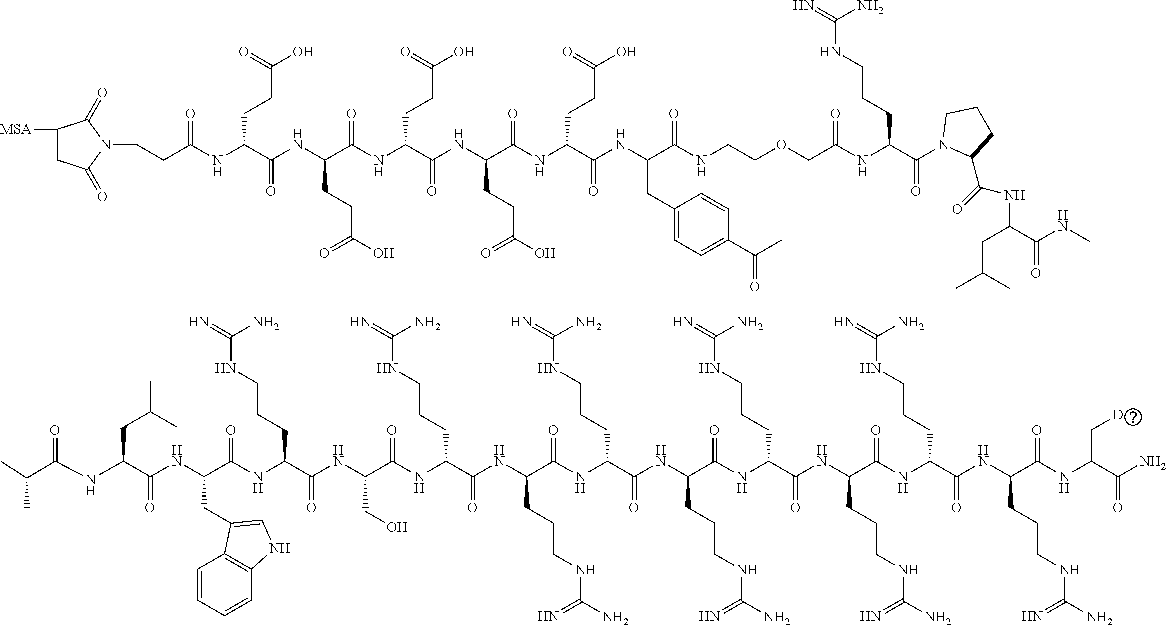 |
| SDM-264 | 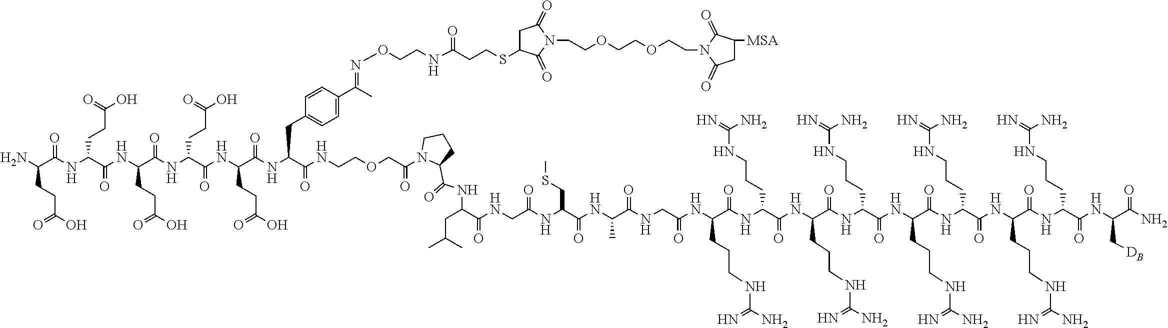 |
| SDM-265 | 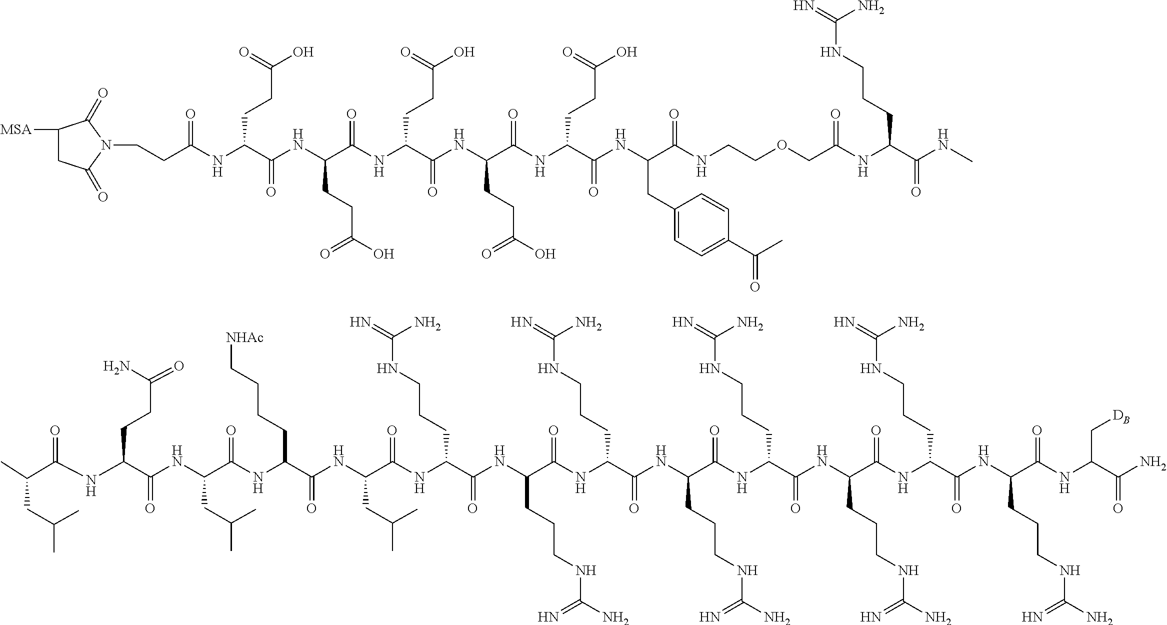 |

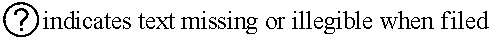

In some embodiments, the selective delivery molecule described herein is selected from SDM-173, SDM-178, SDM-181, SDM-182, SDM-189, SDM-190, SDM-191, SDM-195, SDM-199, SDM-200, SDM-210, SDM-211, SDM-212, SDM-218, SDM-219, SDM-220, SDM-221, SDM-222, SDM-225, SDM-227, SDM-228, SDM-229, SDM-230, SDM-231, SDM-257, and SDM-266.

In some embodiments, the selective delivery molecule described herein is selected from SDM-177, SDM-179, SDM-180, SDM-183, SDM-184, SDM-186, SDM-209, SDM-258, SDM-259, and SDM-260.

In some embodiments, the selective delivery molecule described herein is selected from SDM-185 and SDM-193.

In some embodiments, the selective delivery molecule described herein is selected from SDM-201 and SDM-202.

In some embodiments, the selective delivery molecule described herein is selected from SDM-192 and SDM-236.

In some embodiments, the selective delivery molecule described herein is selected from SDM-187 and SDM-188.

In some embodiments, the selective delivery molecule described herein is selected from SDM-203, SDM-204, SDM-208, SDM-213, SDM-214, SDM-215, SDM-216, SDM-223, SDM-224, SDM-226, SDM-232, SDM-233, SDM-234, and SDM-235.

In some embodiments, the selective delivery molecule described herein is selected from SDM-261, SDM-262, SDM-263, SDM-264, and SDM-265.

In some embodiments, the selective delivery molecule SDM-176 is a non-cleavable control.

Pharmaceutical Compositions

Disclosed herein, in certain embodiments, are pharmaceutical compositions comprising any of the selective delivery molecules as disclosed herein. In some embodiments, the pharmaceutical compositions comprises a selective delivery molecule of Formula (I), (II), (III), (IV) or (V), and a pharmaceutically acceptable carrier.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a pharmaceutical composition disclosed herein further comprises a pharmaceutically acceptable diluent(s), excipient(s), or carriers). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In certain embodiments, a pharmaceutical composition disclosed herein is administered to a subject by any suitable administration route, including but not limited to, parenteral (intravenous, subcutaneous, intraperitoneal, intramuscular, intravascular, intrathecal, intravitreal, infusion, or local) administration.

Formulations suitable for intramuscular, subcutaneous, peritumoral, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules.

In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

Methods of Use

The selective delivery molecule of Formula (I), (II), (III), (IV), or (V) allows the delivery of a therapeutic agent and/or imaging agent to specific cells and/or tissues. In some embodiments, a molecule of Formula (I), (II), (III), (IV), or (V) enables targeted delivery of one or more cargos (e.g., therapeutic agents or imaging agents) to a cell tissue.

Disclosed herein, in certain embodiments, are methods of delivering the selective delivery molecule to a tissue of interest, comprising contacting the tissue of interest with a compound of Formula (I), (II), (III), (IV), or (V).

In some embodiments, the selective delivery molecule of Formula (I), (II), (III), (IV), or (V) allows the delivery of a therapeutic agent and/or imaging agent to a tissue of interest. In some embodiments, the tissue of interest is cancerous tissue (or, cancer). In some embodiments, the cancerous tissue comprises breast cancer tissue, colorectal cancer tissue, squamous cell carcinoma tissue, skin cancer tissue, prostate cancer tissue, melanoma tissue, thyroid cancer tissue, ovarian cancer tissue, cancerous lymph node tissue, cervical cancer tissue, lung cancer tissue, pancreatic cancer tissue, head and neck cancer tissue, esophageal cancer tissue, or sarcoma tissue. In some embodiments, the cancerous tissue comprises breast cancer tissue, colon cancer tissue, squamous cell carcinoma tissue, prostate cancer tissue, melanoma tissue, or thyroid cancer tissue. In some embodiments, the cancerous tissue is breast cancer tissue. In some embodiments, the cancerous tissue is inflamed breast cancer tissue. In some embodiments, the cancerous tissue is colon cancer tissue. In some embodiments, the cancerous tissue is prostate cancer tissue. In some embodiments, the cancerous tissue is ovarian cancer tissue. In some embodiments, the cancerous tissue is thyroid cancer tissue. In some embodiments, the cancerous tissue is sarcoma tissue. In some embodiments, the cancerous tissue is soft sarcoma tissue. In some embodiments, the cancerous tissue is fibrosarcoma tissue. In some embodiments, the cancerous tissue is skin cancer tissue. In some embodiments, the cancerous tissue is squamous cell carcinoma tissue. In some embodiments, the cancerous tissue is cancerous lymph node tissue.

In some embodiments, the cancerous tissue is tissue affected by AIDS-related cancers (e.g., AIDS-related lymphoma), anal cancer, basal cell carcinoma, bile duct cancer (e.g., extrahepatic), bladder cancer, bone cancer, (osteosarcoma and malignant fibrous histiocytoma), breast cancer, cervical cancer, colon cancer, colorectal cancer, endometrial cancer (e.g., uterine cancer), ependymoma, esophageal cancer, eye cancer (e.g., intraocular melanoma and retinoblastoma), gastric (stomach) cancer, germ cell tumor, (e.g., extracranial, extragonadal, ovarian), head and neck cancer, leukemia, lip and oral cavity cancer, liver cancer, lung cancer (e.g., small cell lung cancer, non-small cell lung cancer, adenocarcinoma of the lung, and squamous carcinoma of the lung), ovarian cancer, pancreatic cancer, pituitary tumor, prostate cancer, renal cancer, sarcoma, skin cancer, small intestine cancer, squamous cell cancer, testicular cancer, throat cancer, thyroid cancer, urethral cancer, post-transplant lymphoproliferative disorder (PTLD), lymphoid cancer, or B-cell cancer.

In some embodiments, the cancerous tissue is tissue affected by precursor B-cell cancers (e.g., precursor B-lymphoblastic leukemia/lymphoma), peripheral B-cell cancers (e.g., B-cell chronic lymphocytic leukemia/prolymphocytic), leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, or Waldenstrom's macroglobulinemia.

In some embodiments, the cancerous tissue is afiected by the cancer is a T-cell and/or putative NIC-cell cancer. In some embodiments, the cancer is precursor T-cell cancer (precursor T-lymphoblastic lymphoma/leukemia) and peripheral T-cell and NIC-cell cancers (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic yfl T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, or Hodgkin's like).

In some embodiments, the tissue of interest is an inflamed tissue. In some embodiments, some embodiments, the inflamed tissue is the result if acute or chronic inflammation. In some embodiments, the inflamed tissue is caused by an inflammatory disease is or is associated with an inflammatory disease. In some embodiments, the inflamed tissue is caused by an inflammatory disease is or is associated with rheumatoid arthritis, osteoarthritis, inflammatory bowel disease, Crohn's disease, ulcerative colitis, sepsis, erythema nodosum leprosum, multiple sclerosis, psoriasis, systemic lupus erythematosis, type I diabetes, atherosclerosis, encephalomyelitis, Alzheimer's disease, stroke, traumatic brain injury, Parkinson's disease or septic shock.

### Therapeutic Uses

The selective delivery molecule of Formula (I), (II), (III), (IV), or (V) allows the targeted delivery of a therapeutic agent or imagine agent to specific cells and/or tissues (e.g., cancerous tissues). In some embodiments, targeted delivery of a therapeutic agent or imaging agent to a cell or tissue enables a medical professional to treat a specific tissue.

In some embodiments, targeted delivery of a therapeutic agent or imaging agent to a cell or tissue enables a medical professional to treat a specific tissue (e.g., cancerous tissue). In some embodiments, targeted delivery of a therapeutic agent or imaging agent to a cell or tissue decreases the dosage of the therapeutic agent. In some embodiments, targeted delivery of a therapeutic agent or imaging agent to a cell or tissue decreases contact of the therapeutic agent with healthy tissue. In some embodiments, targeted delivery of a therapeutic agent or imaging agent to a cell or tissue decreases unwanted side-effects arising from use of high concentrations of a therapeutic agent or contact. In some embodiments, targeted delivery of a therapeutic agent or imaging agent to a cell or tissue decreases unwanted side-effects arising from contact between the therapeutic agent and healthy tissue.

In some embodiments, the selective delivery molecule of Formula (I), (II), (III), (IV), or (V) is employed for the treatment of cancer.

In some embodiments, the cancer is AIDS-related cancers (e.g., AIDS-related lymphoma), anal cancer, basal cell carcinoma, bile duct cancer (e.g., extrahepatic), bladder cancer, bone cancer, (osteosarcoma and malignant fibrous histiocytoma), breast cancer, cervical cancer, colon cancer, colorectal cancer, endometrial cancer (e.g., uterine cancer), ependymoma, esophageal cancer, eye cancer (e.g., intraocular melanoma and retinoblastoma), gastric (stomach) cancer, germ cell tumor, (e.g., extracranial, extragonadal, ovarian), head and neck cancer, leukemia, lip and oral cavity cancer, liver cancer, lung cancer (e.g., small cell lung cancer, non-small cell lung cancer, adenocarcinoma of the lung, and squamous carcinoma of the lung), ovarian cancer, pancreatic cancer, pituitary tumor, prostate cancer, renal cancer, sarcoma, skin cancer, small intestine cancer, squamous cell cancer, testicular cancer, throat cancer, thyroid cancer, urethral cancer, and post-transplant lymphoproliferative disorder (PTLD). In some embodiments, the cancer is inflammatory breast cancer. In some embodiments, the cancer is triple negative breast cancer. In some embodiments, the cancer is sarcoma. In some embodiments, the cancer is soft sarcoma. In some embodiments, the cancer is fibrosarcoma. In some embodiments, the cancer is ovarian cancer.

In some embodiments, the cancer is breast cancer. In some instances, the breast cancer comprises invasive ductal carcinoma (IDC), invasive lobular carcinoma (ILC), ductal carcinoma in situ (DCIS), inflammatory breast cancer, lubular carcinoma in situ (LCIS), male breast cancer, molecular subtypes of breast cancer, Paget's disease of the Nipple, phyliodes tumors of the breast, and metastatic breast cancer. In some cases, IDC is further subdivided into tubular carcinoma of the breast, medullary carcinoma of the breast, mucinous carcinoma of the breast, papillary carcinoma of the breast, and cribriform carcinoma of the breast. In some cases, the molecular subtypes of breast cancer comprise luminal A, luminal B, triple-negative/basal-like, HER2-enriched, or normal-like breast cancer.

In some embodiments, the cancer is a lymphoid cancer (e.g., lymphoma).

In some embodiments, the cancer is a B-cell cancer. In some embodiments, the cancer is precursor B-cell cancers (e.g., precursor B-lymphoblastic leukemia/lymphoma) and peripheral B-cell cancers (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/−monocytoid B cells)), splenic marginal zone lymphoma (e.g., +/−villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the cancer is a T-cell and/or putative NK-cell cancer. In some embodiments, the cancer is precursor T-cell cancer (precursor T-lymphoblastic lymphoma/leukemia) and peripheral T-cell and NK-cell cancers (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic yd T-cell lymphoma, and subcutaneous panniculitic T-cell lymphoma), angioimmunoblastic T-cell lymphoma (AILD), angiocentric lymphoma, intestinal T-cell lymphoma (e.g., +/−enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the cancer is Hodgkin's disease.

In some embodiments, the cancer is leukemia. In some embodiments, the cancer is chronic myelocytic I (granulocytic) leukemia, chronic myelogenous, and chronic lymphocytic leukemia (CLL), acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia).

In some embodiments, the cancer is a liquid tumor or plasmacytoma. In some embodiments, the cancer is extramedullary plasmacytoma, a solitary myeloma, and multiple myeloma. In some embodiments, the plasmacytoma is multiple myeloma.

In some embodiments, the cancer is lung cancer.

In some embodiments, the cancer is prostate cancer. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is stage A prostate cancer (the cancer cannot be felt during a rectal exam). In some embodiments, the prostate cancer is stage B prostate cancer (i.e., the tumor involves more tissue within the prostate, it can be felt during a rectal exam, or it is found with a biopsy that is done because of a high PSA level). In some embodiments, the prostate cancer is stage C prostate cancer (i.e., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy. In some embodiments, the prostate cancer is substantially refractory to hormone therapy. In some embodiments, the prostate cancer is refractory to chemotherapy. In some embodiments, the prostate cancer is metastatic prostate cancer. In some embodiments, the individual is a human who has a gene, genetic mutation, or polymorphism associated with prostate cancer (e g., RNASEL/HPC1, ELAC2/HPC2, SR-A/MSR1, CHEK2, BRCA2, PON1, OGGI, MIC-1, TLR4, and PTEN) or has one or more extra copies of a gene associated with prostate cancer. In some embodiments, the prostate cancer is HER2 positive. In some embodiments, the prostate cancer is HER2 negative.

In some embodiments, the cancer is characterized by circulating tumor cells. In some embodiments, the cancer has metastasized and is characterized by circulating tumor cells.

In some embodiments, a tissue of interest is a tissue with upregulated protease activity (e.g., a tissue undergoing inflammatory response).

In some embodiments, the selective delivery molecule of Formula (I), (II), (III), (IV), or (V) is employed for the treatment of inflammation or an inflammatory disease. In some embodiments, the inflammation is chronic inflammation. In some embodiments, the inflammation is acute inflammation. In some embodiments, inflammation or inflammatory disease is or is associated with rheumatoid arthritis, osteoarthritis, inflammatory bowel disease, Crohn's disease, ulcerative colitis, sepsis, erythema nodosum leprosum, multiple sclerosis, psoriasis, systemic lupus erythematosis, type I diabetes, atherosclerosis, encephalomyelitis, Alzheimer's disease, stroke, traumatic brain injury, Parkinson's disease or septic shock.

In some embodiments, the selective delivery molecule of Formula (I), (II), (III), (IV), or (V) is employed for the treatment of an autoimmune disease. In some embodiments, the autoimmune disease is Celiac disease, diabetes mellitus type 1, Sarcoidosis, systemic lupus erythematosus (SLE), Sjögren's syndrome, Churg-Strauss Syndrome, Hashimoto's thyroiditis, Graves' disease, idiopathic thrombocytopenic purpura, Addison's Disease, rheumatoid arthritis (RA), Polymyositis (PM), or Dermatomyositis (DM).

### Imaging Uses

The imaging compound of Formula (I), (II), (III), (IV), or (V) allows the targeted delivery of imaging agent to specific cells and/or tissues (e.g., cancerous tissues). In some embodiments, the imaging compounds enable targeted delivery of one or more imaging agents to a cell or tissue. In some embodiments, targeted delivery of an imaging agent to a cell or tissue enables a medical professional to visualize/image a specific tissue.

In some embodiments, targeted delivery of an imaging agent to a cell or tissue enables a medical professional to visualize/image a specific tissue (e.g., cancerous tissue). In some embodiments, targeted delivery of an imaging agent to a cell or tissue enables a medical professional to remove (or, surgically excise) the tissue of interest (e.g., cancerous tissue). In some embodiments, targeted delivery of an imaging agent to a cell or tissue enables a medical professional to remove (or, surgically excise) the tissue of interest (e.g., cancerous tissue) with a decrease in surgical margins. In some embodiments, targeted delivery of an imaging agent to a cell or tissue enables a medical professional to remove (or, surgically excise) a tumor/cancerous tissue and decreases the chance that some of the tumor/cancerous tissue will not be removed. In some embodiments, targeted delivery of an imaging agent to a cell or tissue enables a medical professional to maximally debulk a tumor/cancerous tissue. In some embodiments, targeted delivery of an imaging agent to cancerous tissue decreases the chances of an unnecessary operations and re-operations. In some embodiments, the cancerous tissue is breast cancer tissue. In some embodiments, the cancerous tissue is colon cancer tissue. In some embodiments, the cancerous tissue is prostate cancer tissue. In some embodiments, the cancerous tissue is ovarian cancer tissue. In some embodiments, the cancerous tissue is thyroid cancer tissue. In some embodiments, the cancerous tissue is sarcoma tissue. In some embodiments, the cancerous tissue is soft sarcoma tissue. In some embodiments, the cancerous tissue is fibrosarcoma tissue. In some embodiments, the cancerous tissue is skin cancer tissue. In some embodiments, the cancerous tissue is squamous cell carcinoma tissue. In some embodiments, the cancerous tissue is cancerous lymph node tissue. In some embodiments, the cancerous tissue is breast cancer tissue. In some embodiments, the cancerous tissue is inflamed breast cancer tissue. In some embodiments, the cancerous tissue is inflamed breast cancer tissue.

In some embodiments, targeted delivery of an imaging agent to a cell or tissue enables a medical professional to more accurately sample (e.g., biopsy (e.g., excision biopsy, incision, biopsy, aspiration biopsy, or needle biopsy)) tissue of interest (e.g., cancerous tissue). In some embodiments, targeted delivery of an imaging agent to a cell or tissue enables a medical professional to visualize/image a specific tissue (e.g., cancerous tissue) within an excised tissue containing healthy tissue. Enabling identification of target tissue (e.g., cancerous tissue) can guide the pathologist on where to section of pathological evaluation and decreases the chances of a pathologist missing unhealthy tissue (e.g., cancerous tissue) and sampling healthy tissue which may produce a false negative. In some embodiments, tissue (e.g., cancerous tissue) removed following use of a compound of Formula (I), (II), (III), (IV), or (V) is used to prepare a pathology section or slide. In some embodiments, cancerous tissue removed following use of a compound of Formula (I), (II), (III), (IV), or (V) is used to prepare a pathology section or slide which is used to diagnose a tissue as malignant or benign.

In some embodiments, targeted delivery of an imaging agent to cancerous breast tissue enables a medical professional to accurately stage cancer enabling medical treatment decisions. In some embodiments, targeted delivery of an imaging agent to cancerous tissue enables a medical professional to observe the size of a tumor (cancerous tissue) or the spread (e.g., metastatic lesions) of cancerous tissue.

In some embodiments, targeted delivery of an imaging agent to a cell or tissue enables a medical professional to design an efficacious treatment regimen.

In some embodiments, a selective delivery molecule according to Formula (I), (II), (III), (IV), or (V) comprising an imaging agent is employed in guided surgery. In some embodiments, the selective delivery molecule preferentially localized to cancerous, or other pathological tissues with up-regulated protease activity (e.g. tissues undergoing inflammatory response). In some embodiments, a selective delivery molecule according to Formula (I), (II), (III), (IV), or (V) comprising an imaging agent is employed in a guided surgery to remove colorectal cancer. In some embodiments, guided surgery employing the selective delivery molecule allows a surgeon to excise as little healthy (i.e., non-cancerous) tissue as possible. In some embodiments, guided surgery employing the selective delivery molecule allows a surgeon to visualize and excise more cancerous tissue than the surgeon would have been able to excise without the presence of the selective delivery molecule. In some embodiments, the surgery is fluorescence-guided surgery.

Disclosed herein is a method of visualizing a tissue of interest in an individual in need thereof, comprising administering to the individual a selective delivery molecule with an imaging agent Z², and visualizing Z². In some embodiments, the tissue of interest is cancerous tissue. In some embodiments, the tissue of interest is: breast cancer tissue, colorectal cancer tissue, squamous cell carcinoma tissue, skin cancer tissue, prostate cancer tissue, melanoma tissue, thyroid cancer tissue, ovarian cancer tissue, cancerous lymph node tissue, cervical cancer tissue, lung cancer tissue, pancreatic cancer tissue, head and neck cancer tissue, esophageal cancer tissue, or sarcoma. In some embodiments, the method further comprisines surgically removing the tissue of interest from the individual. In some embodiments, the tissue of interest is cancer. In some embodiments, the visualizing Z² is used to guide surgery, reduce positive margins, to stage cancer tissue, to stage lymph nodes, to reduce reoperations or allows a surgeon to minimize the removal of healthy tissue.

### EXAMPLES

The following examples are provided for illustrative purposes only and not to limit the scope of the claims provided herein.

### I. Chemical Synthesis

### Materials and Methods

All reaction solvents were freshly opened Aldrich “Sure-Seal” quality. All the reagents were reagent-grade and used without further purification unless otherwise indicated. HPLC-grade acetonitrile was purchased from Fisher Scientific (Phillipsburg, Pa.). Water used in HPLC was collected through Milli-Q water purification system (Millipore, Bedford, Mass.). PBS-EDTA buffer was purchased from Teknova (Hollister, Calif.). α-Mercaptoethyl-ω-methoxy, poly-oxyethylene (average molecular weight around 2,000, 5,000, 20,000 and 40,000) [mPEG(2K)-SH, mPEG(5K)-SH, mPEG(20K)-SH, mPEG(40K)-SH] and α-aminoxyl-ω-methoxy, polyoxyethylene (average molecular weight approximately 2,000, 5,000, 20,000 and 40,000) [mPEG(2K)-ONH₂, mPEG(5K)-ONH₂, mPEG(20K)-ONH₂, mPEG(40K)-ONH₂] were purchased from NOF America Corporation (Irvine, Calif.). Mouse serum albumin (MSA) was purchased from Sigma or Innovative Research (Novi, Mich.).

LC-MS analysis was carried out on a Waters 2695 separation module equipped with a Waters 2487 dual λ absorbance detector in combination with Finnigan LCQ Deca XP mass spectrometer. The equipment is associated with Xcalibur analytical software and a Peeke Scientific column (Titan 200 5 μm, C18-MC, 50×2.1 mm) or a Phenomenex column (Kinetex 5 μm, EVO C18 100 A, 50×4.6 mm).

Preparation HPLCs were carried out on a Waters PrepLC System equipped with a Waters 2487 dual λ absorbance detector, Fraction Collector III, Masslynx software and a Thermo Scientific column (Hypersil Gold C18, 5μ, 250×10 mm) or a Phenomenex column (luna, C18(2), 5μ, 100 A AX 150×30 mm). The mobile phase consisted of a water (0.05% TFA)(solvent A)/acetonitrile (0.05% TFA)(solvent B) gradient unless otherwise specified. Centrifugation was carried out at 4° C. on an Eppendorf centrifuge 5417R or a Beckman Microfuge® 18. Lyophilization was carried out on a Labconco FreeZone 4.5.

### Example 1: General Procedure for the Synthesis of SDM-154, SDM-270, SDM-311, and SDM-366

The following peptides were synthesized using standard Fmoc chemistry. After cleavage from the CTC resin in DMF (95% TFA, 2.5% thioanisole, 2.5% H2O), the peptides were precipitated and washed by cold tert-butyl methyl ester. Purification of the precipitate by RP-HPLC afforded product.

| SDM | Molecular weight | MS(ESI) m/e |
|---|---|---|
| SDM-154 | 1238.51 | 619.8 [M + 2H]²⁺, 1238.4 [M + H]⁺ |
| SDM-270 | 1251.75 | 1252.4 [M + H]⁺ |
| SDM-311 | 1238.6 | 1238.6 [M + H]⁺ |
| SDM-366 | 1240.6 | 1241.2 [M + 2H]²⁺, 1262.9 [M + Na]⁺ |

### Example 2: Synthesis of SDM-173

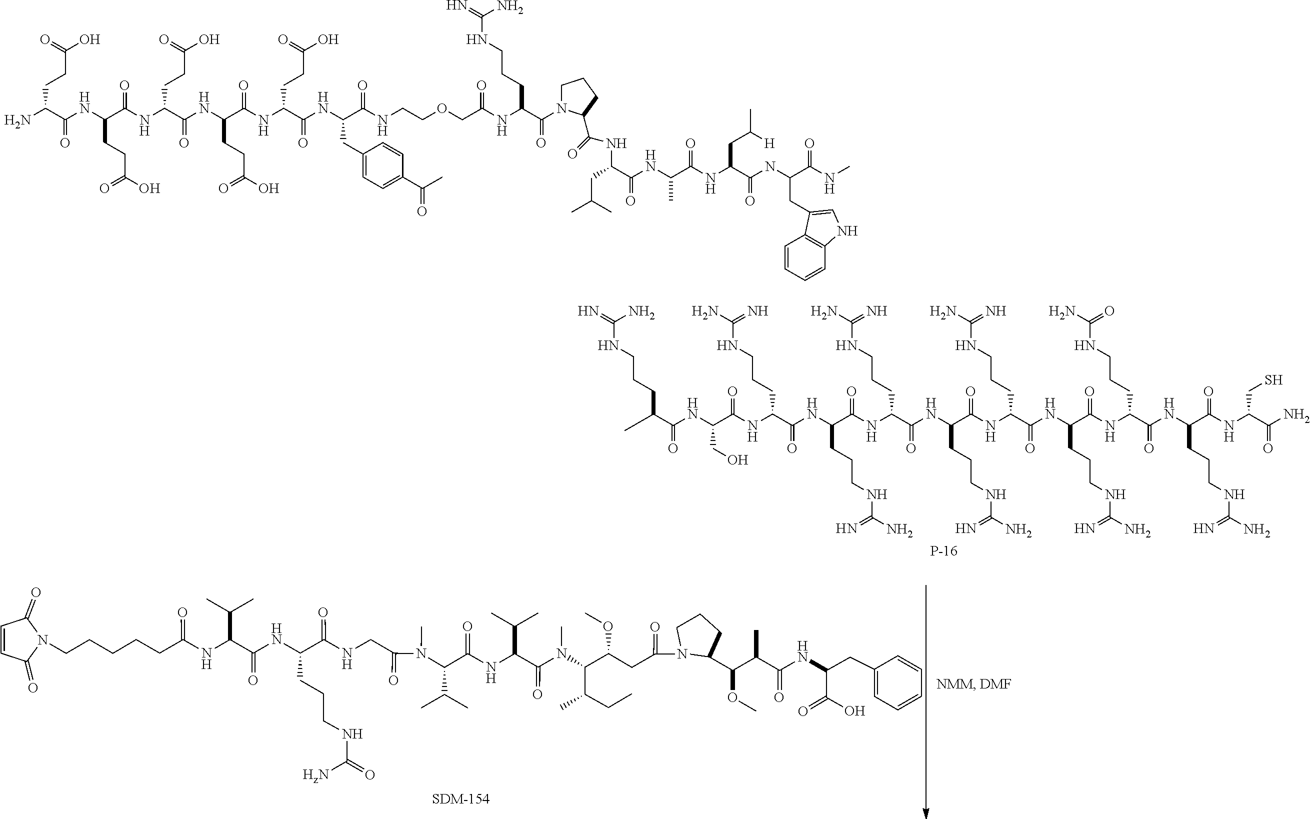

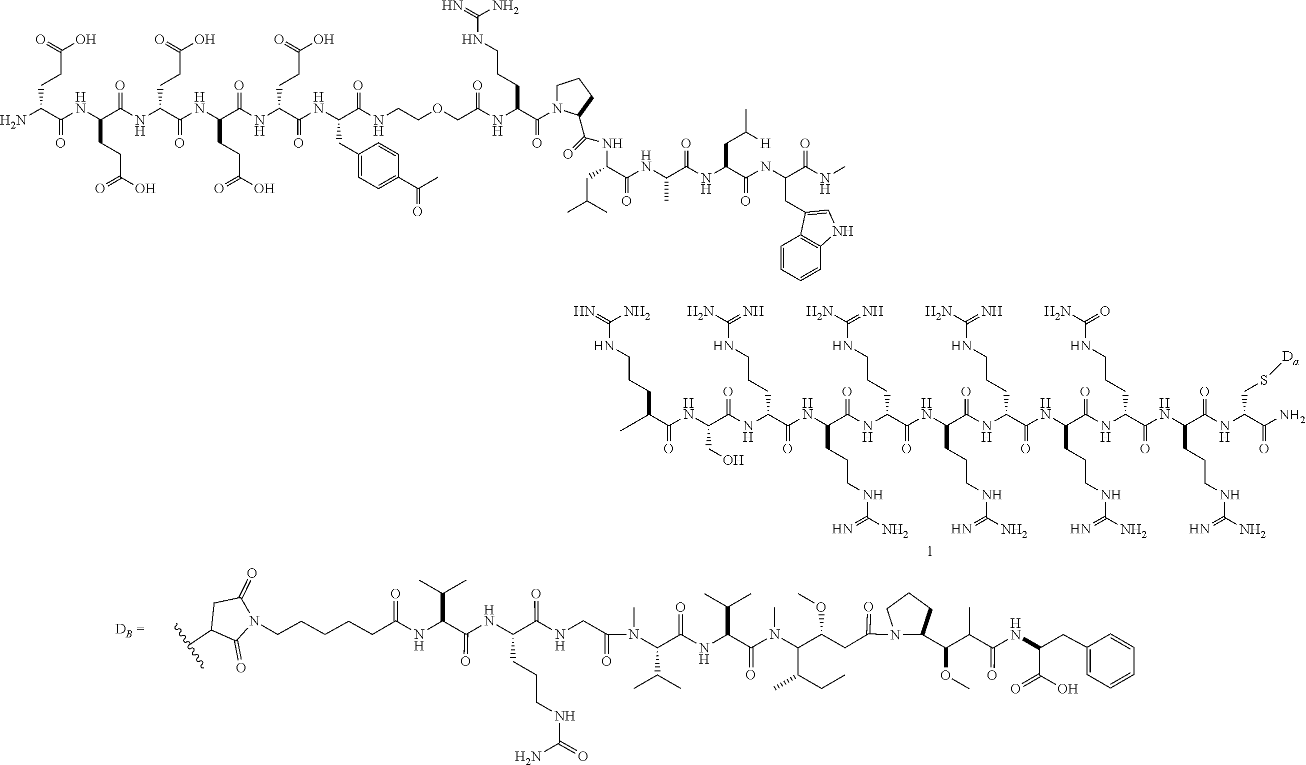

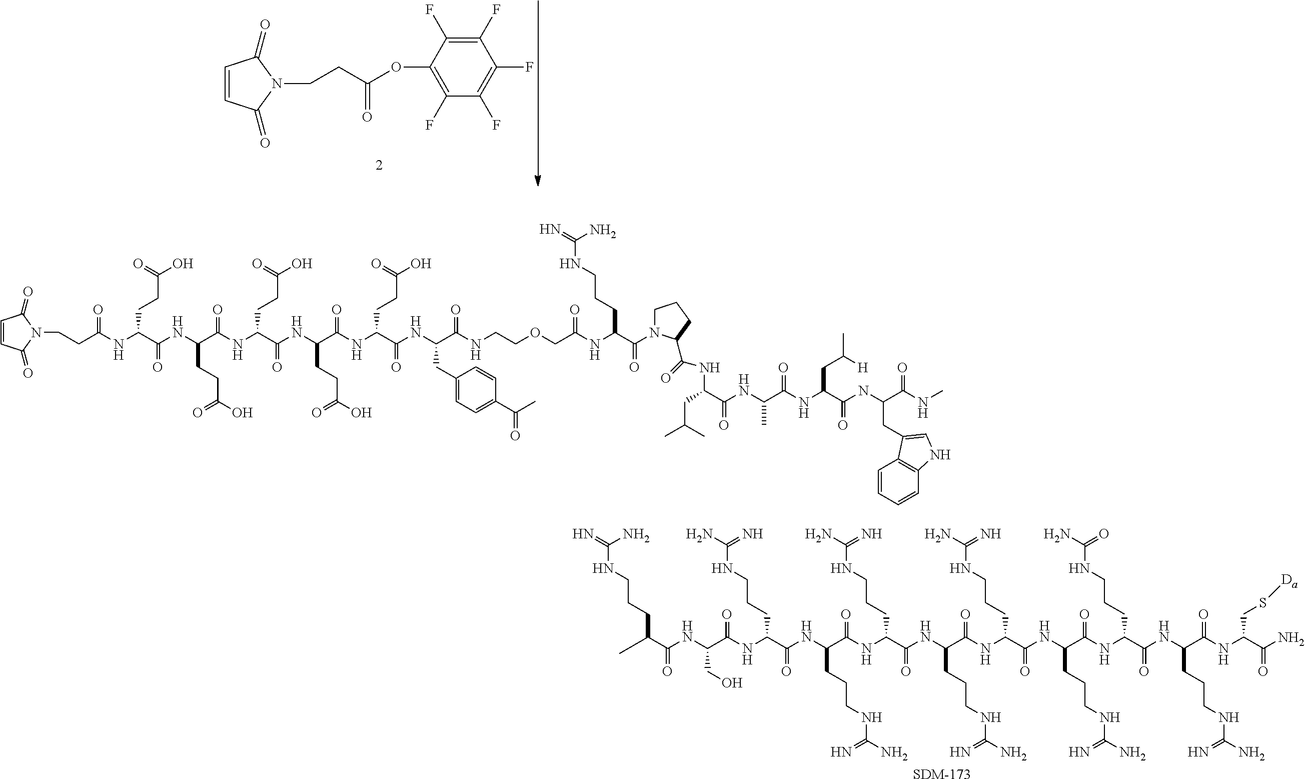

To a stirred solution of peptide P-16 (30.0 mg, 6.7 μmol) and SDM-154 (9.0 mg, 6.7 μmol) in DMF (0.7 mL) was added N-methylmorpholine (NMM, 4.0 μL, 36.4 μmol). The reaction mixture was stirred at room temperature for 1.5 h. Then 3-maleimidopropionic acid pentafluorophenyl ester 2 (5.0 mg, 15 μmol) was added. The mixture was stirred at room temperature for 15 h. Purification by RP-HPLC afforded SDM-173 as a white powder after lyophilization (32 mg, 83%). MS (ESI): m/e 1169.08 [M+4H]⁴⁺, 1559.16 [M+3H]³⁺.

### Example 3: Synthesis of SDM-178

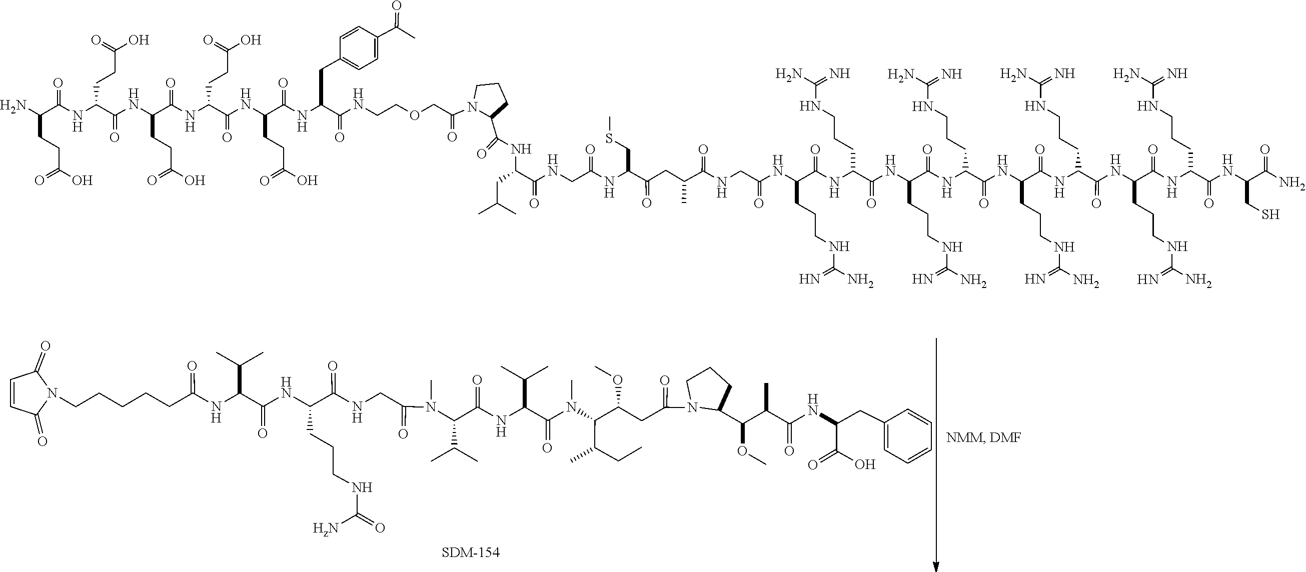

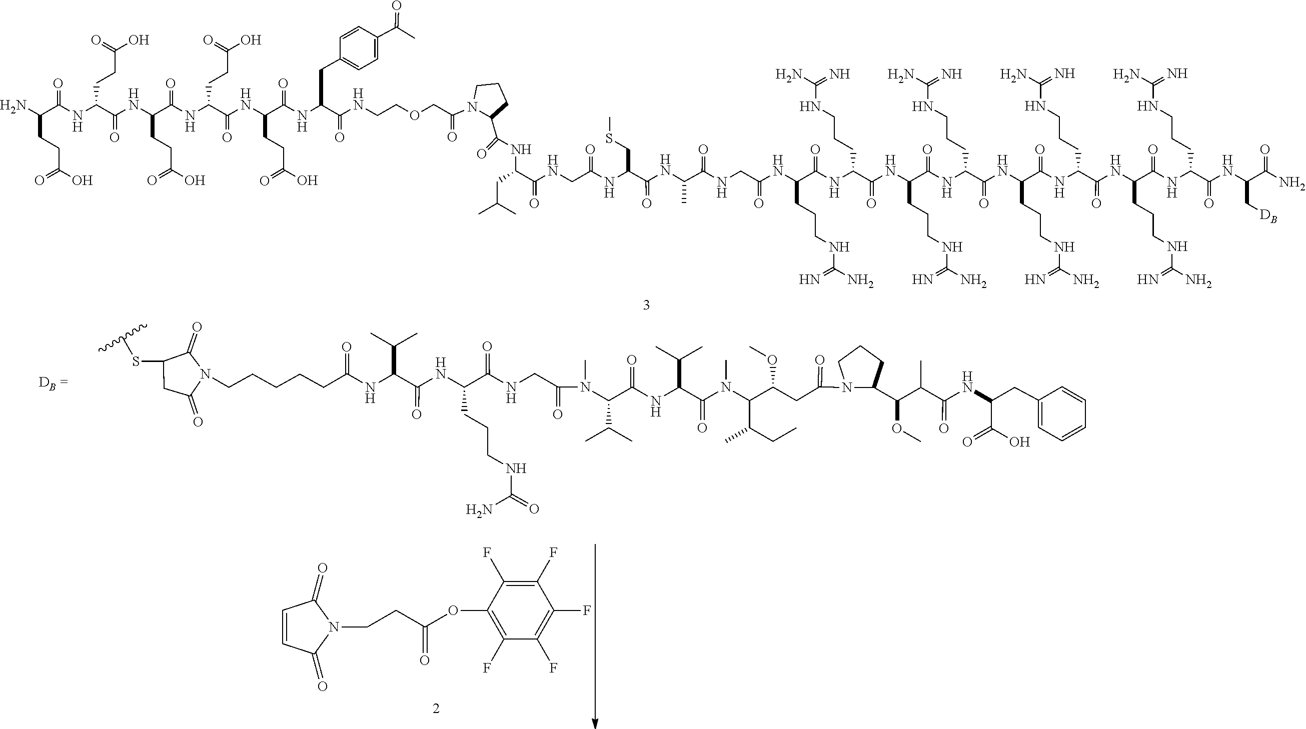

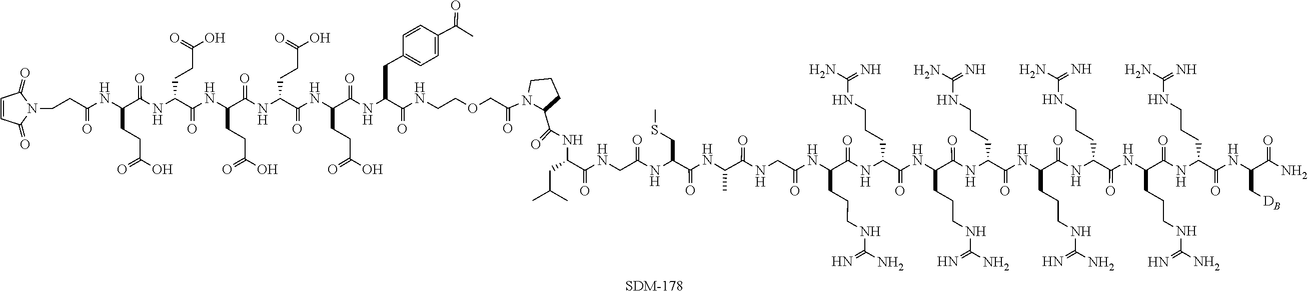

To a stirred solution of peptide P-3 (60.0 mg, 14.8 μmol) and SDM-154 (20.0 mg, 14.8 μmol) in DMF (0.7 mL) was added N-methylmorpholine (NMM, 15.0 μL, 137 μmol). The reaction mixture was stirred at room temperature for 0.5 h. Then 3-maleimidopropionic acid pentafluorophenyl ester 2 (6.0 mg, 17.9 μmol)) was added. The mixture was stirred at room temperature for 15 h. Purification by RP-HPLC afforded SDM-178 as a white powder after lyophilization (72 mg, 90%). MS (ESI): m/e 1403.30 [M+3H]³⁺.

### Example 4: Synthesis of SDM-179

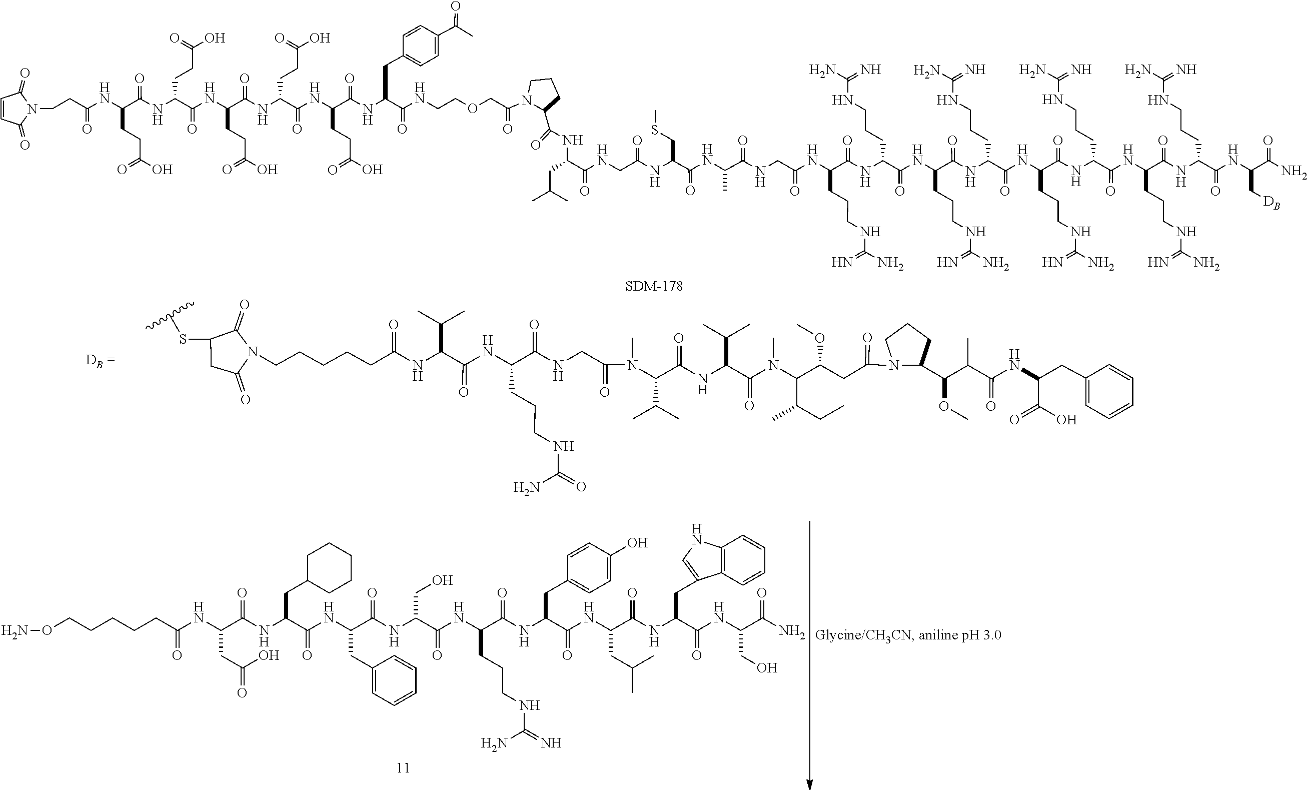

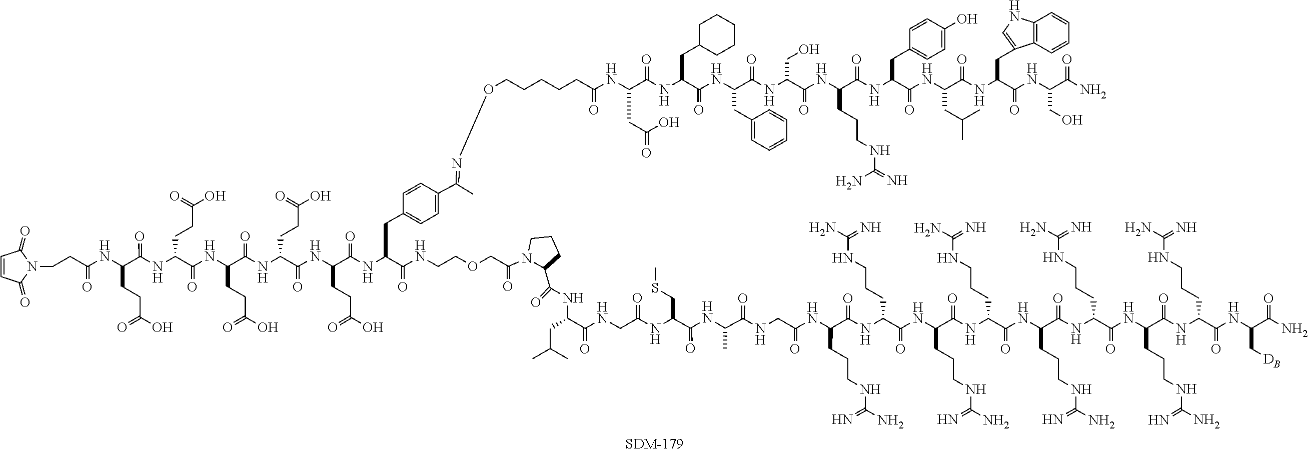

To a stirred solution of SDM-178 (12.8 mg, 2.1 μmol) in a glycine buffer (1.0 mL, 0.1 M, 20 mM aniline, pH 3.0) and acetonitrile (0.5 mL) was added 11 (7.0 mg, 4.4 μmol). The reaction mixture was stirred at room temperature for 15 h. Purification by RP-HPLC afforded SDM-179 (12.7 mg, 79%). MS (ESI): m/e 1580.38 [M+4H]⁴⁺.

### Example 4: Synthesis of SDM-180

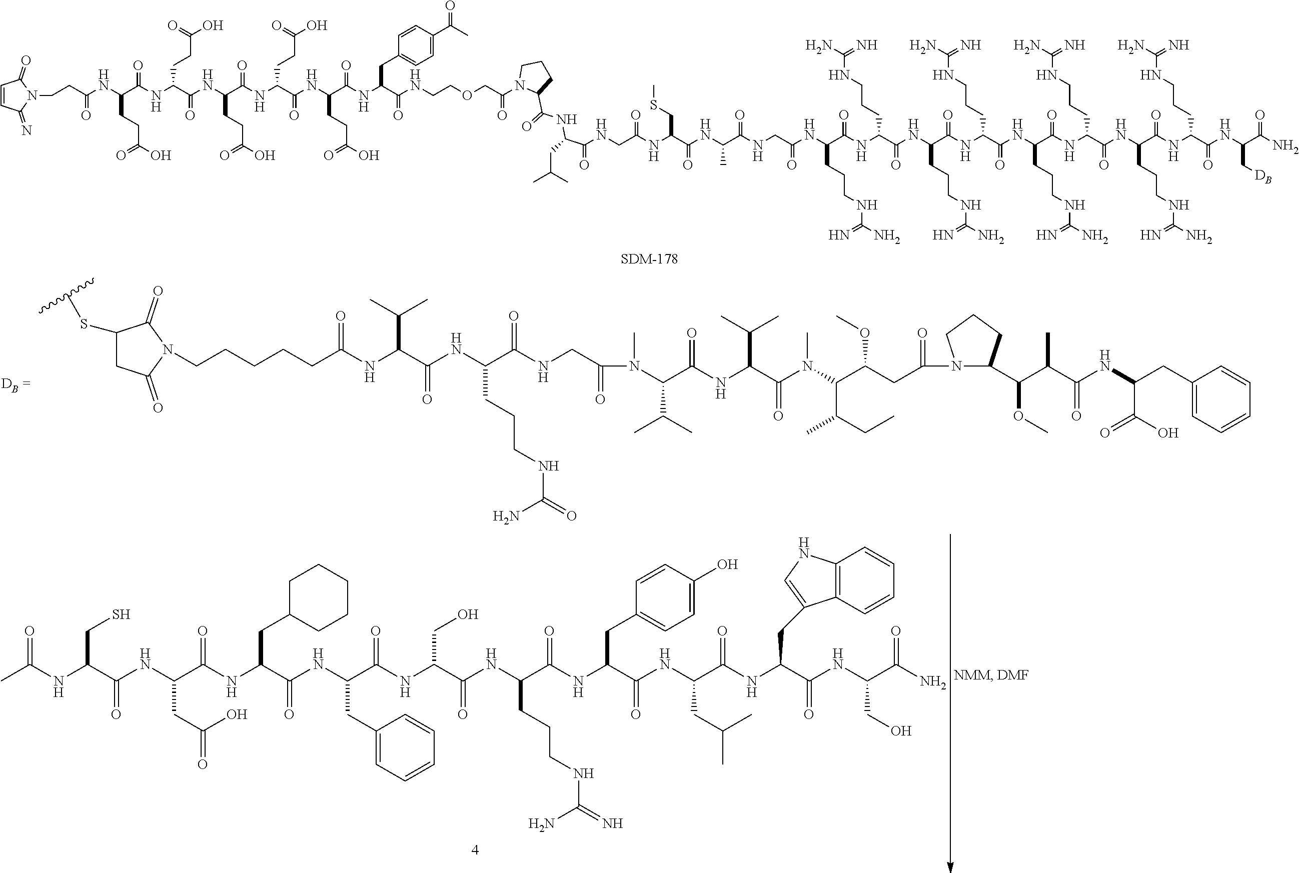

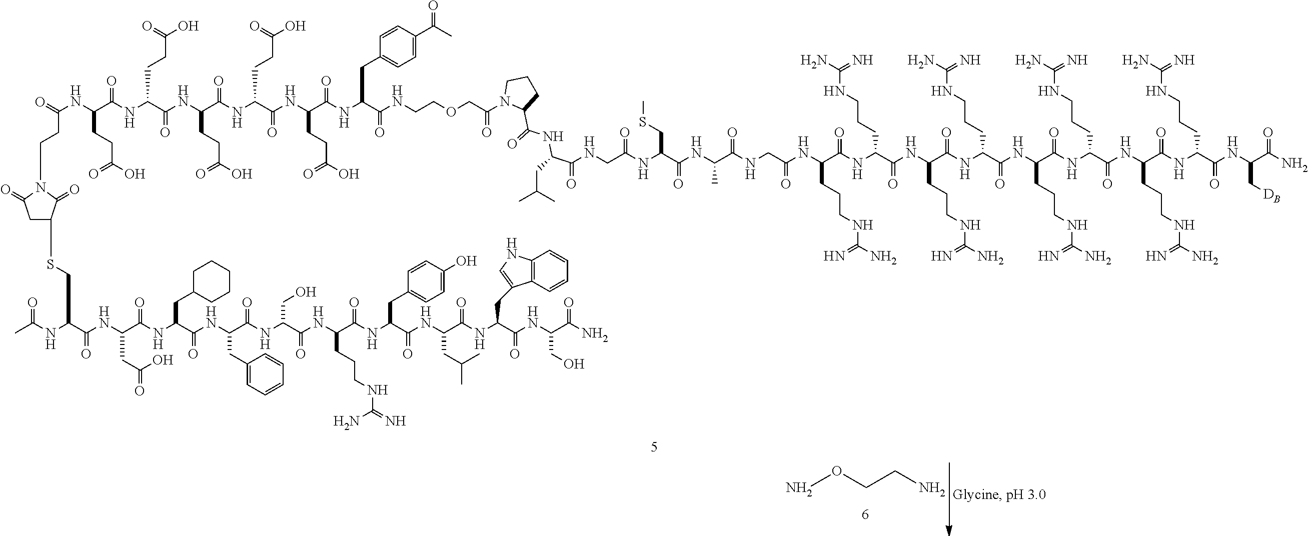

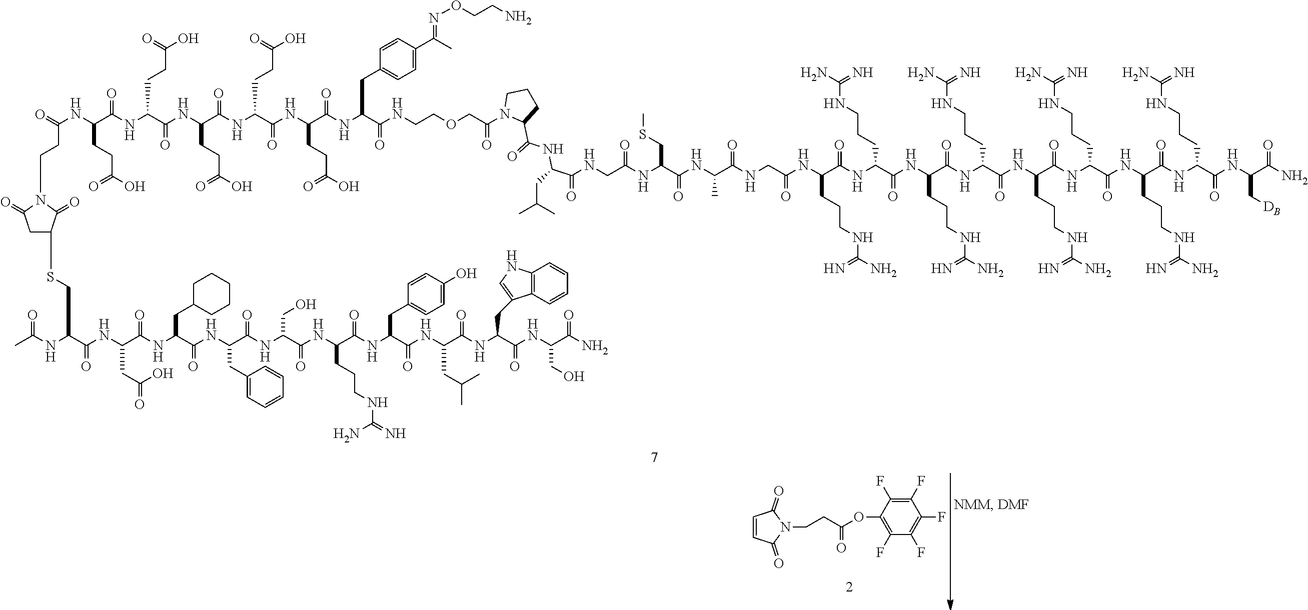

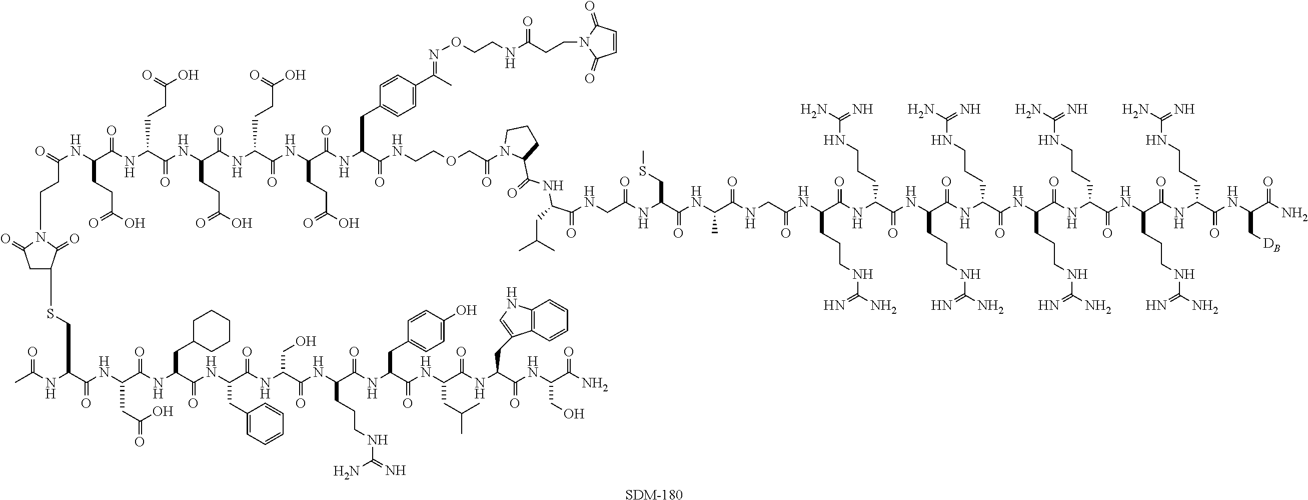

### Synthesis of Intermediate 5

To a stirred solution of peptide SDM-178 (34.0 mg, 5.1 μmol) and Compound 4 (15.5 mg, 10.4 μmol) in DMF (1.0 mL) was added N-methylmorpholine (NMM, 3.0 μL, 27.3 μmol). The reaction mixture was stirred at room temperature for 1 h and purified by RP-HPLC to afford Intermediate 5 (42.7 mg, 91%). MS (ESI): m/e 1860.44 [M+3H]³⁺.

### Synthesis of Intermediate 7

To a stirred solution of Intermediate 5 (36.3 mg, 7.1 μmol) in a glycine buffer (1.0 mL, 0.1 M, 20 mM aniline, pH 3.0) and acetonitrile (1.0 mL) was added oxyamine 6 (1.5 mg, 19.7 μmol). The reaction mixture was stirred at room temperature for 20 h. Purification by RP-HPLC afforded Intermediate 7 (32.0 mg, 94%). MS (ESI): m/e 1879.47 [M+3H]³⁺.

### Synthesis of SDM-180

To the mixture of Intermediate 7 (28.1 mg, 4.1 μmol) and 3-maleimidopropionic acid pentafluorophenyl ester 2 (2.0 mg, 6.0 μmol) in DMF (0.7 mL) was added N-methylmorpholine (NMM, 7.0 μL, 63.8 μmol). The mixture was stirred at room temperature overnight. Purification by RP-HPLC afforded SDM-180 as a white powder after lyophilization (25.1 mg, 89%). MS (ESI): m/e 1447.98 [M+4H]⁴⁺.

### Example 5: Synthesis of SDM-183

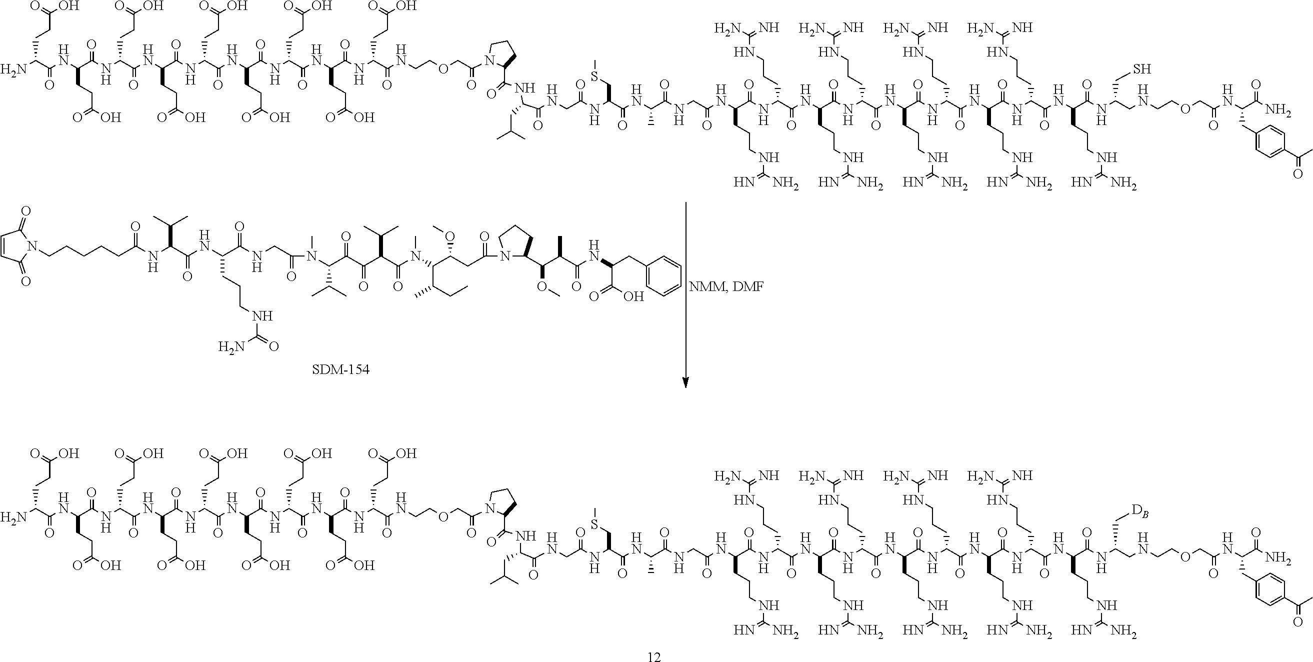

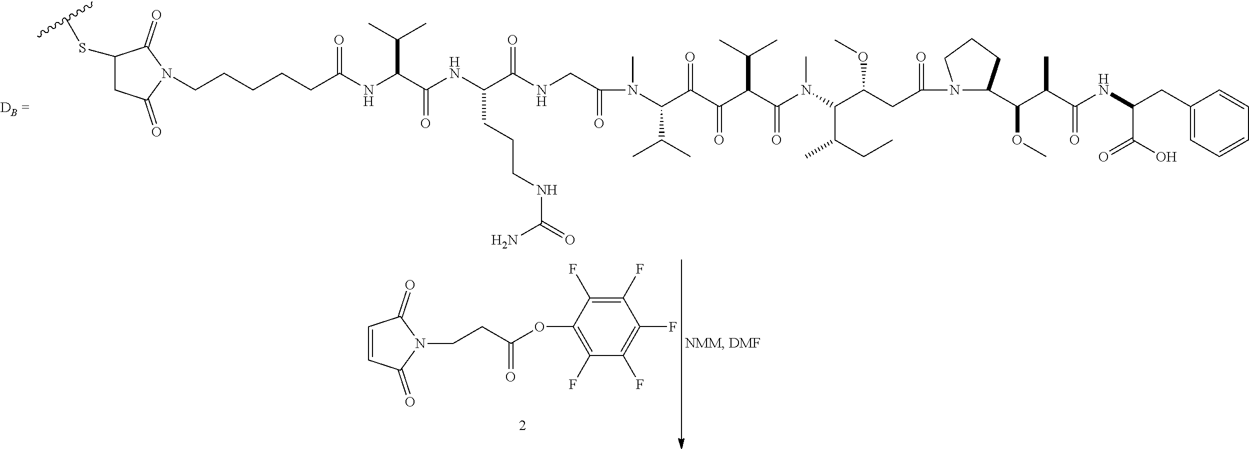

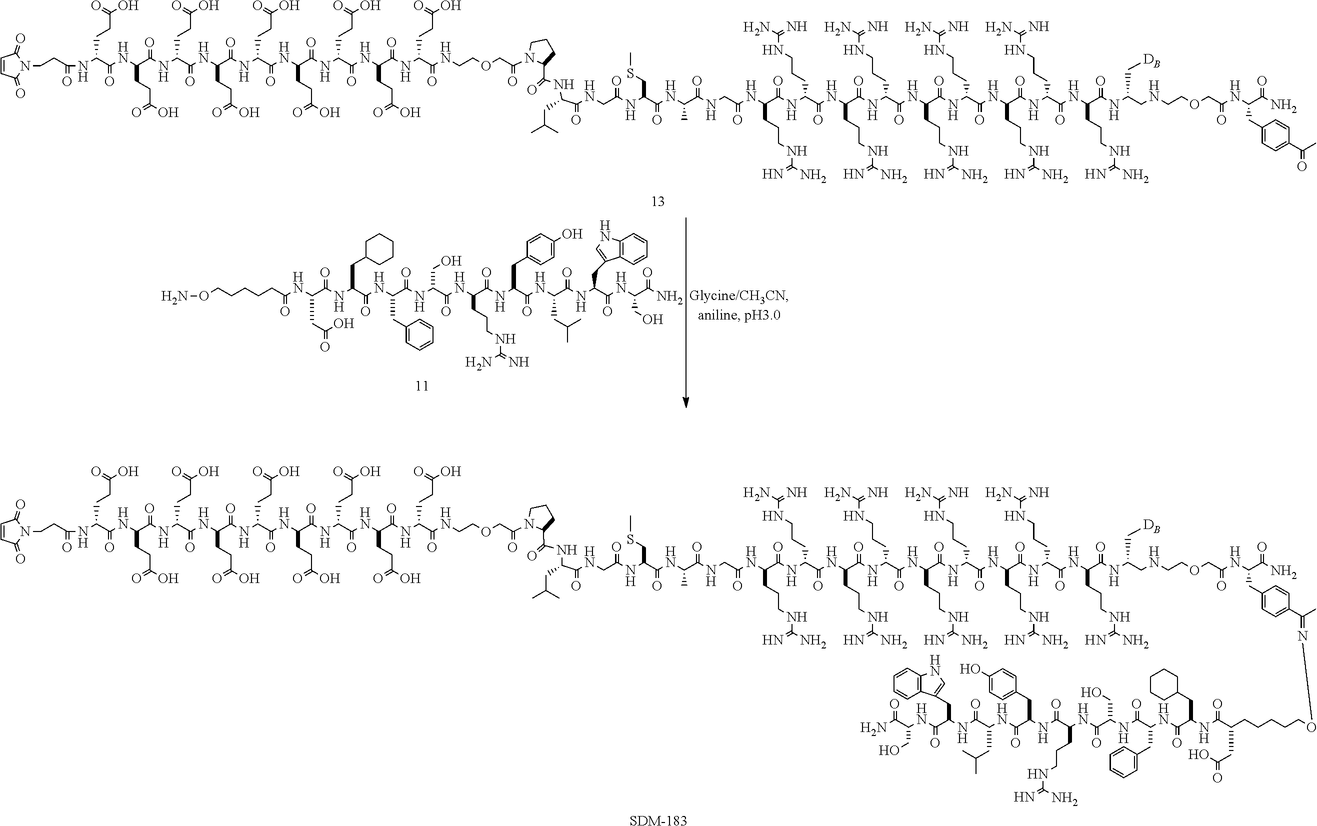

### Synthesis of Intermediate 13

To a stirred solution of peptide P-7 (49.1 mg, 12.4 μmol) and SDM-154 (18.0 mg, 12.6 μmol) in DMF (1.0 mL) was added N-methylmorpholine (NMM, 40 μL, 0.36 mmol). The reaction mixture was stirred at room temperature for 20 h. Then 3-maleimidopropionic acid pentafluorophenyl ester 2 (6.0 mg, 17.9 μmol)) was added. The mixture was stirred at room temperature for 24 h. Purification by RP-HPLC afforded 13 as a white powder after lyophilization (52 mg, 70%). MS (ESI): m/e 1246.41 [M+4H]⁴⁺, 1661.11 [M+3H]³⁺.

### Synthesis of SDM-183

To a solution of Intermediate 13 (12.8 mg, 2.1 μmol) in a glycine buffer (1.0 mL, 0.1 M, 20 mM aniline, pH 3.0) and acetonitrile (0.5 mL) was added 11 (7.0 mg, 4.4 μmol). The reaction mixture was stirred at room temperature for 15 h. Purification by RP-HPLC afforded SDM-183 as a white powder after lyophilization (12.7 mg, 81%). MS (ESI): m/e 1580.38 [M+4H]⁴⁺.

### Example 7: Synthesis of SDM-184

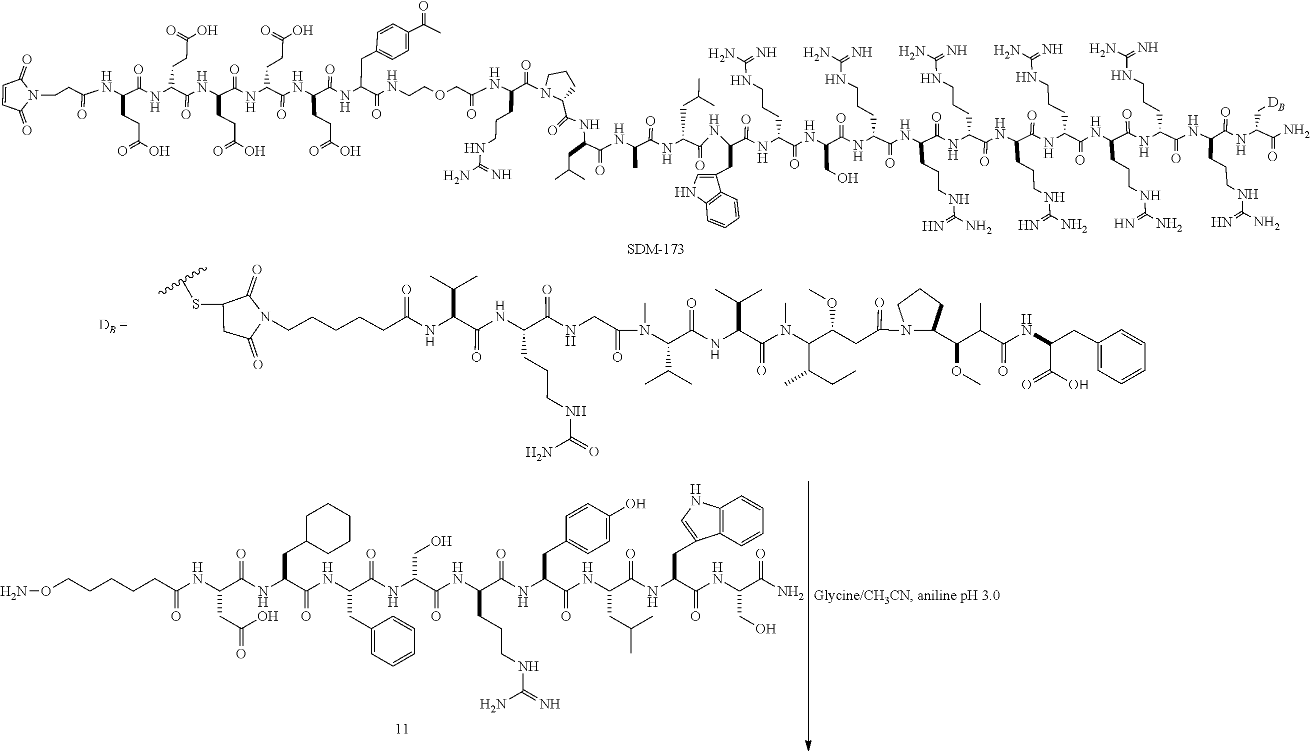

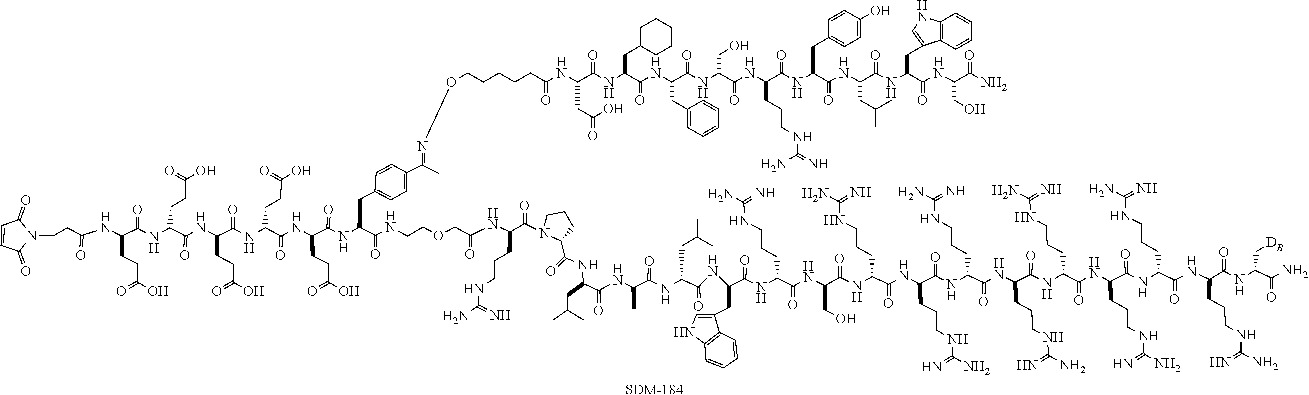

To a mixture of SDM-173(25.0 mg, 4.3 μmol) in a glycine buffer (1.0 mL, 0.1 M, 20 mM aniline, pH 3.0) and acetonitrile (0.5 mL) was added peptide 11 (15.0 mg, 9.5 μmol). The reaction mixture was stirred at room temperature for 15 h. Purification by RP-HPLC afforded SDM-184 as a white powder after lyophilization (26 mg, 85%). MS (ESI): m/e 1503.92 [M+4H]⁴⁺.

### Example 8: Synthesis of SDM-188

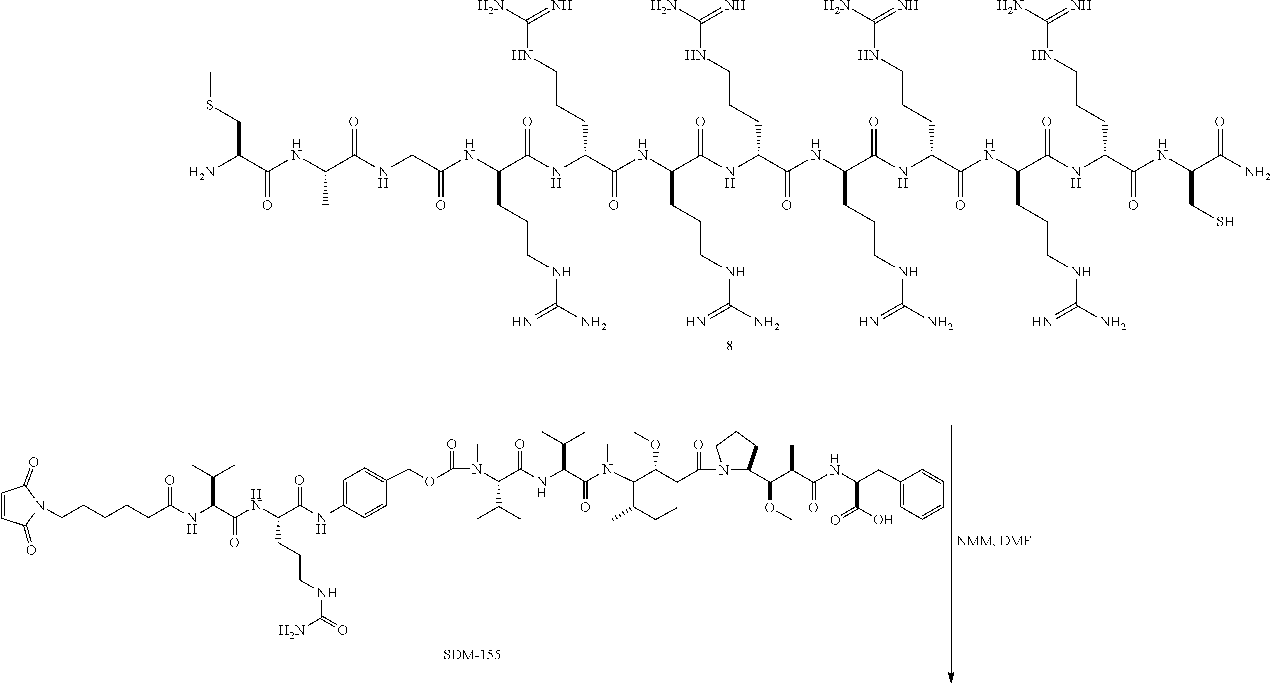

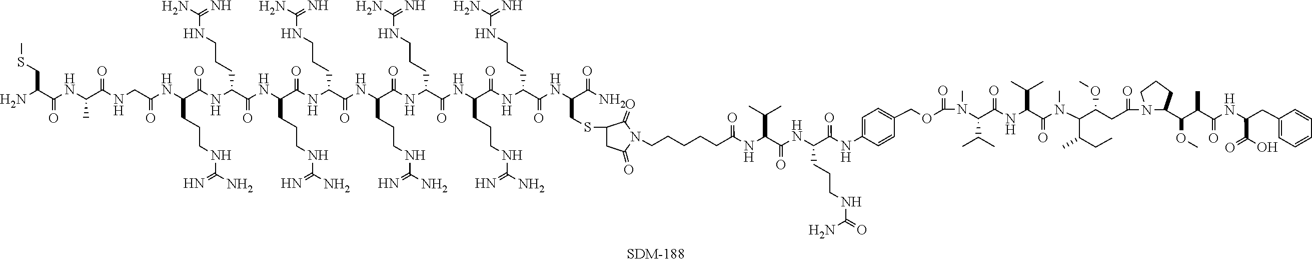

To a stirred solution of peptide 8 (10.0 mg, 3.8 μmol) and SDM-155 (6.0 mg, 4.5 μmol) in DMF (0.7 mL) was added N-methylmorpholine (NMM, 6.0 μL, 55 μmol). The reaction mixture was stirred at room temperature for 0.5 h. Purification by RP-HPLC afforded SDM-188 as a white powder after lyophilization (12.7 mg, 85%). MS (ESI): m/e 982.86 [M+3H]³⁺.

### II. Biological Evaluation

### Example 1: Test Compound Activity in Cancer Cell Viability Assay Using Human HT1080 Fibrosarcoma Cells

Method: Plate cells one day prior to start of assay in 96 well black with clear bottom plates (Corning #3603), 2×10⁴ cells per well in 100 μL of standard growth media (according to ATCC). Grow cells to 90% confluency.

Before start of assay rinse cells IX with DPBS. Add compounds in a 3-fold serial dilution in triplicate (range e.g. 8.1 μM-3.7 nM), 100 μL per well in standard growth media.

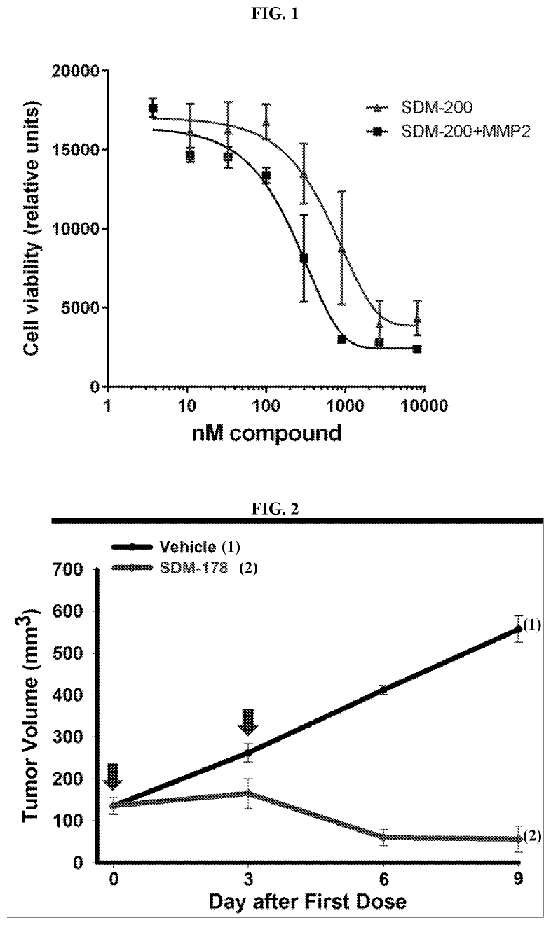

Incubate four days then assay cell viability as described below. Remove media and rinse IX with DPBS, then add 100 μL per well 10% Prestoblue Cell viability reagent (Thermo Fisher Scientific #A13261) in complete media. Return to incubator for two hours then read plate on SpectraMax M2e, bottom read excitation 555 emission 585 cut-off 570 which measures number of live cells. Cell viability was plotted versus compound concentration, as shown in FIG. 1, and data were fit to an exponential decay curve and EC₅₀ were determined. In most cases, SDMs were also tested in activated form by pre-treatment with protease that activates the compound. In FIG. 1, this is shown for SDM-200 where it was activated with MMP2 to generate the more active form of SDM. In Table 9 this is represented as ACT SDM-200.

FIG. 1 provides an example of SDM compound dose responses and EC₅₀ determination of therapeutic compounds in cell viability assay with HT1080 fibrosarcoma cells.

Table 9 provides cellular activity data for SDMs (compound EC₅₀ for reduction of viable cells).

TABLE 9

| SDM Compound | Activity (EC50) |
|---|---|
| SDM-178 | Med |
| ACT SDM-178 | High |
| SDM-180 | High |
| SDM-183 | High |
| SDM-185 | High |
| SDM-186 | High |
| SDM-188 | High |
| SDM-189 | Med |
| SDM-190 | Low |
| ACT-SDM-190 | High |
| SDM-200 | Med |
| ACT SDM-200 | High |
| SDM-218 | High |
| ACT SDM-218 | High |
| SDM-223 | Med |
| ACT SDM-223 | High |
| SDM-227 | High |
| ACT SDM-227 | High |
| SDM-233 | High |
| ACT SDM-233 | High |

EC₅₀ Efficacy: High defined as < 300 nM;
Med defined as 300-1500 nM;
Low defined as > 1500 nM

### Example 2: Human Fibrosarcoma Tumor Xenograft Therapeutic Models

Female athymic nude mice (8-10 weeks old) purchased from Charles River (Wilmington, 01887, MA) were used after 4-7 day of acclimatization period. Institutional Animal Care and Use Committee (IACUC) approved protocols #EB11-002-009 and CP-17-11. Human fibrosarcoma tumor cells from ATCC (CCL-121™) were grown using standard cell culture techniques. Tumor cells (1×10⁸ tumor cells/mL) were suspended in DPBS/Matrigel™ (1:1 vol) and implanted subcutaneously (50 μL tumor cell suspension/mouse) into the upper mammary fat pad or right flank of mice with individual identification number (ear tag). Four to seven days later when the average tumor volume was about 50-100 mm³, mice were randomized into different experimental groups based on identical averaged (Mean±SEM) tumor volume and dosed with (i) vehicle and (ii) different doses of test compounds. For each involved tumor-bearing mouse, the tumor volume (mm³) was measured using a caliper and calculated as follows: tumor volume=width²×length/2. Test compounds were administered intravenously (tail vein) twice (two administrations 3 days apart) in conscious restrained tumor-bearing mice throughout the study. Individual tumor volume, body weight as well as clinical signs were recorded during the study. Each study was terminated on day 12 post-dosing initiation. Mice were euthanized by intracardiac ketamine-xylazine overdose after recording the last individual body weight and tumor volume. For each experimental group and study day, the tumor volumes from all involved mice were averaged and expressed as Mean±SEM. For each test compound-treated group, the percentage of tumor growth inhibition was evaluated 6 days after the cessation of test compound administration and calculated using the value in vehicle-treated group as 0% inhibition according to the following formula: (tumor volume vehicle−day 9)−(tumor volume test compound−day 9)/(tumor volume vehicle−day 9)−(tumor volume vehicle−day 0)×100.

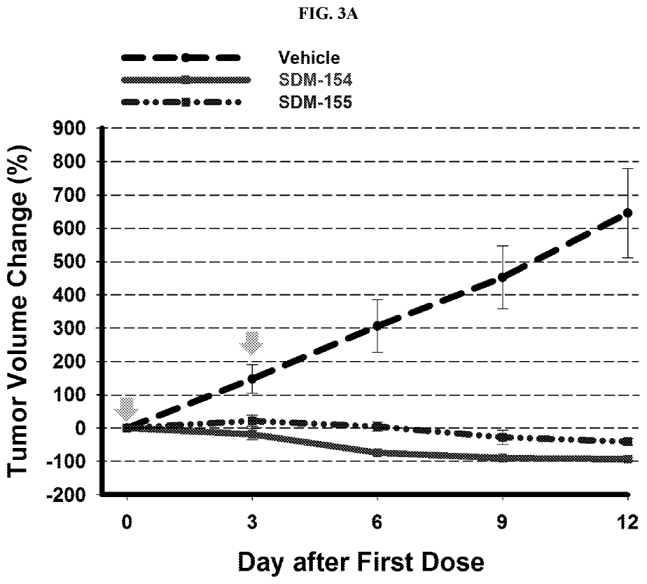

FIG. 2 provides an example of active SDMs in human fibrosarcoma xenograft model demonstrating complete block of tumor growth compared to vehicle control. The arrows indicate IV dosing of SDM and error bars are standard errors of the means. SDM-178 was dosed at 2 mg/kg.

Table 10 provides efficacy data for SDM Compounds in HT1080 human fibrosarcoma subcutaneous tumor xenograft model. All compounds were dosed at 0.6 to 3.0 mg/kg of cytotoxic payload component.

TABLE 10

| SDM | |
|---|---|
| Compound | Efficacy |
| SDM-173 | High |
| SDM-178 | High |
| SDM-179 | High |
| SDM-180 | High |
| SDM-181 | Medium |
| SDM-183 | Medium |
| SDM-184 | High |
| SDM-185 | Medium |
| SDM-191 | High |
| SDM-200 | High |
| SDM-204 | High |
| SDM-209 | High |
| SDM-210 | High |
| SDM-218 | High |
| SDM-227 | High |
| SDM-258 | Medium |
| SDM-261 | High |
| SDM-262 | High |
| SDM-263 | High |
| SDM-264 | High |
| SDM-265 | High |
| SDM-347 | Low |

Efficacy defined as percent tumor growth inhibition versus vehicle
High: ≥80% inhibition
Medium: ≥29% inhibition to < 80% inhibition
Low: <29% inhibition

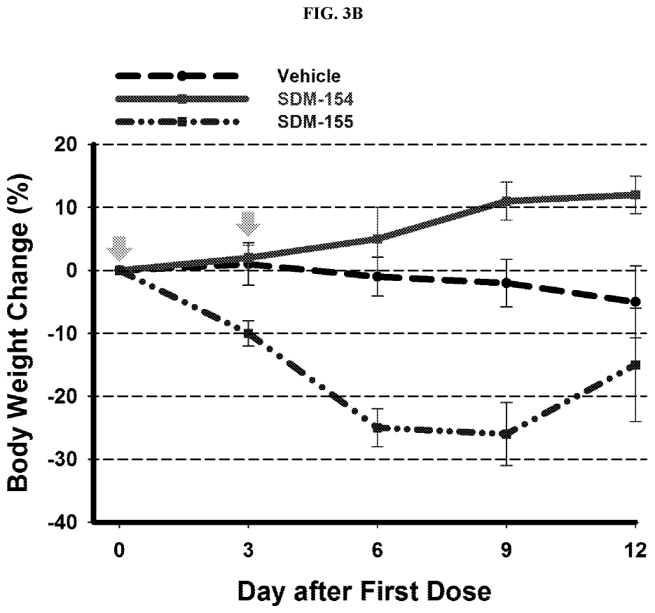

FIGS. 3A and 3B each provide an example of the efficacy of the modified therapeutic agent Y -D disclosed herein, as compared with non-modified therapeutic agent D. SDM-154 and SDM-155 are shown below, and are not compounds of the disclosure. However, comparing the efficacy of SDM-154 and SDM-155 provides useful insight into the efficacy of the compounds of the disclosure, as Y-D is an active cytotoxic agent disclosed herein. SDM-154 and SDM-155 were evaluated in a human HT1080 fibrosarcoma xenograft model. FIG. 3A shows the efficacy of each SDM with respect to tumor volume as a function of time normalized to day 1. Compounds were doses twice, via IV, on days 0 and 3 as indicated by downward arrow. The error bars are standard deviations. Both SDM 154 and SDM 155 were dosed at a 1 mg/kg of the cytotoxic agent component. From these results, SDM 154 can be seen to reduce tumor volume more than SDM-155. FIG. 3B shows each SDM's effect on body weight normalized to day 1. Monitoring body weight shows a measure of tolerability and toxicity. The data show that SDM-155 is not tolerated and has significant weight loss (>20% weight loss) compared to SDM-154 and the vehicle.

Table 11 shows SDM-154 and SDM-155. SDM 154 is a compound of the disclosure. SDM-155 is not a compound of the disclosure.

TABLE 11

| | |
|---|---|
| Compound Name | Chemical Structure 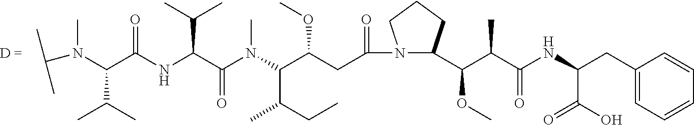 |
| SDM-154 | 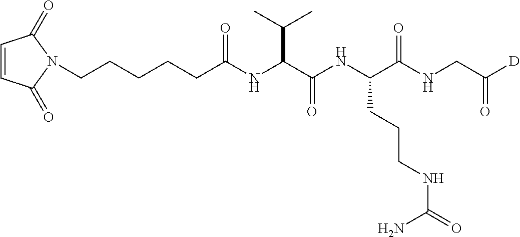 [SMILES: [2H]C(=O)CNC(=O)[C@H](CCCNC(N)=O)NC(=O)[C@@H](NC(=O)CCCCCN1C(=O)C=CC1=O)C(C)C] |
| SDM-155 | 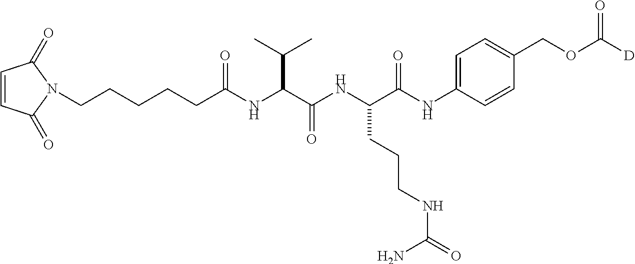 [SMILES: [2H]C(=O)OCc1ccc(NC(=O)[C@H](CCCNC(N)=O)NC(=O)[C@@H](NC(=O)CCCCCN2C(=O)C=CC2=O)C(C)C)cc1] |

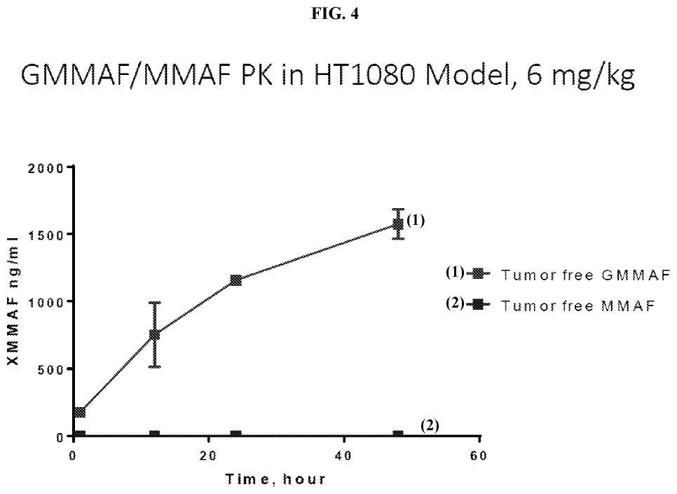

SDM-154 was dosed at 6.0 mg/kg in the HT1080 human fibrosarcoma subcutaneous tumor xenograft model described above. The generation of MMAF and glycine-MMAF (GMMAF) was monitored, the results of which are presented in FIG. 4. The MMAF and GMMAF observed was not bound to the citrulline linker present in SDM-154. The data show that GMMAF is present in high concentration, while MMAF concentrations remain negligible. In vitro HT-1080 tumor homogenate experiments have yielded analogous results.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

What is claimed is:

1. A selective delivery molecule of Formula I, having the structure:
[(S)_{w}-A-c_{A}-(c_{P}-M)ᵤ-X—B-c_{B}-D_{B}]-(N—Z)ᵥ Formula I
wherein,
S is an electrophilic substituent bound to the amino terminus of A or c_{A};
A is a peptide with a sequence comprising 5 to 9 acidic amino acids;
c_{A} is a bond or a single amino acid linker;
cₚ is a linker;
M is a macromolecule;
X is a cleavable linker;
B is a peptide with a sequence comprising 5 to 20 basic amino acids;
c_{B} is a single amino acid linker;
D_{B} is a therapeutic agent or an imaging agent;
each N is independently selected from a bond and a linker;
each Z is independently a peptide with a sequence comprising 3 to 10 amino acids;
v is 1 or 2; and
w and u are independently 0 or 1;
wherein each N is independently bound to c_{A}, c_{B}, or the amino terminus of A, S is bound to the amino terminus of A, and cₚ is bound to c_{A}; and
wherein if S is present, S and N are not bound at the same position; and if cₚ is present, cₚ and N are not bound at the same position.

2. The selective delivery molecule of claim 1, wherein X is cleavable by an extracellular protease.

3. The selective delivery molecule of claim 1, wherein Z comprises a receptor binding peptide.

4. The selective delivery molecule of any one of the claims 1-3, wherein Z comprises a urokinase type plasminogen activator receptor (uPAR) peptide.

5. The selective delivery molecule of any one of the claims 1-4, wherein Z comprises SRSRY, SRNRY, SRGRY, SQSRY, SQNRY, SQGRY, PRSRY, PRNRY, PRGRY, PQSRY, PQNRY, or PQGRY.

6. The selective delivery molecule of claim 1, wherein Z comprises a series of 4 Phe residues.

7. The selective delivery molecule of claim 1, wherein Z is a peptide with a sequence selected from the group consisting of:

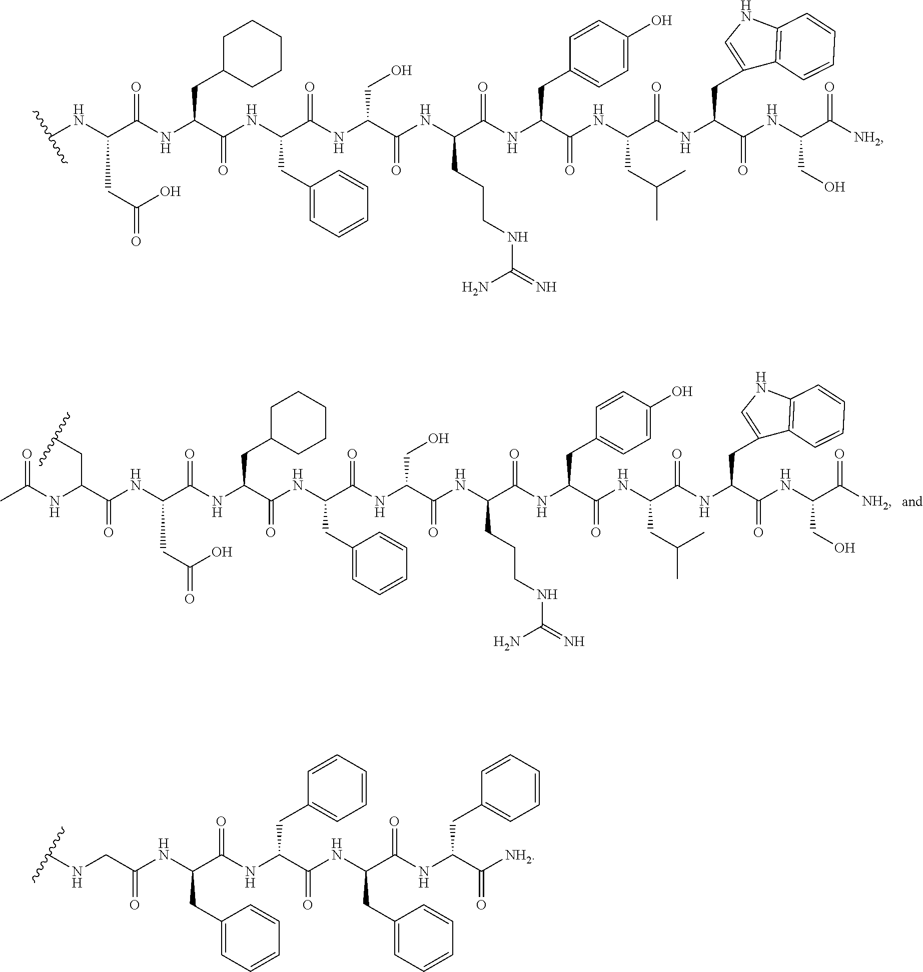

8. The selective delivery molecule of claim 1, wherein if v is 2, the two N are not identical.

9. The selective delivery molecule of claim 1, wherein N is a bond or a linker selected from the group consisting of:

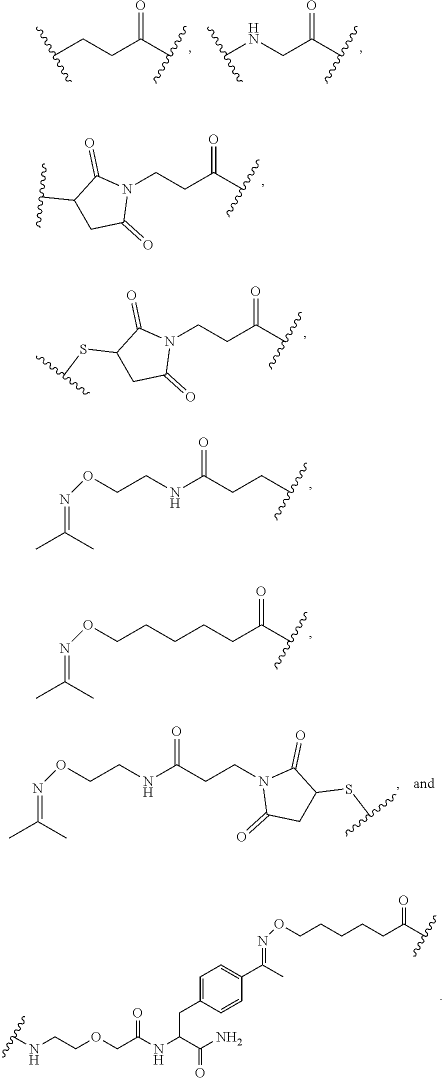

10. The selective delivery molecule of claim 1, wherein c_{A} is a bond,

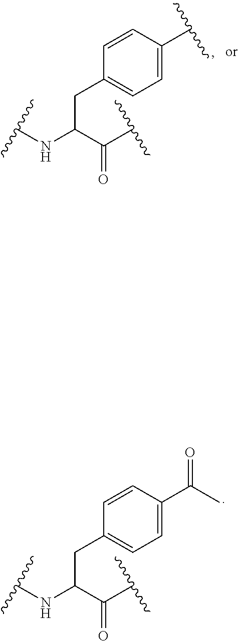 [SMILES: *NC(Cc1ccc(*)cc1)C(*)=O ; *NC(Cc1ccc(C(C)=O)cc1)C(*)=O]

11. The selective delivery molecule of claim 1, wherein cₚ is selected from:

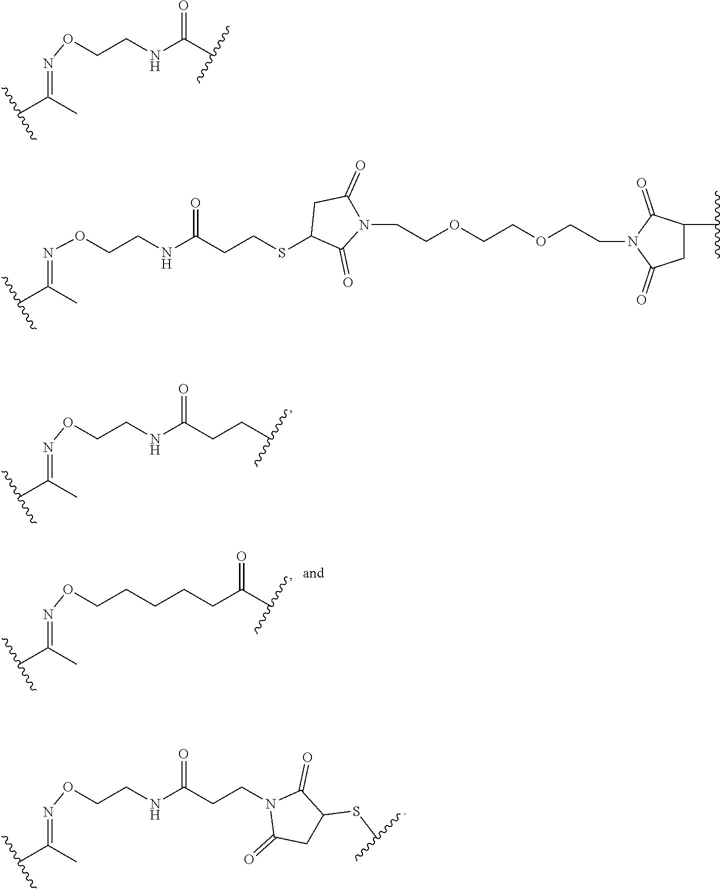 [SMILES: *C(=O)NCCO/N=C(/*)C ; */C(C)=N/OCCNC(=O)CCSC1CC(=O)N(CCOCCOCCN2C(=O)CC(*)C2=O)C1=O ; *CCC(=O)NCCO/N=C(/*)C ; *C(=O)CCCCCO/N=C(/*)C ; *SC1CC(=O)N(CCC(=O)NCCO/N=C(/*)C)C1=O]

12. The selective delivery molecule of claim 1, wherein M is a polyethylene glycol substituent.

13. The selective delivery molecule of claim 1, wherein X comprises PLGLAG, PLG-C(me)-AG, RPLALWRS, ESPAYYTA, DPRSFL, PPRSFL, RLQLKL, or RLQLK(Ac)L.

14. The selective delivery molecule of claim 1, wherein X comprises —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, or —NHCH₂CH₂OCH₂CH₂OCH₂C(O)NHCH₂CH₂OCH₂CH₂OCH₂C(O)—.

15. The selective delivery molecule of claim 1, wherein B is a peptide with a sequence comprising 7 to 9 basic amino acids.

16. The selective delivery molecule of claim 1, wherein S is an N-maleimide or N-succinamide substituent.

17. The selective delivery molecule of claim 1, wherein S is 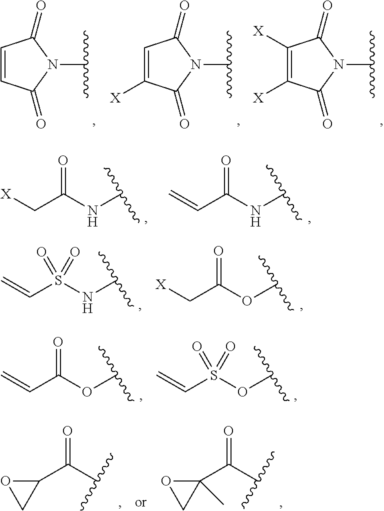
wherein each X is independently —Cl, —Br, —I, or —S-phenyl.

18. The selective delivery molecule of claim 1, wherein S is

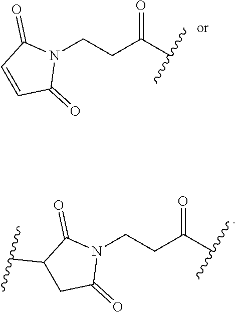 [SMILES: *C(=O)CCN1C(=O)C=CC1=O ; *C(=O)CCN1C(=O)CC(*)C1=O]

19. The selective delivery molecule of claim 1, wherein S is bound to the amino terminus of A.

20. The selective delivery molecule of claim 1, wherein S is further conjugated to an albumin.

21. The selective delivery molecule of claim 1, wherein c_{B} is

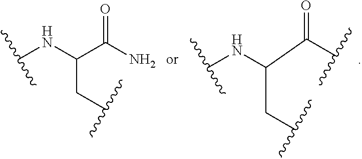 [SMILES: *CC(N*)C(N)=O ; *CC(N*)C(*)=O]

22. The selective delivery molecule of claim 1, wherein D_{B} is an auristatin-related therapeutic agent or a cyanine-related imaging agent.

23. The selective delivery molecule of claim 1,
wherein,
c_{A} is a bond, 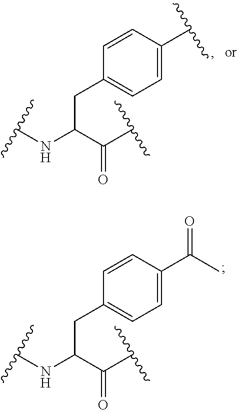 [SMILES: *NC(Cc1ccc(*)cc1)C(*)=O ; *NC(Cc1ccc(C(C)=O)cc1)C(*)=O]
cₚ is selected from: 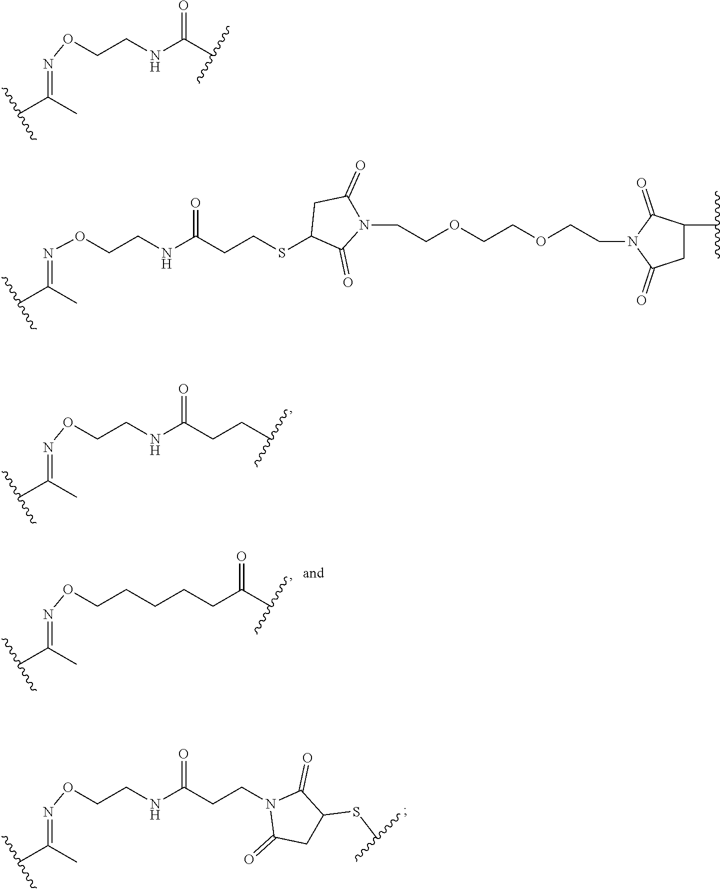 [SMILES: *C(=O)NCCO/N=C(/*)C ; */C(C)=N/OCCNC(=O)CCSC1CC(=O)N(CCOCCOCCN2C(=O)CC(*)C2=O)C1=O ; *CCC(=O)NCCO/N=C(/*)C ; *C(=O)CCCCCO/N=C(/*)C ; *SC1CC(=O)N(CCC(=O)NCCO/N=C(/*)C)C1=O]
M is a polyethylene glycol substituent;
X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—;
B is a peptide with a sequence comprising 7 to 9 basic amino acids;
c_{B} is 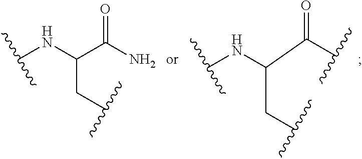 [SMILES: *CC(N*)C(N)=O ; *CC(N*)C(*)=O]
D_{B} is an auristatin-related therapeutic agent or a cyanine-related imaging agent;
N is a bond or a linker selected from the group consisting of: 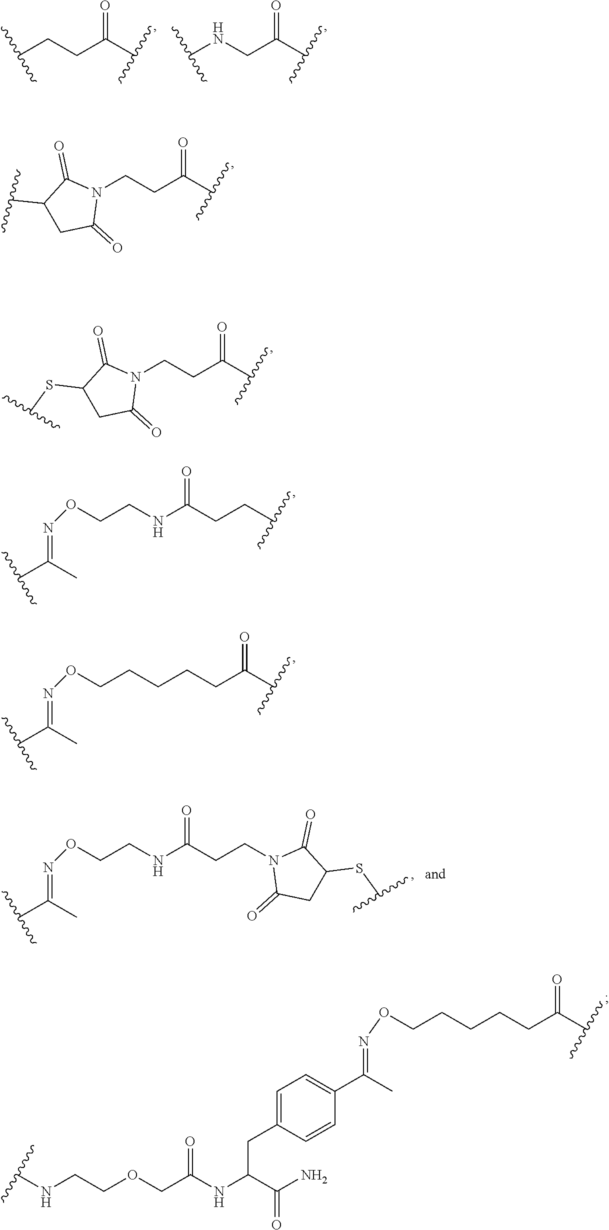
Z is a peptide with a sequence selected from the group consisting of:

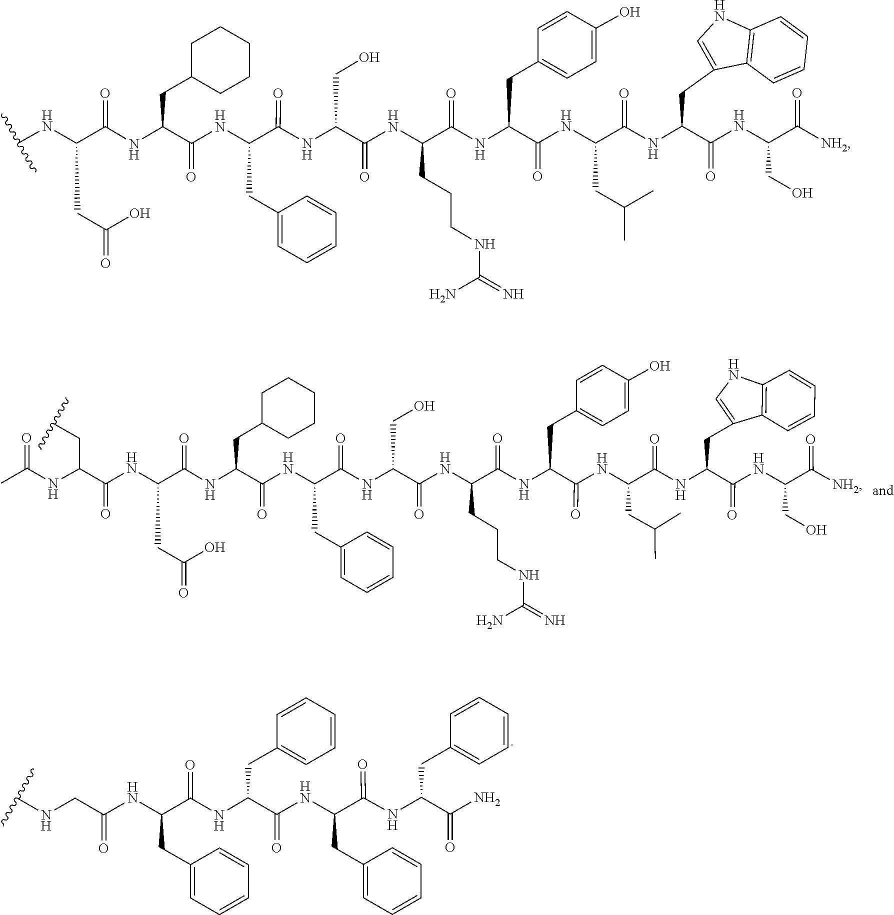

24. The selective delivery molecule of claim 1, wherein D_{B} is G-T-Q-Y-D;
G is selected from the following substituents: 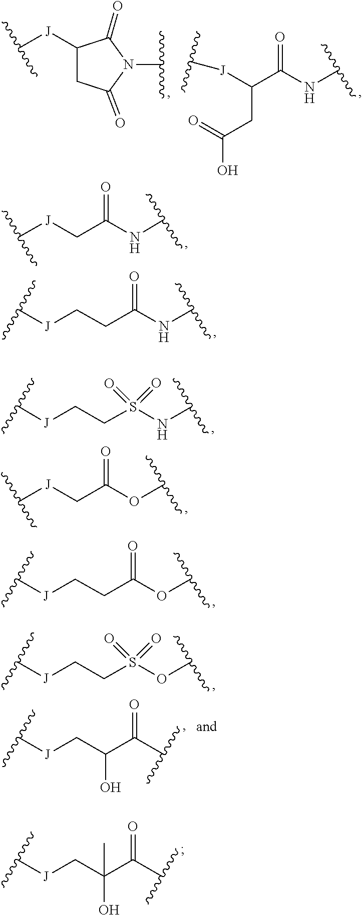
J is —O—, —NH—, or —S—;
T is an optionally substituted C₁-C₈ alkylene, optionally substituted C₁-C₈ alkylene-C(O)—, optionally substituted C₃-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—CH₂)ₘC(O)—;
R^{1B} is —H, —CH₃, —CH₂CH₃, or —CH₂CH₂NH₂;
each n is independently an integer ranging from 1 to 25;
each m is independently an integer ranging from 1 to 10;
Q is a bond or 1-3 amino acids selected from the group consisting of: 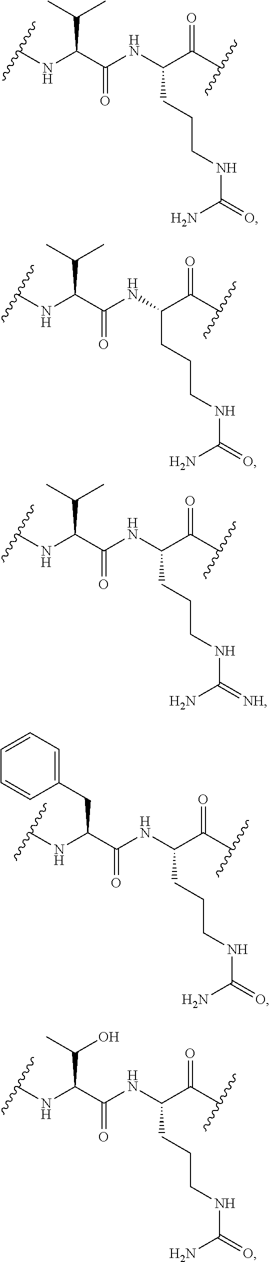 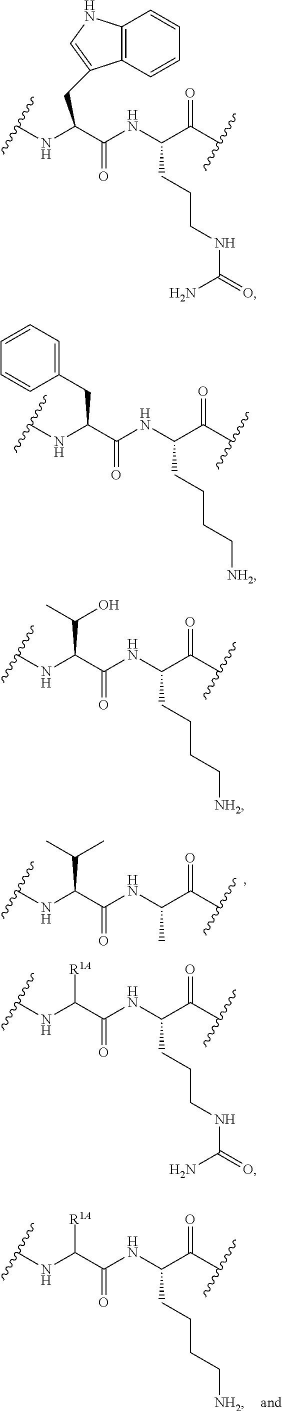 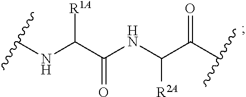
R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂, —CH₂CH₂CH₂NHC(═NH)NH₂, or —CH₂CH₂CH₂NHC(O)NH₂;
Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—;
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
D is U or Z²;
U is a fragment having the structure of Formula (IA) or Formula (IB): 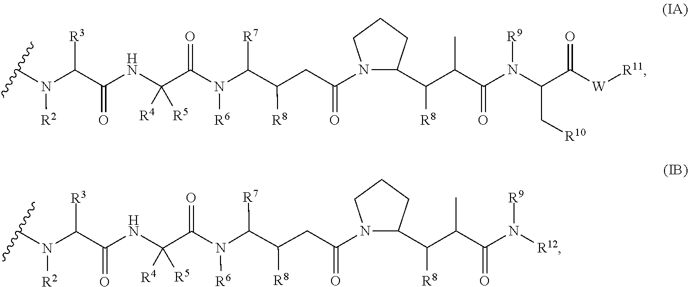
wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is an integer ranging between 0 to 6;
t is an integer ranging between 0 to 6;
Z² is a fragment having the structure of Formula (IC): 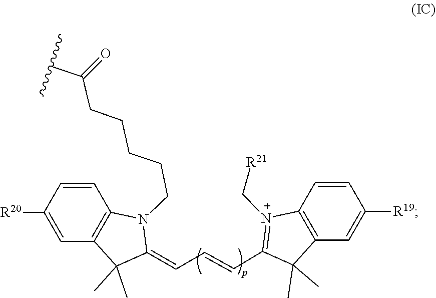
wherein,
each R¹⁹ and R²⁰ are independently —H, —SO₃⁻, —SO₃H, or C₁-C₈ alkyl, wherein at least one of R¹⁹ and R²⁰ is —SO₃⁻;
R²¹ is —H or C₁-C₈ alkyl; and
p is an integer ranging from 0 to 3.

25. The selective delivery molecule of claim 23, wherein G is selected from the following substituents:

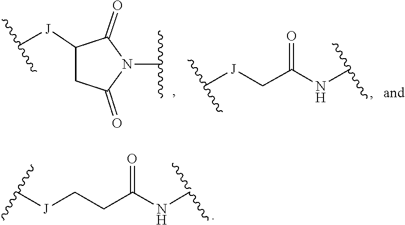

26. The selective delivery molecule of any one of claims 23-25, wherein J is —O— or —S—.

27. The selective delivery molecule of any one of claims 23-26, wherein T is an optionally substituted C₁-C₈ alkylene-C(O)—.

28. The selective delivery molecule of any one of claims 23-27, wherein Q is selected from the following substituents:

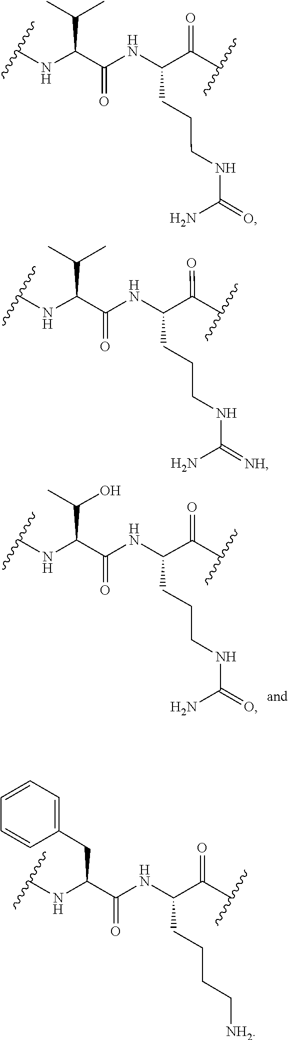

29. The selective delivery molecule of any one of claims 23-28, wherein Y is a bond, —NHCH₂C(O)—, —NHCH(CH₃)C(O)—, or —NHCH₂CH₂—.

30. The selective delivery molecule of any one of claims 23-29, wherein
R² is —H or optionally substituted C₁-C₈ alkyl;
R³ is —H, or optionally substituted C₁-C₈ alkyl;
R⁴ is —H, or optionally substituted C₁-C₈ alkyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, or optionally substituted C₃-C₈ carbocyclyl;
each R⁸ is independently selected from —H, —OH, optionally substituted C₁-C₈ alkyl, optionally substituted G-C₈ carbocyclyl, and —O-(optionally substituted C₁-C₈ alkyl)-;
R⁹ is —H;
R¹⁰ is optionally substituted C₆-C₁₀ aryl;
W is —O—;
R¹¹ is —H; and
R¹² is —C(R⁸)₂—C(R⁸)₂—(C₆-C₁₀ aryl).

31. The selective delivery molecule of claim 23,
wherein,
G is selected from the following substituents: 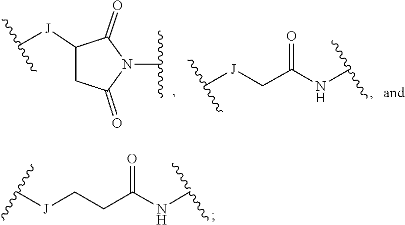
J is —O— or —S—;
T is an optionally substituted C₁-C₈ alkylene-C(O)—;
Q is selected from the following substituents: 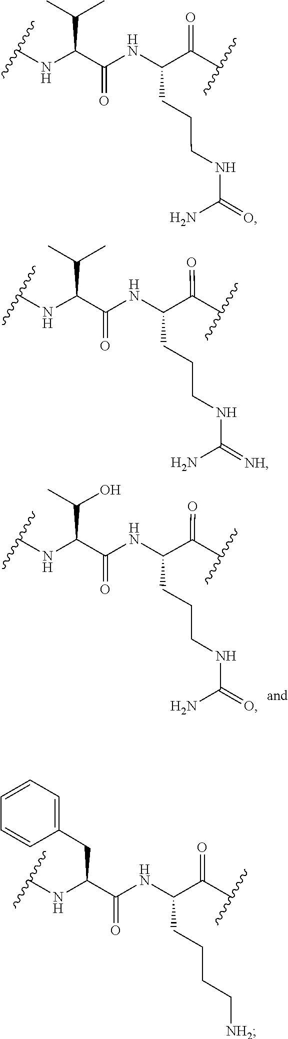
Y is a bond, —NHCH₂C(O)—, —NHCH(CH₃)C(O)—, or —NHCH₂CH₂—;
R² is —H or optionally substituted C₁-C₈ alkyl;
R³ is —H, or optionally substituted C₁-C₈ alkyl;
R⁴ is —H, or optionally substituted C₁-C₈ alkyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, or optionally substituted C₃-C₈ carbocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl)-;
R⁹ is —H;
R¹⁰ is optionally substituted C₆-C₁₀ aryl;
W is —O—;
R¹¹ is —H; and
R¹² is —C(R⁸)₂—C(R⁸)₂—(C₆-C₁₀ aryl).

32. The selective delivery molecule of claim 31, wherein U is monomethyl auristatin F (MMAF).

33. The selective delivery molecule of claim 31, wherein U is monomethyl auristatin E (MMAE).

34. The selective delivery molecule of claim 31, wherein Z² is cyanine-5, or a derivative thereof.

35. The selective delivery molecule of claim 1, wherein the selective delivery molecule is: SDM-177, SDM-179, SDM-180, SDM-183, SDM-184, SDM-186, SDM-209, SDM-258, SDM-259, or SDM-260.

36. A selective delivery molecule of Formula II, having the structure:
M²ₒ-S_{w}-A-c_{A}-(c_{P}-M¹)ᵤ-X—B-c_{B}-G-T-Q-Y-D Formula II
wherein,
M¹ and M² are each independently a macromolecule;
S is an N-maleimide substituent or N-succinamide linker bound to the amino terminus of A;
A is a peptide with a sequence comprising 5 to 9 acidic amino acids;
c_{A} is a bond or a single amino acid;
cₚ is a linker bound to c_{A};
X is a cleavable linker;
B is a peptide with a sequence comprising 5 to 20 basic amino acids;
c_{B} is a single amino acid;
G is a linker;
T is a spacer;
Q is a bond or 1-3 amino acids;
Y is 1-3 amino acids or an amino alkylene;
D is an auristatin related therapeutic agent; and
w, u, and o are independently 0 or 1.

37. The selective delivery molecule of claim 36, wherein M² is a polyethylene glycol substituent or an albumin substituent.

38. The selective delivery molecule of claim 36 or 37, wherein if M² is absent, S comprises

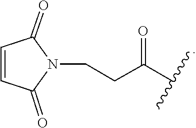 [SMILES: *C(=O)CCN1C(=O)C=CC1=O]

39. The selective delivery molecule of claim 36 or 37, wherein if M² is present, M²-S is selected from: 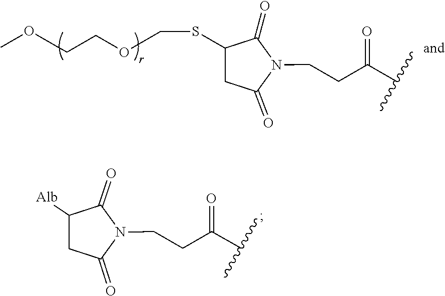
wherein Alb is an albumin protein and r is independently an integer ranging from 40-1,100.

40. The selective delivery molecule of any one of claims 36-39, wherein cₚ is selected from:

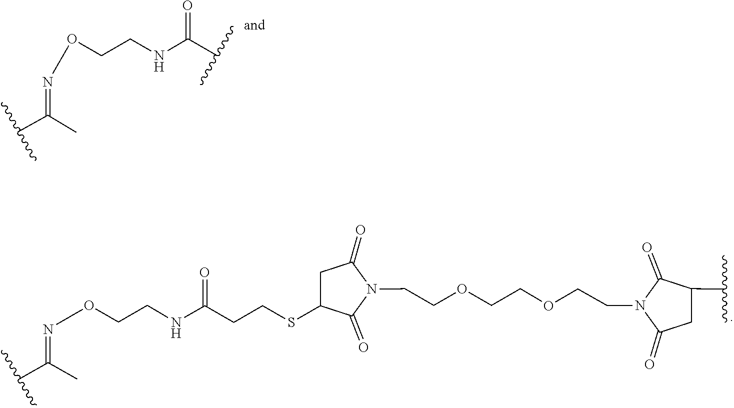 [SMILES: *C(=O)NCCO/N=C(/*)C ; */C(C)=N/OCCNC(=O)CCSC1CC(=O)N(CCOCCOCCN2C(=O)CC(*)C2=O)C1=O]

41. The selective delivery molecule of any one of claims 36-40, wherein cₚ-M¹ is selected from: 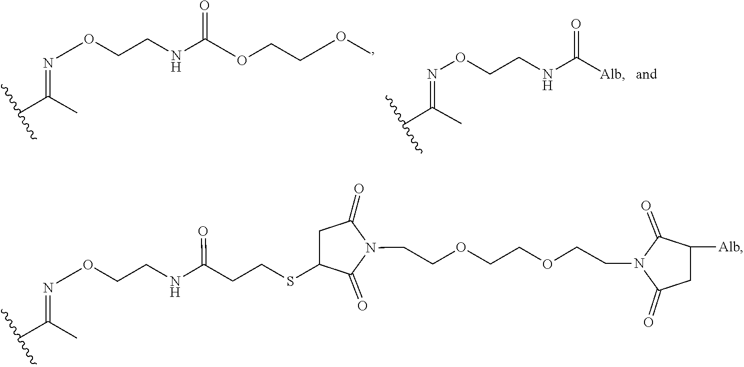
wherein Alb is an albumin protein and each r is independently an integer ranging from 40-1,100.

42. The selective delivery molecule of any one of claims 36-41, wherein X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—.

43. The selective delivery molecule of any one of claims 36-42, wherein c_{B} is

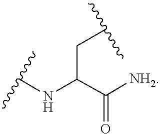 [SMILES: *CC(N*)C(N)=O]

44. The selective delivery molecule of any one of claims 36-43, wherein B is a peptide with a sequence comprising 7 to 9 basic amino acids.

45. The selective delivery molecule of any one of claims 36-44, wherein G is selected from the following substituents:

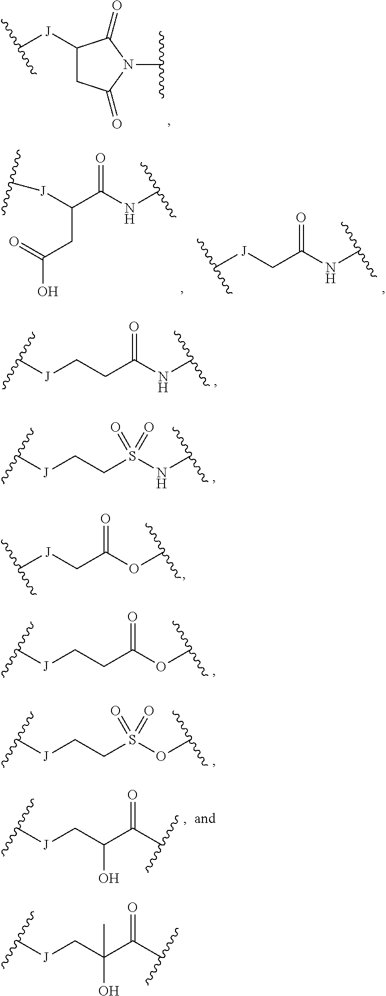

46. The selective delivery molecule of claim 45, wherein J is —O—, —NH—, or —S—.

47. The selective delivery molecule of any one of claims 36-46, wherein T is an optionally substituted C₁-C₈ alkylene, optionally substituted C₁-C₈ alkylene-C(O)—, optionally substituted C₃-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—(CH₂)ₘC(O)— wherein R^{1B} is —H, —CH₃, —CH₂CH₃, or —CH₂CH₂NH₂.

48. The selective delivery molecule of any one of claims 36-47, wherein Q is a bond or selected from the group consisting of: 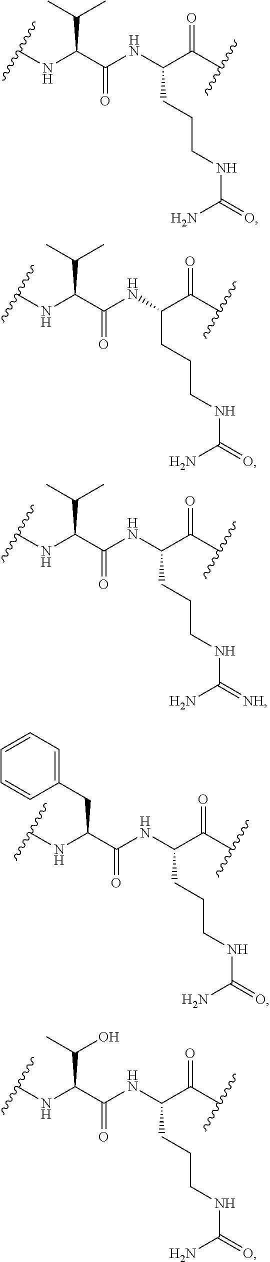 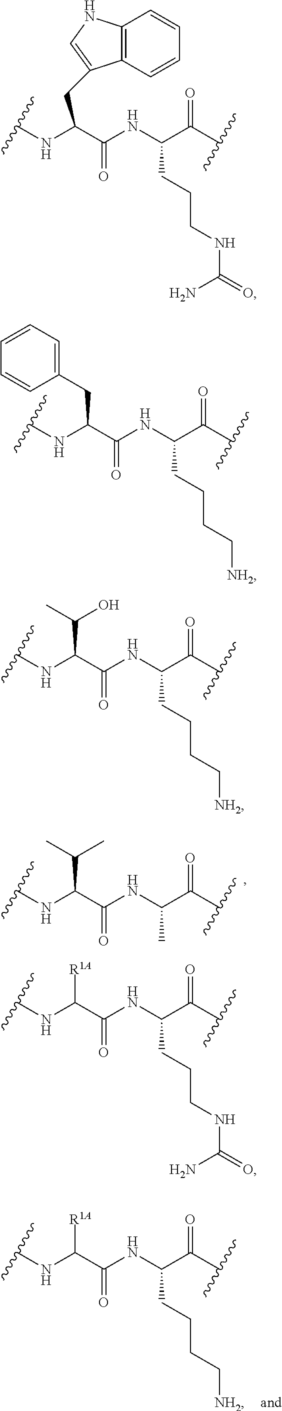 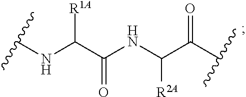
wherein,
R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl; and
R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂, —CH₂CH₂CH₂NHC(═NH)NH₂, or —CH₂CH₂CH₂NHC(O)NH₂.

49. The selective delivery molecule of any one of claims 36-48, wherein Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—; wherein,
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH.

50. The selective delivery molecule of any one of claims 36-49, wherein D is U.

51. The selective delivery molecule of any one of claims 36-50, wherein U is a fragment having the structure of Formula (IIA) or Formula (IIB): 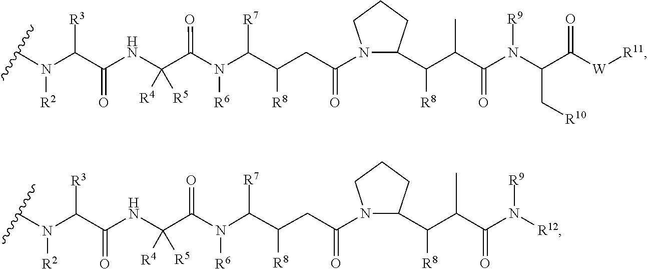
wherein,
R² is —H or optionally substituted C₁-C₈ alkyl;
R³ is —H, optionally substituted C₁-C₈ alkyl, optionally substituted C₁-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is and integer ranging between 0 to 6; and
t is and integer ranging between 0 to 6.

52. The selective delivery molecule of claim 36,
wherein,
M²-S is selected from the group consisting of: 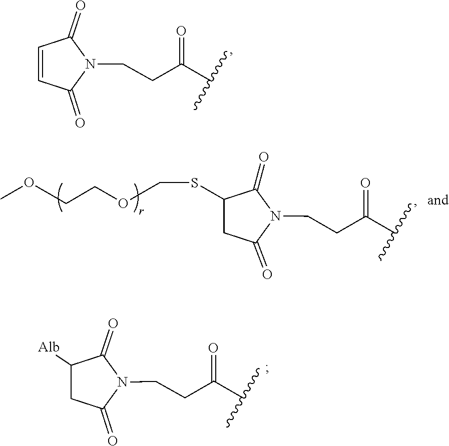
c_{A} is a bond, 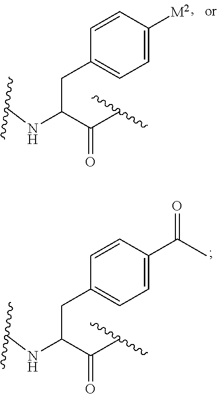
wherein,
cₚ-M¹ is selected from the group consisting of: 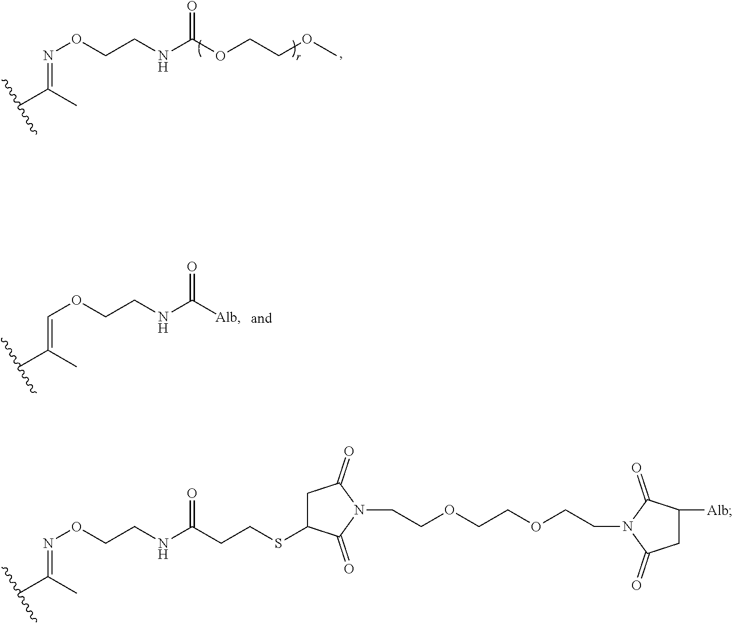
Alb is an albumin protein;
each r is independently an integer ranging from 40-1,100;
X is a cleavable linker selected from —NHCH₂CH₂OCH₂C(O)-Arg-Pro-Leu-Ala-Leu-Trp-Arg-Ser-, —NHCH₂CH₂OCH₂C(O)-Asp-Pro-Arg-Ser-Phe-Leu-, —NHCH₂CH₂OCH₂C(O)-Pro-Leu-Gly-Cys(Me)-Ala-Gly-, —NHCH₂CH₂OCH₂C(O)-Arg-Leu-Gln-Leu-Lys(Ac)-Leu-, and —NHCH₂CH₂OCH₂CH₂OCH₂C(O)NHCH₂CH₂OCH₂CH₂OCH₂C(O)—;
c_{B} is 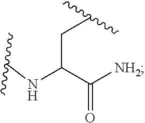 [SMILES: *CC(N*)C(N)=O]
B is a peptide with a sequence comprising 7 to 9 basic amino acids;
G is selected from the following substituents: 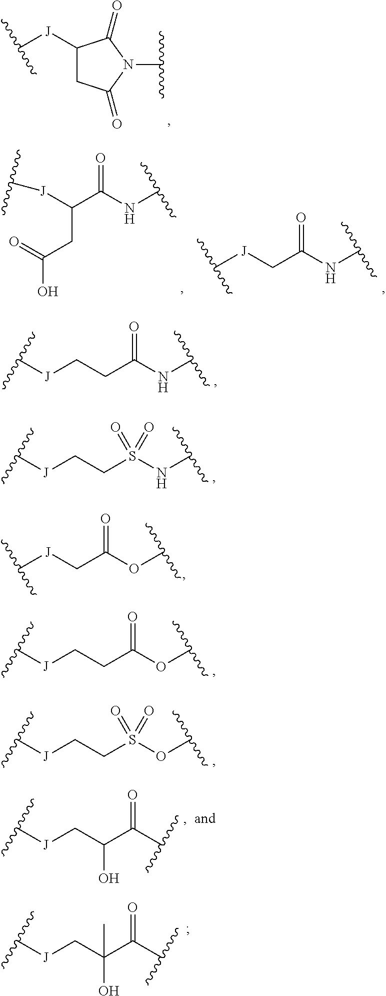
J is —O—, —NH—, or —S—;
T is an optionally substituted C₁-C₈ alkylene, optionally substituted C₁-C₈ alkylene-C(O)—, optionally substituted C₁-C₈ carbocyclylene, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene, optionally substituted C₆-C₁₀ arylene —C(O)—, —(CH₂—CH₂—O)ₙ—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, —(CH₂—CH₂—NR^{1B})ₙ—, or —(CH₂—CH₂—NR^{1B})ₙ—(CH₂)ₘC(O)—;
R^{1B} is —H, —CH₃, —CH₂CH₃, or —CH₂CH₂NH₂;
each n is independently an integer ranging from 1 to 25;
each m is independently an integer ranging from 1 to 10;
Q is a bond or selected from the group consisting of: 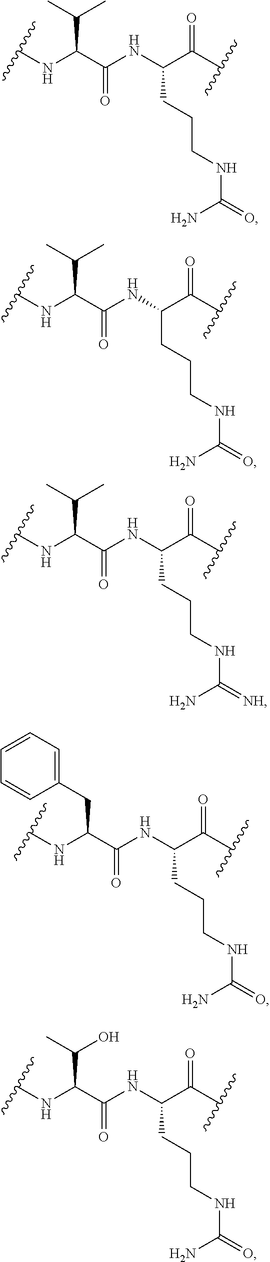 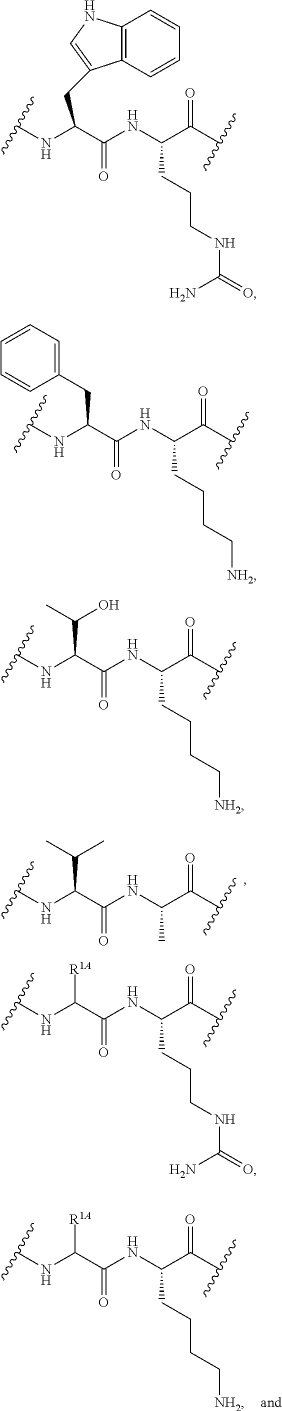 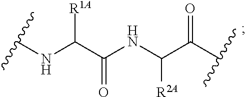
R^{1A} is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R^{2A} is —H, optionally substituted C₁-C₈ alkyl, optionally substituted C₃-C₈ carbocyclyl, optionally substituted C₆-C₁₀ aryl, optionally substituted C₇-C₁₂ aralkyl, optionally substituted C₃-C₈ heterocyclyl, amino substituted C₁-C₈ alkyl, —CH₂CH₂CH₂CH₂NH₂, —CH₂CH₂CH₂NHC(═NH)NH₂, or —CH₂CH₂CH₂NHC(O)NH₂;
Y is a bond, —NHCH₂C(O)—, —NHCH₂CH₂—, —OCH₂CH₂—, —NHCH₂S(O)₂—, —NHCR^{2B}R^{3B}C(O)—, —NHCR^{2B}R^{3B}CH₂—, or —NHCH₂C(O)NHCH₂CH₂NH—;
R^{2B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
R^{3B} is —H, -halogen, —CH₃, —CH₂CH₃, —CH(OH)CH₃, —CH₂OH, —CF₃, —CH(CH₃)₂, —CH₂CH₂C(O)OH, or —CH₂CH₂CH₂NHC(═NH)NH₂;
D is U;
U is a fragment having the structure of Formula (IIA) or Formula (IIB): 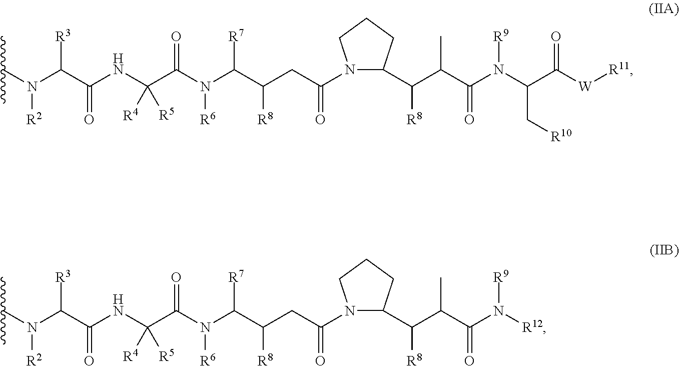
wherein,
R² is —H or optionally substituted C₁-C₈ alkyl;
R³ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁴ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
R⁵ is —H or —CH₃;
or R⁴ and R⁵ jointly form an optionally substituted C₃-C₈ carbocyclyl;
R⁶ is —H or optionally substituted C₁-C₈ alkyl;
R⁷ is —H, optionally substituted C₁-C₈ alkyl, optionally substituted C₃-C₈ carbocyclyl, optionally substituted C₆-C₁₀ aryl, optionally substituted C₇-C₁₂ aralkyl, or optionally substituted C₃-C₈ heterocyclyl;
each R⁸ is independently selected from —H, —OH, optionally substituted C₁-C₈ alkyl, optionally substituted C₃-C₈ carbocyclyl, and —O-(optionally substituted C₁-C₈ alkyl);
R⁹ is —H or optionally substituted C₁-C₈ alkyl;
R¹⁰ is optionally substituted C₆-C₁₀ aryl or optionally substituted C₃-C₈ heterocyclyl;
W is —O—, —S—, or —NR¹²—, wherein R¹² is —H or optionally substituted C₁-C₈ alkyl;
R¹¹ is —H, optionally substituted C₁-C₂₀ alkyl, C₆-C₁₀ aryl, C₃-C₈ heterocyclyl, —(R¹³O)ₜ—R¹⁴, or —(R¹³)O)ₜ—CH(R¹⁵)₂;
R¹² is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
R¹³ is optionally substituted C₁-C₈ alkyl;
R¹⁴ is —H or optionally substituted C₁-C₈ alkyl;
each occurrence of R¹⁵ is independently —H, —COOH, —(CH₂)_{q}—N(R¹⁶)₂, —(CH₂)_{q}—SO₃H, or —(CH₂)_{q}—SO₃-(optionally substituted C₁-C₈ alkyl);
each occurrence of R¹⁶ is independently —H, optionally substituted C₁-C₈ alkyl, or —(CH₂)_{q}—COOH;
R¹⁸ is —C(R⁸)₂—C(R⁸)₂—(C₆-C₁₀ aryl), —C(R⁸)₂—C(R⁸)₂—(C₃-C₈ heterocyclyl), or —C(R⁸)₂—C(R⁸)₂—(C₃-C₈ carbocyclyl);
q is an integer ranging between 0 to 6; and
t is an integer ranging between 0 to 6.

53. The selective delivery molecule of claim 52, wherein G is selected from the following substituents:

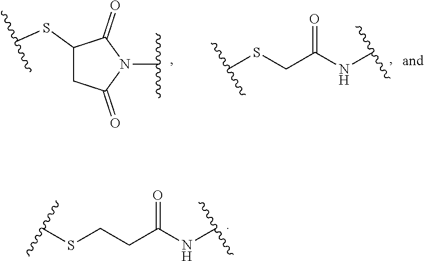 [SMILES: *SC1CC(=O)N(*)C1=O ; *NC(=O)CS* ; *NC(=O)CCS*]

54. The selective delivery molecule of any one of claims 52-53, wherein T is an optionally substituted C₁-C₈ alkylene-C(O)—, optionally substituted C₃-C₈ carbocyclylene-C(O)—, optionally substituted C₁-C₈ alkylene-C(O)NHCH₂C(O)—, optionally substituted C₁-C₈ alkylene-C(O)—(NHCH₂C(O))ₙ—, optionally substituted C₆-C₁₀ arylene —C(O)—, —(CH₂—CH₂—O)ₙ—(CH₂)ₘC(O)—, optionally substituted C₆-C₁₀ arylene-C(O)NH—(CH₂—CH₂—O)ₙ—(CH₂)ₘC(O)—, optionally substituted C₁-C₈ alkylene —C(O)NH—(CH₂—CH₂—O)ₙ—(CH₂)ₘC(O)—, or —(CH₂—CH₂—NR^{1B})ₙ—(CH₂)ₘC(O)—.

55. The selective delivery molecule of any one of claims 52-54, wherein Q is a bond or

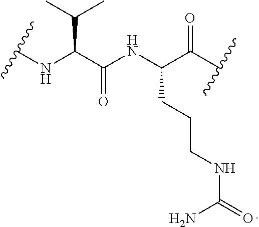 [SMILES: *N[C@H](C(=O)N[C@@H](CCCNC(N)=O)C(*)=O)C(C)C]

56. The selective delivery molecule of any one of claims 52-55, wherein Y is —NHCH₂C(O)- or —NHCH(CH₃)C(O)—.

57. The selective delivery molecule of claim 52, wherein U is monomethyl auristatin F (MMAF).

58. The selective delivery molecule of claim 52, wherein U is monomethyl auristatin E (MMAE).

59. The selective delivery molecule of claim 52,
wherein,
M²-S is selected from the group consisting of: 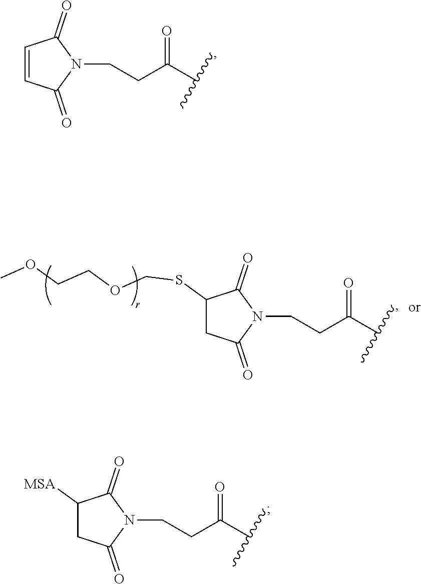
c_{A} is a bond, 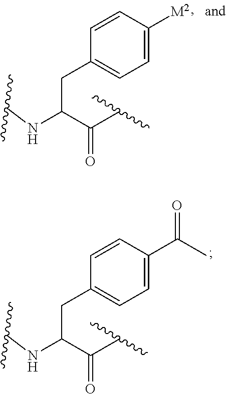
wherein,
cₚ-M¹ is selected from the group consisting of: 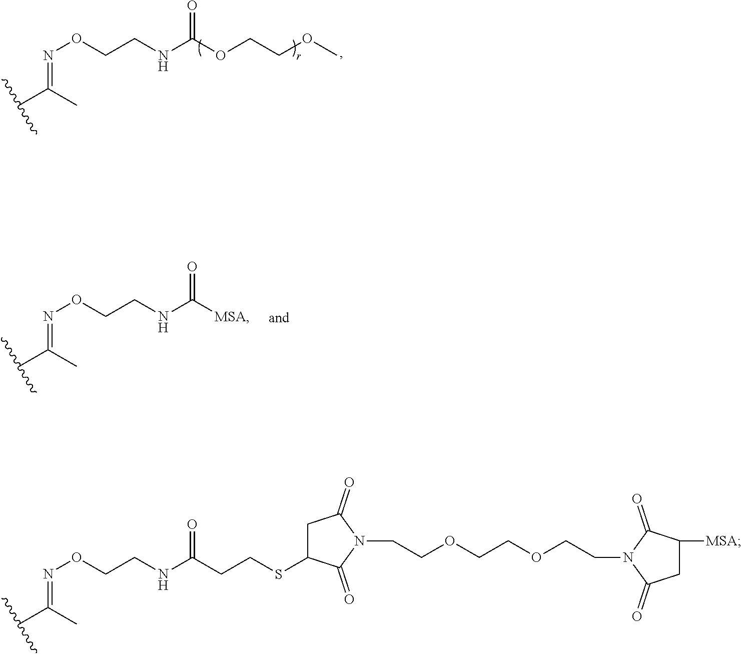
each r is independently an integer ranging from 40-1,100;
MSA is mouse serum albumin;
G is selected from the following substituents: 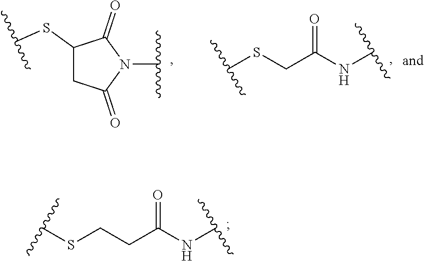 [SMILES: *SC1CC(=O)N(*)C1=O ; *NC(=O)CS* ; *NC(=O)CCS*]
T is an optionally substituted C₁-C₈ alkylene-C(O)—;
Q is a bond or 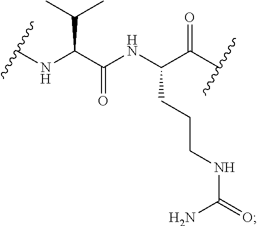 [SMILES: *N[C@H](C(=O)N[C@@H](CCCNC(N)=O)C(*)=O)C(C)C]
Y is —NHCH₂C(O)- or —NHCH(CH₃)C(O)—;
D is U; and
U is MMAE or MMAF.

60. The selective delivery molecule of claim 36, wherein the selective delivery molecule is: SDM-173, SDM-178, SDM-181, SDM-182, SDM-189, SDM-190, SDM-191, SDM-195, SDM-199, SDM-200, SDM-210, SDM-211, SDM-212, SDM-218, SDM-219, SDM-220, SDM-221, SDM-222, SDM-225, SDM-227, SDM-228, SDM-229, SDM-230, SDM-231, SDM-257, or SDM-266.

61. The selective delivery molecule of any one of the preceding claims, wherein A and B do not have an equal number of acidic and basic amino acids.

62. The selective delivery molecule of any one of the preceding claims, wherein the number of basic amino acids in B is greater than the number of acidic amino acids in A.

63. The selective delivery molecule of any one of the preceding claims, wherein A and B are independently selected from natural amino acids, unnatural amino acids, or a combination thereof.

64. The selective delivery molecule of any one of the preceding claims, wherein A is a peptide comprising 5 or 9 consecutive glutamates.

65. The selective delivery molecule of any one of the preceding claims, wherein A is a peptide comprising 5 consecutive glutamates.

66. The selective delivery molecule of any one of the preceding claims, wherein A is a peptide comprising 9 consecutive glutamates.

67. The selective delivery molecule of any one of the preceding claims, wherein B is a peptide comprising 8 or 9 consecutive arginines.

68. The selective delivery molecule of any one of the preceding claims, wherein B is a peptide comprising 8 consecutive arginines.

69. The selective delivery molecule of any one of the preceding claims, wherein B is a peptide comprising 9 consecutive arginines.

70. The selective delivery molecule of any one of the claim 1,2, or 36, wherein X is cleavable by a matrix metalloproteinase.

71. The selective delivery molecule of any one of the claims 1, 2, 36, or 70, wherein X comprises an amino acid sequence that is cleavable by MMP2, MMP7, MMP9, or MMP14.

72. A selective delivery molecule of Formula III, having the structure:
[G-T-c_{A}-Q-Y-D]-(—N—Z)ᵥ Formula III
wherein
G is a linker or a reactive group;
T is a spacer;
c_{A} is a bond or a single amino acid;
Q is a bond or 1-3 amino acids;
Y is 1-3 amino acids or an amino alkylene;
D is an auristatin related therapeutic agent or an imaging agent;
each N is independently a bond or a linker;
Z is a peptide with a sequence comprising 3 to 10 amino acids or polyethylene glycol substituent;
wherein each N is independently bound to G or c_{A}; and
v is 1 or 2.

73. The selective delivery molecule of claim 72, wherein G is a reactive group.

74. The selective delivery molecule of claim 72, wherein the reactive group is an N-maleimide.

75. The selective delivery molecule of claim 72, wherein G is 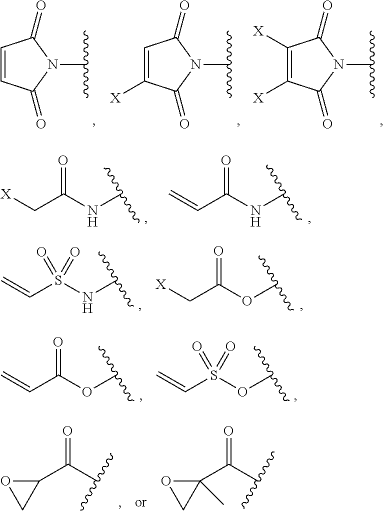
wherein each X is independently —Cl, —Br, —I, or —S-phenyl.

76. The selective delivery molecule of claim 72, wherein G is

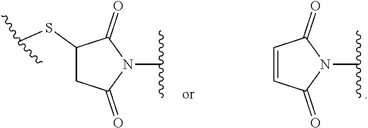 [SMILES: *SC1CC(=O)N(*)C1=O ; *N1C(=O)C=CC1=O]

77. The selective delivery molecule of any one of claims 72-76, wherein T is an optionally substituted C₁-C₈ alkylene-C(O)—.

78. The selective delivery molecule of any one of claims 72-77, wherein c_{A} is

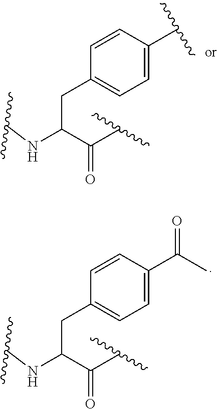 [SMILES: *NC(Cc1ccc(*)cc1)C(*)=O ; *NC(Cc1ccc(C(C)=O)cc1)C(*)=O]

79. The selective delivery molecule of any one of claims 72-78, wherein Q is

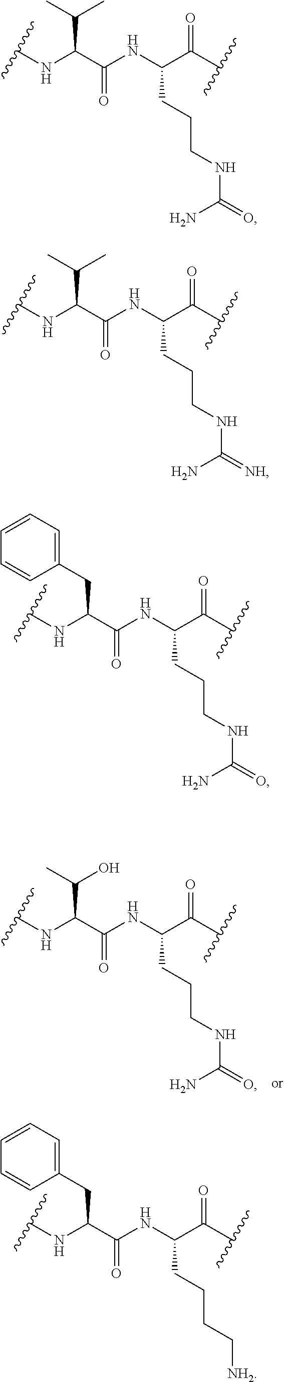 [SMILES: *N[C@H](C(=O)N[C@@H](CCCNC(N)=O)C(*)=O)C(C)C ; *N[C@H](C(=O)N[C@@H](CCCNC(=N)N)C(*)=O)C(C)C ; *N[C@@H](Cc1ccccc1)C(=O)N[C@@H](CCCNC(N)=O)C(*)=O ; *N[C@H](C(=O)N[C@@H](CCCNC(N)=O)C(*)=O)C(C)O ; "or" ; *N[C@@H](Cc1ccccc1)C(=O)N[C@@H](CCCCN)C(*)=O]

80. The selective delivery molecule of any one of claims 72-79, wherein Y is —NHCH₂C(O)- or —NHCH(CH₃)C(O)—.

81. The selective delivery molecule of any one of claims 72-80, wherein D is MMAE or MMAF.

82. The selective delivery molecule of any one of claims 72-81, wherein N is a bond,

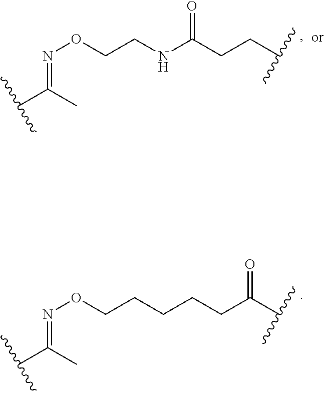 [SMILES: *CCC(=O)NCCO/N=C(/*)C ; *C(=O)CCCCCO/N=C(/*)C]

83. The selective delivery molecule of any one of claims 72-82, wherein Z is 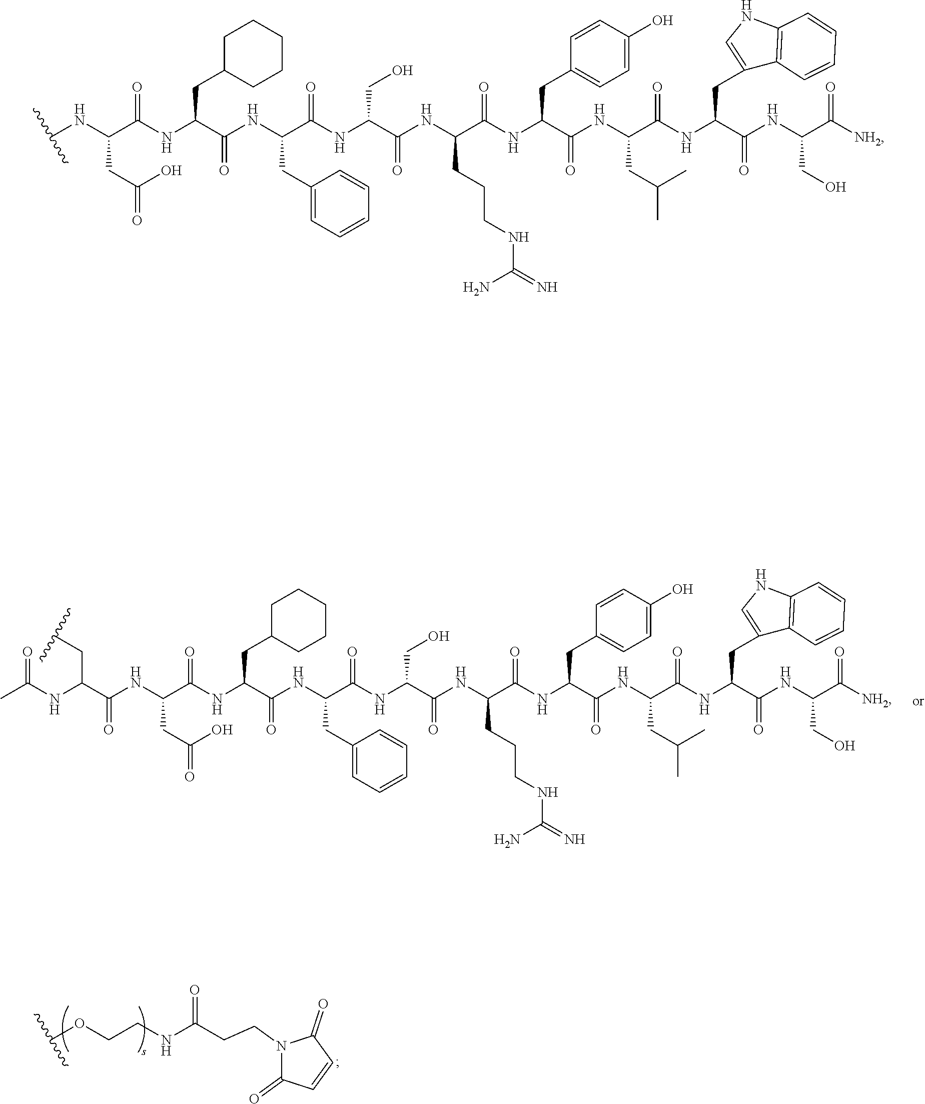
and
wherein s is 1 to 20.

84. The selective delivery molecule of claim 72, wherein the selective delivery molecule is: SDM-185 or SDM-193.

85. A selective delivery molecule, wherein the selective delivery molecule is SDM-201, SDM-202, SDM-203, SDM-204, SDM-208, SDM-213, SDM-214, SDM-215, SDM-216, SDM-223, SDM-224, SDM-226, SDM-232, SDM-234, SDM-235, SDM-261, SDM-262, SDM-263, SDM-264, or SDM-265.

86. A selective delivery molecule, wherein the selective delivery molecule is SDM-187 or SDM-188.

87. A selective delivery molecule, wherein the selective delivery molecule is SDM-192 or SDM-236

88. A pharmaceutical composition comprising of compound of Formula (I), or a pharmaceutically acceptable salt thereof, as provided in claims 1-35, and a pharmaceutically acceptable excipient.

89. A pharmaceutical composition comprising of compound of Formula (II), or a pharmaceutically acceptable salt thereof, as provided in claims 36-71, and a pharmaceutically acceptable excipient.

90. A pharmaceutical composition comprising of compound of Formula (III), or a pharmaceutically acceptable salt thereof, as provided in claims 72-83, and a pharmaceutically acceptable excipient.

91. A method of treating cancer in an individual in need thereof comprising administering to the individual a pharmaceutical composition comprising a compound, or a pharmaceutically acceptable salt thereof, as provided in any one of claims 1-71, 85, or 86, and a pharmaceutically acceptable excipient.

92. A method of visualizing a tissue of interest in an individual in need thereof, comprising administering to the individual a pharmaceutical composition comprising a compound, or a pharmaceutically acceptable salt thereof, as provided in claim 87, and a pharmaceutically acceptable excipient.

93. The method of claim 92, wherein the individual has a cancer.

94. The method of claim 92, further comprising surgically removing the tissue of interest from the individual.

95. The method of claim 92, wherein the visualizing is used to guide surgery, reduce positive margins, to stage cancer tissue, to stage lymph nodes, to reduce reoperations or allows a surgeon to minimize the removal of healthy tissue.

96. The method of any one of the preceding claims, wherein the cancer is breast cancer, colorectal cancer, squamous cell carcinoma, skin cancer, prostate cancer, melanoma, thyroid cancer, ovarian cancer, cervical cancer, lung cancer, pancreatic cancer, head and neck cancer, esophageal cancer, or sarcoma.

97. The method of any one of the preceding claims, wherein the pharmaceutical composition is administered intravenously.
